SEQUENCE LISTING (78 sequences)

SEQ ID NO: 1 - DNA, 1802 bp, Mus musculus
  misc_feature (1802)..(1802): n=a, c, g, or t
ggaacccaggagctcgcagaagccggtcaggagctcgcagaagccggtcgcgctcccagcctgcccgaaacccatgatccagggctgtctcgagctgcggctggagggggggtccggctctgcatggccccggctgctgctatgacttctttgccactcggtgtcaaagtggaggactccgccttcgccaagcctgctgggggaggcgttggccaagcccccggggctgctgcggccaccgcaaccgccatgggcacagatgaggagggggccaagcccaaagtgcccgcttcactcctgcccttcagcgtggaggccctcatggccgatcacaggaagcccggggccaaggagagcgtcctggtggcctccgaaggggctcaggcagcgggtggctcggtgcagcacttgggcacccggcccgggtctctgggcgccccggatgcgccctcctcgccgcggcctctcggccatttctcagtcggaggactcctcaagctgccagaagatgctctggtgaaggccgaaagccccgagaaactagatcggaccccgtggatgcagagtccccgcttctccccgcccccagccagacggctgagtcccccagcatgcaccctacgcaagcacaagaccaaccgcaagcccaggacgcctttcaccacagctcagctgctggctctggagcgcaagttccgccagaagcagtacctgtctattgccgagcgcgcggaattctccagctcgctcagcctcaccgagacccaggtgaagatctggttccagaaccgtcgcgctaaggccaagagactgcaggaggcggagctggagaagctgaagatggccgcgaaacccatgttgccgcctgctgccttcggcctctcttttcctcttggcggtcctgcagctgcgggcgcctcactctacagtgcctctggccctttccagcgcgccgcgctgcctgtagcgcccgtgggactctacaccgcccatgtaggctacagcatgtaccacctgacttaggtgggtccagagtcacctccctgtggtgccatcccctccccagccacctctttgagcagagcagcgggagtccttcctaggaagctctgctgccctataccacctggtcccttctcttaaaccccttgctacacacttcctcctggttgtcgcttcctaaaccttcctcatctgaccccttctgggaagaaaaagaattggtcggaagatgttcaggtttttcgagttttttctagatttacatgcgcaagttataaaatgtggaaactaaggatgcagaggccaagagatttatccgtggtccccagcagaattagaggctgaaggagaccagaggccaaaaggactagaggccatgagactccatcagctgcttccggtcctgaaaccaggcaggacttgcacagagaaattgctaagctaatcggtgctccaagagatgagcccagccctatagaaagcaagagcccagctccttccactgtcaaactctaagcgctttggcagcaaagcattgctctgagggggcagggcgcatgctgctgcttcaccaaggtaggttaaagagactttcccaggaccagaaaaaaagaagtaaaaaaaaaaaaaaaaaaaaaaaaaaaaaaacaaatctgttctattaacagtacattttcgtggctctcaagcatcccttttgaagggactggtgtgtactatgtaatatactgtatatttgaaattttattatcatttatattatagctatatttgttaaataaattaattttaagctacan

SEQ ID NO: 2 - protein, 299 aa, Mus musculus
MAPAAAMTSLPLGVKVEDSAFAKPAGGGVGQAPGAAAATATAMGTDEEGAKPKVPASLLPFSVEALMADHRKPGAKESVLVASEGAQAAGGSVQHLGTRPGSLGAPDAPSSPRPLGHFSVGGLLKLPEDALVKAESPEKLDRTPWMQSPRFSPPPARRLSPPACTLRKHKTNRKPRTPFTTAQLLALERKFRQKQYLSIAERAEFSSSLSLTETQVKIWFQNRRAKAKRLQEAELEKLKMAAKPMLPPAAFGLSFPLGGPAAAGASLYSASGPFQRAALPVAPVGLYTAHVGYSMYHLT

SEQ ID NO: 3 - DNA, 1806 bp, Rattus norvegicus
ggaacccaggagctcgcagaagccggtcaggagctcgcagaagccggtcgcgctcccagcctgcccgaaacccatgacccagggctgtcccgagcgccgtctgaagtgggggtccggctctgcagggccccggctgctgctatgacttctttgccactcggtgtcaaagtggaggactccgccttcgccaagcctgctgggggaggcgctgcccaggcccccggggctgctgcggccactgcaaccgccatgggcacagatgaggagggcgccaagcccaaagtgcccgcttcactcctgcccttcagcgtggaggccctcatggccgatcacaggaagcccggggcaaaggagagcgtcctggtggcttccgaaggggctcaggcggcgggtggctcggtgcagcacttgggcacccggcccgggtctctgggcgccccggacgcgccctcctcgccggggcctctcggccatttctctgttgggggactcctcaagctgccagaagatgctctggtgaaggccgagagcccggagaagctagatcggaccccgtggatgcagagtccccgcttctccccgcccccagccaggcggctgagtcccccggcctgcaccctacgcaagcacaagaccaaccgcaagcccaggacgcccttcaccacggctcagctactggctctggagcgcaagttccgccagaagcagtacctgtctattgccgagcgcgccgagttctccagctcgctcagccttaccgagacccaggtgaagatctggttccagaaccgccgtgctaaggccaagagactgcaggaggccgagttggagaagttgaagatggccgcgaagccaatgttgccgcctgctgcgttcggcctctcctttcctcttgggggtcctgcagcggtggctgcagctgccggcgcctcactctacagtgcctctggccctttccagcgcgccgcgctgcctgtagcgcccgtgggactctacaccgcccacgtaggctacagcatgtaccacctgacataggtgggcccagagtcacctccctgtggtgccatcccttccccagccacctcttctaggcagcgggagtccttcctaggaagctctgctgcccaacaccacctggccccttctcttaaacccttcgctacacagttcctcctggccatcgcatcttaaaattcctcctccctcttccgaccccttctgggaagaaaaaaaagtggccggaagtgtctaggtttttcgagaaaaatttatatttacacgtgcgagttataaatgtggaaactgggggatgcaaaggcgaagagatttatccgtggtccccagcagaattaaaggctgaaggagaccagaggccaaaaggactagaggccatgagactccatcagctgcttccggtcctgaaaccaggcaggactgcacagagaaattgttatttggtgctccaagagacgagcccagccctatagaaagcaaggagcacagctccttccattgtcagactccaaacgcattgcagcaaagcattgctctgagggggcagggtgcatgctgctggttcacgaaggtaggttgaagagactttcccaggaccagaaaaaaagaagttaaaaaacaaaatctgtttctatttaacagtacattttcgtggctctcaaacatcccttttgaagggatcgtgtgtactatgtaatatactgtatatttgaaattttattatcatttatattatagctatatttgttaaataaattaattttaagctacaaaaaaaaaaaaaaaaaaaaa

SEQ ID NO: 4 - protein, 297 aa, Rattus norvegicus
MTSLPLGVKVEDSAFAKPAGGGAAQAPGAAAATATAMGTDEEGAKPKVPASLLPFSVEALMADHRKPGAKESVLVASEGAQAAGGSVQHLGTRPGSLGAPDAPSSPGPLGHFSVGGLLKLPEDALVKAESPEKLDRTPWMQSPRFSPPPARRLSPPACTLRKHKTNRKPRTPFTTAQLLALERKFRQKQYLSIAERAEFSSSLSLTETQVKIWFQNRRAKAKRLQEAELEKLKMAAKPMLPPAAFGLSFPLGGPAAVAAAAGASLYSASGPFQRAALPVAPVGLYTAHVGYSMYHLT

SEQ ID NO: 5 - DNA, 1713 bp, Homo sapiens
gcgcgagtgctcccgggaactctgcctgcgcggcggcagcgaccggaggccaggcccagcacgccggagctggcctgctggggaggggcgggaggcgcgcgcgggagggtccgcccggccaggccccgggccctcgcagaggccggccgcgctcccagcccgcccggagcccatgcccggcggctggccagtgctgcggcagaagggggggcccggctctgcatggccccggctgctgacatgacttctttgccactcggtgtcaaagtggaggactccgccttcggcaagccggcggggggaggcgcgggccaggcccccagcgccgccgcggccacggcagccgccatgggcgcggacgaggagggggccaagcccaaagtgtccccttcgctcctgcccttcagcgtggaggcgctcatggccgaccacaggaagccgggggccaaggagagcgccctggcgccctccgagggcgtgcaggcggcgggtggctcggcgcagccactgggcgtcccgccggggtcgctgggagccccggacgcgccctcttcgccgcggccgctcggccatttctcggtggggggactcctcaagctgccagaagatgcgctcgtcaaagccgagagccccgagaagcccgagaggaccccgtggatgcagagcccccgcttctccccgccgccggccaggcggctgagccccccagcctgcaccctccgcaaacacaagacgaaccgtaagccgcggacgcccttcaccaccgcgcagctgctggcgctggagcgcaagttccgccagaagcagtacctgtccatcgccgagcgcgcggagttctccagctcgctcagcctcactgagacgcaggtgaagatatggttccagaaccgccgcgccaaggcaaagagactacaagaggcagagctggagaagctgaagatggccgccaagcccatgctgccaccggctgccttcggcctctccttccctctcggcggccccgcagctgtagcggccgcggcgggtgcctcgctctacggtgcctctggccccttccagcgcgccgcgctgcctgtggcgcccgtgggactctacacggcccatgtgggctacagcatgtaccacctgacatagagggtcccaggtccccacctgtgggccagccgattcctccagccctggtgctgtacccccgacgtgctcccctgctcggcaccgccagccgccttccctttaaccctcacactgctccagtttcacctctttgctccctgagttcactctccgaagtctgatccctgccaaaaagtggctggaagagtcccttagtactcttctagcatttagatctacactctcgagttaaagatggggaaactgagggcagagaggttaacagatttatctagggtccccagcagaattgacagttgaacagagctagaggccatgtctcctgcatagcttttccctgtcctgacaccaggcaagaaaagcgcagagaaatcggtgtctgacgattttggaaatgagaacaatctcaaaaaaaaaaaaaaaaaaaaaaaaaaaagaaaagagaaaaaaaagactagccagccaggaagatgaatcctagcttcttccattggaaaatttaagacaagttcaacaacaaaacatttgctctggggggcagggaaaacacagatgtgttgcaaaggtaggttgaaggga

SEQ ID NO: 6 - protein, 297 aa, Homo sapiens
MTSLPLGVKVEDSAFGKPAGGGAGQAPSAAAATAAAMGADEEGAKPKVSPSLLPFSVEALMADHRKPGAKESALAPSEGVQAAGGSAQPLGVPPGSLGAPDAPSSPRPLGHFSVGGLLKLPEDALVKAESPEKPERTPWMQSPRFSPPPARRLSPPACTLRKHKTNRKPRTPFTTAQLLALERKFRQKQYLSIAERAEFSSSLSLTETQVKIWFQNRRAKAKRLQEAELEKLKMAAKPMLPPAAFGLSFPLGGPAAVAAAAGASLYGASGPFQRAALPVAPVGLYTAHVGYSMYHLT

SEQ ID NO: 7 - DNA, 1601 bp, Axolotl
tcggagtgaaggccgaggagtcgcccgtcctaagcaagcagaggatgcagaccggcctgagctccggggcggaccgaggagccccagaaacccaagctgccggccatcctgccatttagcgtggaggccctcatggctgaccgcaggccgacggtcagagaccgtgagcggtgcagccccgcggggacccagctgcccgggccctcgcaaaccagccccaggctaggggggcacctctcaggaccggagtcccctggatccgctctccatgaacagacactattccatgggtggcttactgcacttaccagaagaggctcttgcgaagccgagagcccggacagccaggagaggaacccgtggatgcagagccccaaattctccccaccctcagcaaggaggctgagcccaccggcctgcactctccggaagcacaagaccaaccggaagccgcggacgccgttcaccacgtcgcagctgctggccctggagcggaagttccggcagaagcagtacctgtccatcgcggagcgcgccgagttctcgggctccctcagcctgaccgagacgcaggtcaagatctggttccagaaccgccgcgccaaggccaagcggctgcaggaggccgagctggagaagctcaagatggccgccaagcccatgatgccgccggccttcggcatctccttccccctcggctctccagtgcacgcggcctccctgtacgggccctccggccccttccacagacccagcatgcccatgtcgcccatgggactgtacgccgctcacatgggctacagcatgtaccacctgacataagggcgccgcagacccaccacagaccattcatgcagcacttttctgatgttgggccctgcccacgtctgccattggtggcactcaggcatgcatgccaaccacgttggaaagaaccgagagcgtgattcggtggcaggaagaggggggttgtgcatgcccattggctctcatcgcaatgaaggaacgctatgccaggcattgcacacctttaacaagttgaacaaggacaatgttttgtgtcgtgaaggagcgcctcccacttctgaataatagagagatggcatgtgtgcaccagcctgaaatacgccaggcgttttggattttcacagtgtgttcaacacctgtagagggaactgaaacatatttgtgagaagttcacgtttggacatacagttcctcacacgtggtttacagaaaagtccagcatttcagcagctcaacctggctcagcaccattcaatacagaaagcccgacatcttgttgtatggccgcatgaattagttcacatcaccgggaaatgtcatgagttctaagaagatgactttttataaataaagcgctatcgaaaatgctcctcaaaagtgccaccagacacacgtggaaaggcaacagaacttgtcaacgaatcactgtgcttcactgtttcccttgcgtgtggatgttcctacactcgtcccttgggagcaggggatccgtactatgtaatatactgtatatttgaaaaaaatattatcatttatattatagctatatttgttaaataaattaattttaagct

SEQ ID NO: 8 - protein, 229 aa, Axolotl
MADRRPTVRDRERCSPAGTQLPGPSQTSPRLGGHLSGPESPGSALHEQTLFHGWLTALTRRGSCEAESPDSQERNPWMQSPKFSPPSARRLSPPACTLRKHKTNRKPRTPFTTSQLLALERKFRQKQYLSIAERAEFSGSLSLTETQVKIWFQNRRAKAKRLQEAELEKLKMAAKPMMPPAFGISFPLGSPVHAASLYGPSGPFHRPSMPMSPMGLYAAHMGYSMYHLT

SEQ ID NO: 9 - DNA, 1453 bp, Mus musculus
aagcttcctctttaaacaatcggctttaattacgccttagattttgagtttgggcgattataacccttgagggatcgcctaataacaactctgctgactgctcctgtaattaactcctaatttatttcaaacggggcgggggaagggccccaagcctctccagggagagccaatcggtggcgagcgtccgtggcgtcaggagcagggccgtcgccagttggttgagccgagtctcccacttcccctcggaggacaggctgggctcccagcgcgcccctgccggctccccccccaaaagttggagtcttcgcttgagagttgccagcggagtcgcgcgccgacagctacgcggcgcagaaagtcatggcttctccgactaaaggcggtgacttgtttttttcgtcggatgaggagggccccgcggtactggccggcccgggtcctgggcctggaggagccgagggcagcgcagaggagcgcagggtcaaggtctccagcctgcccttcagcgtggaggcgctcatgtccgacaagaagccgcccaaggaatcgcccgcggtgccacccgactgcgcctcggctggcgctgtcctgcggccgctgctgctgccgggacacggcgtccgggacgctcacagtcccgggcctctcgtcaagcccttcgagaccgcctcggtcaagtcggaaaattccgaagacggagcaccgtggatacaggagcccggcagatactccccgccgcccagacatatgagccccaccacctgcaccctgaggaaacacaagaccaaccggaagccacgcacacccttcaccacatcccagcttctagccttggagcgcaagttccgccagaaacagtacctgtccatagcagagcgggccgagttctccagctctctgaaccttacagagacccaggtcaaaatctggttccagaaccgaagggctaaggcgaaaagactgcaagaggcggaactggaaaagctgaaaatggctgccaagcctatgctgccctcaggcttcagtctgcccttccctatcaactcacccctgcaagcagcatccatatacggcgcatcctaccccttccatagacctgtgctccccatcccgcctgttggactctatgccacgccggttggatatggcatctaccatgtatcctaaggaagaccagatggaccagactccaggatggatgtttgcataaaagcatccccctccctctccgagaaggtggtgccaactctgctcctgaatgcgagccttgcattgtcaccctaagcgacagggccacttgatacagagtgaatttgttatttaggtgagaggcactaagacctgttttgttttcataattttccaaatgccccctttcctctcacaaatattggctctgctagtttttatgtataaatatataataaaatataagactttttatatgcca

SEQ ID NO: 10 - protein, 268 aa, Mus musculus
MASPTKGGDLFFSSDEEGPAVLAGPGPGPGGAEGSAEERRVKVSSLPFSVEALMSDKKPPKESPAVPPDCASAGAVLRPLLLPGHGVRDAHSPGPLVKPFETASVKSENSEDGAPWIQEPGRYSPPPRHMSPTTCTLRKHKTNRKPRTPFTTSQLLALERKFRQKQYLSIAERAEFSSSLNLTETQVKIWFQNRRAKAKRLQEAELEKLKMAAKPMLPSGFSLPFPINSPLQAASIYGASYPFHRPVLPIPPVGLYATPVGYGIYHVS

SEQ ID NO: 11 - DNA, 420 bp, Rattus norvegicus
atgagccccaccacctgcaccctgaggaaacacaagaccaacaggaagccacgcacaccgttcaccacgtcccagcttctagccttggagcgcaagttccgccagaaacagtacctctccatcgcagagcgggccgagttctccagctctctgaaccttacagaaacccaggtcaaaatctggttccagaaccgaagggctaaggcaaaaagactgcaggaggcggaactggaaaagctgaaaatggctgccaaacctatgctgccctcgggcttcagtctgcccttccctatcaactcccccttgcaagcggcatccatatacagcgcctcctaccccttccatagacctgtgcttcccatcccgcctgtgggactctatgccacgccggtgggatatggcatgtaccatctatcctaa

SEQ ID NO: 12 - protein, 139 aa, Rattus norvegicus
MSPTTCTLRKHKTNRKPRTPFTTSQLLALERKFRQKQYLSIAERAEFSSSLNLTETQVKIWFQNRRAKAKRLQEAELEKLKMAAKPMLPSGFSLPFPINSPLQAASIYSASYPFHRPVLPIPPVGLYATPVGYGMYHLS

SEQ ID NO: 13 - DNA, 2808 bp, Homo sapiens
ggggggggggggcagcctctcgggaagagccaatcaggggcgagcgtcttctcgtcgcacgaggcccggcgcggattggcggcgcgcgtctcccacttcccctcggaggaaaggctcagctcccagcgcgcccctcccgtctccgcagcaaaaaagtttgagtcgccgctgccgggttgccagcggagtcgcgcgtcgggagctacgtagggcagagaagtcatggcttctccgtccaaaggcaatgacttgttttcgcccgacgaggagggcccagcagtggtggccggaccaggcccggggctggggggcgccgcgggggccgcggaggagcgccgcgtcaaggtctccagcctgcccttcagcgtggaggcgctcatgtccgacaagaagccgcccaaggagtcgcccgctgtgcctcccgaaggcgcctcggccggggcccacctgcggccactgctgctgtcggggcaccgcgctcgggaagcgcacagccccgggccgctggtgaagcccttcgagaccgcctcggtcaagtcgggaaattcagaagatggagcggcgtggatgcaggaacccggccgatattcgccgccgccaagacatatgagccctaccacctgcaccctgaggaaacacaagaccaatcggaagccgcgcacgccctttaccacatcccagctcctcgccctggagcgcaagttccgtcagaaacagtacctctccattgcagagcgtgcagagttctccagctctctgaacctcacagagacccaggtcaaaatctggttccagaaccgaagcgccaaggcgaaaagactgcaggaggcggaactggaaaagctgaaaatggctgcaaaacctatgctaccctccagcttcagtctccccttccccatcagctcgcccctgcaggcagcgtccatatacgcagcatcctacccgttccatagacctgtgcttcccatcccgcccgtgggactctatgccacgccagtgggatatggcatgtaccacctgtcctaaggaagaccagatcaatagactccatgatggatgcttgtttcaaagggtttcctctccctctccacaaaggcatagccagccagtactcctgcgctgctaagccctcgacgttgcaccccaccccctctaacggctagctgacagggccacaccacatagctgaaatttcgttctgtaggcggaggcaccaagccctgcttttcttggtgtaacttccagagtccccccttttttcccttgcacaaaagcttggctctgatggtttttttggcatgatgtatatatatatatacgaaaaatactacagaccctttttatcagcagacgtaaaaattcaaattattttaaaaggcaaaatttatatacatatgtgctttttttctatatctcaccttcccaaaaagacacatgtgtaagtccatttgttgtattttcttaaagagggagacaaattcggaggagcgccgcgtcaaggtctccagcctgcccttcagcgtggaggcgctcatgtccgatttgcaaaaatgtgctaaagtcaatgatttttaccgggattattgacttctgcttatacaagaagccgcccaaggagtcgcccgctgtgcctcccgaaggcgcctcggccggcctgcggaaaaacaaaagaaaacagacacaatgcagcagccagaaaatattagatatggagagattatggccactgctgctgaccggccacggcgtccgggaagcgcacagccccgggccgctggtgatcaaagtgaacccacatcatatttctgcattttacttgcattaaaagaaacctctttataagcccttcgagaccgcctcggtcaagtcgggaaattcagaagatggagcggcgtggatgctacatacgttgttcctatctcccgcccacgcccacacatatttttaaagtttttaggaacccggccgatattcgccgccgccaagacatatgagccctaccacctgcaccctgacctttttaagaatatttttgtaagaccaatacctgggatgagaagaatccgtagactgccggaaacacaagaccaatcggaagccgcgcacgccctttaccacatcccagctcctcgccctggaggtgaggtagaaaaattagaaatacttcctaattcttctcaaggctgttggtaactttggagcgcaagttccgtcagaaacagtacctctccattgcagagcgtgcagagttctctatttcagataattggagagtaaaatgttaaaacctgtgagaggattgtacagctctctgaacctcacagacccaggtcaaaaggttctgagaaatactaggtacattcatcctcacagattgcaaaggtgctttgggtgggggtttagtaattttctgcttaaaaaatgagtatcttgtaaccattacctatatctaaatattcttgaacaattagtagatccagaaagaaaaaaaaaatatgcttctctgtgtgtgtacctgttgtatgtcctaacttattagaaaaattttatatctttttacatgtggggggcagaaggtaaagcatgtttgacttgtgaaaatgggatgtcaaacagccataagttccctggtattcaccttcctgtccatctgtcccctccatcggtatacctttatccctttgaaagggtgcttgtacaatttgatatattttattgaagagttatctcttattctgaattaaattaagcatttgttttattctgaattaaattaagcatttgttttattgcagtaaagtttgtcca

SEQ ID NO: 14 - protein, 267 aa, Homo sapiens [sequence omitted]

SEQ ID NO: 15 - DNA, 2216 bp, Mus musculus
gaattccggcagagcccaaggtcctagcaaaataaaccgccgctgggtcccacgcgtcccagccgggcggctagccccgcacccgccatcccatgcatggcccgggcgacattcgacatgaacgcggcggggctcgaagctcgcggcgggggccacacagagcacggaccactccccttcagcgtcgagtctctgctggaggctgagcgcgtaccgggctccgagtctggggagctgggggtggagaggccgctcggcgcctcgaagcctggagcctggccccctccagtcgcgcactcttgtcccccacgtgccccgagccctccaccctgcaccctccgcaaacacaaaaccaatcgcaaaccacggacgccattcaccaccgcgcagctgctggcgcttgagcgcaagtttcaccagaagcaatacttatccattgcggagcgcgccgagttctccagcagcttgagcctcactgagactcaggtcaagatctggtttcagaaccgccgagccaaagccaagcggctgcaggaagctgagctggagaagctgaagctggcagcgaagccactactgccggcggcgtttgccctgcccttcccgctgggcacccagctgcacagctccgcggccactttcggcggcaacgcggttcctgggatcctcgccgggcctgtcgcggcctatggcatgtactacttgtcttagatcatggaatcaagccctaacctcttggttaggctcagggttgcgcacagttctgcctgccgctgatgctcctggtaacttttgacttttcattttagtcttccggggggggggggggggggggtttatacggtgttatacttgatgactggagcctatttatagacatcttacaaatactttaaaccttcctaaaaccttgcccacatcaatctgtttttctgaaagaaaacacaccgtcatatagtccttgaaacagacaaaagcagttcttcctatttcttacaaagcagtcgacaaagtatttctggagtgggtgtgctccctgtattcactctgcccggacaccgaggacttcagagaggctcgattgagctggcataagaaaacaaattccataagaagaaatgaaaataaaaaatgagataatcggacggttagaaattactgtctctccaactgttgtgaccaaatggaaacttttacgctttttaggttttataccattattatttttttttttagagtatatgtcgttcaaggagctagaggcaaacgctggatggacttggaagtaggggaggggactgttggcgccatgccctaatgtaagggtggctgctgcccccagcatctcctgagcttcttcgaaaagagcctgctggggggagacagagggctgaaggcagcagggaccaacctctagcctcttgccagcaggtgtccagtcaataagagaagaaagtgagagttgagcatctcagatctcccattgtgggtgcggtaggaggtgaggcctgtgaaggtcccagaagagttactctgagtgccggctgcctctgtaattctggtgctccgtagaaaagcggaggacgttgtttaggaatccggacttgtacctttattctacctttatccgaggaaagaaaacaggccatctgagctgttctgtcctatcctgttcggtctcaatcctatcatgcaagattgaggcccttagggtacctgacaacgggcactaatacaccccatggctgtttccacaggacacttttgctgcaatctagagactttagctggagtgtgttgagggctcacaacagtcccctggagtcacagcccaggacctttgacaggccctcctgccagccttcgggcaggtgaggaagaacctgcctgcagagggctgctggttgtgtgttcatgagaacatggaatttcacagtgatagatggcccagggttaggatgctctggtggggggaagggaaggactgggctatctcggcagtccaagccacgtccaggaaaactgctgtcccgtgaccttcactggtccatcagctccaaagtaaaaccgaaaagctaatcttgcatggaattgcctattgatacgtattgacagtcttctgccctgtgtctcttggaaaaattggaaaccagataggtttggttttgttttgagaccaaatcgtcactatgtagggggggggggggggggaattc

SEQ ID NO: 16 - protein, 204 aa, Mus musculus
MARATFDMNAAGLEARGGGHTEHGPLPFSVESLLEAERVPGSESGELGVERPLGASKPGAWPPPVAHSCPPRAPSPPPCTLRKHKTNRKPRTPFTTAQLLALERKFHQKQYLSIAERAEFSSSLSLTETQVKIWFQNRRAKAKRLQEAELEKLKLAAKPLLPAAFALPFPLGTQLHSSAATFGGNAVPGILAGPVAAYGMYYLS

SEQ ID NO: 17 - DNA, 1541 bp, Mus musculus
cgcactcctccccctgctcgaggctgtgtgtcagcacttggctggagacttcttgaacttgccgggagagtgacttgggctccccacttcgcgccggtgtcctcgcccggcggatccagtcttgccgcctccagcccgatcacctctcttcctcagcccgctggcccaccccaagacacagttccctacagggagaacacccggagaaggaggaggaggcgaagaaaagcaacagaagcccagttgctgctccaggtccctcggacagagctttttccatgtggagactctctcaatggacgtgccccctagtgcttcttagacggactgcggtctcctaaagccgcaggtcgaccatggtggccgggacccgctgtcttctagtgttgctgcttccccaggtcctcctgggcggcgcggccggcctcattccagagctgggccgcaagaagttcgccgcggcatccagccgacccttgtcccggccttcggaagacgtcctcagcgaatttgagttgaggctgctcagcatgtttggcctgaagcagagacccacccccagcaaggacgtcgtggtgcccccctatatgctagatctgtaccgcaggcactcaggccagccaggagcgcccgccccagaccaccggctggagagggcagccagccgcgccaacaccgtgcgcacgttccatcaactagaagccgtggaggaacttccagagatgagtgggaaaacggcccggcgcttcttcttcaatttaagttctgtccccagtgacgagtttctcacatctgcagaactccagatcttccgggaacagatacaggaagctttgggaaacagtagtttccagcaccgaattaatatttatgaaattataaagcctgcagcagccaacttgaaatttcctgtgaccagactattggacaccaggttagtgaatcagaacacaagtcagtgggagagcttcgacgtcaccccagctgtgatgcggtggaccacacagggacacaccaaccatgggtttgtggtggaagtggcccatttagaggagaacccaggtgtctccaagagacatgtgaggattagcaggtctttgcaccaagatgaacacagctggtcacagataaggccattgctagtgacttttggacatgatggaaaaggacatccgctccacaaacgagaaaagcgtcaagccaaacacaaacagcggaagcgcctcaagtccagctgcaagagacaccctttgtatgtggacttcagtgatgtggggtggaatgactggatcgtggcacctccgggctatcatgccttttactgccatggggagtgtccttttccccttgctgaccacctgaactccactaaccatgccatagtgcagactctggtgaactctgtgaattccaaaatccctaaggcatgctgtgtccccacagagctcagcgcaatctccatgttgtacctagatgaaaatgaaaaggttgtgctaaaaaattatcaggacatggttgtggagggctgcgggtgtcgttag

SEQ ID NO: 18 - protein, 394 aa, Mus musculus
MVAGTRCLLVLLLPQVLLGGAAGLIPELGRKKFAAASSRPLSRPSEDVLSEFELRLLSMFGLKQRPTPSKDVVVPPYMLDLYRRHSGQPGAPAPDHRLERAASRANTVRTFHQLEAVEELPEMSGKTARRFFFNLSSVPSDEFLTSAELQIFREQIQEALGNSSFQHRINIYEIIKPAAANLKFPVTRLLDTRLVNQNTSQWESFDVTPAVMRWTTQGHTNHGFVVEVAHLEENPGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 19 - DNA, 1547 bp, Homo sapiens
ggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatata

SEQ ID NO: 20 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 21 - DNA, 1263 bp, Mus musculus
atggactgttattatgccttgttttctgtcaacaccatgattcctggtaaccgaatgctgatggtcgttttattatgccaagtcctgctaggaggcgcgagccatgctagtttgatacctgagaccgggaagaaaaaagtcgccgagattcagggccacgcgggaggacgccgctcagggcagagccatgagctcctgcgggacttcgaggcgacacttctacagatgtttgggctgcgccgccgtccgcagcctagcaagagcgccgtcattccggattacatgagggatctttaccggctccagtctggggaggaggaggaggaagagcagagccagggaaccgggcttgagtacccggagcgtcccgccagccgagccaacactgtgaggagtttccatcacgaagaacatctggagaacatcccagggaccagtgagagctctgcttttcgtttcctcttcaacctcagcagcatcccagaaaatgaggtgatctcctcggcagagctccggctctttcgggagcaggtggaccagggccctgactgggaacagggcttccaccgtataaacatttatgaggttatgaagcccccagcagaaatggttcctggacacctcatcacacgactactggacaccagactagtccatcacaatgtgacacggtgggaaactttcgatgtgagccctgcagtccttcgctggacccgggaaaagcaacccaattatgggctggccattgaggtgactcacctccaccagacacggacccaccagggccagcatgtcagaatcagccgatcgttacctcaagggagtggagattgggcccaactccgccccctcctggtcacttttggccatgatggccggggccataccttgacccgcaggagggccaaacgtagtcccaagcatcacccacagcggtccaggaagaagaataagaactgccgtcgccattcactatacgtggacttcagtgacgtgggctggaatgattggattgtggccccacccggctaccaggccttctactgccatggggactgtccctttccactggctgatcacctcaactcaaccaaccatgccattgtgcagaccctagtcaactctgttaattctagtatccctaaggcctgttgtgtccccactgaactgagtgccatttccatgttgtacctggatgagtatgacaaggtggtgttgaaaaattatcaggagatggtggtagaggggtgtggatgccgctga

SEQ ID NO: 22 - protein, 420 aa, Mus musculus
MDCYYALFSVNTMIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEEQSQGTGLEYPERPASRANTVRSFHHEEHLENIPGTSESSAFRFLFNLSSIPENEVISSAELRLFREQVDQGPDWEQGFHRINIYEVMKPPAEMVPGHLITRLLDTRLVHHNVTRWETFDVSPAVLRWTREKQPNYGLAIEVTHLHQTRTHQGQHVRISRSLPQGSGDWAQLRPLLVTFGHDGRGHTLTRRRAKRSPKHHPQRSRKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 23 - DNA, 1400 bp, Homo sapiens
gaggcactgcttggaagcaattgtagagcaatacagctcttgacaaactcgtgtcgaacatcagtgactgttgaagggaatgaggcaaacatatctacggaatgctgatggtcgttttattatgccaagtcctgctaggaggcgcgagccatgctagtttgatacctgagacggggaagaaaaaagtcgccgagattcagggccacgcgggaggacgccgctcagggcagagccatgagctcctgcgggacttcgaggcgacacttctgcagatgtttgggctgcgccgccgcccgcagcctagcaagagtgccgtcattccggactacatgcgggatctttaccggcttcagtctggggaggaggaggaagagcagatccacagcactggtcttgagtatcctgagcgcccggccagccgggccaacaccgtgaggagcttccaccacgaagaacatctggagaacatcccagggaccagtgaaaactctgcttttcgtttcctctttaacctcagcagcatccctgagaacgaggtgatctcctctgcagagcttcggctcttccgggagcaggtggaccagggccctgattgggaaaggggcttccaccgtataaacatttatgaggttatgaagcccccagcagaagtggtgcctgggcacctcatcacacgactactggacacgagactggtccaccacaatgtgacacggtgggaaacttttgatgtgagccctgcggtccttcgctggacccgggagaagcagccaaactatgggctagccattgaggtgactcacctccatcagactcggacccaccagggccagcatgtcaggattagccgatcgttacctcaagggagtgggaattgggcccagctccggcccctcctggtcacctttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggtactgaaaaattatcaggagatggtagtagagggatgtgggtgccgctgagatcaggcagtccttgaggatagacagatatacacaccacacacacacaccacatacaccacacacacacgttcccatccactcacccac

SEQ ID NO: 24 - protein, 402 aa, Homo sapiens
MLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 25 - DNA, 1872 bp, Mus musculus
ctgcagcaagtgacctcgggtcgtggaccgctgccctgccccctccgctgccacctggggcggcgcgggcccggtgccccggatcgcgcgtagagccggcgcgatgcacgtgcgctcgctgcgcgctgcggcgccacacagcttcgtggcgctctgggcgcctctgttcttgctgcgctccgccctggccgatttcagcctggacaacgaggtgcactccagcttcatccaccggcgcctccgcagccaggagcggcgggagatgcagcgggagatcctgtccatcttagggttgccccatcgcccgcgcccgcacctccagggaaagcataattcggcgcccatgttcatgttggacctgtacaacgccatggcggtggaggagagcgggccggacggacagggcttctcctacccctacaaggccgtcttcagtacccagggcccccctttagccagcctgcaggacagccacttcctcactgacgccgacatggtcatgagcttcgtcaacctagtggaacatgacaaagaattcttccaccctcgataccaccatcgggagttccggtttgatctttccaagatccccgagggcgaacgggtgaccgcagccgaattcaggatctataaggactacatccgggagcgatttgacaacgagaccttccagatcacagtctatcaggtgctccaggagcactcaggcagggagtcggacctcttcttgctggacagccgcaccatctgggcttctgaggagggctggttggtgtttgatatcacagccaccagcaaccactgggtggtcaaccctcggcacaacctgggcttacagctctctgtggagaccctggatgggcagagcatcaaccccaagttggcaggcctgattggacggcatggaccccagaacaagcaacccttcatggtggccttcttcaaggccacggaagtccatctccgtagtatccggtccacggggggcaagcagcgcagccagaatcgctccaagacgccaaagaaccaagaggccctgaggatggccagtgtggcagaaaacagcagcagtgaccagaggcaggcctgcaagaaacatgagctgtacgtcagcttccgagaccttggctggcaggactggatcattgcacctgaaggctatgctgcctactactgtgagggagagtgcgccttccctctgaactcctacatgaacgccaccaaccacgccatcgtccagacactggttcacttcatcaacccagacacagtacccaagccctgctgtgcgcccacccagctcaacgccatctctgtcctctacttcgacgacagctctaatgtcatcctgaagaagtacagaaacatggtggtccgggcctgtggctgccactagctcttcctgagaccctgacctttgcggggccacacctttccaaatcttcgatgtctcaccatctaagtctctcactgcccaccttggcgaggagccaacagaccaacctctcctgagccttcccctcacctccccaaccggaagcatgtaagggttccagaaacctgagcgtgcaggcagctgatgagcgccctttccttctggcacgtgacggacaagatcctaccagctaccacagcaaacgcctaagagcaggaaaaatgtctgccaggaaagtgtccattggccacatggcccctggcgctctgagtctttgaggagtaatcgcaagcctcgttcagctgcagcagaaggaagggcttagccagggtgggcgctggcgtctgtgttgaagggaaaccaagcagaagccactgtaatgatatgtcacaataaaacccatgaatgaaaaaaaaaaaaaaaaaaaaaaaaaaaaa

SEQ ID NO: 26 - protein, 430 aa, Mus musculus
MHVRSLRAAAPHSFVALWAPLFLLRSALADFSLDNEVHSSFIHRRLRSQERREMQREILSILGLPHRPRPHLQGKHNSAPMFMLDLYNAMAVEESGPDGQGFSYPYKAVFSTQGPPLASLQDSHFLTDADMVMSFVNLVEHDKEFFHPRYHHREFRFDLSKIPEGERVTAAEFRIYKDYIRERFDNETFQITVYQVLQEHSGRESDLFLLDSRTIWASEEGWLVFDITATSNHWVVNPRHNLGLQLSVETLDGQSINPKLAGLIGRHGPQNKQPFMVAFFKATEVHLRSIRSTGGKQRSQNRSKTPKNQEALRMASVAENSSSDQRQACKKHELYVSFRDLGWQDWIIAPEGYAAYYCEGECAFPLNSYMNATNHAIVQTLVHFINPDTVPKPCCAPTQLNAISVLYFDDSSNVILKKYRNMVVRACGCH

SEQ ID NO: 27 - DNA, 1878 bp, Homo sapiens
gggcgcagcggggcccgtctgcagcaagtgaccgacggccgggacggccgcctgccccctctgccacctggggcggtgcgggcccggagcccggagcccgggtagcgcgtagagccggcgcgatgcacgtgcgctcactgcgagctgcggcgccgcacagcttcgtggcgctctgggcacccctgttcctgctgcgctccgccctggccgacttcagcctggacaacgaggtgcactcgagcttcatccaccggcgcctccgcagccaggagcggcgggagatgcagcgcgagatcctctccattttgggcttgccccaccgcccgcgcccgcacctccagggcaagcacaactcggcacccatgttcatgctggacctgtacaacgccatggcggtggaggagggcggcgggcccggcggccagggcttctcctacccctacaaggccgtcttcagtacccagggcccccctctggccagcctgcaagatagccatttcctcaccgacgccgacatggtcatgagcttcgtcaacctcgtggaacatgacaaggaattcttccacccacgctaccaccatcgagagttccggtttgatctttccaagatcccagaaggggaagctgtcacggcagccgaattccggatctacaaggactacatccgggaacgcttcgacaatgagacgttccggatcagcgtttatcaggtgctccaggagcacttgggcagggaatcggatctcttcctgctcgacagccgtaccctctgggcctcggaggagggctggctggtgtttgacatcacagccaccagcaaccactgggtggtcaatccgcggcacaacctgggcctgcagctctcggtggagacgctggatgggcagagcatcaaccccaagttggcgggcctgattgggcggcacgggccccagaacaagcagcccttcatggtggctttcttcaaggccacggaggtccacttccgcagcatccggtccacggggagcaaacagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccactagctcctccgagaattcagaccctttggggccaagtttttctggatcctccattgctcgccttggccaggaaccagcagaccaactgccttttgtgagaccttcccctccctatccccaactttaaaggtgtgagagtattaggaaacatgagcagcatatggcttttgatcagtttttcagtggcagcatccaatgaacaagatcctacaagctgtgcaggcaaaacctagcaggaaaaaaaaacaacgcataaagaaaaatggccgggccaggtcattggctgggaagtctcagccatgcacggactcgtttccagaggtaattatgagcgcctaccagccaggccacccagccgtgggaggaagggggcgtggcaaggggtgggcacattggtgtctgtgcgaaaggaaaattgacccggaagttcctgtaataaatgtcacaataaaacgaatgaatg

SEQ ID NO: 28 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 29 - DNA, 6802 bp, Homo sapiens
gcggccccagaaaacccgagcgagtagggggcggcgcgcaggagggaggagaactgggggcgcgggaggctggtgggtgtcggggtggagatgtagaagatgtgacgccgcggcccggcgggtgccagattagcggacggctgcccgcggttgcaacgggatcccgggcgctgcagcttgggaggcggctctccccaggcggcgtccgcggagacacccatccgtgaaccccaggtcccgggccgccggctcgccgcgcaccaggggccggcggacagaagagcggccgagcggctcgaggctgggggaccgcgggcgcggccgcgcgctgccgggcgggaggctggggggccggggccggggccgtgccccggagcgggtcggaggccggggccggggccgggggacggcggctccccgcgcggctccagcggctcggggatcccggccgggccccgcagggaccatggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctgattttaatggccacatctaatctcatttcacatgaaagaagaagtatattttagaaatttgttaatgagagtaaaagaaaataaatgtgtatagctcagtttggataattggtcaaacaattttttatccagtagtaaaatatgtaaccattgtcccagtaaagaaaaataacaaaagttgtaaaatgtatattctcccttttatattgcatctgctgttacccagtgaagcttacctagagcaatgatctttttcacgcatttgctttattcgaaaagaggcttttaaaatgtgcatgtttagaaacaaaatttcttcatggaaatcatatacattagaaaatcacagtcagatgtttaatcaatccaaaatgtccactatttcttatgtcattcgttagtctacatgtttctaaacatataaatgtgaatttaatcaattcctttcatagttttataattctctggcagttccttatgatagagtttataaaacagtcctgtgtaaactgctggaagttcttccacagtcaggtcaattttgtcaaacccttctctgtacccatacagcagcagcctagcaactctgctggtgatgggagttgtattttcagtcttcgccaggtcattgagatccatccactcacatcttaagcattcttcctggcaaaaatttatggtgaatgaatatggctttaggcggcagatgatatacatatctgacttcccaaaagctccaggatttgtgtgctgttgccgaatactcaggacggacctgaattctgattttataccagtctcttcaaaaacttctcgaaccgctgtgtctcctacgtaaaaaaagagatgtacaaatcaataataattacacttttagaaactgtatcatcaaagattttcagttaaagtagcattatgtaaaggctcaaaacattaccctaacaaagtaaagttttcaatacaaattctttgccttgtggatatcaagaaatcccaaaatattttcttaccactgtaaattcaagaagcttttgaaatgctgaatatttctttggctgctacttggaggcttatctacctgtacatttttggggtcagctctttttaacttcttgctgctctttttcccaaaaggtaaaaatatagattgaaaagttaaaacattttgcatggctgcagttcctttgtttcttgagataagattccaaagaacttagattcatttcttcaacaccgaaatgctggaggtgtttgatcagttttcaagaaacttggaatataaataattttataattcaacaaaggttttcacattttataaggttgatttttcaattaaatgcaaatttgtgtggcaggatttttattgccattaacatatttttgtggctgctttttctacacatccagatggtccctctaactgggctttctctaattttgtgatgttctgtcattgtctcccaaagtatttaggagaagccctttaaaaagctgccttcctctaccactttgctggaaagcttcacaattgtcacagacaaagatttttgttccaatactcgttttgcctctatttttcttgtttgtcaaatagtaaatgatatttgcccttgcagtaattctactggtgaaaaacatgcaaagaagaggaagtcacagaaacatgtctcaattcccatgtgctgtgactgtagactgtcttaccatagactgtcttacccatcccctggatatgctcttgttttttccctctaatagctatggaaagatgcatagaaagagtataatgttttaaaacataaggcattcatctgccatttttcaattacatgctgacttcccttacaattgagatttgcccataggttaaacatggttagaaacaactgaaagcataaaagaaaaatctaggccgggtgcagtggctcatgcctatattccctgcactttgggaggccaaagcaggaggatcgcttgagcccaggagttcaagaccaacctggtgaaaccccgtctctacaaaaaaacacaaaaaatagccaggcatggtggcgtgtacatgtggtctcagatacttgggaggctgaggtgggagggttgatcacttgaggctgagaggtcaaggttgcagtgagccataatcgtgccactgcagtccagcctaggcaacagagtgagactttgtctcaaaaaaagagaaattttccttaataagaaaagtaatttttactctgatgtgcaatacatttgttattaaatttattatttaagatggtagcactagtcttaaattgtataaaatatcccctaacatgtttaaatgtccatttttattcattatgctttgaaaaataattatggggaaatacatgtttgttattaaatttattattaaagatagtagcactagtcttaaatttgatataacatctcctaacttgtttaaatgtccatttttattctttatgcttgaaaataaattatggggatcctatttagctcttagtaccactaatcaaaagttcggcatgtagctcatgatctatgctgtttctatgtcgtggaagcaccggatgggggtagtgagcaaatctgccctgctcagcagtcaccatagcagctgactgaaaatcagcactgcctgagtagttttgatcagtttaacttgaatcactaactgactgaaaattgaatgggcaaataagtgcttttgtctccagagtatgcgggagacccttccacctcaagatggatatttcttccccaaggatttcaagatgaattgaaatttttaatcaagatagtgtgctttattctgttgtattttttattattttaatatactgtaagccaaactgaaataacatttgctgttttataggtttgaagaacataggaaaaactaagaggttttgtttttatttttgctgatgaagagatatgtttaaatatgttgtattgttttgtttagttacaggacaataatgaaatggagtttatatttgttatttctattttgttatatttaataatagaattagattgaaataaaatataatgggaaataatctgcagaatgtgggtttcctggtgtttcctctgactctagtgcactgatgatctctgataaggctcagctgctttatagttctctggctaatgcagcagatactcttcctgccagtggtaatacgattttttaagaaggcagtttgtcaattttaatcttgtggatacctttatactcttagggtattattttatacaaaagccttgaggattgcattctattttctatatgaccctcttgatatttaaaaaacactatggataacaattcttcatttacctagtattatgaaagaatgaaggagttcaaacaaatgtgtttcccagttaactagggtttactgtttgagccaatataaatgtttaactgtttgtgatggcagtattcctaaagtacattgcatgttttcctaaatacagagtttaaataatttcagtaattcttagatgattcagcttcatcattaagaatatcttttgttttatgttgagttagaaatgccttcatatagacatagtctttcagacctctactgtcagttttcatttctagctgctttcagggttttatgaattttcaggcaaagctttaatttatactaagcttaggaagtatggctaatgccaacggcagtttttttcttcttaattccacatgactgaggcatatatgatctctgggtaggtgagttgttgtgacaaccacaagcactttttttttttttaaagaaaaaaaggtagtgaatttttaatcatctggactttaagaaggattctggagtatacttaggcctgaaattatatatatttggcttggaaatgtgtttttcttcaattacatctacaagtaagtacagctgaaattcagaggacccataagagttcacatgaaaaaaatcaattcatttgaaaaggcaagatgcaggagagaggaagccttgcaaacctgcagactgctttttgcccaatatagattgggtaaggctgcaaaacataagcttaattagctcacatgctctgctctcacgtggcaccagtggatagtgtgagagaattaggctgtagaacaaatggccttctctttcagcattcacaccactacaaaatcatcttttatatcaacagaagaataagcataaactaagcaaaaggtcaataagtacctgaaaccaagattggctagagatatatcttaatgcaatccattttctgatggattgttacgagttggctatataatgtatgtatggtattttgatttgtgtaaaagttttaaaaatcaagctttaagtacatggacatttttaaataaaatatttaaagacaatttagaaaattgccttaatatcattgttggctaaatagaataggggacatgcatattaaggaaaaggtcatggagaaataatattggtatcaaacaaatacattgatttgtcatgatacacattgaatttgatccaatagtttaaggaataggtaggaaaatttggtttctatttttcgatttcctgtaaatcagtgacataaataattcttagcttattttatatttccttgtcttaaatactgagctcagtaagttgtgttaggggattatttctcagttgagactttcttatatgacattttactatgttttgacttcctgactattaaaaataaatagtagaaacaattttcataaagtgaagaattatataatcactgctttataactgactttattatatttatttcaaagttcatttaaaggctactattcatcctctgtgatggaatggtcaggaatttgttttctcatagtttaattccaacaacaatattagtcgtatccaaaataacctttaatgctaaactttactgatgtatatccaaagcttctccttttcagacagattaatccagaagcagtcataaacagaagaataggtggtatgttcctaatgatattatttctactaatggaataaactgtaatattagaaattatgctgctaattatatcagctctgaggtaatttctgaaatgttcagactcagtcggaacaaattggaaaatttaaatttttattcttagctataaagcaagaaagtaaacacattaatttcctcaacatttttaagccaattaaaaatataaaagatacacaccaatatcttcttcaggctctgacaggcctcctggaaacttccacatatttttcaactgcagtataaagtcagaaaataaagttaacataactttcactaacacacacatatgtagatttcacaaaatccacctataattggtcaaagtggttgagaatatattttttagtaattgcatgcaaaatttttctagcttccatcctttctccctcgtttcttctttttttgggggagctggtaactgatgaaatcttttcccaccttttctcttcaggaaatataagtggttttgtttggttaacgtgatacattctgtatgaatgaaacattggagggaaacatctactgaatttctgtaatttaaaatattttgctgctagttaactatgaacagatagaagaatcttacagatgctgctataaataagtagaaaatataaatttcatcactaaaatatgctattttaaaatctatttcctatattgtatttctaatcagatgtattactcttattatttctattgtatgtgttaatgattttatgtaaaaatgtaattgcttttcatgagtagtatgaataaaattgattagtttgtgttttcttgtctcccgaaaaaaaaaaaaaaaaaaaaaaaaaaaaaa

SEQ ID NO: 30 - protein, 210 aa, Homo sapiens
MGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 31 - DNA, 1219 bp, Homo sapiens
gggagcgggcgagtaggagggggcgccgggctatatatatagcggcctcggcctcgggcgggcctggcgctcagggaggcgcgcactgctcctcagagtcccagctccagccgcgcgctttccgcccggctcgccgctccatgcagccggggtagagcccggcgcccgggggccccgtcgcttgcctcccgcacctcctcggttgcgcactcccgcccgaggtcggccgtgcgctcccgcgggacgccacaggcgcagctctgccccccagcttcccgggcgcactgaccgcctgaccgacgcacgccctcgggccgggatgtcggggcccgggacggccgcggtagcgctgctcccggcggtcctgctggccttgctggcgccctgggcgggccgagggggcgccgccgcacccactgcacccaacggcacgctggaggccgagctggagcgccgctgggagagcctggtggcgctctcgttggcgcgcctgccggtggcagcgcagcccaaggaggcggccgtccagagcggcgccggcgactacctgctgggcatcaagcggctgcggcggctctactgcaacgtgggcatcggcttccacctccaggcgctccccgacggccgcatcggcggcgcgcacgcggacacccgcgacagcctgctggagctctcgcccgtggagcggggcgtggtgagcatcttcggcgtggccagccggttcttcgtggccatgagcagcaagggcaagctctatggctcgcccttcttcaccgatgagtgcacgttcaaggagattctccttcccaacaactacaacgcctacgagtcctacaagtaccccggcatgttcatcgccctgagcaagaatgggaagaccaagaaggggaaccgagtgtcgcccaccatgaaggtcacccacttcctccccaggctgtgaccctccagaggacccttgcctcagcctcgggaagcccctgggagggcagtgcgagggtcaccttggtgcactttcttcggatgaagagtttaatgcaagagtaggtgtaagatatttaaattaattatttaaatgtgtatatattgccaccaaattatttatagttctgcgggtgtgttttttaattttctggggggaaaaaaagacaaaacaaaaaaccaactctgacttttctggtgcaacagtggagaatcttaccattggatttctttaacttgt

SEQ ID NO: 32 - protein, 206 aa, Homo sapiens
MSGPGTAAVALLPAVLLALLAPWAGRGGAAAPTAPNGTLEAELERRWESLVALSLARLPVAAQPKEAAVQSGAGDYLLGIKRLRRLYCNVGIGFHLQALPDGRIGGAHADTRDSLLELSPVERGVVSIFGVASRFFVAMSSKGKLYGSPFFTDECTFKEILLPNNYNAYESYKYPGMFIALSKNGKTKKGNRVSPTMKVTHFLPRL

SEQ ID NO: 33 - DNA, 987 bp, Homo sapiens
gcggcgcggcgagcacgacgttccacgggacccgcggagccgcgtcgtgatcgccgccggcctcccgcacccgcaccctctccgctcgcgccctgctcagcgcgtcctcccgcggcggcccgcgggacggcgtgacccgccgggctctcggtgccccggggccgcgcgccatgggcagcccccgctccgcgctgagctgcctgctgttgcacttgctggtcctctgcctccaagcccagcatgtgagggagcagagcctggtgacggatcagctcagccgccgcctcatccggacctaccaactctacagccgcaccagcgggaagcacgtgcaggtcctggccaacaagcgcatcaacgccatggcagaggacggcgaccccttcgcaaagctcatcgtggagacggacacctttggaagcagagtccgagtccgaggagccgagacgggcctctacatctgcatgaacaagaaggggaagctgatcgccaagagcaacggcaaaggcaaggactgcgtcttcacggagattgtgctggagaacaactacacagcgctgcagaatgccaagtacgagggctggtacatggccttcacccgcaagggccggccccgcaagggctccaagacgcggcagcaccagcgtgaggtccacttcatgaagcggctgccccggggccaccacaccaccgagcagagcctgcgcttcgagttcctcaactacccgcccttcacgcgcagcctgcgcggcagccagaggacttgggcccccgagccccgataggtgctgcctggccctccccacaatgccagaccgcagagaggctcatcctgtagggcacccaaaactcaagcaagatgagctgtgcgctgctctgcaggctggggaggtgctgggggagccctgggttccggttgttgatattgtttgctgttgggtttttgctgtttttttttttttttttttttttaaaacaaaagaggct

SEQ ID NO: 34 - protein, 204 aa, Homo sapiens
MGSPRSALSCLLLHLLVLCLQAQHVREQSLVTDQLSRRLIRTYQLYSRTSGKHVQVLANKRINAMAEDGDPFAKLIVETDTFGSRVRVRGAETGLYICMNKKGKLIAKSNGKGKDCVFTEIVLENNYTALQNAKYEGWYMAFTRKGRPRKGSKTRQHQREVHFMKRLPRGHHTTEQSLRFEFLNYPPFTRSLRGSQRTWAPEPR

SEQ ID NO: 35 - DNA, 627 bp, Homo sapiens
atgtggaaatggatactgacacattgtgcctcagcctttccccacctgcccggctgctgctgctgctgctttttgttgctgttcttggtgtcttccgtccctgtcacctgccaagcccttggtcaggacatggtgtcaccagaggccaccaactcttcttcctcctccttctcctctccttccagcgcgggaaggcatgtgcggagctacaatcaccttcaaggagatgtccgctggagaaagctattctctttcaccaagtactttctcaagattgagaagaacgggaaggtcagcgggaccaagaaggagaactgcccgtacagcatcctggagataacatcagtagaaatcggagttgttgccgtcaaagccattaacagcaactattacttagccatgaacaagaaggggaaactctatggctcaaaagaatttaacaatgactgtaagctgaaggagaggatagaggaaaatggatacaatacctatgcatcatttaactggcagcataatgggaggcaaatgtatgtggcattgaatggaaaaggagctccaaggagaggacagaaaacacgaaggaaaaacacctctgctcactttcttccaatggtggtacactcatag

SEQ ID NO: 36 - protein, 208 aa, Homo sapiens
MWKWILTHCASAFPHLPGCCCCCFLLLFLVSSVPVTCQALGQDMVSPEATNSSSSSFSSPSSAGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

SEQ ID NO: 37 - protein, 663 aa, Homo sapiens
ACCTCTCCAGCGATGGGAGCCGCCCGCCTGCTGCCCAACCTCACTCTGTGCTTACAGCTGCTGATTCTCTGCTGTCAAACTCAGGGGGAGAATCACCCGTCTCCTAATTTTAACCAGTACGTGAGGGACCAGGGCGCCATGACCGACCAGCTGAGCAGGCGGCAGATCCGCGAGTACCAACTCTACAGCAGGACCAGTGGCAAGCACGTGCAGGTCACCGGGCGTCGCATCTCCGCCACCGCCGAGGACGGCAACAAGTTTGCCAAGCTCATAGTGGAGACGGACACGTTTGGCAGCCGGGTTCGCATCAAAGGGGCTGAGAGTGAGAAGTACATCTGTATGAACAAGAGGGGCAAGCTCATCGGGAAGCCCAGCGGGAAGAGCAAAGACTGCGTGTTCACGGAGATCGTGCTGGAGAACAACTATACGGCCTTCCAGAACGCCCGGCACGAGGGCTGGTTCATGGCCTTCACGCGGCAGGGGCGGCCCCGCCAGGCTTCCCGCAGCCGCCAGAACCAGCGCGAGGCCCACTTCATCAAGCGCCTCTACCAAGGCCAGCTGCCCTTCCCCAACCACGCCGAGAAGCAGAAGCAGTTCGAGTTTGTGGGCTCCGCCCCCACCCGCCGGACCAAGCGCACACGGCGGCCCCAGCCCCTCACGTAG

SEQ ID NO: 38 - protein, 216 aa, Homo sapiens
MGAARLLPNLTLCLQLLILCCQTQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEFVGSAPTRRTKRTRRPQPLT

SEQ ID NO: 39 - DNA, 1546 bp, Homo sapiens
cacggccggagagacgcggaggaggagacatgagccggcgggcgcccagacggagcggccgtgacgctttcgcgctgcagccgcgcgccccgaccccggagcgctgacccctggccccacgcagctccgcgcccgggccggagagcgcaactcggcttccagacccgccgcgcatgctgtccccggactgagccgggcagccagcctcccacggacgcccggacggccggccggccagcagtgagcgagcttccccgcaccggccaggcgcctcctgcacagcggctgccgccccgcagcccctgcgccagcccggagggcgcagcgctcgggaggagccgcgcggggcgctgatgccgcagggcgcgccgcggagcgccccggagcagcagagtctgcagcagcagcagccggcgaggagggagcagcagcagcggcggcggcggcggcggcggcggcggaggcgcccggtcccggccgcgcggagcggacatgtgcaggctgggctaggagccgccgcctccctcccgcccagcgatgtattcagcgccctccgcctgcacttgcctgtgtttacacttcctgctgctgtgcttccaggtacaggtgctggttgccgaggagaacgtggacttccgcatccacgtggagaaccagacgcgggctcgggacgatgtgagccgtaagcagctgcggctgtaccagctctacagccggaccagtgggaaacacatccaggtcctgggccgcaggatcagtgcccgcggcgaggatggggacaagtatgcccagctcctagtggagacagacaccttcggtagtcaagtccggatcaagggcaaggagacggaattctacctgtgcatgaaccgcaaaggcaagctcgtggggaagcccgatggcaccagcaaggagtgtgtgttcatcgagaaggttctggagaacaactacacggccctgatgtcggctaagtactccggctggtacgtgggcttcaccaagaaggggcggccgcggaagggccccaagacccgggagaaccagcaggacgtgcatttcatgaagcgctaccccaaggggcagccggagcttcagaagcccttcaagtacacgacggtgaccaagaggtcccgtcggatccggcccacacaccctgcctaggccaccccgccgcggcccctcaggtcgccctggccacactcacactcccagaaaactgcatcagaggaatatttttacatgaaaaataaggaagaagctctatttttgtacattgtgtttaaaagaagacaaaaactgaaccaaaactcttggggggaggggtgataaggattttattgttgacttgaaacccccgatgacaaaagactcacgcaaagggactgtagtcaacccacaggtgcttgtctctctctaggaacagacaactctaaactcgtccccagaggaggacttgaatgaggaaaccaacactttgagaaaccaaagtcctttttcccaaaggttctgaaaggaaaaaaaaaaaaaaacaaaaaaaaaaaaaaaa

SEQ ID NO: 40 - protein, 207 aa, Homo sapiens
MYSAPSACTCLCLHFLLLCFQVQVLVAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA

SEQ ID NO: 41 - DNA, 3343 bp, Homo sapiens
gaattcgggatgtggagctggaagtgcctcctcttctgggctgtgctggtcacagccacactctgcaccgctaggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctcgggcagtgacaccacctacttctccgtcaatgtttcagatgctctcccctcctcggaggatgatgatgatgatgatgactcctcttcagaggagaaagaaacagataacaccaaaccaaaccccgtagctccatattggacatccccagaaaagatggaaaagaaattgcatgcagtgccggctgccaagacagtgaagttcaaatgcccttccagtgggaccccaaaccccacactgcgctggttgaaaaatagcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctgactgccacccacacgccctccccagactccaccgtcagctgtaaccctcacccacagcccctgctgggcccaccacctgtccgtccctgtcccctttcctgctggcaggagccggctgcctaccaggggccttcctgtgtggcctgccttcaccccactcagctcacctctccctccacctcctctccacctgctggtgagaggtgcaaagaggcagatctttgctgccagccacttcatcccctcccagatgttggaccaacacccctccctgccacagcatcgcctggagggcagggagtgggagccaatgaacaggcatgcaagtgagagcttcctgagctttctctgtcggtttggtctgttttgccttcacccataagcccctcgcactctggtggcaggtgccttgtcctcagggctacagcagtagggaggtcagtgcttcgtgcctcgattgaaggtgacctctgccccagataggtggtgcagtggcttattaattccgatactagtttgctttgctgaccaaatgcctggtaccagaggatggtgaggcgaaggccaggttgggggcagtgttgtggccctggggccagccccaaactgggggctctgtatatagctatgaagaaaacacaaagtgtataaatctgagtatatatttacatgtctttttaaaagggtcgttaccagagatttacccatcgggtaagatgctcctggtggctgggaggcatcagttgctatatattaaaaacaaaaaagaaaaaaaaggaaaatgtttttaaaaaggtcatatattttttgctacttttgctgttttatttttttaaattatgttctaaactcgtgccgctcgtgccgaattc

SEQ ID NO: 42 - protein, 820 aa, Homo sapiens [sequence omitted]

SEQ ID NO: 43 - DNA, 3248 bp, Homo sapiens
ggtaccgtaaccatggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcaccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtggtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgaggaatacttggacctcagccaacctctcgaacagtattcacctagttaccctgacacaagaagttcttgttcttcaggagatgattctgttttttctccagaccccatgccttacgaaccatgccttcctcagtatccacacataaacggcagtgttaaaacatgaatgactgtgtctgcctgtccccaaacaggacagcactgggaacctagctacactgagcagggagaccatgcctcccagagcttgttgtctccacttgtatatatggatcagaggagtaaataattggaaaagtaatcagcatatgtgtaaagatttatacagttgaaaacttgtaatcttccccaggaggagaagaaggtttctggagcagtggactgccacaagccaccatgtaacccctctcacctgccgtgcgtactggctgtggaccagtaggactcaaggtggacgtgcgttctgccttccttgttaattttgtaataattggagaagatttatgtcagcacacacttacagagcacaaatgcagtatataggtgctggatgtatgtaaatatattcaaattatgtataaatatatattatatatttacaaggagttattttttgtattgattttaaatggatgtcccaatgcacctagaaaattggtctctctttttttaatagctatttgctaaatgctgttcttacacataatttcttaattttcaccgagcagaggtggaaaaatacttttgctttcagggaaaatggtataacgttaatttattaataaattggtaatatacaaaacaattaatcatttatagttttttttgtaatttaagtggcatttctatgcaggcagcacagcagactagttaatctattgcttggacttaactagttatcagatcctttgaaaagagaatatttacaatatatga

SEQ ID NO: 44 - protein, 821 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 45 - DNA, 2184 bp, Homo sapiens
cgcgcgctgcctgaggacgccgcggcccccgcccccgccatgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaacggcagcaaggtgggcccggacggcacaccctacgttaccgtgctcaaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacaaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagtacctggacctgtcggcgcctttcgagcagtactccccgggtggccaggacacccccagctccagctcctcaggggacgactccgtgtttgcccacgacctgctgcccccggccccacccagcagtgggggctcgcggacgtgaagggccactggtccccaacaatgtgaggggtccctagcagccctccctgctgctggtgca

SEQ ID NO: 46 - protein, 694 aa, Homo sapiens
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 47 - DNA, 3015 bp, Homo sapiens
ccgaggagcgctcgggctgtctgcggaccctgccgcgtgcaggggtcgcggccggctggagctgggagtgaggcggcggaggagccaggtgaggaggagccaggaaggcagttggtgggaagtccagcttgggtccctgagagctgtgagaaggagatgcggctgctgctggccctgttgggggtcctgctgagtgtgcctgggcctccagtcttgtccctggaggcctctgaggaagtggagcttgagccctgcctggctcccagcctggagcagcaagagcaggagctgacagtagcccttgggcagcctgtgcggctgtgctgtgggcgggctgagcgtggtggccactggtacaaggagggcagtcgcctggcacctgctggccgtgtacggggctggaggggccgcctagagattgccagcttcctacctgaggatgctggccgctacctctgcctggcacgaggctccatgatcgtcctgcagaatctcaccttgattacaggtgactccttgacctccagcaacgatgatgaggaccccaagtcccatagggacctctcgaataggcacagttacccccagcaagcaccctactggacacacccccagcgcatggagaagaaactgcatgcagtacctgcggggaacaccgtcaagttccgctgtccagctgcaggcaaccccacgcccaccatccgctggcttaaggatggacaggcctttcatggggagaaccgcattggaggcattcggctgcgccatcagcactggagtctcgtgatggagagcgtggtgccctcggaccgcggcacatacacctgcctggtagagaacgctgtgggcagcatccgctataactacctgctagatgtgctggagcggtccccgcaccggcccatcctgcaggccgggctcccggccaacaccacagccgtggtgggcagcgacgtggagctgctgtgcaaggtgtacagcgatgcccagccccacatccagtggctgaagcacatcgtcatcaacggcagcagcttcggagccgacggtttcccctatgtgcaagtcctaaagactgcagacatcaatagctcagaggtggaggtcctgtacctgcggaacgtgtcagccgaggacgcaggcgagtacacctgcctcgcaggcaattccatcggcctctcctaccagtctgcctggctcacggtgctgccagaggaggaccccacatggaccgcagcagcgcccgaggccaggtatacggacatcatcctgtacgcgtcgggctccctggccttggctgtgctcctgctgctggccgggctgtatcgagggcaggcgctccacggccggcacccccgcccgcccgccactgtgcagaagctctcccgcttccctctggcccgacagttctccctggagtcaggctcttccggcaagtcaagctcatccctggtacgaggcgtgcgtctctcctccagcggccccgccttgctcgccggcctcgtgagtctagatctacctctcgacccactatgggagttcccccgggacaggctggtgcttgggaagcccctaggcgagggctgctttggccaggtagtacgtgcagaggcctttggcatggaccctgcccggcctgaccaagccagcactgtggccgtcaagatgctcaaagacaacgcctctgacaaggacctggccgacctggtctcggagatggaggtgatgaagctgatcggccgacacaagaacatcatcaacctgcttggtgtctgcacccaggaagggcccctgtacgtgatcgtggagtgcgccgccaagggaaacctgcgggagttcctgcgggcccggcgccccccaggccccgacctcagccccgacggtcctcggagcagtgaggggccgctctccttcccagtcctggtctcctgcgcctaccaggtggcccgaggcatgcagtatctggagtcccggaagtgtatccaccgggacctggctgcccgcaatgtgctggtgactgaggacaatgtgatgaagattgctgactttgggctggcccgcggcgtccaccacattgactactataagaaaaccagcaacggccgcctgcctgtgaagtggatggcgcccgaggccttgtttgaccgggtgtacacacaccagagtgacgtgtggtcttttgggatcctgctatgggagatcttcaccctcgggggctccccgtatcctggcatcccggtggaggagctgttctcgctgctgcgggagggacatcggatggaccgacccccacactgccccccagagctgtacgggctgatgcgtgagtgctggcacgcagcgccctcccagaggcctaccttcaagcagctggtggaggcgctggacaaggtcctgctggccgtctctgaggagtacctcgacctccgcctgaccttcggaccctattccccctctggtggggacgccagcagcacctgctcctccagcgattctgtcttcagccacgaccccctgccattgggatccagctccttccccttcgggtctggggtgcagacatgagcaaggctcaaggctgtgcaggcacataggctggtggccttgggccttggggctcagccacagcctgacacagtgctcgaccttgatagcatggggcccctggcccagagttgctgtgccgtgtccaagggccgtgcccttgcccttggagctgccgtgcctgtgtcctgatggcccaaatgtcagggttctgctcggcttcttggaccatggcgcttagtccccatcccgggtttggctgagcctggctggagagctgctatgctaaacctcctgcctcccaataccagcaggaggttctgggcctctgaaccccctttccccacacctccccctgctgctgctgccccagcgtcttgacgggagcattggcccctgagcccagagaagctggaagcctgccgaaaacaggagcaaatggcgttttataaattatttttttgaaataaa

SEQ ID NO: 48 - protein, 802 aa, Homo sapiens
MRLLLALLGVLLSVPGPPVLSLEASEEVELEPCLAPSLEQQEQELTVALGQPVRLCCGRAERGGHWYKEGSRLAPAGRVRGWRGRLEIASFLPEDAGRYLCLARGSMIVLQNLTLITGDSLTSSNDDEDPKSHRDLSNRHSYPQQAPYWTHPQRMEKKLHAVPAGNTVKFRCPAAGNPTPTIRWLKDGQAFHGENRIGGIRLRHQHWSLVMESVVPSDRGTYTCLVENAVGSIRYNYLLDVLERSPHRPILQAGLPANTTAVVGSDVELLCKVYSDAQPHIQWLKHIVINGSSFGADGFPYVQVLKTADINSSEVEVLYLRNVSAEDAGEYTCLAGNSIGLSYQSAWLTVLPEEDPTWTAAAPEARYTDIILYASGSLALAVLLLLAGLYRGQALHGRHPRPPATVQKLSRFPLARQFSLESGSSGKSSSSLVRGVRLSSSGPALLAGLVSLDLPLDPLWEFPRDRLVLGKPLGEGCFGQVVRAEAFGMDPARPDQASTVAVKMLKDNASDKDLADLVSEMEVMKLIGRHKNIINLLGVCTQEGPLYVIVECAAKGNLREFLRARRPPGPDLSPDGPRSSEGPLSFPVLVSCAYQVARGMQYLESRKCIHRDLAARNVLVTEDNVMKIADFGLARGVHHIDYYKKTSNGRLPVKWMAPEALFDRVYTHQSDVWSFGILLWEIFTLGGSPYPGIPVEELFSLLREGHRMDRPPHCPPELYGLMRECWHAAPSQRPTFKQLVEALDKVLLAVSEEYLDLRLTFGPYSPSGGDASSTCSSSDSVFSHDPLPLGSSSFPFGSGVQT

SEQ ID NO: 49 - DNA, 2368 bp, Homo sapiens
gcggtgccgcccgccgtggccgcctcagcccaccagccgggaccgcgagccatgctgtccgccgcccgcccccagggttgttaaagccagactgcgaactctcgccactgccgccaccgccgcgtcccgtcccaccgtcgcgggcaacaaccaaagtcgccgcaactgcagcacagagcgggcaaagccaggcaggccatggggctctgggcgctgttgcctggctgggtttctgctacgctgctgctggcgctggccgctctgcccgcagccctggctgccaacagcagtggccgatggtggggtattgtgaacgtagcctcctccacgaacctgcttacagactccaagagtctgcaactggtactcgagcccagtctgcagctgttgagccgcaaacagcggcgtctgatacgccaaaatccggggatcctgcacagcgtgagtggggggctgcagagtgccgtgcgcgagtgcaagtggcagttccggaatcgccgctggaactgtcccactgctccagggccccacctcttcggcaagatcgtcaaccgaggctgtcgagaaacggcgtttatcttcgctatcacctccgccggggtcacccattcggtggcgcgctcctgctcagaaggttccatcgaatcctgcacgtgtgactaccggcggcgcggccccgggggccccgactggcactgggggggctgcagcgacaacattgacttcggccgcctcttcggccgggagttcgtggactccggggagaaggggcgggacctgcgcttcctcatgaaccttcacaacaacgaggcaggccgtacgaccgtattctccgagatgcgccaggagtgcaagtgccacgggatgtccggctcatgcacggtgcgcacgtgctggatgcggctgcccacgctgcgcgccgtgggcgatgtgctgcgcgaccgcttcgacggcgcctcgcgcgtcctgtacggcaaccgcggcagcaaccgcgcttcgcgagcggagctgctgcgcctggagccggaagacccggcccacaaaccgccctccccccacgacctcgtctacttcgagaaatcgcccaacttctgcacgtacagcggacgcctgggcacagcaggcacggcagggcgcgcctgtaacagctcgtcgcccgcgctggacggctgcgagctgctctgctgcggcaggggccaccgcacgcgcacgcagcgcgtcaccgagcgctgcaactgcaccttccactggtgctgccacgtcagctgccgcaactgcacgcacacgcgcgtactgcacgagtgtctgtgaggcgctgcgcggactcgcccccaggaaacgctctcctcgagccctcccccaaacagactcgctagcactcaagacccggttattcgcccacccgagtacctccagtcacactccccgcggttcatacgcatcccatctctcccacttcctcctacctggggactcctcaaaccacttgcctggggcggcatgaaccctcttgccatcctgatggacctgccccggacctacctccctccctctccgcgggagaccccttgttgcactgccccctgcttggccaggaggtgagagaaggatgggtcccctccgccatggggtcggctcctgatggtgtcattctgcctgctccatcgcgccagcgacctctctgcctctcttcttcccctttgtcctgcgttttctccgggtcctcctaagtcccttcctattctcctgccatgggtgcagaccctgaacccacacctgggcatcagggcctttctcctccccacctgtagctgaagcaggaggttacagggcaaaagggcagctgtgatgatgtggaaatgaggttgggggaaccagcagaaatgcccccattctcccagtctctgtcgtggagccattgaacagctgtgagccatgcctccctgggccacctcctaccccttcctgtcctgcctcctcatcagtgtgtaaataatttgcactgaaacgtggatacagagccacgagtttggatgttgtaaataaaactatttattgtgctgggtcccagcctggtttgcaaagaccacctccaacccaacccaatccctctccactcttctctcctttctccctgcagccttttctggtccctcttctctcctcagtttctcaaagatgcgtttgcctcctggaatcagtatttccttccactgtagctattagcggctcctcgcccccaccagtgtagcatcttcctctgcagaataaaatctctatttttatcgatgacttggtggcttttccttgaatccagaacacaaccttgtttgtggtgtcccctatcctccccttttaccactcccag

SEQ ID NO: 50 - protein, 370 aa, Homo sapiens
MGLWALLPGWVSATLLLALAALPAALAANSSGRWWGIVNVASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL

SEQ ID NO: 51 - DNA, 2301 bp, Homo sapiens
agcagagcggacgggcgcgcgggaggcgcgcagagctttcgggctgcaggcgctcgctgccgctggggaattgggctgtgggcgaggcggtccgggctggcctttatcgctcgctgggcccatcgtttgaaactttatcagcgagtcgccactcgtcgcaggaccgagcggggggcgggggcgcggcgaggcggcggccgtgacgaggcgctcccggagctgagcgcttctgctctgggcacgcatggcgcccgcacacggagtctgacctgatgcagacgcaagggggttaatatgaacgcccctctcggtggaatctggctctggctccctctgctcttgacctggctcacccccgaggtcaactcttcatggtggtacatgagagctacaggtggctcctccagggtgatgtgcgataatgtgccaggcctggtgagcagccagcggcagctgtgtcaccgacatccagatgtgatgcgtgccattagccagggcgtggccgagtggacagcagaatgccagcaccagttccgccagcaccgctggaattgcaacaccctggacagggatcacagcctttttggcagggtcctactccgaagtagtcgggaatctgcctttgtttatgccatctcctcagctggagttgtatttgccatcaccagggcctgtagccaaggagaagtaaaatcctgttcctgtgatccaaagaagatgggaagcgccaaggacagcaaaggcatttttgattggggtggctgcagtgataacattgactatgggatcaaatttgcccgcgcatttgtggatgcaaaggaaaggaaaggaaaggatgccagagccctgatgaatcttcacaacaacagagctggcaggaaggctgtaaagcggttcttgaaacaagagtgcaagtgccacggggtgagcggctcatgtactctcaggacatgctggctggccatggccgacttcaggaaaacgggcgattatctctggaggaagtacaatggggccatccaggtggtcatgaaccaggatggcacaggtttcactgtggctaacgagaggtttaagaagccaacgaaaaatgacctcgtgtattttgagaattctccagactactgtatcagggaccgagaggcaggctccctgggtacagcaggccgtgtgtgcaacctgacttcccggggcatggacagctgtgaagtcatgtgctgtgggagaggctacgacacctcccatgtcacccggatgaccaagtgtgggtgtaagttccactggtgctgcgccgtgcgctgtcaggactgcctggaagctctggatgtgcacacatgcaaggcccccaagaacgctgactggacaaccgctacatgaccccagcaggcgtcaccatccaccttcccttctacaaggactccattggatctgcaagaacactggacctttgggttctttctggggggatatttcctaaggcatgtggcctttatctcaacggaagccccctcttcctccctgggggccccaggatggggggccacacgctgcacctaaagcctaccctattctatccatctcctggtgttctgcagtcatctcccctcctggcgagttctctttggaaatagcatgacaggctgttcagccgggagggtggtgggcccagaccactgtctccacccaccttgacgtttcttctttctagagcagttggccaagcagaaaaaaaagtgtctcaaaggagctttctcaatgtcttcccacaaatggtcccaattaagaaattccatacttctctcagatggaacagtaaagaaagcagaatcaactgcccctgacttaactttaacttttgaaaagaccaagacttttgtctgtacaagtggttttacagctaccacccttagggtaattggtaattacctggagaagaatggctttcaatacccttttaagtttaaaatgtgtatttttcaaggcatttattgccatattaaaatctgatgtaacaaggtggggacgtgtgtcctttggtactatggtgtgttgtatctttgtaagagcaaaagcctcagaaagggattgctttgcattactgtccccttgatataaaaaatctttagggaatgagagttccttctcacttagaatctgaagggaattaaaaagaagatgaatggtctggcaatattctgtaactattgggtgaatatggtggaaaataatttagtggatggaatatcagaagtatatctgtacagatcaagaaaaaaaggaagaataaaattcctatatcat

SEQ ID NO: 52 - protein, 360 aa, Homo sapiens
MNAPLGGIWLWLPLLLTWLTPEVNSSWWYMRATGGSSRVMCDNVPGLVSSQRQLCHRHPDVMRAISQGVAEWTAECQHQFRQHRWNCNTLDRDHSLFGRVLLRSSRESAFVYAISSAGVVFAITRACSQGEVKSCSCDPKKMGSAKDSKGIFDWGGCSDNIDYGIKFARAFVDAKERKGKDARALMNLHNNRAGRKAVKRFLKQECKCHGVSGSCTLRTCWLAMADFRKTGDYLWRKYNGAIQVVMNQDGTGFTVANERFKKPTKNDLVYFENSPDYCIRDREAGSLGTAGRVCNLTSRGMDSCEVMCCGRGYDTSHVTRMTKCGCKFHWCCAVRCQDCLEALDVHTCKAPKNADWTTAT

SEQ ID NO: 53 - DNA, 1506 bp, Homo sapiens
gcgcttctgacaagcccgaaagtcatttccaatctcaagtggactttgttccaactattgggggcgtcgctccccctcttcatggtcgcgggcaaacttcctcctcggcgcctcttctaatggagccccacctgctcgggctgctcctcggcctcctgctcggtggcaccagggtcctcgctggctacccaatttggtggtccctggccctgggccagcagtacacatctctgggctcacagcccctgctctgcggctccatcccaggcctggtccccaagcaactgcgcttctgccgcaattacatcgagatcatgcccagcgtggccgagggcgtgaagctgggcatccaggagtgccagcaccagttccggggccgccgctggaactgcaccaccatagatgacagcctggccatctttgggcccgtcctcgacaaagccacccgcgagtcggccttcgttcacgccatcgcctcggccggcgtggccttcgccgtcacccgctcctgcgccgagggcacctccaccatttgcggctgtgactcgcatcataaggggccgcctggcgaaggctggaagtggggcggctgcagcgaggacgctgacttcggcgtgttagtgtccagggagttcgcggatgcgcgcgagaacaggccggacgcgcgctcggccatgaacaagcacaacaacgaggcgggccgcacgactatcctggaccacatgcacctcaaatgcaagtgccacgggctgtcgggcagctgtgaggtgaagacctgctggtgggcgcagcctgacttccgtgccatcggtgacttcctcaaggacaagtatgacagcgcctcggagatggtagtagagaagcaccgtgagtcccgaggctgggtggagaccctccgggccaagtactcgctcttcaagccacccacggagagggacctggtctactacgagaactcccccaacttttgtgagcccaacccagagacgggttcctttggcacaagggaccggacttgcaatgtcacctcccacggcatcgatggctgcgatctgctctgctgtggccggggccacaacacgaggacggagaagcggaaggaaaaatgccactgcatcttccactggtgctgctacgtcagctgccaggagtgtattcgcatctacgacgtgcacacctgcaagtagggcaccagggcgctgggaaggggtgaagtgtgtggctgggcggattcagcgaagtctcatgggaagcaggacctagagccgggcacagccctcagcgtcagacagcaaggaactgtcaccagccgcacgcgtggtaaatgacccagacccaactcgcctgtggacggggaggctctccctctctctcatcttacatttctcaccctactctggatggtgtgtggtttttaaagaagggggctttctttttagttctctagggtctgataggaacagacctgaggcttatctttgcacatgttaaagaaaaaaaaaaaaa

SEQ ID NO: 54 - protein, 355 aa, Homo sapiens
MEPHLLGLLLGLLLGGTRVLAGYPIWWSLALGQQYTSLGSQPLLCGSIPGLVPKQLRFCRNYIEIMPSVAEGVKLGIQECQHQFRGRRWNCTTIDDSLAIFGPVLDKATRESAFVHAIASAGVAFAVTRSCAEGTSTICGCDSHHKGPPGEGWKWGGCSEDADFGVLVSREFADARENRPDARSAMNKHNNEAGRTTILDHMHLKCKCHGLSGSCEVKTCWWAQPDFRAIGDFLKDKYDSASEMVVEKHRESRGWVETLRAKYSLFKPPTERDLVYYENSPNFCEPNPETGSFGTRDRTCNVTSHGIDGCDLLCCGRGHNTRTEKRKEKCHCIFHWCCYVSCQECIRIYDVHTCK

SEQ ID NO: 55 - DNA, 4428 bp, Homo sapiens
ttaaggaaatccgggctgctcttccccatctggaagtggctttccccacatcggctcgtaaactgattatgaaacatacgatgttaattcggagctgcatttcccagctgggcactctcgcgcgctggtccccggggcctcgccccccaccccctgcccttccctcccgcgtcctgcccccatcctccaccccccgcgctggccaccccgcctccttggcagcctctggcggcagcgcgctccactcgcctcccgtgctcctctcgcccatggaattaattctggctccacttgttgctcggcccaggttggggagaggacggagggtggccgcagcgggttcctgagtgaattacccaggagggactgagcacagcaccaactagagaggggtcagggggtgcgggactcgagcgagcaggaaggaggcagcgcctggcaccagggctttgactcaacagaattgagacacgtttgtaatcgctggcgtgccccgcgcacaggatcccagcgaaaatcagatttcctggtgaggttgcgtgggtggattaatttggaaaaagaaactgcctatatcttgccatcaaaaaactcacggaggagaagcgcagtcaatcaacagtaaacttaagagacccccgatgctcccctggtttaacttgtatgcttgaaaattatctgagagggaataaacatcttttccttcttccctctccagaagtccattggaatattaagcccaggagttgctttggggatggctggaagtgcaatgtcttccaagttcttcctagtggctttggccatatttttctccttcgcccaggttgtaattgaagccaattcttggtggtcgctaggtatgaataaccctgttcagatgtcagaagtatatattataggagcacagcctctctgcagccaactggcaggactttctcaaggacagaagaaactgtgccacttgtatcaggaccacatgcagtacatcggagaaggcgcgaagacaggcatcaaagaatgccagtatcaattccgacatcgacggtggaactgcagcactgtggataacacctctgtttttggcagggtgatgcagataggcagccgcgagacggccttcacatacgccgtgagcgcagcaggggtggtgaacgccatgagccgggcgtgccgcgagggcgagctgtccacctgcggctgcagccgcgccgcgcgccccaaggacctgccgcgggactggctctggggcggctgcggcgacaacatcgactatggctaccgctttgccaaggagttcgtggacgcccgcgagcgggagcgcatccacgccaagggctcctacgagagtgctcgcatcctcatgaacctgcacaacaacgaggccggccgcaggacggtgtacaacctggctgatgtggcctgcaagtgccatggggtgtccggctcatgtagcctgaagacatgctggctgcagctggcagacttccgcaaggtgggtgatgccctgaaggagaagtacgacagcgcggcggccatgcggctcaacagccggggcaagttggtacaggtcaacagccgcttcaactcgcccaccacacaagacctggtctacatcgaccccagccctgactactgcgtgcgcaatgagagcaccggctcgctgggcacgcagggccgcctgtgcaacaagacgtcggagggcatggatggctgcgagctcatgtgctgcggccgtgggtacgaccagttcaagaccgtgcagacggagcgctgccactgcaagttccactggtgctgctacgtcaagtgcaagaagtgcacggagatcgtggaccagtttgtgtgcaagtagtgggtgccacccagcactcagccccgctcccaggacccgcttatttatagaaagtacagtgattctggtttttggtttttagaaatattttttatttttccccaagaattgcaaccggaaccattttttttcctgttaccatctaagaactctgtggtttattattaatattataattattatttggcaataatgggggtgggaaccacgaaaaatatttattttgtggatctttgaaaaggtaatacaagacttcttttggatagtatagaatgaagggggaaataacacataccctaacttagctgtgtgggacatggtacacatccagaaggtaaagaaatacattttctttttctcaaatatgccatcatatgggatgggtaggttccagttgaaagagggtggtagaaatctattcacaattcagcttctatgaccaaaatgagttgtaaattctctggtgcaagataaaaggtcttgggaaaacaaaacaaaacaaaacaaacctcccttccccagcagggctgctagcttgctttctgcattttcaaaatgataatttacaatggaaggacaagaatgtcatattctcaaggaaaaaaggtatatcacatgtctcattctcctcaaatattccatttgcagacagaccgtcatattctaatagctcatgaaatttgggcagcagggaggaaagtccccagaaattaaaaaatttaaaactcttatgtcaagatgttgatttgaagctgttataagaattgggattccagatttgtaaaaagacccccaatgattctggacactagattttttgtttggggaggttggcttgaacataaatgaaatatcctgtattttcttagggatacttggttagtaaattataatagtagaaataatacatgaatcccattcacaggtttctcagcccaagcaacaaggtaattgcgtgccattcagcactgcaccagagcagacaacctatttgaggaaaaacagtgaaatccaccttcctcttcacactgagccctctctgattcctccgtgttgtgatgtgatgctggccacgtttccaaacggcagctccactgggtcccctttggttgtaggacaggaaatgaaacattaggagctctgcttggaaaacagttcactacttagggatttttgtttcctaaaacttttattttgaggagcagtagttttctatgttttaatgacagaacttggctaatggaattcacagaggtgttgcagcgtatcactgttatgatcctgtgtttagattatccactcatgcttctcctattgtactgcaggtgtaccttaaaactgttcccagtgtacttgaacagttgcatttataaggggggaaatgtggtttaatggtgcctgatatctcaaagtcttttgtacataacatatatatatatatacatatatataaatataaatataaatatatctcattgcagccagtgatttagatttacagcttactctggggttatctctctgtctagagcattgttgtccttcactgcagtccagttgggattattccaaaagttttttgagtcttgagcttgggctgtggccccgctgtgatcataccctgagcacgacgaagcaacctcgtttctgaggaagaagcttgagttctgactcactgaaatgcgtgttgggttgaagatatctttttttcttttctgcctcacccctttgtctccaacctccatttctgttcactttgtggagagggcattacttgttcgttatagacatggacgttaagagatattcaaaactcagaagcatcagcaatgtttctcttttcttagttcattctgcagaatggaaacccatgcctattagaaatgacagtacttattaattgagtccctaaggaatattcagcccactacatagatagctttttttttttttttttttttttaataaggacacctctttccaaacaggccatcaaatatgttcttatctcagacttacgttgttttaaaagtttggaaagatacacatcttttcatacccccccttaggaggttgggctttcatatcacctcagccaactgtggctcttaatttattgcataatgatatccacatcagccaactgtggctctttaatttattgcataatgatattcacatcccctcagttgcagtgaattgtgagcaaaagatcttgaaagcaaaaagcactaattagtttaaaatgtcacttttttggtttttattatacaaaaaccatgaagtactttttttatttgctaaatcagattgttcctttttagtgactcatgtttatgaagagagttgagtttaacaatcctagcttttaaaagaaactatttaatgtaaaatattctacatgtcattcagatattatgtatatcttctagcctttattctgtacttttaatgtacatatttctgtcttgcgtgatttgtatatttcactggtttaaaaaacaaacatcgaaaggcttattccaaatggaagatagaatataaaataaaacgttacttgtaaaaaaaaaaaa

SEQ ID NO: 56 - protein, 365 aa, Homo sapiens
MAGSAMSSKFFLVALAIFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 57 - DNA, 1899 bp, Homo sapiens
cagaattttctcacataaatactgaggaagaccctgccctctcctcactcctctggacttggccctgagctggacctggtccactggggtaggcagggcgatggggaacctgtttatgctctgggcagctctgggcatatgctgtgctgcattcagtgcctctgcctggtcagtgaacaatttcctgataacaggtcccaaggcctatctgacctacacgactagtgtggccttgggtgcccagagtggcatcgaggagtgcaagttccagtttgcttgggaacgctggaactgccctgaaaatgctcttcagctctccacccacaacaggctgagaagtgctaccagagagacttccttcatacatgctatcagctctgctggagtcatgtacatcatcaccaagaactgtagcatgggtgacttcgaaaactgtggctgtgatgggtcaaacaatggaaaaacaggaggccatggctggatctggggaggctgcagcgacaatgtggaatttggggaaaggatctccaaactctttgtggacagtttggagaaggggaaggatgccagagccctgatgaatcttcacaacaacagggccggcagactggcagtgagagccaccatgaaaaggacatgcaaatgtcatggcatctctgggagctgcagcatacagacatgctggctgcagctggctgaattccgggagatgggagactacctaaaggccaagtatgaccaggcgctgaaaattgaaatggataagcggcagctgagagctgggaacagcgccgagggccactgggtgcccgctgaggccttccttcctagcgcagaggcggaactgatctttttagaggaatcaccagattactgtacctgcaattccagcctgggcatctatggcacagagggtcgtgagtgcctacagaacagccacaacacatccaggtgggagcgacgtagctgtgggcgcctgtgcactgagtgtgggctgcaggtggaagagaggaaaactgaggtcataagcagctgtaactgcaaattccagtggtgctgtacggtcaagtgtgaccagtgtaggcatgtggtgagcaagtattactgcgcacgctccccaggcagtgcccagtccctgggtaagggcagtgcctgataataccccacacaagttcacttgattaattgcatcagtggaaggggacatagcttctctcttagagagaacagattggaaagcaatcggaaaattgcagttttggtctgtagtcctcatgatatctgctatcagtggggaaaatggaggcccaagattctacagcatattcctggcggggctgaaattggaacctgggcctcctgactttggcagacccccatttcatctttcctgcaaactactttcccatctttgtgcctgtacttatgcagctttctacagggagagtttggtttggggtctatatctagagggaccttcaaagtatttgttcctttaaatttcagaccatgtccaacccagctgtgctgctgggaatcaggagaatagaagcaaaaaacgaaagagttctgttcagacttctgaagagcagcctgtggctacaaatctatgctgataaatgagattgagaactcaactgtattttgccataaatgcttctaagatatatccagctgggacttctattactccctttggaaaccttaagatcaaaaagggaataagaaacccttcttctgtatcccaataatccaccaggataaaggagaaactagaaatatgcaactcccttgatttcagtgtttggcaggtaacaaaaaattgagacccagacactggtcaacaggaaaacaatacagactcccagaattagaaagtgttattttaatgcaacctag

SEQ ID NO: 58 - protein, 351 aa, Homo sapiens
MGNLFMLWAALGICCAAFSASAWSVNNFLITGPKAYLTYTTSVALGAQSGIEECKFQFAWERWNCPENALQLSTHNRLRSATRETSFIHAISSAGVMYIITKNCSMGDFENCGCDGSNNGKTGGHGWIWGGCSDNVEFGERISKLFVDSLEKGKDARALMNLHNNRAGRLAVRATMKRTCKCHGISGSCSIQTCWLQLAEFREMGDYLKAKYDQALKIEMDKRQLRAGNSAEGHWVPAEAFLPSAEAELIFLEESPDYCTCNSSLGIYGTEGRECLQNSHNTSRWERRSCGRLCTECGLQVEERKTEVISSCNCKFQWCCTVKCDQCRHVVSKYYCARSPGSAQSLGKGSA

SEQ ID NO: 59 - DNA, 1927 bp, Homo sapiens
taacccgccgcctccgctctccccggctgcaggcggcgtgcaggaccagcggcggccgtgcaggcggaggacttcggcgcggctcctcctgggtgtgaccccgggcgcgcccgccgcgcgacgatgagggcgcggccgcaggtctgcgaggcgctgctcttcgccctggcgctccagaccggcgtgtgctatggcatcaagtggctggcgctgtccaagacaccatcggccctggcactgaaccagacgcaacactgcaagcagctggagggtctggtgtctgcacaggtgcagctgtgccgcagcaacctggagctcatgcacacggtggtgcacgccgcccgcgaggtcatgaaggcctgtcgccgggcctttgccgacatgcgctggaactgctcctccattgagctcgcccccaactatttgcttgacctggagagagggacccgggagtcggccttcgtgtatgcgctgtcggccgccgccatcagccacgccatcgcccgggcctgcacctccggcgacctgcccggctgctcctgcggccccgtcccaggtgagccacccgggcccgggaaccgctggggaggatgtgcggacaacctcagctacgggctcctcatgggggccaagttttccgatgctcctatgaaggtgaaaaaaacaggatcccaagccaataaactgatgcgtctacacaacagtgaagtggggagacaggctctgcgcgcctctctggaaatgaagtgtaagtgccatggggtgtctggctcctgctccatccgcacctgctggaaggggctgcaggagctgcaggatgtggctgctgacctcaagacccgatacctgtcggccaccaaggtagtgcaccgacccatgggcacccgcaagcacctggtgcccaaggacctggatatccggcctgtgaaggactcggaactcgtctatctgcagagctcacctgacttctgcatgaagaatgagaaggtgggctcccacgggacacaagacaggcagtgcaacaagacatccaacggaagcgacagctgcgaccttatgtgctgcgggcgtggctacaacccctacacagaccgcgtggtcgagcggtgccactgtaagtaccactggtgctgctacgtcacctgccgcaggtgtgagcgtaccgtggagcgctatgtctgcaagtgaggccctgccctccgccccacgcaggagcgaggactctgctcaaggaccctcagcaactggggccaggggcctggagacactccatggagctctgcttgtgaattccagatgccaggcatgggaggcggcttgtgctttgccttcacttggaagccaccaggaacagaaggtctggccaccctggaaggagggcaggacatcaaaggaaaccgacaagattaaaaataacttggcagcctgaggctctggagtgcccacaggctggtgtaaggagcggggcttgggatcggtgagactgatacagacttgacctttcagggccacagagaccagcctccgggaaggggtctgcccgccttcttcagaatgttctgcgggaccccctggcccaccctggggtctgagcctgctgggcccaccacatggaatcactagcttgggttgtaaatgttttcttttgttttttgctttttcttcctttgggatgtggaagctacagaaatatttataaaacatagctttttctttggggtggcacttctcaattcctctttatatattttatatatataaatatatatgtatatatataatgatctctattttaaaactagctttttaagcagctgtatgaaataaatgctgagtgagccccagcccgcccctgcagttcccggcctcgtcaagtgaactcggcagaccctggggctggcagagggagctctccagtttccaggca

SEQ ID NO: 60 - protein, 354 aa, Homo sapiens
MRARPQVCEALLFALALQTGVCYGIKWLALSKTPSALALNQTQHCKQLEGLVSAQVQLCRSNLELMHTVVHAAREVMKACRRAFADMRWNCSSIELAPNYLLDLERGTRESAFVYALSAAAISHAIARACTSGDLPGCSCGPVPGEPPGPGNRWGGCADNLSYGLLMGAKFSDAPMKVKKTGSQANKLMRLHNSEVGRQALRASLEMKCKCHGVSGSCSIRTCWKGLQELQDVAADLKTRYLSATKVVHRPMGTRKHLVPKDLDIRPVKDSELVYLQSSPDFCMKNEKVGSHGTQDRQCNKTSNGSDSCDLMCCGRGYNPYTDRVVERCHCKYHWCCYVTCRRCERTVERYVCK

SEQ ID NO: 61 - DNA, 1639 bp, Homo sapiens
atcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggattcgtcaggaacaggacatttcacctcaggagtgcgggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatattaaaaaaaaaaaaaaaaaa

SEQ ID NO: 62 - protein, 433 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKDSSGTGHFTSGVRVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 63 - DNA, 3362 bp, Homo sapiens
aagcctctcggtctgtggcagcagcgttggcccggccccgggagcggagagcgaggggaggcggagacggaggaaggtctgaggagcagcttcagtccccgccgagccgccaccgcaggtcgaggacggtcggactcccgcggcgggaggagcctgttcccctgagggtatttgaagtataccatacaactgttttgaaaatccagcgtggacaatggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaaatcatcctttagctgtattgtctgaacttgcattgtgattggcctgtagagttgctgagagggctcgaggggtgggctggtatctcagaaagtgcctgacacactaaccaagctgagtttcctatgggaacaattgaagtaaactttttgttctggtcctttttggtcgaggagtaacaatacaaatggattttgggagtgactcaagaagtgaagaatgcacaagaatggatcacaagatggaatttagcaaaccctagccttgcttgttaaaatttttttttttttttttttaagaatatctgtaatggtactgactttgcttgctttgaagtagctctttttttttttttttttttttttttttgcagtaactgttttttaagtctctcgtagtgttaagttatagtgaatactgctacagcaatttctaatttttaagaattgagtaatggtgtagaacactaattaattcataatcactctaattaattgtaatctgaataaagtgtaacaattgtgtagcctttttgtataaaatagacaaatagaaaatggtccaattagtttcctttttaatatgcttaaaataagcaggtggatctatttcatgtttttgatcaaaaactatttgggatatgtatgggtagggtaaatcagtaagaggtgttatttggaaccttgttttggacagtttaccagttgccttttatcccaaagttgttgtaacctgctgtgatacgatgcttcaagagaaaatgcggttataaaaaatggttcagaattaaacttttaattcatt

SEQ ID NO: 64 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 65 - DNA, 3084 bp, Homo sapiens
aagatctaaaaacggacatctccaccgtgggtggctcctttttctttttctttttttcccacccttcaggaagtggacgtttcgttatcttctgatccttgcaccttcttttggggaaacggggcccttctgcccagatcccctctcttttctcggaaaacaaactactaagtcggcatccggggtaactacagtggagagggtttccgcggagacgcgccgccggaccctcctctgcactttggggaggcgtgctccctccagaaccggcgttctccgcgcgcaaatcccggcgacgcggggtcgcggggtggccgccggggcagcctcgtctagcgcgcgccgcgcagacgcccccggagtcgccagctaccgcagccctcgccgcccagtgcccttcggcctcggggcgggcgcctgcgtcggtctccgcgaagcgggaaagcgcggcggccgccgggattcgggcgccgcggcagctgctccggctgccggccggcggccccgcgctcgcccgccccgcttccgcccgctgtcctgctgcacgaacccttccaactctcctttcctcccccacccttgagttacccctctgtctttcctgctgttgcgcgggtgctcccacagcggagcggagattacagagccgccgggatgccccaactctccggaggaggtggcggcggcgggggggacccggaactctgcgccacggacgagatgatccccttcaaggacgagggcgatcctcagaaggaaaagatcttcgccgagatcagtcatcccgaagaggaaggcgatttagctgacatcaagtcttccttggtgaacgagtctgaaatcatcccggccagcaacggacacgaggtggccagacaagcacaaacctctcaggagccctaccacgacaaggccagagaacaccccgatgacggaaagcatccagatggaggcctctacaacaagggaccctcctactcgagttattccgggtacataatgatgccaaatatgaataacgacccatacatgtcaaatggatctctttctccacccatcccgagaacatcaaataaagtgcccgtggtgcagccatcccatgcggtccatcctctcacccccctcatcacttacagtgacgagcacttttctccaggatcacacccgtcacacatcccatcagatgtcaactccaaacaaggcatgtccagacatcctccagctcctgatatccctactttttatcccttgtctccgggtggtgttggacagatcaccccacctcttggctggcaaggtcagcctgtatatcccatcacgggtggattcaggcaaccctacccatcctcactgtcagtcgacacttccatgtccaggttttcccatcatatgattcccggtcctcctggtccccacacaactggcatccctcatccagctattgtaacacctcaggtcaaacaggaacatccccacactgacagtgacctaatgcacgtgaagcctcagcatgaacagagaaaggagcaggagccaaaaagacctcacattaagaagcctctgaatgcttttatgttatacatgaaagaaatgagagcgaatgtcgttgctgagtgtactctaaaagaaagtgcagctatcaaccagattcttggcagaaggtggcatgccctctcccgtgaagagcaggctaaatattatgaattagcacggaaagaaagacagctacatatgcagctttatccaggctggtctgcaagagacaattatggtaagaaaaagaagaggaagagagagaaactacaggaatctgcatcaggtacaggtccaagaatgacagctgcctacatctgaaacatggtggaaaacgaagctcattcccaacgtgcaaagccaaggcagcgaccccaggacctcttctggagatggaagcttgttgaaaacccagactgtctccacggcctgcccagtcgacgccaaaggaacactgacatcaattttaccctgaggtcactgctagagacgctgatccataaagacaatcactgccaacccctctttcgtctactgcaagagccaagttccaaaataaagcataaaaaggttttttaaaaggaaatgtaaaagcacatgagaatgctagcaggctgtggggcagctgagcagcttttctccccccatatctgcgtgcacttcccagagcatcttgcatccaaacctgtaacctttcggcaaggacggtaacttggctgcatttgcctgtcatgcgcaactggagccagcaaccagctatccatcagcaccccagtggaggagttcatggaagagttccctctttgtttctgcttcatttttctttcttttcttttctcctaaagcttttatttaacagtgcaaaaggatcgtttttttttgcttttttaaacttgaatttttttaatttacactttttagttttaattttcttgtatattttgctagctatgagcttttaaataaaattgaaagttctggaaaagtttgaaataatgacataaaaagaagccttctttttctgagacagcttgtctggtaagtggcttctctgtgaattgcctgtaacacatagtggcttctccgcccttgtaaggtgttcagtagagctaaataaatgtaatagccaaaccccactctgttggtagcaattggcagccctatttcagtttattttttcttctgttttcttcttttctttttttaaacagtaaaccttaacagatgcgttcagcagactggtttgcagtgaattttcatttctttccttatcacccccttgttgtaaaaagcccagcacttgaattgttattactttaaatgttctgtatttgtatctgtttttattagccaattagtgggattttatgccagttgttaaaatgagcattgatgtacccattttttaaaaaagcaagcacagcctttgcccaaaactgtcatcctaacgtttgtcattccagtttgagttaatgtgctgagcatttttttaaaagaagctttgtaataaaacatttttaaaaattgtcatttaaaaaaaaaaaaaaaaaa

SEQ ID NO: 66 - protein, 399 aa, Homo sapiens
MPQLSGGGGGGGGDPELCATDEMIPFKDEGDPQKEKIFAEISHPEEEGDLADIKSSLVNESEIIPASNGHEVARQAQTSQEPYHDKAREHPDDGKHPDGGLYNKGPSYSSYSGYIMMPNMNNDPYMSNGSLSPPIPRTSNKVPVVQPSHAVHPLTPLITYSDEHFSPGSHPSHIPSDVNSKQGMSRHPPAPDIPTFYPLSPGGVGQITPPLGWQGQPVYPITGGFRQPYPSSLSVDTSMSRFSHHMIPGPPGPHTTGIPHPAIVTPQVKQEHPHTDSDLMHVKPQHEQRKEQEPKRPHIKKPLNAFMLYMKEMRANVVAECTLKESAAINQILGRRWHALSREEQAKYYELARKERQLHMQLYPGWSARDNYGKKKKRKREKLQESASGTGPRMTAAYI

SEQ ID NO: 67 - DNA, 4306 bp, Homo sapiens
cacacggactacaggggagttttgttgaagttgcaaagtcctggagcctccagagggctgtcggcgcagtagcagcgagcagcagagtccgcacgctccggcgaggggcagaagagcgcgagggagcgcggggcagcagaagcgagagccgagcgcggacccagccaggacccacagccctccccagctgcccaggaagagccccagccatggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgccctctgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccagactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatctgagggcgccaggcaggcgggcgccaccgccacccgcagcgagggcggagccggccccaggtgctcccctgacagtccctcctctccggagcattttgataccagaagggaaagcttcattctccttgttgttggttgttttttcctttgctctttcccccttccatctctgacttaagcaaaagaaaaagattacccaaaaactgtctttaaaagagagagagagaaaaaaaaaatagtatttgcataaccctgagcggtgggggaggagggttgtgctacagatgatagaggattttataccccaataatcaactcgtttttatattaatgtacttgtttctctgttgtaagaataggcattaacacaaaggaggcgtctcgggagaggattaggttccatcctttacgtgtttaaaaaaaagcataaaaacattttaaaaacatagaaaaattcagcaaaccatttttaaagtagaagagggttttaggtagaaaaacatattcttgtgcttttcctgataaagcacagctgtagtggggttctaggcatctctgtactttgcttgctcatatgcatgtagtcactttataagtcattgtatgttattatattccgtaggtagatgtgtaacctcttcaccttattcatggctgaagtcacctcttggttacagtagcgtagcgtggccgtgtgcatgtcctttgcgcctgtgaccaccaccccaacaaaccatccagtgacaaaccatccagtggaggtttgtcgggcaccagccagcgtagcagggtcgggaaaggccacctgtcccactcctacgatacgctactataaagagaagacgaaatagtgacataatatattctatttttatactcttcctatttttgtagtgacctgtttatgagatgctggttttctacccaacggccctgcagccagctcacgtccaggttcaacccacagctacttggtttgtgttcttcttcatattctaaaaccattccatttccaagcactttcagtccaataggtgtaggaaatagcgctgtttttgttgtgtgtgcagggagggcagttttctaatggaatggtttgggaatatccatgtacttgtttgcaagcaggactttgaggcaagtgtgggccactgtggtggcagtggaggtggggtgtttgggaggctgcgtgccagtcaagaagaaaaaggtttgcattctcacattgccaggatgataagttcctttccttttctttaaagaagttgaagtttaggaatcctttggtgccaactggtgtttgaaagtagggacctcagaggtttacctagagaacaggtggtttttaagggttatcttagatgtttcacaccggaaggtttttaaacactaaaatatataatttatagttaaggctaaaaagtatatttattgcagaggatgttcataaggccagtatgatttataaatgcaatctccccttgatttaaacacacagatacacacacacacacacacacacacacaaaccttctgcctttgatgttacagatttaatacagtttatttttaaagatagatccttttataggtgagaaaaaaacaatctggaagaaaaaaaccacacaaagacattgattcagcctgtttggcgtttcccagagtcatctgattggacaggcatgggtgcaaggaaaattagggtactcaacctaagttcggttccgatgaattcttatcccctgccccttcctttaaaaaacttagtgacaaaatagacaatttgcacatcttggctatgtaattcttgtaatttttatttaggaagtgttgaagggaggtggcaagagtgtggaggctgacgtgtgagggaggacaggcgggaggaggtgtgaggaggaggctcccgaggggaaggggcggtgcccacaccggggacaggccgcagctccattttcttattgcgctgctaccgttgacttccaggcacggtttggaaatattcacatcgcttctgtgtatctctttcacattgtttgctgctattggaggatcagttttttgttttacaatgtcatatactgccatgtactagttttagttttctcttagaacattgtattacagatgccttttttgtagttttttttttttttatgtgatcaattttgacttaatgtgattactgctctattccaaaaaggttgctgtttcacaatacctcatgcttcacttagccatggtggacccagcgggcaggttctgcctgctttggcgggcagacacgcgggcgcgatcccacacaggctggcgggggccggccccgaggccgcgtgcgtgagaaccgcgccggtgtccccagagaccaggctgtgtccctcttctcttccctgcgcctgtgatgctgggcacttcatctgatcgggggcgtagcatcatagtagtttttacagctgtgttattctttgcgtgtagctatggaagttgcataattattattattattattataacaagtgtgtcttacgtgccaccacggcgttgtacctgtaggactctcattcgggatgattggaatagcttctggaatttgttcaagttttgggtatgtttaatctgttatgtactagtgttctgtttgttattgttttgttaattacaccataatgctaatttaaagagactccaaatctcaatgaagccagctcacagtgctgtgtgccccggtcacctagcaagctgccgaaccaaaagaatttgcaccccgctgcgggcccacgtggttggggccctgccctggcagggtcatcctgtgctcggaggccatctcgggcacaggcccaccccgccccacccctccagaacacggctcacgcttacctcaaccatcctggctgcggcgtctgtctgaaccacgcgggggccttgagggacgctttgtctgtcgtgatggggcaagggcacaagtcctggatgttgtgtgtatcgagaggccaaaggctggtggcaagtgcacggggcacagcggagtctgtcctgtgacgcgcaagtctgagggtctgggcggcgggcggctgggtctgtgcatttctggttgcaccgcggcgcttcccagcaccaacatgtaaccggcatgtttccagcagaagacaaaaagacaaacatgaaagtctagaaataaaactggtaaaaccccaaaaaaaaaaaaaaaa

SEQ ID NO: 68 - protein, 295 aa, Homo sapiens
MEHQLLCCEVETIRRAYPDANLLNDRVLRAMLKAEETCAPSVSYFKCVQKEVLPSMRKIVATWMLEVCEEQKCEEEVFPLAMNYLDRFLSLEPVKKSRLQLLGATCMFVASKMKETIPLTAEKLCIYTDNSIRPEELLQMELLLVNKLKWNLAAMTPHDFIEHFLSKMPEAEENKQIIRKHAQTFVALCATDVKFISNPPSMVAAGSVVAAVQGLNLRSPNNFLSYYRLTRFLSRVIKCDPDCLRACQEQIEALLESSLRQAQQNMDPKAAEEEEEEEEEVDLACTPTDVRDVDI

SEQ ID NO: 69 - DNA, 1474 bp, Homo sapiens
agccctcccagtttccgcgcgcctctttggcagctggtcacatggtgagggtgggggtgagggggcctctctagcttgcggcctgtgtctatggtcgggccctctgcgtccagctgctccggaccgagctcgggtgtatggggccgtaggaaccggctccggggccccgataacgggccgcccccacagcaccccgggctggcgtgagggtctcccttgatctgagaatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagtgagcaatggagtggctgccatggaaggaagaaaagctgccatttcccttctggacactgagagggcaatctttgcctttatctctgaggctatggagggtcctcctccatctttctacagagattactttgctgccttaatgacattcccctcccacctctccttttgaggcttctccttctccttcccatttctctacactaaggggtatgttccctcttgtccctttccctacctttatatttggggtccttttttatacaggaaaaacaaaacaaagaaataatggtcttttttttttttttaaaaaaaaaaaaaaaaaaaaaaaaaaaaaaaa

SEQ ID NO: 70 - protein, 303 aa, Homo sapiens
MATSRYEPVAEIGVGAYGTVYKARDPHSGHFVALKSVRVPNGGGGGGGLPISTVREVALLRRLEAFEHPNVVRLMDVCATSRTDREIKVTLVFEHVDQDLRTYLDKAPPPGLPAETIKDLMRQFLRGLDFLHANCIVHRDLKPENILVTSGGTVKLADFGLARIYSYQMALTPVVVTLWYRAPEVLLQSTYATPVDMWSVGCIFAEMFRRKPLFCGNSEADQLGKIFDLIGLPPEDDWPRDVSLPRGAFPPRGPRPVQSVVPEMEESGAQLLLEMLTFNPHKRISAFRALQHSYLHKDEGNPE

SEQ ID NO: 71 - DNA, 897 bp, Homo sapiens
tgtgtgggggtctgcttggcggtgagggggctctacacaagcttcctttccgtcatgccggcccccaccctggctctgaccattctgttctctctggcaggtcatgatgatgggcagcgcccgagtggcggagctgctgctgctccacggcgcggagcccaactgcgccgaccccgccactctcacccgacccgtgcacgacgctgcccgggagggcttcctggacacgctggtggtgctgcaccgggccggggcgcggctggacgtgcgcgatgcctggggccgtctgcccgtggacctggctgaggagctgggccatcgcgatgtcgcacggtacctgcgcgcggctgcggggggcaccagaggcagtaaccatgcccgcatagatgccgcggaaggtccctcagacatccccgattgaaagaaccagagaggctctgagaaacctcgggaaacttagatcatcagtcaccgaaggtcctacagggccacaactgcccccgccacaacccaccccgctttcgtagttttcatttagaaaatagagcttttaaaaatgtcctgccttttaacgtagatataagccttcccccactaccgtaaatgtccatttatatcattttttatatattcttataaaaatgtaaaaaagaaaaacaccgcttctgccttttcactgtgttggagttttctggagtgagcactcacgccctaagcgcacattcatgtgggcatttcttgcgagcctcgcagcctccggaagctgtcgacttcatgacaagcattttgtgaactagggaagctcaggggggttactggcttctcttgagtcacactgctagcaaatggcagaaccaaagctcaaataaaaataaaataattttcattcattcactc

SEQ ID NO: 72 - protein, 105 aa, Homo sapiens
MMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSDIPD

SEQ ID NO: 73 - DNA, 1515 bp, Homo sapiens
cccaacctggggcgacttcaggtgtgccacattcgctaagtgctcggagttaatagcacctcctccgagcactcgctcacggcgtccccttgcctggaaagataccgcggtccctccagaggatttgagggacagggtcggagggggctcttccgccagcaccggaggaagaaagaggaggggctggctggtcaccagagggtggggcggaccgcgtgcgctcggcggctgcggagagggggagagcaggcagcgggcggcggggagcagcatggagccggcggcggggagcagcatggagccggcggcggggagcagcatggagccttcggctgactggctggccacggccgcggcccggggtcgggtagaggaggtgcgggcgctgctggaggcgggggcgctgcccaacgcaccgaatagttacggtcggaggccgatccaggtgggtagaaggtctgcagcgggagcaggggatggcgggcgactctggaggacgaagtttgcaggggaattggaatcaggtagcgcttcgattctccggaaaaaggggaggcttcctggggagttttcagaaggggtttgtaatcacagacctcctcctggcgacgccctgggggcttgggaaaccaaggaagaggaatgaggagccacgcgcgtacagatctctcgaatgctgagaagatctgaaggggggaacatatttgtattagatggaagtcatgatgatgggcagcgcccgagtggcggagctgctgctgctccacggcgcggagcccaactgcgccgaccccgccactctcacccgacccgtgcacgacgctgcccgggagggcttcctggacacgctggtggtgctgcaccgggccggggcgcggctggacgtgcgcgatgcctggggccgtctgcccgtggacctggctgaggagctgggccatcgcgatgtcgcacggtacctgcgcgcggctgcggggggcaccagaggcagtaaccatgcccgcatagatgccgcggaaggtccctcagacatccccgattgaaagaaccagagaggctctgagaaacctcgggaacttagatcatcagtcaccgaaggtcctacagggccacaactgcccccgccacaacccaccccgctttcgtagttttcatttagaaaatagagcttttaaaaatgtcctgccttttaacgtagatatatgccttcccccactaccgtaaatgtccatttatatcattttttatatattcttataaaaatgtaaaaaagaaaaacaccgcttctgccttttcactgtgttggagttttctggagtgagcactcacgccctaagcgcacattcatgtgggcatttcttgcgagcctcgcagcctccggaagctgtcgacttcatgacaagcattttgtgaactagggaagctcaggggggttactggcttctcttgagtcacactgctagcaaatggcagaaccaaagctcaaataaaaataaaataattttcattcattcactc

SEQ ID NO: 74 - protein, 116 aa, Homo sapiens
MEPAAGSSMEPSADWLATAAARGRVEEVRALLEAGALPNAPNSYGRRPIQVGRRSAAGAGDGGRLWRTKFAGELESGSASILRKKGRLPGEFSEGVCNHRPPPGDALGAWETKEEE

SEQ ID NO: 75 - DNA, 2140 bp, Homo sapiens
agctgaggtgtgagcagctgccgaagtcagttccttgtggagccggagctgggcgcggattcgccgaggcaccgaggcactcagaggaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtactggagcagaccaccccgcctgccctcatggcccctctgacctgcactggggagcccgtctcagtgttgagccttttccctctttggctcccctgtaccttttgaggagccccagcttacccttcttctccagctgggctctgcaattcccctctgctgctgtccctcccccttgtctttcccttcagtaccctctcatgctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttaaaaaaaaaaaaaaaaaaa

SEQ ID NO: 76 - protein, 164 aa, Homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 77 - DNA, 2986 bp, Homo sapiens
ctcacggctctgcgactccgacgccggcaaggtttggagagcggctgggttcgcgggacccgcgggcttgcacccgcccagactcggacgggctttgccaccctctccgcttgcctggtcccctctcctctccgccctcccgctcgccagtccatttgatcagcggagactcggcggccgggccggggcttccccgcagcccctgcgcgctcctagagctcgggccgtggctcgtcggggtctgtgtcttttggctccgagggcagtcgctgggcttccgagaggggttcgggccgcgtaggggcgctttgttttgttcggttttgtttttttgagagtgcgagagaggcggtcgtgcagacccgggagaaagatgtcaaacgtgcgagtgtctaacgggagccctagcctggagcggatggacgccaggcaggcggagcaccccaagccctcggcctgcaggaacctcttcggcccggtggaccacgaagagttaacccgggacttggagaagcactgcagagacatggaagaggcgagccagcgcaagtggaatttcgattttcagaatcacaaacccctagagggcaagtacgagtggcaagaggtggagaagggcagcttgcccgagttctactacagacccccgcggccccccaaaggtgcctgcaaggtgccggcgcaggagagccaggatgtcagcgggagccgcccggcggcgcctttaattggggctccggctaactctgaggacacgcatttggtggacccaaagactgatccgtcggacagccagacggggttagcggagcaatgcgcaggaataaggaagcgacctgcaaccgacggtaatgaccctttcccaaccatagaatgtgtttggggccccgctttgcctgctggagggtgttaaccttagcttgcttttcggcgtattctgatttagctttgggagagctaactttattggtcttaggtgttcagtgctacctggcccactgcttgtctgtttgtgacttttaagtcagaaactggagatggtaagatccgataatttccctaacttaatacatcgcggtccctctcactagcaactcctaggtatgtgacaaagttgggatgtttatcaacggtccgcctcctggctagggaaagagctctggggcggagaatgcactttctgttttttgaaaacaacctcattttgtgcccttaaaagccactggggatgacggatccaggattgtgggtggaggtagtgggtttttcatcccctgactatggggccaacttctgccagccattgttttttctaataaagattgtgtgttctttttaaaaatttcccctgcgcttagattcttctactcaaaacaaaagagccaacagaacagaagaaaatgtttcagacggttccccaaatgccggttctgtggagcagacgcccaagaagcctggcctcagaagacgtcaaacgtaaacagctcggtgggttgatcactaaaggagcacgcactggaacccggggccttcagacctcacgatacctgatcttactggttgctggcaaattaaaagcttatggggttttgttttgtttatacttcgtgaggtcaaaaaagtagcaatggggaaggctggggatacggtaattcctcagagtttctatgcccagagatactttctcttcaaactgttgaccagagcagctacttgtaacccaggccccatcgggtaggaaggtcgtttccctgtgagtcccactaaaacgtgttgggagcaataggttctttgcccatccgaacaagaactagggtactccctcagtccgaattaatgagaattaatttcctagaggttcagcttgagtcggtaacagattttgagccatacatggaaaaatggcaaatacatgattaagtttcaattttgagggggaatgtttggtagaaattgctcatctttggttatgcaagggattagagatgtgaataggatggtatgttgtgttctttgacattttaataaactgtcactttccctgttgtctcctaagtttggagagagaaggaaccagtatttgcaaaaaaccaaatggaaagataaaaaagttactaaagtttctacagaatttctggtaacactgaagttgcaaagcagaagttaaattaactcttgtcagtaagcaatccaggaacacgtcagccagtgtatgctaattgtgccgtaacagggtgatttggatatttgtaggggaaatgggtagtaaatatcaagactggtgaccgtaggtcagcccagcacaaaggaagtggagatttttccatgcacaagaatctgatcactgtaaatagctaatttgaataattcagtccccagataaccaacatgggttggttattcataataaactacatattttaatagtttattagcttcctttagaccaagactgtgacctctttattttctaaagcacacacgtagtttagcatatgaggcgataaaatattgatgttaactttttaaatccccagttataaaaattttaaaataacagggattaaggtgagattcaggtttgttgtgtctttaaattgtatatgtgacttcacatatctttttcagcgcttatacaaaacggcactatagaacctccattttacagcaccatatgaagtgggaaaattaggtgaaaattttcctgaagcaaccttaacatgcgcagccagcccttgttggtttgtgacttgtggcctagctcatcagatgagccacgagaatcagacctggattttgatctggccctgttctgacatgcaatgaggcatttgtagcatttagtaatattgctagttcaaagaatactagaaatattagtaagaacctattcaaaagtattcatgagtattttctgcatatgaatcaggaattagaatattttgaaaatgatgttaataaaattttcctctggaaggcctttata

SEQ ID NO: 78 - protein, 198 aa, Homo sapiens
MSNVRVSNGSPSLERMDARQAEHPKPSACRNLFGPVDHEELTRDLEKHCRDMEEASQRKWNFDFQNHKPLEGKYEWQEVEKGSLPEFYYRPPRPPKGACKVPAQESQDVSGSRPAAPLIGAPANSEDTHLVDPKTDPSDSQTGLAEQCAGIRKRPATDDSSTQNKRANRTEENVSDGSPNAGSVEQTPKKPGLRRRQT

(19) United States
(12) Patent Application Publication
(10) Pub. No.: US 2008/0227738 A1
(43) Pub. Date: Sep. 18, 2008

(54) Compositions and methods for cell dedifferentiation and tissue regeneration

(75) Inventors: Mark T. Keating, Chestnut Hill, MA (US); Shannon J. Odelberg, Salt Lake City, UT (US); Kenneth D. Poss, Chapel Hill, NC (US)

Correspondence Address: ROPES & GRAY LLP, PATENT DOCKETING 39/41, ONE INTERNATIONAL PLACE, BOSTON, MA, 02110-2624, US

(73) Assignee: University of Utah Research Foundation, Salt Lake City, UT (US)

(21) Appl. No.: 11/880,435

(22) Filed: Jul. 20, 2007

Related U.S. Application Data
(63) Continuation of application No. 10/302,812, filed on Nov. 22, 2002, now abandoned.
Application No. 10/302,812 is a continuation-in-part of application No. 10/275,828, filed on Apr. 4, 2003, now abandoned.
(60) Provisional application No. 60/204,080, filed on May 12, 2000.
(60) Provisional application No. 60/204,081, filed on May 12, 2000.
(60) Provisional application No. 60/204,082, filed on May 12, 2000.

(51) Int. Cl. A61K 31/70; C12N 5/06; C12Q 1/02; A61P 43/00

(52) U.S. Cl. 514/44; 435/377; 435/29

(57) ABSTRACT

The present invention provides methods and compositions to dedifferentiate a cell. The ability of the methods and compositions of the present invention to promote the dedifferentiation of differentiated cells, including terminally differentiated cells, can be used to promote regeneration of tissues and organs in vivo. The ability of the methods and compositions of the present invention to promote the dedifferentiation of differentiated cells, including terminally differentiated cells, can further be used to produce populations of stem or progenitor cells which can be used to promote regeneration of tissues and/or organs damaged by injury or disease. Accordingly, the present invention provides novel methods for the treatment of a wide range of injuries and diseases that affect many diverse cell types.

### RELATED APPLICATIONS

This application is a continuation-in-part of U.S. patent application Ser. No. 10/275,828, international filing date May 14, 2001, which is a national stage filing under 35 U.S.C. 371 of PCT application PCT/US01/5582, filed May 14, 2001, which claims the benefit of priority from U.S. Provisional Application Nos.: 60/204,080; 60/204,081; and 60/204,082; all filed May 12, 2000, the specifications of all of which are incorporated by reference herein in their entirety. PCT Application PCT/US01/15582 was published under PCT Article 21(2) in English.

### BACKGROUND OF THE INVENTION

The present invention is directed to compositions that promote cellular dedifferentiation and tissue regeneration. It also is directed to methods of inducing cellular dedifferentiation, proliferation, and regeneration.

Morgan (Morgan, 1901) coined the term epimorphosis to refer to the regenerative process in which cellular proliferation precedes the development of a new anatomical structure. Adult urodeles, e.g., newts or axolotls, are known to be capable of regenerating limbs, tail, upper and lower jaws, retinas, eye lenses, dorsal crest, spinal cord, and heart ventricles (Becker et al., 1974; Brockes, 1997; Davis et al., 1990), while teleost fish, such as Danio rerio, (zebrafish), are known to regenerate their fins and spinal cord (Johnson and Weston, 1995; Zottoli et al., 1994). Echinoderms and crustaceans are likewise capable of regeneration. However, with the exception of liver, mammals, such as humans, lack this remarkable regenerative capability.

Mammals typically heal an injury, whether induced from trauma or degenerative disease, by replacing the missing tissue with scar tissue. Wound healing, which is distinct from tissue regeneration, results in scar tissue that has none of the specific functions of the cell types that it replaced, except the qualities of tissue integrity and strength. For example, cardiac injuries, such as from a heart attack, result in cardiac muscle that dies. Instead of new cardiac muscle replacing the dead cells, scar tissue forms. The burden of contraction, once shouldered by the now missing cells, is passed on to surrounding areas, thus increasing the workload of existing cells. For optimal cardiac performance, the dead tissue would need to be replaced with cardiac cells (regeneration).

The molecular and cellular mechanisms that govern epimorphic regeneration are incompletely defined. The first step in this process is the formation of a wound epithelium, which occurs within the first 24 hours following amputation. The second step involves the dedifferentiation of cells proximal to the amputation plane. These cells proliferate to form a mass of pluripotent cells, known as the regeneration blastema, which will eventually redifferentiate to form the lost structure. Although cellular dedifferentiation has been demonstrated in newts, terminally-differentiated mammalian cells are thought to be incapable of reversing the differentiation process (Andres and Walsh, 1996; Walsh and Perlman, 1997). Several mechanisms could explain the lack of cellular plasticity in mammalian cells: (1) the extracellular factors that initiate dedifferentiation are not adequately expressed following amputation; (2) the intrinsic cellular signaling pathways for dedifferentiation are absent; (3) differentiation factors are irreversibly expressed in mammalian cells; and (4) structural characteristics of mammalian cells make dedifferentiation impossible.

Though differentiated, newt myotubes are not locked into a Gₒ/G₁ state (Hay and Fischman, 1961; Tanaka et al., 1997) and thus are capable of dedifferentiation. In contrast, mammalian skeletal muscle cells are thought to be terminally-differentiated (Andres and Walsh, 1996; Walsh and Perlman, 1997). Normal (non-transformed, non-oncogenic) mammalian myotubes have not been observed to reenter the cell cycle or dedifferentiate in vitro or in vivo. In contrast, oncogenic mammalian cells have been observed to re-enter the cell cycle and proliferate (Endo and Nadal-Ginard, 1989; Endo and Nadal-Ginard, 1998; Iujvidin et al., 1990; Novitch et al., 1996; Schneider et al., 1994; Tiainen et al., 1996). However, these cells are abnormal and cannot participate in regeneration. The ability to dedifferentiate non-oncogenic mammalian cells is a long-sought goal, which the current invention achieves.

While artificial organs, organ transplants, prostheses and other means to substitute for missing tissues, organs, and appendages have improved the quality of life of many who suffer from a wide range of diseases and injuries, the current methods used to create such organs and prostheses are fraught with complications and high costs. For example, those lucky enough to receive tissue and organ transplants must be administered expensive anti-rejection drugs for the life of the transplant. In addition to their expense, prostheses suffer from an inability to replace the full function of the missing appendage.

In addition, current bio-mediated tissue and organ replacement techniques also suffer from significant disadvantages. Tissue engineering, the approach of replacing tissue by culturing cells in vitro onto a biomaterial substrate and then transplanting to an individual (a mammalian, preferably a human, subject), is hampered by cost and time. Additionally, such tissue engineering approaches often result in formation of a structure that does not have all of the intrinsic functions and morphology of the tissue it replaces. Likewise, an approach that exploits stem cells ex vivo is similarly hampered by cost and time. Stem cells must be purified from bone marrow, aborted fetuses, or other appropriate sources, manipulated in vitro, and then introduced into an individual. In addition to the high costs likely involved in currently contemplated stem cell based approaches, such methods' also present significant practical, ethical and regulatory limitations in terms of finding a readily accessible source of stem cells.

The current invention overcomes the limitations of the prior art, and provides methods and compositions for dedifferentiating cells in vivo or in vitro. Methods for dedifferentiating cells allow, for the first time, the development of methods to regenerate mammalian tissues that resemble the endogenous tissues that were damaged by injury or disease. The methods and compositions detailed herein have a diverse range of applications and offer unique treatments for injuries and diseases for which there are currently few satisfactory therapeutic options.

### BRIEF SUMMARY OF THE INVENTION

The invention provides compositions and methods for dedifferentiating cells in vivo and in vitro. The invention also provides compositions and methods for the regeneration of cells, tissue and organs in vivo and in vitro. The present inventors have now discovered that an extract from newt, as Well as purified components therefrom, can be used to achieve this and other objectives as discussed herein.

In one aspect, the invention provides a method of dedifferentiating a differentiated mammalian cell. The method comprises administering an amount of one or more agents effective to promote dedifferentiation of a differentiated mammalian cell. Agents for use in this method have one or more of the following functions: increase the expression and/or activity of a G1 Cdk complex, decrease expression of one or more markers of differentiation, promote cell cycle reentry, or increase the expression of one or more progenitor or stem cell markers.

In a second aspect, the invention provides a method of regenerating mammalian cells, tissues and/or organs. The method comprises contacting differentiated mammalian cells with an amount of an agent effective to dedifferentiate said mammalian cells. Following dedifferentiation, the dedifferentiated mammalian cells are capable of redifferentiating to regenerate said mammalian cells, tissues and/or organs.

In a third aspect, the invention provides a method of screening to identify and/or characterize a dedifferentiation agent. The method comprises contacting a cell with one or more agents, and comparing dedifferentiation of said cell in the presence of said one or more agents in comparison to the absence of said one or more agents. An agent that promotes dedifferentiation of a cell is a dedifferentiation agent.

In a fourth aspect, the invention provides a kit comprising one or more dedifferentiation agents, and instructions for their use.

In a fifth aspect, the invention provides pharmaceutical compositions of one or more dedifferentiation agents formulated in a pharmaceutically acceptable carrier.

In a sixth aspect, the invention provides a method of conducting a drug discovery business.

In a seventh aspect, the invention provides a method of conducting a regenerative medicine business.

In an eighth aspect, the invention provides a method of conducting a gene therapy business.

In a ninth aspect, the invention provides use of an agent which increases the mitotic activity of a G1 Cdk complex in the manufacture of a medicament for promoting dedifferentiation of differentiated mammalian cells.

In a tenth aspect, the invention provides use of an expression construct encoding a protein or transcript which upregulates the activity of a G1 phase cyclin dependent kinase (cdk) in the manufacture of medicament for causing dedifferentiation of cells in a patient.

In an eleventh aspect, the invention provides a packaged pharmaceutical comprising: a preparation of expression constructs encoding a protein or transcript which upregulates the activity of a G1 phase cyclin dependent kinase (cdk); a pharmaceutically acceptable carrier, and instructions, written and/or pictorial, describing the use of the preparation for causing dedifferentiation of cells in a patient.

### DETAILED DESCRIPTION OF THE INVENTION

The present invention provides methods and compositions for dedifferentiating cells. Although previously thought to be committed to their differentiated fate, differentiated cells can be dedifferentiated. In certain embodiments, terminally differentiated cells can be dedifferentiated. The compositions for use in the methods of the present invention to dedifferentiate a cell include, but are not limited to, peptides, polypeptides, nucleic acids, small organic molecules, antisense oligonucleotides, RNAi constructs, ribozymes, antibodies, or combinations of these. As used herein, any agent capable of dedifferentiating at least one cell type is a dedifferentiation factor. Furthermore, compositions for use in the methods of the present invention include regeneration extracts. Such extracts are derived from a regenerating tissue (e.g., a regenerating newt limb) and are capable of inducing dedifferentiation. These extracts must comprise at least one dedifferentiation factor, however it is recognized that extracts may be used to dedifferentiate cells with or without knowledge as to the identity of the specific components in the extract that mediate dedifferentiation. The invention contemplates that dedifferentiation factors, either isolated factors or extracts containing dedifferentiation factors, can be used to dedifferentiate cells in vitro, in vivo, and ex vivo. The invention further contemplates that agents that dedifferentiate one or more cell type can be used to regenerate damaged cells and/or tissues. The invention still further contemplates that methods and compositions that promote the regeneration of damaged cells and tissues, whether those cells were damaged by disease or injury, can be used in the treatment of a vast array of diseases and injuries.

Based on the results summarized herein which demonstrated that terminally differentiated mammalian cells can be induced to dedifferentiate, the present invention contemplates the use of a variety of dedifferentiation agents. Such dedifferentiation agents include nucleic acids, peptides, polypeptides, small organic molecules, antibodies, antisense oligonucleotides, RNAi constructs, or ribozymes, and dedifferentiation may be achieved by contacting a cell, in vivo or in vitro, with one or more dedifferentiation factors for a time sufficient to induce dedifferentiation. Methods for promoting dedifferentiation provide, for the first time, methods of promoting regeneration of mammalian cells and tissues damaged by injury or disease.

Without being bound by theory, the present invention contemplates a number of methods and compositions which can be used to dedifferentiate a cell. Exemplary dedifferentiation factors include “regeneration extracts” (RE; referring to an extract from any animal that regenerates, preferably newt, most preferably, RNLE, hRNLE, and RNLE-purified components), growth factors (GFs), msx1, msx2, BMPs, Wnts, FGFs, cyclinD1, and Cdk4. Additionally, the invention contemplates that msx1, msx2, BMPs, Wnts, FGFs, cyclinD1, and Cdk4 are components of various signaling pathways, and thus further exemplary dedifferentiation factors include one or more agents that promote BMP signaling, Wnt signaling, or FGF signaling, or one or more agents that relieve an inhibitor of any of these signaling pathways. Furthermore, dedifferentiation agents include agents which promote msx1 or msx2 expression, agents which inhibit msx3 expression, agents that promote cyclinD1 expression and/or activity, agents that promote Cdk4 expression and/or activity, and agents that inhibit p16 and/or p21 expression and/or activity. Any of the above cited agents which promote dedifferentiation are also referred to throughout as Regeneration/Dedifferentiation Factors (RDF) or Dedifferentiation Factors.

### I. Embodiments

The following embodiments are given as examples of various ways to practice the invention. Many different versions will be immediately apparent to one of skill in the various arts to which this invention pertains.

### A. In Vivo

The compositions of the invention can be used in vivo to dedifferentiate cells. A cell is contacted with an amount of one or more dedifferentiation agents effective to dedifferentiate the cell. Dedifferentiation in vivo can be measured by any of a number of methods including, but not limited to, assaying a decrease in expression of one or more markers of differentiation (e.g., markers of differentiation specific to the particular cell type), assaying an increase in proliferation, assaying an increase in expression of markers of a progenitor cell phenotype, observing changes in cell behavior and/or morphology.

In one embodiment, in vivo dedifferentiation occurs at a site of injury or disease. Without being bound by theory, injury is an early step in regeneration in organisms and cell types that endogenously use regeneration to repair cell and tissue damage. Accordingly, it is possible that factors present at the site of injury may bias a cell toward dedifferentiation. Dedifferentiation of cells at the site of an injury, whether trauma or disease-induced, is an early step in the regeneration of cells, tissue and organs.

Whether the methods of the present invention are used to dedifferentiate cells in vivo at a site of injury, or at another site that has not been damaged by injury or disease, the end result is the same: dedifferentiated cells have regressed in a developmental pathway. In one embodiment, such cells may resemble pluripotent, or even totipotent, stem cells. In another embodiment, such cells have dedifferentiated and regressed to an earlier developmental time but do not resemble stem cells.

To further illustrate, regenerating newt limb extract (RNLE), its humanized form (hRNLE), dedifferentiation factors purified from RNLE, one or more dedifferentiation factors, one or more agents that promote signal transduction through a signal transduction pathway that increases dedifferentiation, or one or more agents that inhibit expression or activity of a factor that inhibits dedifferentiation is applied or administered to an animal in vivo. In one embodiment, the one or more agents are administered at the site of injury. Administration at the site of injury can be at the time of, or soon after injury. In some cases, these compositions may be applied to an injury after some healing with scar tissue has occurred. If healing has already begun to occur, the method of inducing dedifferentiation may optionally include re-injuring.

When the dedifferentiation factor comprises more than one component, these components may be administered at the same time or sequentially. Moreover, the specific route of administration of the agent or agents will differ based on the location to which the agent is delivered, as well as the specific agent being administered (e.g., nucleic acid, polypeptide, small organic molecule, antibody, etc). Furthermore, application of the particular agent or agents may be continuous, instant, or re-applied over a time course during dedifferentiation.

Without being bound by theory, following application of one or more dedifferentiation factors, and subsequent dedifferentiation of cells in vivo, the dedifferentiated cells can redifferentiate to help repair cellular damage. Such redifferentiation may be promoted entirely by in vivo signals, or redifferentiation along a desired developmental path may be further influenced by administration of redifferentiation factors (one or more agents that influence differentiation of dedifferentiated cells along a particular developmental fate).

As outlined above, dedifferentiation agents also include particular growth factors, including but not limited to, FGF, IGF-1′, and IGF-II. Exemplary growth factors include members of the FGF family, including but not limited to FGF2 (SEQ ID NO: 30), FGF4 (SEQ ID NO: 32), FGF8 (SEQ ID NO: 34), FGF10 (SEQ ID NO: 36), FGF 17 (SEQ ID NO: 38) and FGF 18 (SEQ ID NO: 40). The invention contemplates the use of nucleic acids encoding one or more FGF family members, polypeptides corresponding to one or more FGF family members, and agents which promote FGF signaling. Exemplary agents that promote FGF signaling include small organic molecules that bind to FGF and increase, for example, its affinity for an FGF receptor, small organic molecules that bind to an FGF receptor (FGFR) and promote FGF signal transduction, or small organic molecules that bind to an intracellular component of the FGF pathway and promote FGF signaling.

There are currently over 20 mammalian FGFs and these growth factors signal via one or more of four identified FGF receptors (FGFR). The amino acid sequences corresponding to human FGFR1, 2, 3 and 4 are provided in SEQ ID NO: 42, 44, 46 and 48 respectively. Although FGF signaling typically requires the binding of an FGF family member to an FGFR, mutations can be made in the FGFR that cause the receptor to either be unresponsive to signaling (e.g., dominant negative FGFR) or to promote signaling independent of the presence of bound ligand (e.g., activated FGFR). The present invention contemplates that nucleic acids and polypeptides corresponding to an activated FGFR, for example, an activated FGFR1, FGFR2, FGFR3, or FGFR4, can be a dedifferentiation factor, and can be used to dedifferentiate cells in vivo.

Further dedifferentiation agents include BMP family members. Exemplary BMP family members include BMP2 (SEQ ID NO: 18 and 20), BMP4 (SEQ ID NO: 22 and 24) and BMP7 (SEQ ID NO: 26 and 28). The invention contemplates the use of nucleic acids encoding one or more BMP family member, polypeptides corresponding to one or more BMP family member, agents which promote BMP signaling, and agents that decrease the expression and/or activity of one or more inhibitor of BMP signaling. Exemplary agents that promote BMP signaling include small organic molecules that bind to one or more BMP polypeptide and increase, for example, its affinity for a BMP receptor, small organic molecules that bind to a BMP receptor and promote BMP signal transduction, or small organic molecules that bind to an intracellular component of the BMP pathway and promote BMP signaling. Intracellular components of the BMP signaling pathway that may be manipulated (e.g., through overexpression of the corresponding nucleic acid or polypeptide, or via manipulation of a small organic molecule that binds to the intracellular component and promotes BMP signaling) include SMADs (e.g., SMAD1 (GenBank Accession No. U59423), SMAD2 (GenBank Accession No. AF027964), SMAD4 (GenBank Accession No. NM_{—}005359)).

Additionally, BMP signaling is modulated by a family of negative regulators including gremlin (see, for example, Gen Bank Accession No. AF110137), noggin (see, for example, Gen Bank Accession No. NM_{—}005450), follistatin (see, for example, Gen Bank Accession No. AH001463), and chordin (see, for example, Gen Bank Accession Nos. AF209928, AF283325, AF209930, AF209929). Administration of an agent that decreases the expression and/or activity of gremlin, noggin, follistatin and/or chordin would increase BMP signaling. Agents that decrease the expression and/or activity of gremlin, noggin, follistatin, and/or chordin include small organic molecules that bind to and inhibit the expression and/or activity of one or more of gremlin, noggin, follistatin or chordin, antisense oligonucleotides that hybridize under stringent conditions to a nucleic acid encoding, gremlin, noggin, follistatin or chordin; RNAi constructs that hybridize under stringent conditions to a nucleic acid encoding, gremlin, noggin, follistatin or chordin; ribozymes that bind to and inhibit the expression and/or activity of gremlin, noggin, follistatin or chordin; and antibodies that bind to and inhibit the activity of gremlin, noggin, follistatin or chordin.

Further dedifferentiation agents include Wnt family members. Exemplary Wnt family members include, but are not limited to, Wnt1 (SEQ ID NO: 50), Wnt2 (SEQ ID NO: 52), Wnt3 (SEQ ID NO: 54), Wnt5a (SEQ ID NO: 56), Wnt8 (SEQ-ID NO: 58), and Wnt11 (SEQ ID NO: 60). The invention contemplates the use of nucleic acids encoding one or more Wnt family member, polypeptides corresponding to one or more Wnt family member, agents which promote Wnt signaling, and agents that decrease the expression and/or activity of one or more inhibitor of Wnt signaling. Exemplary agents that promote Wnt signaling include small organic molecules that bind to one or more Wnt polypeptide and increase, for example, its affinity for a Wnt receptor, small organic molecules that bind to a Wnt receptor (e.g., frizzled) and promote Wnt signal transduction, or small organic molecules that bind to an intracellular component of the Wnt pathway and promote Wnt signaling. Intracellular components of the Wnt signaling pathway that may be manipulated (e.g., through overexpression of the corresponding nucleic acid or polypeptide, or via manipulation of a small organic molecule that binds to the intracellular component and promotes Wnt signaling) include disheveled, β-catenin (SEQ ID NO: 64), and Lef1 (SEQ ID NO: 66).

Additionally, Wnt signaling can be negatively regulated at several levels. For example, a family of extracellular factors exist that resemble the Wnt receptor frizzled. These extracellular factor include FrzA, Frzb, and sizzled. Because these extracellular factors resemble Wnt receptors, Wnt polypeptides may bind to these factors. However, this binding does not result in activation of Wnt signal transduction. Exemplary human homologs of these extracellular factors are provided in Gen Bank Accession Nos. NM_{—}003012 and NM_{—}001463. Accordingly, the present invention contemplates that agents that inhibit the expression and/or activity of one or more Frzb family extracellular factors would increase Wnt signaling. Agents that decrease the expression and/or activity of one or more Frzb family members include small organic molecules that bind to and inhibit the expression and/or activity of one or more Frzb family members; antisense oligonucleotides that hybridize under stringent conditions to a nucleic acid encoding a Frzb family member; RNAi constructs that hybridize under stringent conditions to a nucleic acid encoding a Frzb family member, ribozymes that bind to and inhibit the expression and/or activity of Frzb family members; and antibodies that bind to and inhibit the activity of a Frzb family member.

In addition to negative regulation of Wnt signaling extracellularly, Wnt signaling is regulated intracellularly by GSK3β (SEQ ID NO: 62). Accordingly, the invention contemplates that agents which inhibit the expression and/or activity of GSK3β can promote Wnt signaling. Exemplary agents that inhibit the expression and/or activity of GSK3β include a nucleic acid or polypeptide corresponding to a dominant negative GSK3β, a small organic molecule that binds to and inhibits expression and/or activity of GSK3β, an antisense oligonucleotide that hybridizes under stringent conditions to a nucleic acid encoding GSK3β (SEQ ID NO: 61), an RNAi construct that hybridizes under stringent conditions to a nucleic acid encoding GSK3β (SEQ ID NO: 61), or an antibody that binds to and inhibits expression and/or activity of GSK3β.

The invention further contemplates the use of particular intracellular factors. Exemplary intracellular factors include msx1 and msx2. Exemplary agents include nucleic acids encoding msx1 and/or msx2 (for example, SEQ ID NO: 1, 3, 5, 7, 9, 11, or 13), polypeptides corresponding to msx1 and/or msx2 (for example, SEQ ID NO: 2, 4, 6, 8, 10, 12, or 14). Further exemplary agents include nucleic acids, peptides, polypeptides, and small organic molecules that induce the expression of msx1 and/or msx2, or increase the activity of msx1 and/or msx2.

In addition to agents that promote expression and/or activity of msx1 and/or msx2, the invention contemplates that agents which inhibit the expression or activity (e.g., antagonists of msx1 and/or rsx2) can be used to promote dedifferentiation. By way of example, msx3 (see, for example, SEQ ID NO: 16) is known to inhibit the activity of msx1, and possibly of msx2. Accordingly, methods that decrease the expression and/or activity of msx3 can be used to effectively increase the activity of msx1 or msx2. Exemplary agents that inhibit the expression and/or activity of msx3 include small organic molecules that bind to and inhibit expression and/or activity of msx3, antisense oligonucleotides that hybridize under stringent conditions to SEQ ID. NO: 16, RNAi constructs that hybridize under stringent conditions to SEQ ID NO: 16, and antibodies that bind to and inhibit the activity and/or expression of msx3.

The invention contemplates that any of the dedifferentiation agents described herein can be administered alone, or in combination with one or more additional dedifferentiation agent. Such combinations of dedifferentiation agents can promote dedifferentiation via the same mechanism (e.g., two or more agents which promote dedifferentiation by promoting expression of msx1 and/or msx2). Similarly, combinations of dedifferentiation agents can promote dedifferentiation via separate mechanisms (e.g., one or more agents which promote dedifferentiation by promoting expression of msx1 and/or msx2 plus one or more agents which promote dedifferentiation by promoting Wnt signal transduction). When the invention provides methods of dedifferentiating cells by administering combinations of agents, one of skill in the art will appreciate that the agents can be administered simultaneously or consecutively.

### B. Ex Vivo/In Vitro

The invention contemplates that any of the dedifferentiation factors outlined above for administration to promote dedifferentiation in vivo can be used to promote dedifferentiation in vitro/ex vivo.

The compositions and methods of the invention may be applied to a procedure wherein differentiated cells are removed from the a subject, dedifferentiated in culture, and then either reintroduced into that individual or, while still in culture, manipulated to redifferentiate along specific differentiation pathways (e.g., adipocytes, chondrocytes, neurons, glia, osteogenic cells, skeletal muscle, cardiac muscle, etc). Such redifferentiated cells could then be introduced to the individual. In one embodiment, the method comprises removing differentiated cells from an injured subject. Cells dedifferentiated from cells harvested from an injured subject can later be returned to the injured subject to treat an injury or degenerative disease. The dedifferentiated cells can be reintroduced at the cite or injury, or the cells can be reintroduced at a cite distant from the injury. Similarly, cells can be harvested from an injured subject, dedifferentiated in vitro, redifferentiated in vitro, and transplanted back to the subject to treat an injury or degenerative disease.

The invention contemplates that the in vitro methods described herein can be used for autologous transplantation of dedifferentiated or redifferentiated cells (e.g., the cells are harvested from and returned to the same individual). The invention further contemplates that the in vitro methods described herein can be used for non-autologous transplantations. In one embodiment, the transplantation occurs between a genetically related donor and recipient. In another embodiment, the transplantation occurs between a genetically un-related donor and recipient. In any of the foregoing embodiments, the invention contemplates that dedifferentiated cells can be expanded in culture and stored for later retrieval and use. Similarly, the invention contemplates that redifferentiated cells can be can be expanded in culture and stored for later retrieval and use.

Cells may be removed from a subject by any method known in the medical arts that is appropriate to the location of the desired cells. Cells are then cultured in vitro, where they may be dedifferentiated using any of the methods and compositions of the invention, including applying one or more of any of the dedifferentiation factors described in detail herein. Any cell culture methods known in the arts may be used, or if unknown, one of skill in the art may easily determine the appropriate culture conditions. If desired, the cells may be expanded before reintroducing back to an individual. In one example, the individual has an injury or degenerative disease, and the dedifferentiated or redifferentiated cells are reintroduced at a site of injury. When the dedifferentiated or redifferentiated cells are administered to repair cell damage due to injury and/or disease, the injury may be recent, in the process of forming scar tissue, or healed. If the injury has resulted in the formation of scar tissue or has begun to heal, the tissue may be re-injured prior to, coincident with, or subsequent to the administration of dedifferentiated or redifferentiated cells. Re-injury may help to promote regeneration resulting from administration of dedifferentiated or redifferentiated cells, however, the invention contemplates that regeneration can occur without re-injury.

### C. Specific Embodiments

1. Dedifferentiation of Cells Using Regenerating Extract.

During the dedifferentiation stage of newt limb regeneration, cleaved muscle cell products near the amputation plane contribute significantly to the formation of the blastema. The dedifferentiated muscle cells reenter the cell cycle and actively synthesize protein all within the first week after amputation. Myoblasts are mononucleated skeletal myocytes that proliferate when cultured in the presence of growth factors. These cells are committed to the myogenic lineage through expression of the muscle regulatory factors myoD and/or myf-5. When grown to confluency and deprived of growth factors, these myocytes enter the terminal differentiation pathway and begin to express, in succession, a number of muscle differentiation factors. These include myogenin, the cdk inhibitor p21/WAF1, activated retinoblastoma protein, and the muscle contractile proteins (e.g., myosin heavy chain and troponin T). The differentiating cells align along their axes and fuse to form terminally-differentiated myotubes capable of muscle contraction.

An extract, RNLE, from early regenerating limb tissue (days 0-5) in newts induced the dedifferentiation of both newt and murine myotubes in culture. Thus, mammalian (murine) myotubes are capable of dedifferentiating in response to dedifferentiation signals received from regenerating newt limbs. Thus, the present invention provides a composition for dedifferentiating mammalian tissue comprising a regeneration extract RNLE extract can therefore be used to dedifferentiate tissue from, for example, humans. RNLE extract may be applied in vivo or to cells in vitro. The invention further contemplates that the regeneration extract contains one or more factors that mediate the dedifferentiation and regeneration of cells (e.g., the extract contains one or more agents that comprise the regeneration activity of the extract). Accordingly, the invention contemplates that the extracts can be screened, and the one or more agents which mediate dedifferentiation and regeneration can be purified. The invention contemplates both the identification of such one or more active agents, as well as the use of these agents to dedifferentiate cells in vitro and/or in vivo.

2. Use of Msx1 to Dedifferentiate Cells

Msx1 is a homeobox-containing transcriptional repressor. Msx1 is expressed in the early regeneration blastema (Simon et. al., 1995), and its expression in the developing mouse limb demarcates the boundary between the undifferentiated (msxl-expressing) and differentiating (no msxl expression) cells (Hill et al., 1989; Robert et al., 1989; Simon et al., 1995). Furthermore, ectopic expression of either murine or human msxl will inhibit in vitro myogenesis in cultured mouse cells (Song et al., 1992; Woloshin et al., 1995).

A method to dedifferentiate cells by expression of msx1 is presented. The nucleic acid and amino acid sequences of mouse (SEQ ID NO: 1 and 2), rat (SEQ ID NO: 3 and 4), human (SEQ ID NO: 5 and 6) and axolotl (SEQ ID NO: 7 and 8) msx1 are provided herein. The present invention demonstrates that the combined effects of growth medium and ectopic msx1 expression can cause mouse C2C12 myotubes to dedifferentiate to a pool of proliferating, pluripotent stem cells that are capable of redifferentiating into several cell types, including chondrocytes, adipocytes, osteogenic cells, and myotubes. Thus, terminally-differentiated mammalian cells, like their urodele counterparts, are capable of dedifferentiating to pluripotent stem cells when challenged with the appropriate signals, as provided herein. Msx1 and msx1 analogs can be applied, for example, to human cells, in vivo and in vitro to induce cellular dedifferentiation.

In addition to the expression of either a nucleic acid encoding an msx1 polypeptide or the expression of an msx1 polypeptide, the invention contemplates that any agent which increase the expression and/or activity of msx1 can be used in the methods of the present invention to promote dedifferentiation. Such agents include nucleic acids, peptides, polypeptides, antibodies, small organic molecules, antisense oligonucleotides, ribozymes, RNAi constructs, and the like.

3. Use of Fibroblast Growth Factors to Promote Tissue Regeneration

The inventors demonstrate herein that Fgf signaling can mediate regeneration. Fgf polypeptides, which bind one or more. Fgf receptors (Fgfr), are involved in mammalian wound healing and tumor angiogenesis and play numerous roles in embryonic development, including induction and/or patterning during organogenesis of the limb, tooth, brain, and heart. Members of the Fgf signaling pathway are expressed in the epidermis as well as mesenchymal tissue during blastema formation and outgrowth stages. The inventors tested the function of Fgf signaling during Zebrafish fin regeneration, using a specific pharmacologic inhibitor of Fgfrl. Use of this agent revealed distinct requirement for Fgf signaling in induction and maintenance of blastemal cells, and suggested an additional role in patterning the regenerate. Thus, Fgf and like factors, may be used to dedifferentiate cells and to regenerate tissue in mammals, including humans.

By way of non-limiting example, the invention provides the nucleic acid and amino acid sequences of FGF polypeptides including FGF-2 (SEQ ID NO: 29 and 30), FGF-4 (SEQ ID. NO: 31 and 32), FGF-8 (SEQ ID NO: 33 and 34), FGF-10 (SEQ ID NO: 35 and 36), FGF-17 (SEQ ID NO: 37 and 38), and FGF-18 (SEQ ID NO: 39 and 40). Additionally, the invention provides the nucleic acid and amino acid sequences of four FGFRs including human FGFR1 (SEQ ID NO: 41 and 42), human FGFR2 (SEQ ID NO: 43′ and 44), human FGFR3 (SEQ ID NO: 45 and 46), and human FGFR4 (SEQ ID NO: 47 and 48).

In addition to methods of dedifferentiating cells by expressing an FGF polypeptide, the invention further contemplates that any agent which promotes FGF signaling can be used to promote dedifferentiation. Such agents include nucleic acids, peptides, polypeptides, small organic molecules, antibodies, ribozymes, RNAi constructs, antisense oligonucleotides, and the like.

4. Stem Cell Production In Vitro

In one embodiment, the invention provides methods to establish stem cells in vitro. Such stem cells are dedifferentiated from cells provided, for example, from an individual or a tissue culture cell line. Dedifferentiation may be achieved by applying an agent which promotes dedifferentiation. These stem cells can then be directed down different differentiation pathways by in vitro manipulation, or by transplanting back into the individual.

In another embodiment, the invention provides methods to establish pluripotent cells in vitro. Such pluripotent cells are derived from cells provided, for example, from a subject or a tissue culture cell line. Pluripotency may be achieved by applying an agent which promotes dedifferentiation to cause cells to dedifferentiate and take on pluripotent characteristics. Such cells can then be directed down different differentiation pathways by in vitro manipulation and then implanted into a subject, or by directly implanting into a subject.

In another embodiment, the invention provides methods to dedifferentiate muscle-derived cells, such that these cells resemble stem or pluripotent cells. In another embodiment, these cells can be driven down other differentiation pathways, such as adipocytes, chondrocytes, myotubes or osteoblasts.

5. Using RDF

Using RE will regenerate injured cells, tissue or organs. At the site of injury, RE may be applied, recapitulating the steps in regeneration seen in newts. Similarly, msx1, msx2, Fgf, agents which promote FGF signaling, agents which promote BMP signaling, agents which promote Wnt signaling, agents which promote expression and/or activity of msx1, agents which promote expression and/or activity of msx2, agents which inhibit expression and/or activity of msx3, agents which promote expression and/or activity of cyclinD1, agents which promote expression and/or activity of Cdk4, agents which inhibit expression and/or activity of p16, and agents which inhibit expression and/or activity of p21 can be used to dedifferentiate cells at the site of injury to promote cell, tissue or organ regeneration. For example, the injured tissue may be in a mammal; the mammal may be a human, and the injured site may be the consequence of trauma or disease.

Degenerative diseases and other medical conditions that might benefit from regeneration therapies include, but are not limited to: atherosclerosis, coronary artery disease, obstructive vascular disease, myocardial infarction, dilated cardiomyopathy, heart failure, myocardial necrosis, valvular heart disease, mitral valve prolapse, mitral valve regurgitation, mitral valve stenosis, aortic valve stenosis, and aortic valve regurgitation, carotid artery stenosis, femoral artery stenosis, stroke, claudication, and aneurysm; cancer-related conditions, such as structural defects resulting from cancer or cancer treatments; the cancers such as, but not limited to, breast, ovarian, lung, colon, prostate, skin, brain, and genitourinary cancers; skin disorders such as psoriasis; joint diseases such as degenerative joint disease, rheumatoid arthritis, arthritis, osteoarthritis, osteoporosis and ankylosing spondylitis; eye-related degeneration, such as cataracts, retinal and macular degenerations such as maturity onset; macular degeneration, retinitis pigmentosa, and Stargardt's disease; auralrelated degeneration, such as hearing loss; lung-related disorders, such as chronic obstructive pulmonary disease, cystic fibrosis, interstitial lung disease, emphysema; metabolic disorders, such as diabetes; genitourinary problems, such as renal failure and glomerulonephropathy; neurologic disorders, such as dementia, Alzheimer's disease, vascular dementia and stroke; and endocrine disorders, such as hypothyroidism. Finally, regeneration therapies from the methods and compositions of the invention may be very useful and beneficial for traumas to skin, bone, joints, eyes, neck, spinal column, and brain, for example, that result in injuries that would normally result in scar formation.

In addition to limb regeneration seen in the newt, like the newt, it is contemplated that other structures in mammals may be regenerated, such as skin, bone, joints, eyes (epithelium, retina, lens), lungs, heart, blood vessels and other vasculature, kidneys, pancreas, reproductive organs, tubular structures of the reproductive system (vas definers, Fallopian tubes) and nervous tissue (stroke, spinal cord injuries). Furthermore, the invention contemplates that the methods and compositions of the invention can be used to differentiate germ cells (e.g., oocytes and sperm) for use in basic and clinical research, fertility and treatments, and contraceptive studies.

### II. Definitions

Unless defined otherwise, all technical and scientific terms have the same meaning as is commonly understood by one of skill in the art to which this invention belongs.

The articles “a” and “an” are used herein to refer to one or to more than one (i.e., to at least one) of the grammatical object of the article. By way of example, “an element” means one element or more than one element.

“Isolated,” with respect to a molecule, means a molecule that has been identified and separated and/or recovered from a component of its natural environment. Contaminant components of its natural environment are materials that interfere with diagnostic or therapeutic use.

“Epimorphosis” refers to the process in which cellular proliferation precedes the development of a new anatomical structure; reproduction or reconstitution of a lost of injured part (neogenesis). While regeneration may recapitulate embryonic development, it may also involve metaplasia, the transformation of one differentiated cell type into another.

A cell that is “totipotent” is one that may differentiate into any type of cell and thus form a new organism or regenerate any part of an organism.

A “pluripotent” cell is one that has an unfixed developmental path, and consequently may differentiate into various specialized types of tissue elements, for example, such as adipocytes, chondrocytes, muscle cells, or osteoclasts. Pluripotent cells resemble totipotent cells in that they are able to develop into other cell types, however, various pluripotent cells may be limited in the number of developmental pathways they may travel.

A “marker” is used to determine the state of a cell. Markers are characteristics, whether morphological or biochemical (enzymatic), particular to a cell type, or molecules expressed by the cell type. Preferably, such markers are proteins, and more preferably, possess an epitope for antibodies or other binding molecules available in the art. However, a marker may consist of any molecule found in a cell, including, but not limited to, proteins peptides and polypeptides), lipids, polysaccharides, nucleic acids and steroids. Additionally, a marker may comprise a morphological or functional characteristic of a cell. Examples of morphological traits include, but are not limited to, shape, size, and nuclear to cytoplasmic ratio. Examples of functional traits include, but are not limited to, the ability to adhere to particular substrates, ability to incorporate or exclude particular dyes, ability to migrate under particular conditions, and the ability to differentiate along particular lineages.

Markers may be detected by any method available to one of skill in the art. In addition to antibodies (and all antibody derivatives) that recognize and bind at least one epitope on a marker molecule, markers may be detected using analytical techniques, such as by protein dot blots, sodium dodecyl sulfate polyacrylamide gel electrophoresis (SDS-PAGE), or any other gel system that separates proteins, with subsequent visualization of the market (such as Western blots), gel filtration, affinity column purification; morphologically, such as fluorescent-activated cell sorting (FACS), staining with dyes that have a specific reaction with a marker molecule (such as ruthenium red and extracellular matrix molecules), specific morphological characteristics (such as the presence of microvilli in epithelia, or the pseudopodia/filopodia in migrating cells, such as fibroblasts and mesenchyme); and biochemically, such as assaying for an enzymatic product or intermediate, or the overall composition of a cell, such as the ratio of protein to lipid, or lipid to sugar, or even the ratio of two specific lipids to each other, or polysaccharides. In the case of nucleic acid markers, any known method may be used. If such a marker is a nucleic acid, PCR, RT-PCR, in situ hybridization, dot blot hybridization, Northern blots, Southern blots and the like may be used, coupled with suitable detection methods. If such a marker is a morphological and/or functional trait, suitable methods include visual inspection using, for example, the unaided eye, a stereomicroscope, a dissecting microscope, a confocal microscope, or an electron microscope.

Regardless of the methods of analysis, a marker, or more usually, a combination of markers, is used to identify a particular cell. Myofibrils, for example, are characteristic of muscle cells; axons characterize neurons, cadherins are typically expressed by epithelial cells, β2integrins are typically expressed by white blood cells of the immune system, a high lipid content is characteristic of oligodendrocytes, and lipid droplets are unique to adipocytes. These examples serve merely to illustrate the use of one or more markers to identify a particular differentiated or undifferentiated cell type.

“Differentiation” describes the acquisition or possession of one or more characteristics or functions different from that of the original cell type. A differentiated cell is one that has a different character or function from the surrounding structures or from the precursor of that cell (even the same cell). The process of differentiation gives rise from a limited set of cells (for example, in vertebrates, the three germ layers of the embryo: ectoderm, mesoderm and endoderm) to cellular diversity, creating all of the many specialized cell types that comprise an individual.

Differentiation is a developmental process whereby cells assume a specialized phenotype, e.g., acquire one or more characteristics or functions distinct from other cell types. In some cases, the differentiated phenotype refers to a cell phenotype that is at the mature endpoint in some developmental pathway. In many, but not all tissues, the process of differentiation is coupled with exit from the cell cycle. In these cases, the cells lose or greatly restrict their capacity to proliferate and such cells are commonly referred to as being “terminally differentiated. However, we note that the term “differentiation” or “differentiated” refers to cells that are more specialized in their fate or function than at a previous point in their development, and includes both cells that are terminally differentiated and cells that, although not terminally differentiated, are more specialized than at a previous point in their development.

“Dedifferentiation” describes the process of a cell “going back” in developmental time. In some cases, a dedifferentiated cell resembles a progenitor cell. In other cases, a dedifferentiated cell acquires one or more characteristics previously possessed by that cell at an earlier developmental time point. An example of dedifferentiation is the temporal loss of epithelial cell characteristics during wounding and healing. Dedifferentiation may occur, in degrees: in the afore-mentioned example of wound healing, dedifferentiation progresses only slightly before the cells redifferentiate to recognizable epithelia. A cell that has greatly dedifferentiated, for example, is one that resembles a stein cell. Dedifferentiated cells can either: (i) remain dedifferentiated and proliferate as a dedifferentiated cell; (ii) redifferentiate along the same developmental L pathway from which the cell had previously dedifferentiated; or (iii) redifferentiate along a developmental pathway distinct from which the cell had previously dedifferentiated.

“Muscle cells” are characterized by their principal role: contraction. Muscle cells are usually elongate and arranged in vivo in parallel arrays. The principal components of muscle cells, related to contraction, are the myofilaments. Two types of myofilaments can be distinguished: (1) those composed primarily of actin, and (2) those composed primarily of myosin. While actin and myosin can be found in many other cell types, enabling such cells, or portions, to be mobile, muscle cells have an enormous number of co-aligned contractile filaments that are used to perform mechanical work.

Muscle tissue can be classified into two major classes based on the appearance and location of the contractile cells: (1) striated muscle, containing cross striations, and (2) smooth muscle, which does not contain any cross striations. Striated muscle can be further subdivided into skeletal muscle and cardiac muscle.

“Skeletal muscle” tissue consists of parallel striated muscle cells, enveloped by connective tissue. Striated muscles cells are also called fibers. Skeletal muscle cells are usually long, multinucleated; and display cross striations.

Occasionally satellite cells, much smaller than the skeletal muscle cells, are associated with the fibers.

“Cardiac muscle” consists of long fibers that, like skeletal muscle, are cross-striated. In addition to the striations, cardiac muscle also contains special cross bands, the intercalated discs, which are absent in skeletal muscle. Also unlike skeletal muscle in which the muscle fiber is a single multinucleated protoplasmic unit, in cardiac muscle the fiber consists of mononucleated (sometimes binucleated) cells aligned end-to-end. Cardiac cells often anastomose and conatin many large mitochondria. Usually, injured cardiac muscle is replaced with fibrous connective tissue, not cardiac muscle.

“Smooth muscle” consists of fusiform cells, 20 to 200 μM long, and in vivo, are thickest at the midregion, and taper at each end. While smooth muscle cells have microfilaments, they are not arranged in the ordered, paracrystalline manner of striated muscle. These cells contain numerous pinocytotic vesicles, and with the sacroplasmic reticulurn, sequester calcium. Smooth muscle cells will contact each other via gap junctions (or nexus). While some smooth muscle cells can divide, such as those found in the uterus, regenerative capacity is limited, and damaged areas are usually repaired by scar formation.

Other “contractile cells” include myofibroblasts, myoepithelial cells, testicle myoid cells, perineurial cells; although these are not usually anatomically classified as muscle cells.

As used herein, “neuronal cell” or “cell of the nervous system” include both neurons and glial cells.

As used herein, “CNS neuron” refers to a neuron whose cell body is located in the” central nervous system. The term is also meant to encompass neurons whose cell body was originally located in the central nervous system (e.g., endogenously located in the CNS), but which have been explanted and cultured ex vivo, as well as the progeny of such cells. Examples of such neurons are motor neurons, interneurons and sensory neurons including retinal ganglion cells, dorsal root ganglion cells and neurons of the spinal cord.

As used herein, “central nervous system” refers to any of the functional regions of the brain, spinal cord, or retina. This definition is used commonly in the art and is based, at least in part, on the common embryonic origin of the structures of the brain and spinal cord from the neural tube.

The “peripheral nervous system” can be distinguished from the central nervous system, at least in part, by its differing origin during embryogenesis. Cells of the peripheral nervous system are derived from the neural crest and include neurons and glia of the sensory, sympathetic and parasympathetic systems.

A “stem cell” describes any precursor cell, whose daughter cells may differentiate into other cell types. In general, a stem cell is a cell capable of extensive proliferation, generating more stem cells (self-renewal) as well as more differentiated progeny. Thus, a single stem cell can generate a clone containing millions of differentiated cells as well as a few stem cells. Stem cells thereby enable the continued proliferation of tissue precursors over a long period of time. Without being bound by theory, it is currently believed that stem cells exist in virtually ever tissue in the adult body, and that such stem cells provide an endogenous mechanism for some level of repair in adult tissues. Exemplary adult stem cells are well known in the art and include, but are not limited to, neural stem cells, neural crest stem cells, hematopoietic stem cells, mesenchymal stem cells, pancreatic stem cells, hepatic stem cells, cardiac stem cells, kidney stem cells, and the like. In addition to adult stem cells resident in virtually every adult tissue, embryonic stem cells and embryonic germ cells are two specific stem cell populations present during specific stages of embryogenesis.

Stem cells may divide asymmetrically, with one daughter retaining the stem state and the other daughter adopting a distinct function or phenotype. Alternatively, some of the stem cells in a population can divide symmetrically into two stem cells, thus maintaining some stem cells in the population as a whole, while other cells in the population give rise only to differentiated progeny. Formally, it is possible that cells that begin as stem cells might proceed toward a differentiated phenotype, but then “reverse” and re-express the stem cell phenotype. Additionally, as indicated by the results described herein, differentiated cells, including terminally differentiated cells can be induced to dedifferentiate, and such dedifferentiation includes dedifferentiation to a stem cell or to a progenitor cell.

Teratocarcinomas also contain stem cells, called embryonal carcinoma cells. Capable of division, they can differentiate into a wide variety of tissues, including gut and respiratory epithelia, muscle, nerve, cartilage, and bone (Gilbert, 1991).

Like stem cells, cells that begin as “progenitor cells” may proceed toward a differentiated phenotype, but then “reverse” and re-express the progenitor cell phenotype. Progenitor cells have a cellular phenotype that is more primitive than a differentiated cell; these cells are at an earlier step along a developmental pathway or progression than fully differentiated cells. Often, progenitor cells also have significant or very high proliferative potential. Progenitor cells may give rise to multiple distinct differentiated cell types or to a single differentiated cell type, depending on the developmental pathway and on the environment in which the cells develop and differentiate.

“Proliferation” refers to an increase in the number of cells in a population by means of cell division. Cell proliferation results from the coordinated activation of multiple signal transduction pathways, often in response to growth factors and other mitogens. Cell proliferation may also be promoted when cells are released from the actions of intra- or extracellular signals and mechanisms that block or down-regulate cell proliferation.

An “isolated nucleic acid” molecule is purified from the setting in which it is found in nature and is separated from at least one contaminant nucleic acid molecule. For example, isolated msx1 molecules are distinguished from the specific msx1 molecule, as it exists in cells. However, we note that in certain embodiments, an isolated molecule, for example an isolated msx1 molecule, may comprise a nucleic acid or amino acid sequence identical to that of a naturally occurring msx1, and such isolated msx1 molecules are still distinguished from msx1 as it exists in cells. An isolated molecule further includes molecules contained in cells that ordinarily express that molecule, wherein the nucleic acid encoding the particular polypeptide is in a chromosomal location different from that in which the nucleic acid is endogenously located in cells.

When the molecule is a “purified polypeptide,” the polypeptide will be purified (1) to obtain at least 15 residues of N-terminal or internal amino acid sequence using a sequenator, or (2) to homogeneity by SDS-PAGE under nonreducing or reducing conditions using Coomassie blue or silver stain. Isolated polypeptides include those expressed heterologously in genetically-engineered cells or expressed in vitro. Ordinarily, isolated polypeptides are prepared by at least one purification step.

Functional equivalents of a polypeptide, a polypeptide fragment, or a variant polypeptide are those polypeptides that retain a biological and/or an immunological activity of the native or naturally-occurring polypeptide. Immunological activity refers to the ability to induce the production of an antibody against an antigenic epitope possessed by a native polypeptide; biological activity refers to a function, either inhibitory or stimulatory, caused by the particular native polypeptide that excludes immunological activity. In the context of the present invention, exemplary biological activities include the ability to promote dedifferentiation of one or more cell types. Further exemplary biological activities include the ability to bind to a particular receptor, the ability to activate transcription of a particular gene, the ability to inhibit transcription of a particular gene, the ability to associate (e.g., directly or indirectly associate) with a particular cofactor, the ability to promote signaling via a particular signal transduction pathway, and the ability to inhibit signaling via another particular signal transduction pathway.

“Derivatives” of nucleic acid sequences or amino acid sequences are formed from the native compounds either directly or by modification or partial substitution. “Analogs” are nucleic acid sequences or amino acid sequences that have a structure similar to, but not identical to, the native compound but differ from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially identical to the nucleic acids or proteins of the invention. In various embodiments, the derivatives or analogs are at least about 70%, 75%, 80%, 85%, 90%, 95%, 97%, 98%, 99%, or greater than 99% identical to a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions (Ausubel et al., 1987).

A “homologous nucleic acid sequence” or “homologous amino acid sequence,” or variations thereof, refer to sequences characterized by homology at the nucleotide level or amino acid level as discussed above. Homologous nucleotide sequences encode those sequences coding for isoforms of a particular sequence. Isoforms can be expressed in different tissues of the same organism as a result of, for example, alternative splicing of RNA. Alternatively, different genes can encode isoforms. Homologous nucleotide sequences include nucleotide sequences encoding a polypeptide from other species, including, but not limited to: vertebrates, and thus can include, e.g., human, frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding a particular protein. Homologous nucleic acid sequences include those nucleic acid sequences that encode conservative amino acid substitutions (see below).

An “open reading frame” (ORF) is a nucleotide sequence that has a start codon (ATG) and terminates with one of the three “stop” codons (TAA, TAG, or TGA). In this invention, however, an ORF may be any part of a coding sequence that may or may not comprise a start codon and a stop codon. For example, the ORF of msx1 gene encodes an msx1 polypeptide. Preferable msx1 ORFs encode at least 30 contiguous amino acids of msx1 polypeptide sequence.

In general, a “growth factor” is a substance that promotes cell growth and development by directing cell maturation and differentiation. Growth factors also mediate tissue maintenance and repair. Growth factors affect cell behavior by binding to specific receptors on the surface of cells. The binding of ligand to a growth factor receptor activates a signal transduction pathway that influences, for example, cell behavior. Growth factors typically exert an affect on cells at very low concentrations.

“Fibroblast growth factors” (Fgfs) belong to a class of growth factors consisting of a large family of short polypeptides that are released extracellularly and bind with heparin to dimerize and activate specific receptor tyrosine kinases (Fgfrs). Fgf signaling is involved in mammalian wound healing and tumor angiogenesis (Ortega et al., 1998; Zetter, 1998) and has numerous roles in embryonic development, including induction and/or patterning during organogenesis of the limb, tooth, brain, and heart (Crossley et al., 1996; Martin, 1998; Ohuchi et al., 1997; Peters and Balling, 1999; Reifers et al., 1998; Vogel et al., 1996; Zhu et al., 1996). Fgfs' can easily be detected using either functional assays (Baird and Klagsbrun, 1991; Moody, 1993) or antibodies (Research Diagnostics; Flanders, N.J. or Promega, Wis.).

Currently, over 20 mammalian FGFs have been identified, and these FGF polypeptides interact with one or more of four FGFRs. The nucleic acid and amino acid sequences of non-limiting examples of FGFs are provided herein: human FGF-2 (SEQ ID NO: 29 and 30); human FGF-4 (SEQ ID NO: 31 and 32); human FGF-8 (SEQ ID NO: 33 and 34); human FGF-10 (SEQ ID NO: 35 and 36); human FGF-17 (SEQ ID NO: 37 and 38); and human FGF-18 (SEQ ID NO: 39 and 40). Similarly, the nucleic acid and amino acid sequences of the human FGFRs are provided herein: FGFR1 (SEQ ID NO: 41 and 42); FGFR2 (SEQ ID NO: 43 and 44); FGFR3 (SEQ ID NO: 45 and 46); and FGFR4 (SEQ ID NO: 47 and 48).

As used herein, the terms “transforming growth factor-beta” and “TGF-β” denote a family of structurally related paracrine polypeptides found ubiquitously in vertebrates, and prototypic of a large family of metazoan growth, differentiation, and morphogenesis factors (see, for review, Massaque et al. (1990) Ann Rev Cell Biol 6:597-641; and Sporn et al. (1992) J Cell Biol 119:1017-1021). Included in this “family are the “bone morphogenetic proteins” or “BMPs”, which refers to proteins isolated from bone, and fragments thereof and synthetic peptides which are involved in a variety of developmental processes. Preparations of BMPs, such as BMP-1, 2, 3, 4, 5, 6, and 7 are described in, for example, PCT publication WO 88/00205 and Wozney (1989) Growth Fact Res 1:267-280.

BMPs polypeptides are involved in a complex signaling cascade initiated by binding of BMP polypeptides to cell surface receptors. Intracellularly, BMP signaling is mediated by SMAD proteins including SMAD 1 and 2, the accessory SMAD (SMAD 4), and inhibitory SMADs which may be involved in limiting the rate or extent of BMP signaling. In addition to positive and negative regulation intracellularly, TGFβ signaling generally and BMP signaling specifically can be negatively regulated extracellularly by the activity of proteins including gremlin, noggin, chordin and follistatin. The nucleic acid and amino acid sequences of exemplary BMP family members are provide herein: mouse BMP-2 (SEQ ID NO: 17 and 18); human BMP-2 (SEQ ID NO: 19 and 20); mouse BMP4 (SEQ ID NO: 21 and 22); human BMP-4 (SEQ ID NO: 23 and 24); mouse BMP-7 (SEQ ID NO: 25 and 26); and human BMP-7 (SEQ ID NO: 27 and 28).

The Wnt gene family encodes secreted ligands that serve key roles in differentiation and development. This family comprises at least 15 vertebrate and invertebrate genes including the Drosophila segment polarity gene wingless. Wnt signaling is involved in a variety of developmental processes including early patterning, neural development, somite formation, cardiac development and kidney development, and inappropriate Wnt signaling can be involved in transformation of cells.

The Wnt signaling pathway is initiated via interaction of a Wnt polypeptide with a transmembrane receptor of the frizzled family. Intracellularly, transduction of the Wnt signal is mediated by both positive and negative regulatory proteins. Positive regulators include disheveled, and the transcription factors β-catenin and Lef-1, and negative regulators include GSK3β. In addition to negative regulation intracellularly, Wnt signaling can be negatively regulated extracellularly by the activity of Frzb related polypeptides. This family of polypeptides, which includes FrzA, Frzb, and sizzled, comprises soluble polypeptides that resemble the ligand binding domain of the Wnt receptor. Wnt polypeptides can bind Frzb related polypeptides, however, such binding does not result in Wnt signal transduction.

There are at least 15 identified Wnt polypeptides. Non-limiting examples of nucleic acid and amino acid sequences corresponding to human Wnt polypeptides' are provided herein: human Wnt1 (SEQ ID NO: 49 and 50); human Wnt2 (SEQ ID NO: 51 and 52); human Wnt3 (SEQ ID NO: 53 and 54); human Wnt5a (SEQ. ID NO: 55 and 56); human Wnt8 (SEQ ID NO: 57 and 58); and human Wnt11 (SEQ ID NO: 59 and 60). Additionally, nucleic acid and amino acid sequences corresponding to intracellular components of the Wnt signaling pathway are provided herein: human GSK3β (SEQ ID NO: 61 and 62); human β-catenin (SEQ ID NO: 63 and 64); and human Lef1 (SEQ ID NO: 65 and 66).

A “mature” form of a polypeptide or protein is the product of a naturally occurring polypeptide or precursor form or proprotein. For example, msxl can encode a mature msxl. The naturally occurring polypeptide, precursor or proprotein includes, for example, the full-length gene product, encoded by the corresponding gene. Alternatively, it may be defined as the polypeptide, precursor or proprotein encoded by an open reading frame described herein. The product “mature” form arises as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from, the operation of only one of these processes, or a combination of any of them.

By way of further example, BMP polypeptides are processed to yield the mature, functional form of the polypeptide. The mature mouse BMP-2 polypeptide corresponds to amino acid residues 294-394 of SEQ ID NO: 18, the mature human BMP-2 polypeptide corresponds to ammo acid residues 296-396 of SEQ ID NO: 20, the mature mouse BMP4 polypeptide corresponds to amino acid residues 320420 of SEQ ID NO: 22, the mature human BMP4 polypeptide corresponds to amino acid residues 302402 of SEQ ID NO: 24, the mature mouse BMP-7 polypeptide corresponds to amino acid residues 329430 of SEQ ID NO: 26, and the mature human BMP-7 polypeptide corresponds to amino acid residues 330431 of SEQ ID NO: 28.

An “active” polypeptide or polypeptide fragment retains a biological and/or an immunological activity similar, but not necessarily identical, to an activity of a naturally-occurring (wild-type) polypeptide of the invention, including mature forms. Biological assays, with or without dose dependency, can be used to determine activity. A nucleic acid fragment encoding a biologically-active portion of a polypeptide can be prepared by isolating a portion of a nucleic acid sequence that encodes a polypeptide having biological activity, expressing the encoded portion of the polypeptide and assessing the activity of the encoded portion of the polypeptide. Immunological activity refers to the ability to induce the production of an antibody against an antigenic epitope possessed by a polypeptide; biological activity refers to a function, either inhibitory or stimulatory, caused by a polypeptide that excludes immunological activity.

“Agents” for use in the methods of the present invention are capable of dedifferentiating a differentiated cell. Such agents are also referred to as “dedifferentiation factors”. Exemplary agents, either alone or in combination with other agents, are capable of dedifferentiating a cell. In one embodiment, a dedifferentiation factor is capable of dedifferentiating a terminally differentiated cell. In another embodiment, a dedifferentiation factor is capable of dedifferentiating a cell which is not terminally differentiated. In yet another embodiment, dedifferentiation (of a terminally differentiated cell or of a non-terminally differentiated cell) is to a stem or progenitor cell state. Dedifferentiation of a cell can be measured in any one of a number of ways including, but not limited to, increase in proliferation, decrease in one or more markers of differentiation, increase in expression of one or more stem or progenitor cell markers, and/or reentry into S phase. One of skill in the art will appreciate that some dedifferentiation factors are capable of dedifferentiating many different differentiated cell types (e.g., skeletal muscle cells, cardiac muscle cells, pancreatic cells, neural cells, epidermal cells, etc.) while other dedifferentiation factors are capable of dedifferentiating only one differentiated cell type, or only capable of dedifferentiating related cell types (e.g., only ectodermally derived cells, only mesenchymal cell types, or only endodermally derived cells).

Agents (e.g, dedifferentiation factors) for use in the methods of the present invention include nucleic acids, peptides, polypeptides, small organic molecules, antibodies, antisense oligonucleotides, RNAi constructs, ribozymes, DNA enzymes, and the like. Without being bound by theory, such agents may function in any one of a number of ways. Exemplary mechanisms by which agents may promote dedifferentiation include: promoting FGF signaling, promoting Wnt signaling, promoting BMP signaling, promoting expression and/or activity of msx1, promoting expression and/or activity of msx2, inhibiting expression and/or activity of msx3, promoting the expression and/or activity of a G₁ Cdk complex, promoting expression and or activity of cyclinD1, promoting expression and or activity of Cdk4, inhibiting expression and/or activity of p16, inhibiting expression and/or activity of p21, inhibiting expression and/or activity of p27, inhibiting expression and/or activity of Rb.

To further illustrate, exemplary agents that promote dedifferentiation and which promote FGF signaling include, but are not limited to: (i) a nucleic acid encoding an FGF polypeptide, (ii) an FGF polypeptide, (iii) a small organic molecule that binds to and promotes FGF signal transduction, (iv) a nucleic acid encoding an activated FGF receptor, (v) an activated FGF receptor polypeptide, (vi) a small organic molecule that binds to an FGF receptor and activates FGF signal transduction. Exemplary agents that promote dedifferentiation and which promote Wnt signaling include, but are not limited to: (i) a nucleic acid encoding a Wnt polypeptide, (ii) a Wnt polypeptide, (iii) a small organic molecule that binds to and promotes Wnt signal transduction, (iv) a nucleic acid encoding an activated Wnt receptor, (v) an activated Wnt receptor polypeptide, (vi) a small organic molecule that binds to a Wnt receptor and promotes Wnt signal transduction, (vii) a small organic molecule that binds to and inhibits the activity of a Wnt antagonist (e.g., Frzb, FrZA, sizzled), (viii) an antibody that binds to and inhibits the activity of a Wnt antagonist, (ix) an antisense oligonucleotide that binds to and inhibits the expression of a Wnt antagonist, (x) an RNAi construct that binds to and inhibits the expression of a Wnt antagonist, (xi) a ribozyme that binds to and inhibits the expression of a Wnt antagonist, (xii) a nucleic acid encoding a dominant negative GSK3β, (xiii) a dominant negative GSK3β polypeptide, (xiv) a small organic molecule that binds to and inhibits the expression and/or activity of GSK3β, (xv) an antisense oligonucleotide that binds to and inhibits the expression of GSK3β, (xvi) an RNAi construct that binds to and inhibits the expression of GSK3β, (xvii) a ribozyme that binds to and inhibits the expression of GSK3β, (xviii) an antibody that binds to and inhibits the expression of GSK3β (xix) a nucleic acid encoding β-catenin, (xx) a β-catenin polypeptide, (xxi) a small organic molecule that binds to and promotes expression and/or activity of β-catenin, (xxii) a nucleic acid encoding Lef-1, (xxiii) a Lef-1 polypeptide, (xxiv) a small organic molecule that binds to an promotes expression and/or activity of Lef-1. Exemplary agents that promote dedifferentiation and which promote BMP signaling include, but are not limited to: (i) a nucleic acid encoding a BMP polypeptide, (ii) a BMP polypeptide, (iii) a nucleic acid encoding an activated BMP receptor, (iv) an activated BMP receptor polypeptide, (v) a small organic molecule that binds to BMP and/or binds to a BMP receptor and promotes BMP signaling, (vi) a small organic molecules that inhibits the expression and/or activity of a BMP antagonist (e.g., noggin, chordin, gremlin, follistatin), (vii) an antisense oligonucleotide that binds to and inhibits the expression and/or activity of a BMP antagonist, (viii) an antibody that binds to and inhibits the expression and/or activity of a BMP antagonist, (ix) an RNAi construct that binds to and inhibits the expression and/or activity of a BMP antagonist, (x) a ribozyme that binds to and inhibits the expression and/or activity of a BMP antagonist, (xi) a nucleic acid encoding a SMAD1 or SMAD2 polypeptide, (xii) a SMAD1 of SMAD2 polypeptide, (xiii) a small organic molecule that binds to a SMAD polypeptide and promotes BMP signal transduction. Exemplary agents that promote dedifferentiation and which promote expression and/or activity of msx1 include, but are not limited to: (i) a nucleic acid encoding a msx1 polypeptide, (ii) an msx1 polypeptide, (iii) a small organic molecule that binds to and promotes the expression and/or activity of msx1. Exemplary agents that promote dedifferentiation and which promote expression and/or activity of msx2 include, but are not limited to: (i) a nucleic acid encoding a msx2 polypeptide, (ii) an msx2 polypeptide, (iii) a small organic molecule that binds to and, promotes the expression and/or activity of msx2. Exemplary agents that promote dedifferentiation and which inhibit expression and/or activity of msx3 include, but are not limited to: (i) a nucleic acid encoding a dominant negative msx3 polypeptide, (ii) a dominant negative msx3 polypeptide, (iii) a small organic molecule that binds to and inhibits the expression and/or activity of msx3, (iv) an antibody that binds to and inhibits the activity and/or expression of msx3, (v) an antisense oligonucleotide that binds to and inhibits the activity and/or expression of msx3, (vi) a ribozyme that binds to and inhibits the activity and/or expression of msx3, and (vii) an RNAi construct that binds to and inhibits the activity and/or expression of msx3. Exemplary agents that promote dedifferentiation and which promote expression and/or activity of a G1 Cdk complexes include, but are not limited to: (i) a nucleic acid encoding a cyclinD1 polypeptide, (ii) a cyclinD1 polypeptide, (iii) a small organic molecule that binds to and promotes the expression and/or activity of cyclinD1. Further exemplary agent include, but are not limited to: (i) a nucleic acid encoding a Cdk4 polypeptide, (ii) a Cdk4 polypeptide, (iii) a small organic molecule that binds to and promotes the expression and/or activity of Cdk4. Exemplary agents that promote dedifferentiation and which inhibit expression and/or activity of p16 include, but are not limited to: (i) a small organic molecule that binds to and inhibits expression and/or activity of p16, (ii) an antibody that binds to and inhibits expression and/or activity of p16, (iii) an antisense oligonucleotide that binds to and inhibits expression and/or activity of p16, (iv) an RNAi construct that binds to and inhibits expression and/or activity of p6, and (v) a ribozyme that binds to and inhibits expression and/or activity of p16. Exemplary agents that promote dedifferentiation and which inhibit expression and/or activity of p21 include, but are not limited to: (i) a small organic molecule that binds to and inhibits expression and/or activity of p21, (ii) an antibody that binds to and inhibits expression and/or activity of p21, (iii) an antisense oligonucleotide that binds to and inhibits expression and/or activity of p21, (iv) an RNAi construct that binds to and inhibits expression and/or activity of p21, and (v) a ribozyme that binds to and inhibits expression and/or activity of p21. Exemplary agents that promote dedifferentiation and which inhibit expression and/or activity of p27 include, but are not limited to: (i) a small organic molecule that binds to and inhibits expression and/or activity of p27, (ii) an antibody that binds to and inhibits expression and/or activity of p27, (iii) an antisense oligonucleotide that binds to and inhibits expression and/or activity of p27, (iv) an RNAi construct that binds to and inhibits expression and/or activity of p27, and (v) a ribozyme that binds to and inhibits expression and/or activity of p27. Exemplary agents that promote dedifferentiation and which inhibit expression and/or activity of Rb include, but are not limited to: (i) a small organic molecule that binds to and inhibits expression and/or activity of Rb, (ii) an antibody that binds to and inhibits expression and/or activity of Rb, (iii) an antisense oligonucleotide that binds to and inhibits expression and/or activity of Rb, (iv) an RNAi construct that binds to and inhibits expression and/or activity of Rb, and (v) a ribozyme that binds to and inhibits expression and/or activity of Rb.

The term “agent” refers to a compound used in the methods of the present invention, as well as to a compound screened by the methods of the present invention. The term agent includes nucleic acids, peptides, proteins, peptidomimetics, small organic molecules, chemical compounds, ribozymes, RNAi constructs (including siRNA), antisense oligonucleotides, DNA enzymes, and antibodies. Preferred agents for use in the subject methods are those which promote dedifferentiation.

Agents used in the methods described herein, as well as agents screened by the methods described herein can be administered and/or screened individually, or can be administered in combination with one or more other agents. Exemplary combinations include, but are not limited to, (i) one or more agents that promote dedifferentiation by promoting FGF signal transduction; (ii) one or more agents that promote dedifferentiation by promoting BMP signal transduction; (iii) one or more agents that promote dedifferentiation by promoting Wnt signal transduction; (iv) one or more agents that promote dedifferentiation by promoting expression of msx1 and/or msx2; (v) one or more agents that promote dedifferentiation by inhibiting expression of msx3; (vi) one or more agents that promote dedifferentiation by increasing expression of cyclinD1; (vii) one or more agents that promote dedifferentiation by increasing the activity of Cdk4; (viii) one or more agents that promote dedifferentiation by inhibiting the activity of p16; and (ix) one or more agents that promote dedifferentiation by inhibiting the activity of p21. The invention further contemplates that combinations of agents to promote dedifferentiation may include combinations of any of the above cited classes of agents, as well as combinations of one or more agents that promote dedifferentiation via a different mechanism or via an unknown mechanism.

The invention further contemplates the screening of libraries to identify and/or characterize dedifferentiation agents. Such libraries may include, without limitation, cDNA libraries (either plasmid based or phage based), expression libraries, combinatorial libraries, chemical libraries, phage display libraries, variegated libraries, and biased libraries. The term “library” refers to a collection of nucleic acids, proteins, peptides, chemical compounds, small organic molecules, or antibodies. Libraries comprising each of these are well known in the art. Exemplary types of libraries include combinatorial, variegated, biased, and unbiased libraries. Libraries can provide a systematic way to screen large numbers of nucleic acids, proteins, peptides, chemical compounds, small organic molecules, or antibodies. Often, libraries are sub-divided into pools containing some fraction of the total species represented in the entire library. These pools can then be screened to identify fractions containing the desired activity. The pools can be further subdivided, and this process can be repeated until either (i) the desired activity can be correlated with a specific species contained within the library, or (ii) the desired activity is lost during further subdivision of the pool of species, and thus is the result of multiple species contained within the library.

Based on the finding disclosed in the present application which indicate that terminally differentiated mammalian cells can be dedifferentiated, the present invention contemplates the identification of additional dedifferentiation agents. In one embodiment, the identified agents function via any one of the following mechanisms (i) the agent promotes dedifferentiation by promoting FGF signal transduction; (ii) the agent promotes dedifferentiation by promoting BMP signal transduction; (iii) the agent promotes dedifferentiation by promoting Wnt signal transduction; (iv) the agent promotes dedifferentiation by promoting expression and/or activity of msx1 and/or msx2; (v) the agent promotes dedifferentiation by inhibiting expression and/or activity of msx3; (vi) the agent promotes dedifferentiation by increasing expression and/or activity of cyclinD1; (vii) the agent promotes dedifferentiation by increasing the expression and/or activity of Cdk4; (viii) the agent promotes dedifferentiation by inhibiting the activity of p16; or (ix) the agent promotes dedifferentiation by inhibiting the activity of p21. In another embodiment, the identified agents promote dedifferentiation via another, perhaps unknown, mechanism. The invention contemplates the identification, characterization, and/or use of agents which promote dedifferentiation, whether by a known or unknown mechanism, and such agents include nucleic acids, peptides, polypeptides, peptidomimetics, small organic molecules, antisense oligonucleotides, RNAi constructs, and antibodies.

As used herein, “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied.

The terms “peptide(s)”, “protein(s)” and “polypeptide(s)” are used interchangeably herein.

The terms “polynucleotide sequence” and “nucleotide sequence” are also used interchangeably herein.

“Recombinant,” as used herein, means that a protein is derived from a prokaryotic or eukaryotic expression system.

The term “wild type” refers to the naturally-occurring polynucleotide sequence encoding a protein, or a portion thereof, or protein sequence, or portion thereof, respectively, as it normally exists in vivo.

The term “mutant” refers to any change in the genetic material of an organism, in particular a change (i.e., deletion, substitution, addition, or alteration) in a wildtype polynucleotide sequence or any change in a wildtype protein sequence. The term “variant” is used interchangeably with “mutant”. Although it is often assumed that a change in the genetic material results in a change of the function of the protein, the terms “mutant” and “variant” refer to a change in the sequence of a wildtype protein regardless of whether that change alters the function of the protein (e.g., increases, decreases, imparts a new function), or whether that change has no effect on the function of the protein (e.g., the mutation or variation is silent).

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

As used herein, the term “gene” or “recombinant gene” refers to a nucleic acid comprising an open reading frame encoding a polypeptide, including both exon and (optionally) intron sequences.

As used herein, the term “vector” refers to a nucleic acid molecule capable of transporting another nucleic acid to which it has been linked. Preferred vectors are those capable of autonomous replication and/or expression of nucleic acids to which they are linked. Vectors capable of directing the expression of genes to which they are operatively linked are referred to herein as “expression vectors”.

A polynucleotide sequence (DNA, RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

“Transcriptional regulatory sequence” is a generic term used throughout the specification to refer to nucleic acid sequences, such as initiation signals, enhancers, and promoters, which induce or control transcription of protein coding sequences with which they are operably linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein.

As used herein, the term “tissue-specific promoter” means a nucleic acid sequence that serves as a promoter, i.e., regulates expression of a selected nucleic acid sequence operably linked to the promoter, and which affects expression of the selected nucleic acid sequence in specific cells of a tissue, such as cells of neural origin, e.g. neuronal cells. The term also covers so-called “leaky” promoters, which regulate expression of a selected nucleic acid primarily in one tissue, but cause expression in other tissues as well.

A “chimeric protein” or “fusion protein” is a fusion of a first amino acid sequence encoding a polypeptide with a second amino acid sequence defining a domain (e.g. polypeptide portion) foreign to and not substantially homologous with any domain of the first polypeptide. A chimeric protein may present a foreign domain which is found (albeit in a different protein) in an organism which also expresses the first protein, or it may be an “interspecies”, “intergenic”, etc. fusion of protein structures expressed by different kinds of organisms.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened to identify compounds that promote dedifferentiation.

The “non-human animals” of the invention include mammals such as rats, mice, rabbits, sheep, cats, dogs, cows, pigs, and non-human primates.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion.

The phrases “systemic administration,” “administered systemically,” “peripheral administration” and “administered peripherally” as used herein mean the administration of a compound, drug or other material other than directly into the central nervous system, such that it enters the animal's system and, thus, is subject to metabolism and other like processes, for example, subcutaneous administration.

The phrase “effective amount” as used herein means that the amount of one or more agent, material or composition comprising one or more agents as described herein which is effective for producing some desired effect in a subject; for example, an amount of the compositions described herein effective to promote dedifferentiation. In one embodiment, an amount effective to promote dedifferentiation also promotes regeneration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

### III. Exemplary Agents

The present invention contemplates that numerous agents can be used to promote dedifferentiation and/or promote regeneration, either in vivo or in vitro. Agents which promote dedifferentiation and/or regeneration, either in vivo or in vitro are useful in the methods of the present invention. Without being bound by theory, such agents include nucleic acids, peptides, polypeptides, small organic molecules, antibodies, antisense oligonucleotides, RNAi constructs, ribozymes, and the like. Furthermore, it is appreciated that an agent which promotes dedifferentiation, whether via a known or an unknown mechanism, is useful in the methods of the present invention. Nevertheless, and without being bound by theory, the invention contemplates that exemplary dedifferentiation agents include: (i) agents that promote FGF signal transduction, (ii) agents that promote BMP signal transduction, (iii) agents that promote Wnt signaling, (iv) agents that promote expression and/or activity of msx1, (v) agents that promote expression and/or activity of msx2, (vi) agents that inhibit activity and/or expression of msx3, (vii) agents that promote expression and/or activity of cyclinD1, (viii) agents that promote expression, and/or activity of Cdk4, (ix) agents that inhibit expression and/or activity of p16, and (x) agents that inhibit expression and/or activity of p21.

A. Classes of Agents

Numerous mechanisms exist to promote or inhibit the expression and/or activity of a particular mRNA or protein. Without being bound by theory, the present invention contemplates any of a number of methods for promoting the expression and/or activity of a particular mRNA or protein, as well as a number of methods for inhibiting the expression and/or activity of a particular mRNA or protein. Still furthermore, the invention contemplates combinatorial methods comprising either (i) the use of two or more agents that decrease the expression and/or activity of a particular mRNA or protein, (ii) the use of one or more agents that decrease the expression and/or activity of a particular mRNA or protein plus the use of one or more agents that decrease the expression and/or activity of a second mRNA or protein, (iii) the use of two or more agents that increase the expression and/or activity of a particular mRNA or protein, (iv) the use of one or more agents that increase the expression and/or activity of a particular mRNA or protein plus the use of one or more agent that increase the expression and/or activity of a second mRNA or protein, (v) the use of one or more agents that increase expression and/or activity of a particular mRNA or protein plus the use of one or more agents that decrease the expression and/or activity of a particular mRNA or protein.

The following are illustrative examples of methods for promoting or inhibiting the expression and/or activity of an mRNA or protein. These examples are in no way meant to be limiting, and one of skill in the art can readily select from among known methods of promoting or inhibiting expression and/or activity.

Antisense oligonucleotides are relatively short nucleic acids that are complementary (or antisense) to the coding strand (sense strand) of the mRNA encoding a particular protein. Although antisense oligonucleotides are typically RNA based, they can also be DNA based. Additionally, antisense oligonucleotides are often modified to increase their stability.

Without being bound by theory, the binding of these relatively short oligonucleotides to the mRNA is believed to induce stretches of double stranded RNA that trigger degradation of the messages by endogenous RNAses. Additionally, sometimes the oligonucleotides are specifically designed to bind near the promoter of the message, and under these circumstances, the antisense oligonucleotides may additionally interfere with translation of the message. Regardless of the specific mechanism by which antisense oligonucleotides function, their administration to a cell or tissue allows the degradation of the mRNA encoding a specific protein. Accordingly, antisense oligonucleotides decrease the expression and/or activity of a particular protein.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule.

The antisense oligonucleotide may comprise at least one modified base moiety which is selected from the group including but not limited to 5-fluorouracil, 5-bromouracil, 5-chlorouracil, 5-iodouracil, hypoxanthine; xanthine, 4-acetylcytosine, 5-(carboxyhydroxytriethyl)uracil, 5-carboxymethylaminomethyl-2-thiouridine, 5-carboxymethylaminomethyluracil, dihydiouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methQxyaminomethyl-2-thiouracil, beta-D-mannosylqueosine, 5-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl)uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an -anomeric oligonucleotide. An anomeric oligonucleotide forms specific double-stranded hybrids with complementary RNA in which, contrary to the usual units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et. al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

The selection of an appropriate oligonucleotide can be readily performed by one of skill in the art. Given the nucleic acid sequence encoding a particular protein, one of skill in the art can design antisense oligonucleotides that bind to that protein, and test these oligonucleotides in an in vitro or in vivo system to confirm that they bind to and mediate the degradation of the mRNA encoding the particular protein. To design an antisense oligonucleotide that specifically binds to and mediates the degradation of a particular protein, it is important that the sequence recognized by the oligonucleotide is unique or substantially unique to that particular protein. For example, sequences that are frequently repeated across protein may not be an ideal choice for the design of an oligonucleotide that specifically recognizes and degrades a particular message. One of skill in the art can design an oligonucleotide, and compare the sequence of that oligonucleotide to nucleic acid sequences that are deposited in publicly available databases to confirm that the sequence is specific or substantially specific for a particular protein.

In another example, it may be desirable to design an antisense oligonucleotide that binds to and mediates the degradation of more than one message. In one example, the messages may encode related proteins such as isoforms or functionally redundant proteins. In such a case, one of skill in the art can align the nucleic acid sequences that encode these related proteins, and design an oligonucleotide that recognizes both messages.

A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

However, it may be difficult to achieve intracellular concentrations of the antisense sufficient to suppress translation on endogenous mRNAs in certain instances. Therefore another approach utilizes a recombinant DNA construct in which the antisense oligonucleotide is placed under the control of a strong pol III or pol II promoter. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. Without being bound by theory, RNAi appears to involve mRNA degradation, however the biochemical mechanisms are currently an active area of research. Despite some mystery regarding the mechanism of action, RNAi provides a useful method of inhibiting gene expression in vitro or in vivo.

As used herein, the term “dsRNA” refers to siRNA molecules, or other RNA molecules including a double stranded feature and able to be processed to siRNA in cells, such as hairpin RNA moieties.

The term “loss-of-function,” as it refers to genes inhibited by the subject RNAi method, refers a diminishment in the level of expression of a gene when compared to the level in the absence of RNAi constructs.

As used herein, the phrase “mediates RNAi” refers to (indicates) the ability to distinguish which RNAs are to be degraded by the RNAi process, e.g., degradation occurs in a sequence-specific manner rather than by a sequence-independent dsRNA response, e.g., a PKR response.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

“RNAi expression vector” (also referred to herein as a “dsRNA-encoding plasmid”) refers to a replicable nucleic acid constructs used to express (transcribe) RNA which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of an nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res. 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleofide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain preferred embodiments, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In other embodiments, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the effects of interferon or PKR are preferred.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

In yet other embodiments, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

Exemplary RNAi constructs that specifically recognize a particular gene, or a particular family of genes can be selected using methodology outlined in detail above with respect to the selection of antisense oligonucleotide. Similarly, methods of delivery RNAi constructs include the methods for delivery antisense oligonucleotides outlined in detail above.

Ribozymes molecules designed to catalytically cleave an mRNA transcripts can also be used to prevent translation of mRNA (See, e.g., PCT International Publication WO90/11364, published Oct. 4, 1990; Sarver et al., 1990, Science 247:1222-1225 and U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO88/04300 by University Patents Inc.; Been and Cech, 1986, Cell, 47:207-216). The Cech-type ribozymes have an eight base pair active site that hybridizes to a target RNA sequence whereafter cleavage of the target RNA takes place. The invention encompasses those Cech-type ribozymes that target eight base-pair active site sequences.

As in the antisense approach, the ribozymes can be composed of modified oligonucleotides (e.g., for improved stability, targeting, etc.) and can be delivered to cells in vitro or in vivo. A preferred method of delivery involves using a DNA construct “encoding” the ribozyme under the control of a strong constitutive pol III or pol II promoter, so that transfected cells will produce sufficient quantities of the ribozyme to destroy targeted messages and inhibit translation. Because ribozymes unlike antisense molecules, are catalytic, a lower intracellular concentration is required for efficiency.

DNA enzymes incorporate some of the mechanistic features of both antisense and ribozyme technologies. DNA enzymes are designed so that they recognize a particular target nucleic acid sequence, much like an antisense oligonucleotide, however much like a ribozyme they are catalytic and specifically cleave the target nucleic acid.

There are currently two basic types of DNA enzymes, and both of these were identified by Santoro and Joyce (see, for example, U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions.

Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantially sequence is a G/C rich of approximately 18 to 22 nucleotides. High G/C content helps insure a stronger interaction between the DNA enzyme and the target sequence.

When synthesizing the DNA enzyme, the specific antisense recognition sequence that will target the enzyme to the message is divided so that it comprises the two arms of the DNA enzyme, and the DNA enzyme loop is placed between the two specific arms.

Methods of making and administering DNA enzymes can be found, for example, in U.S. Pat. No. 6,110,462. Similarly, methods of delivering DNA ribozymes in vitro or in vivo include methods of delivering RNA ribozyme, as outlined in detail above. Additionally, one of skill in the art will recognize that, like antisense oligonucleotide, DNA enzymes can be optionally modified to improve stability and improve resistance to degradation.

Antibodies can be used as inhibitors of the activity of a particular protein. Antibodies can have extraordinary affinity and specificity for particular epitopes. Antibodies that bind to a particular protein in such a way that the binding of the antibody to the epitope on the protein can interfere with the function of that protein. For example, an antibody may inhibit the function of the protein by sterically hindering the proper protein-protein interactions or occupying active sites. Alternatively the binding of the antibody to an epitope on the particular protein may alter the conformation of that protein such that it is no longer able to properly function.

Monoclonal or polyclonal antibodies can be made using standard protocols (See, for example, Antibodies: A Laboratory Manual ed. by Harlow and Lane (Cold Spring Harbor Press: 1988)). A mammal, such as a mouse, a hamster, a rat, a goat, or a rabbit can be immunized with an immunogenic form of the peptide. Techniques for conferring immunogenicity on a protein or peptide include conjugation to carriers or other techniques well known in the art.

Following immunization of an animal with an antigenic preparation of a polypeptide, antisera can be obtained and, if desired, polyclonal antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal-Antibodies and Cancer Therapy, Alan R. Liss, Inc. pp. 77-96). Hybridoma cells can be screened immunochemically for production of antibodies specifically reactive with a particular polypeptide and monoclonal antibodies isolated from a culture comprising such hybridoma cells.

In the context of the present invention, antibodies can be screened and tested to identify those antibodies that can inhibit the function of a particular protein. One of skill in the art will recognize that not every antibody that is specifically immunoreactive with a particular protein will interfere with the function of that protein. However, one of skill in the art can readily test antibodies to identify those that are capable of blocking the function of a particular protein.

The term antibody as used herein is intended to include fragments thereof which are also specifically reactive with a particular polypeptide. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific and chimeric molecules having affinity for a particular protein conferred by at least one CDR region of the antibody.

Both monoclonal and polyclonal antibodies (Ab) directed against a particular polypeptides, and antibody fragments such as Fab, F(ab)₂, Fv and scFv can be used to block the action of a particular protein. Such antibodies' can be used ‘either in an experimental’ context to further understand the role of a particular protein in a biological process, or in a therapeutic context.

In addition to the use of antibodies to inhibit the function of, for example, msx3, p16, p21, gremlin, follistatin, noggin, chordin, Frzb, FrzA, sizzled, or an inhibitory SMAD, the present invention contemplate that antibodies raised against a particular protein can also be used to monitor the expression of that protein in vitro or in vivo (e.g., such antibodies can be used in immunohistochemical staining).

Polypeptides and peptide fragments can either agonize or antagonize the function of a particular protein, and such polypeptides and polypeptide variants can be used to promote dedifferentiation. In some aspects, the polypeptide comprises a bioactive portion of a polypeptide, and expression of that polypeptide in the cell promotes dedifferentiation. In other aspects, the polypeptide comprises an antagonistic variant of a wildtype polypeptide, and this antagonistic variant inhibits the expression and/or activity of a protein that inhibits dedifferentiation. Such an antagonistic polypeptide could be used to dedifferentiate cells by relieving this inhibitory effect.

One of skill in the art can readily make and test wildtype polypeptides, polypeptides variants, and peptide fragments to determine if said polypeptide acts as an agonist or antagonist of the function of the protein. Examples of such variants and fragments include dominant negative mutants of a particular protein.

One of skill in the art can readily make variants comprising an amino acid sequence at least 60%, 70%, 75%, 80%, 85%, 90%, 95%, 98% or 99% identical to a particular polypeptide, and identify variants that either agonize or antagonize the function of the wildtype protein. Further examples of antagonistic variants and antagonistic peptide fragments are described in the present application.

Small organic molecules can agonize or antagonize the function of a particular protein. By small organic molecule is meant a carbon contain molecule having a molecular weight less than 2500 amu, more preferably less than 1500 amu, and even more preferably less than 750 amu. In the context of the present invention, such small organic molecules would be able to promote dedifferentiation by (i) promoting FGF signaling, (ii) promoting BMP signaling, (iii) promoting Wnt signaling, (iv) promoting expression and/or activity of msx1, (v) promoting expression and/or activity of msx2, (vi) promoting expression and/or activity of cyclinD1, or promoting expression and/or activity of Cdk4. Further small organic molecules that promote dedifferentiation do so by (i) inhibiting expression and/or activity of msx3, (ii) inhibiting expression and/activity of p16, or (iii) inhibiting expression and/or activity of p21.

Small organic molecules can be readily identified by screening libraries of organic molecules and/or chemical compounds to identify those compounds that have a desired function. Without being bound by theory, small organic molecules may exert their inhibitory function in any of a number of ways including promoting expression and/or activity of a protein involved in promoting dedifferentiation, promoting signaling via a signaling pathway involved in promoting dedifferentiation, inhibiting expression and/or activity of a protein which inhibits dedifferentiation, inhibiting expression and/or activity of a protein that negatively regulates/suppresses signaling via a signaling pathway involved in promoting dedifferentiation.

In addition to screening readily available libraries to identify small organic molecules with a particular inhibitory function, the present invention contemplates the rational design and testing of small organic molecules that can inhibit the function of a particular protein. For example, based on molecular modeling of the binding site of a particular protein, one of skill in the art can design small molecules that can occupy that binding pocket. Such small organic molecules would be candidate inhibitors of the function of that particular protein. Further rational design can be based on analysis of the ligand binding domain of a particular receptor, the DNA binding domain of a transcription factor, or a cofactor binding domain of a receptor or ligand.

The present invention contemplates a large number of agents that promote dedifferentiation including nucleic acids, peptides, polypeptides, small organic molecules, antisense oligonucleotides, RNAi constructs, antibodies, ribozymes, and DNA enzymes. Exemplary agents include both agents that positively regulate proteins involved in dedifferentiation, as well as agents that negatively regulate proteins that inhibit dedifferentiation. Furthermore, agents for use in the methods of the present invention include agents which promote dedifferentiation, even when said agent promotes dedifferentiation via an unknown mechanism.

Agents that promote dedifferentiation and/or regeneration, either in vivo or in vitro, and can be used in the methods of the present invention have one or more of the following functions: (i) decrease expression of one or more markers of differentiation, (ii) increase expression of one or more markers of a less differentiated state, (iii) increase expression of one or more stem or progenitor cell markers, (iv) promote proliferation, (v) promote reentry of a terminally differentiated cell into the cell cycle.

B. Exemplary Mechanisms to Promote Dedifferentiation

Without being bound by theory, agents which promote dedifferentiation may function via any of a number of mechanism. However, the invention further contemplates the identification and use of agents that function via an unknown or as yet unidentified mechanism.

FGF Signaling

As described in detail herein, FGF signaling promotes dedifferentiation. Accordingly, the invention contemplates that agents which, promote FGF signaling can promote dedifferentiation. Exemplary agents include, but are not limited to, (i) a nucleic acid encoding an FGF polypeptide, (ii) an FGF polypeptide, (iii) a small organic molecule that binds to and promotes FGF signal transduction, (iv) a nucleic acid encoding an activated FGF receptor, (v) an activated FGF receptor polypeptide, (vi) a small organic molecule that binds to an FGF receptor and activates FGF signal transduction.

BMP Signaling

BMP signaling has many effects on cells and tissue, and among the molecular responses to BMP signaling is induction of msx1 expression. Accordingly, methods and compositions which promote BMP signaling can be used to promote dedifferentiation. Exemplary agents that promote dedifferentiation and which promote BMP signaling include, but are not limited to: (i) a nucleic acid encoding a BMP polypeptide, (ii) a BMP polypeptide, (iii) a nucleic acid encoding an activated BMP receptor, (iv) an activated BMP receptor polypeptide, (v) a small organic molecule that binds to BMP and/or binds to a BMP receptor and promotes BMP signaling, (vi) a small organic molecules that inhibits the expression and/or activity of a BMP antagonist (e.g., noggin, chordin, gremlin, follistatin), (vii) an antisense oligonucleotide that binds to and inhibits the expression and/or activity of a BMP antagonist, (viii) an antibody that binds to and inhibits the expression and/or activity of a BMP antagonist, (ix) an RNAi construct that binds to and inhibits the expression and/or activity of a BMP antagonist, (x) a ribozyme that binds to and inhibits the expression and/or activity of a BMP antagonist, (xi) a nucleic acid encoding a SMAD1 or SMAD2 polypeptide, (xii) a SMAD1 of SMAD2 polypeptide, (xiii) a small organic molecule that binds to a SMAD polypeptide and promotes BMP signal transduction.

Wnt Signaling

As described in detail herein, Wnt signaling promotes dedifferentiation. Accordingly, the invention contemplates that agents which promote Wnt signaling can promote dedifferentiation. Exemplary agents that promote dedifferentiation and which promote Wnt signaling include, but are not limited to: (i) a nucleic acid encoding a Wnt polypeptide, (ii) a Wnt polypeptide, (iii) a small organic molecule that binds to and promotes Wnt signal transduction, (iv) a nucleic acid encoding an activated Wnt receptor, (v) an activated Wnt receptor polypeptide, (vi) a small organic molecule that binds to a Wnt receptor and promotes Wnt signal transduction, (vii) a small organic molecule that binds to and inhibits the activity of a Wnt antagonist (e.g., Frzb, FrzA, sizzled), (viii) an antibody that binds to and inhibits the activity of a Wnt antagonist, (ix) an antisense oligonucleotide that binds to and inhibits the expression of a Wnt antagonist, (x) an RNAi construct that binds to and inhibits the expression of a Wnt antagonist, (xi) a ribozyme that binds to and inhibits the expression of a Wnt antagonist, (xii) a nucleic acid encoding a dominant negative GSK3β, (xiii) a dominant negative GSK3β polypeptide, (xiv) a small organic molecule that binds to and inhibits the expression and/or activity of GSK3β, (xv) an antisense oligonucleotide that binds to and inhibits the expression of GSK3β, (xvi) an RNAi construct that binds to and inhibits the expression of GSK3β, (xvii) a ribozyme that binds to and inhibits the expression of GSK3β, (xviii) an antibody that binds to and inhibits the expression of GSK3β (xix) a nucleic acid encoding β-catenin, (xx) a β-catenin polypeptide, (xxi) a small organic molecule that binds to and promotes expression and/or activity of β-catenin, (xxii) a nucleic acid encoding Lef-1, (xxiii) a Lef-1 polypeptide, (xxiv) a small organic molecule that binds to an promotes expression and/or activity of Lef-1.

Msx1

As described herein, expression of msx1 promotes dedifferentiation. Accrodingly, agents which increase the activity and/or expression of msx1 can promote dedifferentiation. Exemplary agents that promote dedifferentiation and which promote expression and/or activity of msx1 include, but are not limited to: (i) a nucleic acid encoding a msx1 polypeptide, (ii) an msx1 polypeptide, (iii) a small organic molecule that binds to and promotes the expression and/or activity of msx1.

Msx2

Msx2 is closely related to msx1, and the functions of these proteins appear to overlap in many systems. Additionally, as is the case with msx1, msx2 expression is induced by BMP signaling. Accordingly, the invention contemplates that agents that increase expression and/or activity of msx2 can promote dedifferentiation. Exemplary agents that promote dedifferentiation and which promote expression and/or activity of msx2 include, but are not limited to: (i) a nucleic acid encoding a msx2 polypeptide, (ii) an msx2 polypeptide, (iii) a small organic molecule that binds to and promotes the expression and/or activity of msx2.

Msx3

Msx3 is related to msx1 and msx2, however, expression of msx3 has been shown to antagonize or inhibit the activity of msx1, and perhaps msx2. Accordingly, agents which inhibit the expression and/or activity of msx3 can be used to effectively increase the expression and/or activity of msx1 and/or msx2, and such inhibitors of msx3 can be used to promote dedifferentiation. Exemplary agents that promote dedifferentiation and which inhibit expression and/or activity of msx3 include, but are not limited to: (i) a nucleic acid encoding a dominant negative msx3 polypeptide, (ii) a dominant negative msx3 polypeptide, (iii) a small organic molecule that binds to and inhibits the expression and/or activity of msx3, (iv) an antibody that binds to and inhibits the activity and/or expression of msx3, (v) an antisense oligonucleotide that binds to and inhibits the activity and/or expression of msx3, (vi) a ribozyme that binds to and inhibits the activity and/or expression of msx3, and (vii) an RNAi construct that binds to and inhibits the activity and/or expression of msx3.

Cell Cycle Regulation

The subject method can be carried out with other agents which produce the same effect as ectopic expression of Msx1 or Msx2. While not being bound by any particular theory, one mechanism by which expression of msx1 or msx2 is believed to promote dedifferentiation is by their ability to upregulate cyclin D1/CDK activity (either by derepressing an inhibitor of cyclinD1, by directly activating expression of cyclin D1, or by directly activating expression and/or activity of Cdk). Accordingly, the present invention also includes methods for inducing dedifferentiation wherein the dedifferentiation agent(s) effect an increase in CDK4, CDK6 and/or CDK2 activity, e.g., to cause cells to exit the G0 phase of cell growth and undergo mitosis or accelerate the progression into or through G1 phase growth. The present application contemplates that methods and compositions that increase the expression and/or activity of a G1 Cdk complex promote dedifferentiation.

The CDKs are subject to multiple levels of control. These proteins are positively regulated by association with cyclins (Evans et al. (1983) Cell 33: 389-396; Swenson et al. (1986) Cell 47: 861-870; Xiong et al. (1991) Cell 65: 691-699; Matsushime et al. (1991) Cell 66: 701-713; Koff et al. (1991) Cell 66: 1217-1228; Lew et al. (1991) Cell 66: 1197-1206) and activating phosphorylation by the cdk activating kinase (CAK) (Solomon et al. (1992) Mol. Biol. Cell 3: 13-27). Negative regulation of the cyclin/cdk(s) is achieved independently by at least two different mechanisms: binding of the inhibitory subunits (p21, p16, p15, p27 and p18) (c.f., Xiong et al. (1993) Nature 366, 701-704; Harper et al. (1993) Cell 75: 805-816; ElDeiry et al. (1993) Cell 75: 817-825; Gu et al. (1993) Nature 366: 707-710; Serrano et al. (1993) Nature 366: 704-707; Hannon et al. (1994) Nature 371: 257-261; Polyak et al. (1994) Cell 78: 59-66; Toyoshima et al. (1994) Cell 78: 67-74; Guan et al. (1994) Genes and Dev. 8: 2939-2950) and by phosphorylation of conservative threonine and tyrosine residues, usually at positions 14 and 15 in cdk(s) (Gould et al. (1989) Nature 342: 81-86; Krek et al. (1991) EMBO J. 10: 3331-3341; Gu et al. (1992) EMBO J. 11: 3995-4005; and Meyerson et al. (1992) EMBO J. 11: 2909-2917).

In certain embodiments, the subject method includes the use of dedifferentiation agents which increase the amount of D type cyclin (or other G1 phase cyclin), such as cyclin D1, in the treated cells. This can be done by any one or more of, for example, (i) inducing expressing of an endogenous cyclin gene, (ii) introducing an exogenous recombinant cyclin gene into the cell, (iii) contacting the cell with a D-type cyclin protein formulated for uptake by the cell, and/or (iv) increasing the intracellular half-life of a cyclin protein. Furthermore, any agent that increasing the expression and/or activity of a D-type cyclin, for example, a small organic molecule that increases the activity and/or expression of a D-type cyclin is contemplated as useful in the methods of the present invention.

The expression of D-type G1 cyclins and their assembly with their catalytic partners, the cyclin-dependent kinases 4 and 6 (CDK4 and CDK6), into active holoenzyme complexes are regulated at least in part by their inherent instability. The mechanisms governing the turnover of D-type cyclins include ubiquitination and proteasomal degradation, which is positively regulated, for example, by phosphorylation on threonine-286 (cyclin D1). Accordingly, the cells can be treated with compounds that inhibit phosphorylation, e.g., phosphorylation of threonine-286 on cyclin D1, inhibit ubiquitination of the cyclin, e.g., inhibit a E3 ligase which targets cyclin D1, and/or inhibit proteasome-mediated degradation of the ubiquitinated cyclin. Merely to illustrate, the cell can be treated with a proteasome inhibitor such as MG132 (Z-Leu-Leu-Leu-CHO). Sustained expression of cyclin D1 and D2 has been observed when cells are incubated with 3 mM or higher H₂O₂ concentrations. While not wishing to be bound by any particular theory, H₂O₂ may reversibly inhibit the ubiquitin-proteasome dependent degradation of cyclin D1 and D2, probably by transiently inhibiting ubiquitination and/or the proteasome. Martinez et al. (2001) Cell Mol Life Sci 58 (7):990. Accordingly, the subject method can include treatment of cells with H₂O₂ or other oxidizing agents.

There are a variety of small molecules which can positively effect the level of cyclin D1/cdk4 complexes, such as the fungal estrogen zearalenone. Such compounds can be used as dedifferentiation agents in the methods of the present invention.

The phosphorylation of CDC2 on Tyr-15 and Thr-14, two residues located in the putative ATP binding site of the kinase, negatively regulates kinase activity. This inhibitory phosphorylation of CDC2 is mediated at least in part by the weel and mikl tyrosine kinases (Russel et al. (1987) Cell 49: 559-567; Lundgren et al. (1991) Cell 64: 1111-1122; Featherstone et al. (1991) Nature 349: 808-811; and Parker et al. (1992) PNAS 89: 2917-2921). These kinases act as mitotic inhibitors, over-expression of which causes cells to arrest in the G2 phase of the cell-cycle. By contrast, loss of function of weel causes a modest advancement of mitosis, whereas loss of both weel and mikl function causes grossly premature mitosis, uncoupled from all checkpoints that normally restrain cell division (Lundgren et al. (1991) Cell 64: 1111-1122).

A stimulatory phosphatase, known as cdc25, is responsible for Tyr-15 and Thr-14 dephosphorylation and serves as a rate-limiting mitotic activator. (Dunphy et al. (1991) Cell 67: 189-196; Lee et al. (1992) Mol Biol. Cell. 3: 73-84; Millar et al. (1991) EMBO J. 10: 4301-4309; and Russell et al. (1986) Cell 45: 145-153). In human's, there are three known cdc25-related genes which share approximately 40-50% amino-acid identity (Sadhu et al. (1990) PNAS 87: 5139-5143; Galaktionov and Beach (1991) Cell 67: 1181-1194; and Nagata et al. (1991) New Biol. 3: 959-968). Human cdc25 genes were recently found to function at G1 and/or S-phase of the cell cycle (Jinno et al. (1994) EMBO J. 13: 1549-1556) in addition to the previously identified G₂ or M-phase functions (Galaktionov and Beach, D. ibid.; Millar, et al. (1991) PNAS 88: 10500-10504).

Given the role of cdc25 in promoting progression through the cell cycle, the invention contemplates that agents which upregulated/promote the expression and/or activity of cdc25 can be used to promote dedifferentiation. Such agents include small organic molecules that increase the expression and/or activity of cdc25, as well as agents which inhibit negative regulators of Wee1. Inhibition of Wee1 would relieve some of the negative regulation of the activity of cdc25, and would thus act to effectively promote the expression and/or activity of cdc25. Exemplary inhibitors of Wee1 expression and/or activity include small organic molecules that inhibit expression and/or activity of Wee1, antisense oligonucleotides that inhibit expression of Wee1, ribozymes that inhibit expression of Wee1, RNAi constructs that that inhibit expression of Wee1, and antibodies that bind to and inhibit the expression and/or activity of Wee1. By way of a non-limiting example, PD0166285 is a newly identified Wee1 inhibitor which abrogates the G₂ checkpoint (Li et al. (2000) Radiation Research 157: 322-330).

Exemplary agents that promote dedifferentiation and which promote expression and/or activity of cyclinD1 include, but are not limited to: (i) a nucleic acid encoding a cyclinD1 polypeptide, (ii) a cyclinD1 polypeptide, (iii) a small organic molecule that binds to and promotes the expression and/or activity of cyclinD1. Exemplary agents that promote dedifferentiation and which promote expression and/or activity of Cdk4 include, but are not limited to: (i) a nucleic acid encoding a Cdk4 polypeptide, (ii) a Cdk4 polypeptide, (iii) a small organic molecule that binds to and promotes the expression and/or activity of Cdk4. Exemplary agents that promote dedifferentiation and which inhibit expression and/or activity of p16 include, but are not limited to: (i) a small organic molecule that binds to and inhibits expression and/or activity of p16, (ii) an antibody that binds to and inhibits expression and/or activity of p16, (iii) an antisense oligonucleotide that binds to and inhibits expression and/or activity of p16, (iv) an RNAi construct that binds to and inhibits expression and/or activity of p16, and (v) a ribozyme that binds to and inhibits expression and/or activity of p16. Exemplary agents that promote dedifferentiation and which inhibit expression and/or activity of p21 include, but are not limited to: (i) a small organic molecule that binds to and inhibits expression and/or activity of p21, (ii) an antibody that binds to and inhibits expression and/or activity of p21, (iii) an antisense oligonucleotide that binds to and inhibits expression and/or activity of p21, (iv) an RNAi construct that binds to and inhibits expression and/or activity of p21, and (v) a ribozyme that binds to and inhibits expression and/or activity of p21. Exemplary agents that promote dedifferentiation and which inhibit expression and/or activity of Wee1 include, but are not limited to: (i) a small organic molecule that binds to and inhibits expression and/or activity of Wee1, (ii) an antibody that binds to and inhibits expression and/or activity of Wee1, (iii) an antisense oligonucleotide that binds to and inhibits expression and/or activity of Wee1, (iv) an RNAi construct that binds to and inhibits expression and/or activity of Wee1, and (v) a ribozyme that binds to and inhibits expression and/or activity of Wee1. Exemplary agents that promote dedifferentiation and which promote expression and/or activity of cdc25 include, but are not limited to: (i) a nucleic acid encoding a cdc25 polypeptide, (ii) a cdc25 polypeptide, (iii) a small organic molecule that binds to and promotes the expression and/or activity of cdc25. Exemplary agents that promote dedifferentiation and which inhibit expression and/or activity of Rb include, but are not limited to: (i) a small organic molecule that binds to and inhibits expression and/or activity of Rb, (ii) an antibody that binds to and inhibits expression and/or activity of Rb, (iii) an antisense oligonucleotide that binds to and inhibits expression and/or activity of Rb, (iv) an RNAi construct that binds to and inhibits expression and/or activity of Rb, and (v) a ribozyme that binds to and inhibits expression and/or activity of Rb.

In any of the foregoing, the application contemplates that agents may be administered alone, or may be administered in combination with one or more other agents. Similarly, in methods of screening for additional agents the application contemplates that agents may be screened singly or in combination with one or more other agents.

As described herein, one aspect of the invention pertains to variants of a wildtype polypeptide, wherein the variant either agonizes or antagonizes the function of the wildtype polypeptide. Furthermore, one aspect of the invention pertains to fragments of a wildtype polypeptide, wherein the fragments either agonize (retain a biological activity of the wildtype polypeptide) or antagonize the function of the wildtype polypeptide.

In addition to agonistic or antagonistic variants and fragments, the invention contemplates nucleic acids comprising nucleotide sequences encoding such agonistic or antagonistic variants and fragments. The term nucleic acid as used herein is intended to include equivalents. The term equivalent is understood to include nucleotide sequences which are functionally equivalent to a particular nucleotide sequence. Equivalent nucleotide sequences will include sequences that differ by one or more nucleotide substitutions, additions or deletions, such as allelic variants, and variation due to degeneracy of the genetic code. Equivalent sequences may also include nucleotide sequences that hybridize under stringent conditions (i.e., equivalent to about 20-27° C. below the melting temperature (Tₘ) of the DNA duplex formed in about 1M salt) to a given nucleotide sequence. Further examples of stringent hybridization conditions include awash step of 0.2×SSC at 65° C.

The present invention contemplates that agonistic and antagonistic variants and peptide variants, for example, variants comprising an amino acid sequence at least 60%, 70%, 75%, 80%, 85%, 90%, 95%, 98%, or 99% identical to an amino acid sequence provided in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, or SEQ ID NO: 78, can be encoded by a nucleic acid sequence. In one embodiment, the nucleic acid sequence is at least 60%, 70%, 75%, 80%, 85%, 90%, 95%, 98%, or 99% identical to a nucleic acid sequence provided in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID. NO: 71, SEQ ID NO: 73, SEQ ID NO: 75, or SEQ ID NO: 77.

In another embodiment, the nucleic acid sequence hybridizes under stringent conditions, including a wash step of 0.2×SSC at 65° to a nucleic acid sequence provided in SEQ ID NO: 1, SEQ ID NO: 3, SEQ ID NO: 5, SEQ ID NO: 7, SEQ ID NO: 9, SEQ ID NO: 1, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID. NO: 67, SEQ ID NO: 69, SEQ ID NO: 71, SEQ ID NO: 73, SEQ ID NO: 75, or SEQ ID NO: 77, or the complement thereof.

The present invention contemplates methods of administering nucleic acids encoding agonistic or antagonistic variants or peptide variants, wherein said nucleic acid promotes dedifferentiation. In a preferred embodiment, administering a nucleic acid encoding an agonistic or antagonistic variant or peptide variant promotes regeneration.

The invention further encompasses the use of nucleic acid molecules that differ from the nucleotide sequences provided in the sequence listing due to degeneracy of the genetic code and thus encode the same polypeptide as that encoded by the nucleotide sequences provided in the sequence listing.

More generally, the invention contemplates the use of nucleic acids that differ, due to the degeneracy of the genetic code, from the nucleotide sequences shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ NO: 45, SEQ ID NO: 47, SEQ ID NO:49, SEQ ID NO:51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, or SEQ ID NO: 77.

“Variant polynucleotides” or “variant nucleic acid sequences” for use in the methods of the present invention include nucleic acid molecules which encode an active polypeptide and that (1) have at least about 80% nucleic acid sequence identity with a nucleotide acid sequence provided in SEQ ID NO: 1, SEQ ID NO: 3, SEQ ID NO:5, SEQ ID NO: 7, SEQ ID NO: 9, SEQ ID NO: 1, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID. NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, or SEQ ID NO: 77; (2) have at least 80% nucleic acid sequence identity with a mature sequence (e.g., not including signal sequences or other sequences that are processed to yield the mature domain of the full-length polypeptide that possess the desired biological activity) provided in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, or SEQ ID NO: 77; or (3) have at least 80% nucleic acid sequence identity with a bioactive fragment of any of the full-length sequences provided in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, or SEQ ID NO: 77. Exemplary variant polynucleotides will have at least about 80% nucleic acid sequence identity, more preferably at least about 81%, 82%, 83%, 84%, 85%, 86%, 87%, 88%, 89%, 90%, 91%, 92%, 93%, 94%, 95%, 96%, 97%, or 98% nucleic acid sequence identity and yet more preferably at least about 99% nucleic acid sequence identity with at least one of the nucleic acid sequences provided herein. Ordinarily, variant polynucleotides for use in the methods of the present invention are at least about 30 nucleotides in length. In another embodiment, variant polynucleotides may be at least about 60, 90, 120, 150, 180, 210, 240, 270, 300, 450, or 600 nucleotides in length. In still other embodiment, variant polynucleotides may be at least about 900 nucleotides in length, or more. Regardless of the length of the polynucleotide variant, said variant is characterized by retaining at least one of the activities of the full-length, native polynucleotide sequence (e.g., the variant is “bioactive”).

“Percent (%) nucleic acid sequence identity” with respect to a nucleic acid sequence identified herein is defined as the percentage of nucleotides in a candidate sequence that are identical with the nucleotides in the sequence of interest, after aligning the sequences and introducing gaps, if necessary, to achieve the maximum percent sequence identity. Alignment for purposes of determining nucleic acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

When nucleotide sequences are aligned, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) can be calculated as follows:

% nucleic acid sequence identity W/Z=100

where W is the number of nucleotides scored as identical matches by the sequence alignment program's or algorithm's alignment of C and D and Z is the total number of nucleotides in D.

When the length of nucleic acid sequence C is not equal to the length of nucleic acid sequence D, the % nucleic acid sequence identity of C to D will not equal the % nucleic acid sequence identity of D to C.

Homologs (i.e., nucleic acids encoding a particular polypeptide but derived from other species) or other related sequences (e.g., paralogs) can be obtained by low, moderate or high stringency hybridization with all or a portion of the particular sequence as a probe using methods well known in the art for nucleic acid hybridization and cloning.

The specificity of single stranded DNA to hybridize complementary fragments is determined by the “stringency” of the reaction conditions. Hybridization stringency increases as the propensity to form DNA duplexes decreases. In nucleic acid hybridization reactions, the stringency can be chosen to either favor specific hybridizations (high stringency), which can be used to identify, for example, full-length clones from a library. Less-specific hybridizations (low stringency) can be used to identify related, but not exact, DNA molecules (homologous, but not identical) or segments.

DNA duplexes are stabilized by: (1) the number of complementary base pairs, (2) the type of base pairs, (3) salt concentration (ionic strength) of the reaction mixture, (4) the temperature of the reaction, and (5) the presence of certain organic solvents, such as formamide which decreases DNA duplex stability. In general, the longer the probe, the higher the temperature required for proper annealing. A common approach is to vary the temperature: higher relative temperatures result in more stringent reaction conditions. (Ausubel et al., 1987) provide an excellent explanation of stringency of hybridization reactions.

To hybridize under “stringent conditions” describes hybridization protocols in which nucleotide sequences at least 60% homologous to each other remain hybridized. Generally, stringent conditions are selected to be about 5° C. lower than the thermal melting point (Tm) for the specific sequence at a defined ionic strength and pH. The Tₘ is the temperature (under defined ionic strength, pH and nucleic acid concentration) at which 50% of the probes complementary to the target sequence hybridize to the target sequence at equilibrium. Since the target sequences are generally present at excess, at Tin, 50% of the probes are occupied at equilibrium.

(a) High Stringency

“Stringent hybridization conditions” conditions enable a probe, primer or oligonucleotide to hybridize only to its target sequence. Stringent conditions are sequence-dependent and will differ. Stringent conditions comprise: (1) low ionic strength and high temperature washes (e.g., 15 mM sodium chloride, 1.5 mM sodium citrate, 0.1% sodium dodecyl sulfate at 50° C.); (2) a denaturing agent during hybridization (e.g., 50% (v/v) formamide, 0.1% bovine serum albumin, 0.1% Ficoll, 0.1% polyvinylpyrrolidone, 50 mM sodium phosphate buffer (pH 6.5; 750 mM sodium chloride, 75 mM sodium citrate at 42° C.); or (3) 50% formamide. Washes typically also comprise 5×SSC (0.75 M NaCl, 75 mM sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. Preferably, the conditions are such that sequences at least about 65%, 70%, 75%, 80%, 85%, 90%, 95%, 98%, or 99% homologous to each other typically remain hybridized to each other. These conditions are presented as examples and are not meant to be limiting.

(b) Moderate Stringency

“Moderately stringent conditions” use washing solutions and hybridization conditions that are less stringent (Sambrook, 1989), such that a polynucleotide will hybridize to the entire, fragments, derivatives or analogs of a sequence. One example comprises hybridization in 6×SSC, 5×Denhardt's solution, 0.5% SDS and 100 mg/ml denatured salmon sperm DNA at 55° C., followed by one or more washes in 1×SSC, 0.1% SDS at 37° C. The temperature, ionic strength, etc., can be adjusted to accommodate experimental factors such as probe length. Other moderate stringency conditions are described in (Ausubel et al., 1987; Kriegler; 1990).

(c) Low Stringency

“Low stringent conditions” use washing solutions and hybridization conditions that are less stringent than those for moderate stringency (Sambrook, 1989), such that a polynucleotide will hybridize to the entire, fragments, derivatives or analogs of a sequence. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency, such as those for cross-species hybridizations are described in (Ausubel et al., 1987; Kriegler, 1990; Shilo and Weinberg, 1981).

In addition to naturally occurring allelic variants of a given nucleic acid sequence, changes can be introduced by mutation of the nucleic acid sequence that incur alterations in the amino acid sequences of the polypeptide encoded by that nucleic acid sequence. Such variant sequences may either possess the same (or nearly the same) function as the native sequence, or such variant sequences may possess a function different from that of the native sequence. For example, such variants may have no function at all, or may function to antagonize the activity of the native polypeptide. One of skill in the art can readily test the function of the variant polypeptide encoded by the variant nucleic acid sequence using any number of in vitro or in vivo assays suitable for the particular polypeptide being tested. For example, a variant of a given ligand can be tested, in vitro or in vivo, for the ability to bind its native receptor, or for its ability to induce expression of particular downstream genes (i.e., to promote, or inhibit particular signal transduction pathways normally modulated by the native polypeptide).

To further illustrate, nucleotide substitutions leading to amino acid substitutions at “nonessential” amino acid residues can be made. A “non-essential” amino acid residue is a residue that can be altered from the wild-type sequence without altering the biological activity of the polypeptide, whereas an “essential” amino acid residue is required for a given biological activity. Often, although not always, an amino acid residue has been highly conserved across species is one that is necessary for the function of the polypeptide. Such amino acid residues are less likely to be amenable to change or substitution without affecting the function of the polypeptide. However, such conserved amino acid residues are excellent candidates for positions whereby sequence variation is likely to result in a polypeptide with a different function from the native polypeptide.

Useful conservative substitutions are shown in Table A, “Preferred substitutions.” Conservative substitutions whereby an amino acid of one class is replaced with another amino acid of the same type fall within the scope of the subject invention. Due to the relatedness in size and charge, substitution of an amino acid residue with another residue from within the same class often does not materially alter the biological activity of the compound. Table B provides additional exemplary amino acid substitutions. Although the substitutions provided in Table B generally are considered to comprise more substantial changes in structure than the substitutions provided in Table A, one of skill in the art can readily make a large number of candidate variant polypeptides and screen for variants having the desired biological activity.

TABLE A
Preferred substitutions

| Original residue | Exemplary substitutions |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| Gly (G) | Pro, Ala |
| His (H) | Asn, Gln, Lys, Arg |
| Ile (I) | Leu, Val, Met, Ala, Phe, |
| | Norleucine |
| Leu (L) | Norleucine, Ile, Val, Met, |
| | Ala, Phe |
| Lys (K) | Arg, Gln, Asn |
| Met (M) | Leu, Phe, Ile |
| Phe (F) | Leu, Val, Ile, Ala, Tyr |
| Pro (P) | Ala |
| Ser (S) | Thr |
| Thr (T) | Ser |
| Trp (W) | Tyr, Phe |
| Tyr (Y) | Trp, Phe, Thr, Ser |
| Val (V) | Ile, Leu, Met, Phe, Ala, |
| | Norleucine |

Non-conservative substitutions that affect (1) the structure of the polypeptide backbone, such as a β-sheet or α-helical conformation, (2) the charge, (3) hydrophobicity, or (4) the bulk of the side chain of the target site may modify the polypeptide's function or immunological identity. Residues are divided into groups based on common side-chain properties as denoted in Table B. Non-conservative substitutions entail exchanging a member of one of these classes for another class. Substitutions may be introduced into conservative substitution sites or more preferably into non-conserved sites.

TABLE B
Amino acid classes

| Class | Amino acids |
|---|---|
| hydrophobic | Norleucine, Met, Ala, Val, Leu, Ile |
| neutral hydrophilic | Cys, Ser, Thr |
| Acidic | Asp, Glu |
| Basic | Asn, Gln, His, Lys, Arg |
| disrupt chain conformation | Gly, Pro |
| aromatic | Trp, Tyr, Phe |

The variant polypeptides can be made using methods known in the art such as oligonucleotide-mediated (site-directed) mutagenesis, alanine scanning, and PCR mutagenesis. Site-directed mutagenesis (Carter, 1986; Zoller and Smith, 1987), cassette mutagenesis, restriction selection mutagenesis (Wells et al., 1985) or other known techniques can be performed on the cloned DNA to produce msx1 variant DNA (Ausubel et al., 1987; Sambrook, 1989).

In one embodiment, the isolated nucleic acid molecule comprises a nucleotide sequence encoding a protein, wherein the protein comprises an amino acid sequence at least about 45%, preferably 60%, more preferably 70%, 80%, 85%, or 90% identical to a native polypeptide sequence. In another embodiment, the amino acid sequence is at least 95%, 97%, 98%, 99%, or greater than 99% identical to a native polypeptide sequence. To illustrate more specifically, the invention contemplates the making of polypeptides at least 45%, 60%, 70%, 80%, 85%, 90%, 95%, 97%, 98%, 99%, or greater than 99% identical to a polypeptide sequence provided in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ED NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, or SEQ ID NO: 78. The invention further contemplates that polypeptides comprising amino acid sequences at least 45%, 60%, 70%, 75%, 80%, 85%, 90%, 95%, 97%, 98%, 99%, or greater than 99% identical to any of the foregoing amino acid sequences, and which polypeptides retain one or more biological activities of the native polypeptide sequence, can be used in the methods of the present invention to dedifferentiate a cell.

One aspect of the invention pertains to the use of, for example, isolated msx1, and biologically active portions, derivatives, fragments, analogs or homologs thereof. However, the proceeding section is applicable to all dedifferentiation agents, and msx1 will be used as an example for illustration purposes Also provided are polypeptide fragments suitable for use as immunogens to raise anti-msx1 Abs. In one embodiment, a native msx1 can be isolated from cells or tissue sources by an appropriate purification scheme using standard protein purification techniques. In another embodiment, msx1 are produced by recombinant DNA techniques. Alternative to recombinant expression, msx1 can be synthesized chemically using standard peptide synthesis techniques.

(a) Msx1 Polypeptides

Msx1 polypeptides include the amino acid sequence of msx1 whose sequence is provided in SEQ ID NO:2, SEQ ID NO: 4, SEQ ID NO: 6, or SEQ ID NO: 8. The invention also includes a mutant or variant protein any of whose residues may be changed from the corresponding residues shown in SEQ ID NO:2, SEQ ID NO: 4, SEQ. ID NO: 6, or SEQ ID NO: 8, while still encoding a protein that maintains msx1 activities and physiological functions, or a functional fragment thereof.

(b) Variant Msx1 Polypeptides

In general, msx1 variants that preserve msx1-like function includes any variant in which residues at a particular position in the sequence have been substituted by other amino acids, and further includes the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

“msx1 polypeptide variant” means an active msx1 polypeptide having at least: (1) about 80% amino acid sequence identity with a full-length native sequence msx1 polypeptide sequence, (2) msx1 polypeptide sequence lacking the signal peptide, (3) an extracellular domain of msx1 polypeptide, with or without the signal peptide, or (4) any other fragment of a full-length msx1 polypeptide sequence. For example, msx1 polypeptide variants include msx1 polypeptides wherein one or more amino acid residues are added or deleted at the N- or C-terminus of the full-length native amino acid sequence. Msx1 polypeptide variant will have at least about 80% amino acid sequence identity, preferably at least about 81% amino acid sequence identity, more preferably at least about 82%, 83%, 84%, 85%, 86%, 87%, 88%, 89%, 90%, 91%, 92%, 93%, 94%, 95%, 96%, 97%, or 98% amino acid sequence identity and most preferably at least about 99% amino acid sequence identity with a full-length native sequence msx 1 polypeptide sequence. Msx1 polypeptide variant may have a sequence lacking the signal peptide, an extracellular domain of msx1 polypeptide, with or without the signal peptide, or any other fragment of a full-length msx1 polypeptide sequence. Ordinarily, msx1 variant polypeptides are at least about amino acids in length, often at least about 20 amino acids in length, more often at least about 30, 40, 50, 60, 70, 80, 90, 100, 150, 200, or 300 amino acids in length, or more.

“Percent (%) amino acid sequence identity” is defined as the percentage of amino acid residues that are identical with amino acid residues in a disclosed msx1 polypeptide sequence in a candidate sequence when the two sequences are aligned. To determine % amino acid identity, sequences are aligned and if necessary, gaps are introduced to achieve the maximum % sequence identity; conservative substitutions are not considered as part of the sequence identity. Amino acid sequence alignment procedures to determine percent identity are well known to those of skill in the art. Often publicly available computer software such as BLAST, BLAST2, ALIGN2 or Megalign (DNASTAR) software is used to align peptide sequences. Those skilled in the art can determine appropriate parameters for measuring alignment, including any algorithms needed to achieve maximal alignment over the full length of the sequences being compared.

When amino acid sequences are aligned, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) can be calculated as:

amino acid sequence identity=X/Y*100

where X is the number of amino acid residues scored as identical matches by the sequence alignment program's or algorithm's alignment of A and B and Y is the total number of amino acid residues in B.

If the length of amino acid sequence A is not equal to the length of amino acid sequence B, the % amino acid sequence identity of A to B will not equal the % amino acid sequence identity of B to A.

(c) Isolated/Purified Polypeptides

An “isolated” or “purified” polypeptide, protein or biologically active fragment is separated and/or recovered from a component of its natural environment. Contaminant components include materials that would typically interfere with diagnostic or therapeutic uses for the polypeptide, and may include enzymes, hormones, and other proteinaceous or non-proteinaceous materials. Preferably, the polypeptide is purified to a sufficient degree to obtain at least 15 residues of N-terminal or internal amino acid sequence. To be substantially isolated, preparations having less than 30% by dry weight of non-msx1 contaminating material (contaminants), more preferably less than 20%, 10% and most preferably less than 5% contaminants. An isolated, recombinantly-produced msx1 or biologically active portion is preferably substantially free of culture medium, i.e., culture medium represents less than 20%, more preferably less than about 10%, and most preferably less than about 5% of the volume of the msx1 preparation. Examples of contaminants include cell debris, culture media, and substances used and produced during in vitro synthesis of msx1.

(d) Chimeric and Fusion Proteins

Fusion polypeptides are useful in expression studies, cell-localization, bioassays, msx1 purification, and for the purposes of the methods of the invention, for intracellular introduction of msx1 by extracellular application. Msx1 “chimeric protein” or “fusion protein” comprises msx1 fused to a non-msx1 polypeptide. A non-msx1 polypeptide is not substantially homologous to msx1. Msx1 fusion protein may include any portion of an entire msx1, including any number of the biologically active portions. Msx1 may be fused to the C-terminus of the GST (glutathione S-transferase) sequences. Such fusion proteins facilitate the purification of a recombinant msx1. In certain host cells, (e.g., mammalian), heterologous signal sequence fusions may ameliorate msx1 expression and/or intracellular uptake. For example, residues of the HIV tat protein can be used to encourage intracellular uptake and nuclear delivery (Frankel et al., U.S. Pat. No. 5,804,604, 1998). Additional exemplary fusions are presented in Table C.

Fusion proteins can be easily created using recombinant methods. A nucleic acid encoding msx1 can be fused in-frame with a non-msx1 encoding nucleic acid, to msx1 NH₂— or COO— -terminus, or internally. Fusion genes may also be synthesized by conventional techniques, including automated DNA synthesizers. PCR amplification, using anchor primers that give rise to complementary overhangs between two consecutive gene fragments that can subsequently be annealed and reamplified to generate a chimeric gene sequence (Ausubel et al., 1987), is also useful. Many vectors are commercially available that facilitate sub-cloning msx1 inframe to a fusion moiety.

TABLE C
Useful fusion polypeptides

| Reporter | in vitro | in vivo. | Notes | Reference |
|---|---|---|---|---|
| Human growth | Radioimmuno- | None | Expensive, | (Selden et al., |
| hormone (hGH) | assay | | insensitive, | 1986) |
| | | | narrow linear | |
| | | | range. | |
| B-glucuronidase | Colorimetric, | Colorimetric | sensitive, | (Gallagher, |
| (GUS) | fluorescent, or | (histo-chemical | broad linear | 1992) |
| | chemi- | staining with | range, non | |
| | luminescent | X-gluc) | iostopic. | |
| Green fluorescent | Fluorescent | fluorescent | can be used in | (Chalfie et al., |
| protein (GFP) and | | | live cells; | 1994) |
| related molecules | | | resists photo | |
| (RFP, BFP, msxl, | | | bleaching | |
| etc.) | | | | |
| Luciferase | bioluminsecent | Bio- | protein is | (de Wet et al., |
| (firefly) | | luminescent | unstable, | 1987) |
| | | | difficult to | |
| | | | reproduce, | |
| | | | signal is brief | |
| Chloramphenicoal | Chromatography, | None | Expensive | (Gorman et |
| acetyltransferase | differential | | radioactive | al., 1982) |
| (CAT) | extraction, | | substrates, | |
| | fluorescent, or | | time | |
| | immunoassay | | consuming, | |
| | | | insensitive, | |
| | | | narrow linear | |
| | | | range | |
| B-galacto-sidase | colorimetric, | Colorimetric | sensitive, | (Alam and |
| | fluorescence, | (histochemical | broad linear | Cook, 1990) |
| | chemi- | staining with | range; some | |
| | luminscence | X-gal), bio- | cells have | |
| | | luminescent in | high | |
| | | live cells | endogenous | |
| | | | activity | |
| Secrete alkaline | colorimetric, | None | Chem- | (Berger et al., |
| phosphatase | bioluminescent, | | iluminscence | 1988) |
| (SEAP) | chemi- | | assay is | |
| | luminescent | | sensitive and | |
| | | | broad linear | |
| | | | range; some | |
| | | | cells have | |
| | | | endogenouse | |
| | | | alkaline | |
| | | | phosphatase | |
| | | | activity | |
| Tat from HIV | Mediates | Mediates | Exploits | (Frankel et |
| | delivery into | delivery into | amino acid | al., U.S. Pat. |
| | cytoplasm and | cytoplasm and | residues of | No. 5,804,604, |
| | nuclei | nuclei | HIV tat | 1998) |
| | | | protein. | |

G. Biochemical

An extract is most simply a preparation that is in a different form than its source. A cell extract may be as simple as mechanically-lysed cells. Such preparations may be clarified by centrifugation or filtration to remove insoluble debris.

Extracts also comprise those preparations that involve the use of a solvent. A solvent may be water, a detergent, or an organic compound, as non-limiting examples. Extracts may be concentrated, removing most of the solvent and/or water; and may also be fractionated, using any method common to those of skill in the art (such as a second extraction, size fractionation by gel filtration or gradient centrifugation, etc.). In addition, extracts may also contain substances added to the mixture to preserve some components, such as the case with protease inhibitors to prolong protein life, or sodium azide to prevent microbial contamination.

Often, cell or tissue extracts are made to isolate a component from the intact source; for example, growth factors, surface proteins, nucleic acids, lipids, polysaccharides, etc., or even different cellular compartments, including Golgi vesicles, lysosomes, nuclei, mitochondria and chloroplasts may be extracted from cells.

### Methods of Expressing Agents

The systems and methods described herein also provide expression vectors containing a nucleic acid encoding an agent that promotes dedifferentiation, operably linked to at least one transcriptional regulatory sequence. Regulatory sequences are art-recognized and are selected to direct expression of the subject proteins. Accordingly, the term transcriptional regulatory sequence includes promoters, enhancers and other expression control elements. Such regulatory sequences are described in Goeddel; Gene Expression Technology: Methods in Enzymology 185, Academic Press, San Diego, Calif. (1990). For instance, any of a wide variety of expression control sequences may be used in these vectors to express nucleic acid sequences encoding the agents of this invention. Such useful expression control sequences, include, for example, a viral LTR, such as the LTR of the Moloney murine leukemia virus, the LTR of the Herpes Simplex virus-1, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage λ, the control regions for fd coat protein, the promoter for 3-phosphoglycerate kinase or other glycolytic enzymes, the promoters of acid phosphatase, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other proteins encoded by the vector, such as antibiotic markers, should also be considered.

Moreover, the gene constructs can be used to deliver nucleic acids encoding the subject polypeptides. Thus, another aspect of the invention features expression vectors for in vivo or in vitro transfection, viral infection and expression of a subject polypeptide in particular cell types.

The application further describes peptides and polypeptide agents for promoting dedifferentiation, as well as methods for producing the subject polypeptides. For example, a host cell transfected with a nucleic acid vector directing expression of a nucleotide sequence encoding the subject polypeptides can be cultured under appropriate conditions to allow expression of the peptide to occur. The polypeptide may be secreted and isolated from a mixture of cells and medium containing the recombinant polypeptide. Alternatively, the peptide may be expressed cytoplasmically and the cells harvested, lysed and the protein isolated. A cell culture includes host cells, media and other by-products. Suitable media for cell culture are well known in the art. The recombinant polypeptide can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for such peptide. In one example, the recombinant polypeptide is a fusion protein containing a domain which facilitates its purification, such as a GST fusion protein. In another example, the subject recombinant polypeptide may include one or more additional domains which facilitate immunodetection, purification, and the like. Exemplary domains include HA, FLAG, GST, His, and the like. Further exemplary domains include a protein transduction domain (PTD) which facilitates the uptake of proteins by cells.

This application also describes a host cell which expresses a recombinant form of the subject polypeptides. The host cell may be a prokaryotic or eukaryotic cell. Thus, a nucleotide sequence derived from the cloning of a protein encoding all or a selected portion (either an antagonistic portion or a bioactive fragment) of the full-length protein, can be used to produce a recombinant form of a polypeptide via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial cells), are standard procedures used in producing other well-known proteins, e.g. insulin, interferons, human growth hormone, IL-1, IL-2, and the like. Similar procedures, or modifications thereof, can be employed to prepare recombinant polypeptides by microbial means or tissue-culture technology in accord with the subject invention. Such methods are used to produce experimentally useful proteins that include all or a portion of the subject nucleic acids.

The recombinant genes can be produced by ligating a nucleic acid encoding a protein, or a portion thereof, into a vector suitable for expression in either prokaryotic cells, eukaryotic cells, or both. Expression vectors for production of recombinant forms of the subject polypeptides include plasmids and other vectors. For instance, suitable vectors for the expression of a polypeptide include plasmids of the types: pBR322-derived plasmids, pEMBL-derived plasmids, pEX-derived plasmids, pGEX-derived plasmids, pTrc-His-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae.

Many mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo, pBacMam-2, and pHyg derived vectors are examples' of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. For other suitable expression systems for both prokaryotic and eukaryotic cells, as well as general recombinant procedures, see Molecular Cloning A Laboratory Manual, 3rd Ed., ed. by Sambrook and Russell (Cold Spring Harbor Laboratory Press: 2001).

In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUWI), and pBlueBac-derived vectors (such as the β-gal containing pBlueBac III).

Techniques for making fusion genes are known to those skilled in the art. The joining of various nucleic acid fragments-coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another example, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence.

Isolated peptidyl portions of proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry.

The recombinant polypeptides of the present invention also include versions of those proteins that are resistant to proteolytic cleavage. Variants of the present invention also include proteins which have been post-translationally modified in a manner different than the authentic protein. Modification of the structure of the subject polypeptides can be for such purposes as enhancing therapeutic or prophylactic efficacy, or stability (e.g., ex vivo shelf life and resistance to proteolytic degradation in vivo).

Advances in the fields of combinatorial chemistry and combinatorial mutagenesis have facilitated the making of polypeptide variants (Wissmanm et al. (1991) Genetics 128: 225-232; Graham et al. (1993) Biochemistry 32: 6250-6258; York et al. (1991) Journal of Biological Chemistry 266: 8495-8500; Reidhaar-Olson et al. (1988) Science 241: 53-57). Given one or more assays for testing polypeptide variants, one can assess whether a given variant functions as an antagonist, or whether a given variant has the same or substantially the same function as the wildtype protein. In the context of the present invention, several methods for assaying the functional activity of potential variants are provided.

To further illustrate, the invention contemplates a method for generating sets of combinatorial mutants, as well as truncation mutants, and is especially useful for identifying potentially useful variant sequences.

The application also describes reducing a protein to generate mimetics, e.g. peptide or non-peptide agents. Mimetics having a desired biological activity can be readily tested in vitro or in vivo.

The present invention also contemplates the use of nucleic acid inhibitors such as antisense oligonucleotide, RNAi constructs, DNA enzymes, and ribozymes. The selection of optimal nucleic acid sequences to promote dedifferentiation by inhibiting the function and/or activity of one or more proteins that inhibit dedifferentiation can be facilitated by the construction and screening of libraries of nucleic acid sequences following similar methodology as outlined in detail above.

Similarly, the present invention also contemplates the use of small organic molecules that either promote the function and/or activity of a protein that promotes dedifferentiation or that inhibits the function and/or activity of a protein that inhibits dedifferentiation. A variety of chemical libraries and libraries of small organic molecules are available, and these can be readily screened for agents with the desired activities.

Constructs comprising the subject agents may be administered in biologically effective carriers, e.g. any formulation or composition capable of effectively delivering the agents to cells in vivo or in vitro. The particular approach can be selected from amongst those well known to one of skill in the art based on the particular agent to be delivered (e.g., DNA enzyme, polypeptide variant, peptidomimetic, RNAi construct, antibody, antisense oligonucleotide, small organic molecule, and the like), the cell type to which delivery is desired, and the route of administration.

Approaches include viral vectors including recombinant retroviruses, adenovirus, adeno-associated virus, herpes simplex virus-1, lentivirus, mammalian baculovirus or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct, electroporation or CaPO₄ precipitation. One of skill in the art can readily select from available vectors and methods of delivery in order to optimize expression in a particular cell type or under particular conditions.

Retrovirus vectors and adeno-associated virus vectors have been frequently used for the transfer of exogenous genes. These vectors can be used to deliver nucleic acids, for example RNAi constructs, as well as to deliver nucleic acids encoding particular proteins such as polypeptide variants. These vectors provide efficient delivery of genes into cells. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes. Thus, recombinant retrovirus can be constructed in which part of the retroviral coding sequence (gag, pol, env) has been replaced by nucleic acid encoding one of the subject proteins rendering the retrovirus replication defective. The replication defective retrovirus is then packaged into virions through the use of a helper virus by standard techniques which can be used to infect a target cell. Protocols for producing recombinant retroviruses and for infecting cells in vitro or in vivo with such viruses can be found in Current Protocols in Molecular Biology, Ausubel, F. M. et al. (eds.) Greene Publishing Associates, (2000), and other standard laboratory manuals. Examples of suitable retroviruses include pBPSTR1, pLJ, pZIP, pWE and pEM which are known to those skilled in the art. Examples of suitable packaging virus lines for preparing both ecotropic and amphotropic retroviral systems include ψCrip, ψCre, ψ2, ψAm, and PA317.

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein; or coupling cell surface receptor ligands to the viral env proteins. Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, can also be used to convert an ecotropic vector into an amphotropic vector.

Moreover, use of retroviral gene delivery can be further enhanced by the use of tissue- or cell-specific transcriptional regulatory sequences which control expression of the gene of the retroviral vector such as tetracycline repression or activation.

Another viral gene delivery system which has been employed utilizes adenovirus-derived vectors. The genome of an adenovirus can be manipulated so that it encodes and expresses a gene product of interest but is inactivated in terms of its ability to replicate in a normal lytic viral life cycle. Suitable adenoviral vectors derived from the adenovirus strain Ad type 5 d1324 or other strains of adenovirus (e.g., Ad2, Ad3, Ad7 etc.) are known to those skilled in the art. Recombinant adenoviruses can be advantageous in certain circumstances in that they can be used to infect a wide variety of cell types, including airway epithelium, endothelial cells, hepatocytes, and muscle cells. Furthermore, the virus particle is relatively stable and amenable to purification and concentration, and as above, can be modified so as to affect the spectrum of infectivity.

Yet another viral vector system is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158: 97-129). It is also one of the few viruses that may integrate its DNA into non-dividing cells, and exhibits a high frequency of stable integration.

Another viral delivery system is based on herpes simplex-1 (HSV-1). HSV-1 based vectors may be especially useful in the methods of the present invention because they have been previously shown to infect neuronal cells. Given that many adult neuronal cells are post-mitotic, and thus have been difficult to infect using some other commonly employed viruses, the use of HSV-1 represents a substantial advance and further underscores the potential utility of viral based systems to facilitate gene expression in the nervous system (Agudo et al. (2002) Human Gene Therapy 13: 665-674; Latchman (2001) Neuroscientist 7: 528-537; Goss et al. (2002) Diabetes 51: 2227-2232; Glorioso (2002) Current Opin Drug Discov Devel 5: 289-295; Evans (2002) Clin Infect Dis 35: 597-605; Whitley (2002) Journal of Clinical Invest 110: 145-151; Lilley (2001) Curr Gene Ther 1: 339-359).

The above cited examples of viral vectors are by no means exhaustive. However, they are provided to indicate that one of skill in the art may select from well known viral vectors, and select a suitable vector for expressing a particular protein in a particular cell type.

In addition to viral transfer methods, such as those illustrated above, non-viral methods can be used. Many nonviral methods of gene transfer rely on normal mechanisms used by cells for the uptake and intracellular transport of macromolecules. Exemplary gene delivery systems of this type include liposomal derived systems, poly-lysine conjugates, and artificial viral envelopes.

It may sometimes be desirable to introduce a nucleic acid directly to a cell, for example a cell in culture or a cell in an animal. Such administration can be done by injection of the nucleic acid (e.g., DNA, RNA) directly at the desired site. Such methods are commonly used in the vaccine field, specifically for administration of “DNA vaccines”, and include condensed DNA (U.S. Pat. No. 6,281,005).

In addition to administration of nucleic acids, the systems and methods described herein contemplate that polypeptides may be administered directly. Some proteins, for example factors that act extracellularly by contacting a cell surface receptor, such as growth factors, may be administered by simply contacting cells with said protein. For example, cells are typically cultured in media which is supplemented by a number of proteins such as FGF, TGFβ, insulin, etc. These proteins influence cells by simply contacting the cells. Such a method similarly pertains to other agents such as small organic molecules and chemical compounds. These agents may either exert their effect at the cell surface, or may be able to permeate the cell membrane without the need for additional manipulation.

In another embodiment, a polypeptide is directly introduced into a cell. Methods of directly introducing a polypeptide into a cell include, but are not limited to, protein transduction and protein therapy. For example, a protein transduction domain (PTD) can be fused to a nucleic acid encoding a particular polypeptide antagonist, and the fusion protein is expressed and purified. Fusion proteins containing the PTD are permeable to the cell membrane, and thus cells can be directly contacted with a fusion protein (Derossi et al. (1994) Journal of Biological Chemistry 269: 10444-10450; Han et al. (2000) Molecules and Cells 6: 728-732; Hall et al. (1996) Current Biology 6: 580-587; Theodore et al. (1995) Journal of Neuroscience 15: 7158-7167).

Although some protein transduction based methods rely on fusion of a polypeptide of interest to a sequence which mediates introduction of the protein into a cell, other protein transduction methods do not require covalent linkage of a protein of interest to a transduction domain. At least two commercially available reagents exist that mediate protein transduction without covalent modification of the protein (Chariot™, produced by Active Motif, www.activemotif.com and Bioporter® Protein Delivery Reagent, produced by Gene Therapy Systems, www.genetherapysystems.com).

Briefly, these protein transduction reagents can be used to deliver proteins, peptides and antibodies directly to cells including mammalian cells. Delivery of proteins directly to cells has a number of advantages. Firstly, many current techniques of gene delivery are based on delivery of a nucleic acid sequence which must be transcribed and/or translated by a cell before expression of the protein is achieved. This results in a time lag between delivery of the nucleic acid and expression of the protein. Direct delivery of a protein decreases this delay. Secondly, delivery of a protein often results in transient expression of the protein in a cell.

As outlined herein, protein transduction mediated by covalent attachment of a PTD to a protein can be used to deliver a protein to a cell. These methods require that individual proteins be covalently appended with PTD moieties. In contrast, methods such as Chariot™ and Bioporter® facilitate transduction by forming a noncovalent interaction between the reagent and the protein. Without being bound by theory, these reagents are thought to facilitate transit of the cell membrane, and following internalization into a cell the reagent and protein complex disassociates so that the protein is free to function in the cell.

### IV. Practicing the Invention

A. RNLE Extract

The following describes the preparation of a regenerating newt limb extract developed for the instant invention. Also see Examples. It will be apparent to one of skill in the art that many variations of the following procedure may yield extracts with similar activities. In general, any extract produced from newts that has at least one of the activities of the extract (see examples) is contemplated by the inventors.

However, any extract comprising regeneration activities can be similarly prepared from any animal that regenerates, for example, urodeles (newt or axolotl) and teleost fish, such as Danio rerio, (zebrafish), or from regenerating mammalian liver. Such extracts will have at least one activity of RE.

For example, adult newts, Notophthalmus viridescens are maintained in a humidified room. Operations are performed on anesthetized animals. Regenerating limb tissue is collected as follows. Forelimbs are amputated by cutting just proximal to the elbow and soft tissue is pushed up the humorus to expose the bone. The bone and soft tissue are trimmed to produce a flat amputation surface. The newts are placed in a sulfamerazine solution overnight and then back into a normal water environment. Early regenerating tissue (days 1, 3, and 5 postamputation) is collected by reamputating the limb 0.5-1.0 mm proximal to the wound epithelium and removing any residual bone. Nonregenerating limb tissue is collected from limbs that had not been previously amputated. Tissue is extracted 2-3 mm proximal to the forelimb elbow and all bones are removed. Immediately after collection, all tissues are flash frozen in liquid nitrogen and stored at −80° C.

Tissues are thawed and all subsequent manipulations are performed at 4° C. or on ice. Six grams of early regenerating tissue from days 1, 3, and 5 (2 grams each) or six grams of nonregenerating tissue are placed separately into appropriate cell culture medium containing three protease inhibitors (for example, leupeptin, A-protinin, and phenylmethylsulfonyl fluoride). Tissues are ground with a tissue homogenizer, hand homogenized, and then briefly sonicated. Cell debris is removed in two centrifugation steps. The nonsoluble lipid layer is aspirated and the remaining supernatant filter sterilized. The protein content is then assayed and the extract stored at −80° C.

B. hRNLE; Identifying Active Components of RNLE

1. Introduction

The invention also comprises a composition that mimics at least one activity of RNLE that comprises human forms of the active molecules. For example, if Fgf is a component of RNLE (a likely possibility; see Examples), a human form of Fgf would be substituted in hRNLE compositions. A “humanized” formulation of RNLE would be advantageous to circumvent provoking an immune response in a human subject in need of a RNLE or RNLE-like composition.

2. Biochemical Approach

To one of skill in the art, it will be apparent how to determine the composition of hRNLE, using RNLE as a starting point and a functional assay based on, for example, regenerating newt limbs, or inducing dedifferentiation of mammalian myotubes. For example, using classic biochemical separation techniques, the components of RNLE can be fractionated and tested in a functional assay. When an activity is found, even if only a partial or subtle effect, then the isolated component is a candidate molecule that comprises an active RNLE. While each component may have a small effect, the sum of all RNLE purified active components will mimic that of RNLE.

3. Genetic Approach

To identify the active components in RNLE, and even the pathway and succession of events in regeneration, a genetic system can be employed. The invention demonstrates that fin regeneration in the genetically-amenable organism of Zebrafish requires Fgf signaling. Using a genetic approach, the individual genes that encode the factors responsible for RNLE-like activity can be identified by mapping and cloning. Once cloned, the Zebrafish gene sequences can be used to identify human homologues, using, for example cDNA or genomic DNA screening of human libraries. Similarly, BLAST searches and other in silico methods may obviate the need for such experimentation for some of the identified genes. In such a way, hRNLE (or that of the organism of choice) may be formulated.

The following outlines one genetic approach. However, one of skill in the art may vary or take a different genetic approach to achieve the same goal. For example, in cases where homozygosity at a mutated gene results in lethality, one of skill in the art may look for mutants with conditional alleles, such as temperature sensitive alleles. In general, a genetic approach requires a suitable organism, such as Zebrafish, and a screen or selection (a screen allows for the identification of a desired mutant among many other undesired mutants; a selection results in only the desired mutants). Fin regeneration in Zebrafish (see Examples) can be used as an easily-scored visual screen. Desirable mutants would be those individuals that either fail to completely regenerate a wild-type (wt) fin, those that regenerate a larger, but otherwise normal, fin, those that regenerate multiple fins, or those that grow back a different body part.

One of skill in the art would start such a screen by first mutagenizing a genetically-defined (pure) population of fish using methods well-known in the art. Mutagens cause various mutations in DNA sequences. Chemical mutagens, such as EMS and ENU, most often cause simple base-pair changes. More drastic mutagens include UV, fast-neutrons, and X-rays, which can also cause base-pair changes, but also small and large deletions and chromosomal rearrangements. One of skill in the art will select a mutagen or mutagen(s) based on factors that include the organism of choice, the gene mapping technologies available, the desired types of mutations, and safety.

Once a population of mutagenized individuals is obtained, an initial screen for fin regeneration can be done in the M1 generation (the first generation after mutagenesis) to look for dominant mutations (those mutated genes that require only one copy to exert its phenotype). Fins would be amputated, and then screened for regenerative capacity, first visually, and if necessary, microscopically (but with live organisms). Dominant mutations, for the purposes of gene mapping and cloning, can be examined by using the wt phenotype as a recessive marker.

However, many mutations will be homozygous recessive. The M1 population is self-crossed (mated) so that homozygous loci are achieved in the M2 population. The screen for fin regeneration is repeated.

As mutant individuals are isolated, it is often desirable to “clean up” their genetic background, especially if many mutations were induced during mutagenesis (one of skill in the art will determine the rate of mutagenesis by, for example, examining a mutagenized population for a mutation). This step eliminates potential multi-gene defects, which are more difficult and potentially confusing to work with. To rid a mutant of “background” mutations, it is crossed with a wt individual (“back-crossed”). The progeny are then self-crossed (“selfed”), and the F2 generation is analyzed for the return of the mutant phenotype. Those lines wherein the mutant phenotype reappears are excellent candidates for further analysis. Preferably, these mutants are backcrossed a second time or more.

To identify the number of genes under examination, the mutants are crossed to each other to identify complementation groups. Complementation occurs when a wild-type phenotype is found in all of the F2 progeny. The simplest interpretation, with the caveat that complementation can occur (or not occur) in a minority of cases for multitudes of reasons, is that the mutated genes are not the same gene in the parents. If complementation does not occur, then this result usually indicates that the two parents have mutations in the same gene. Each complementation group indicates a single gene. All lines are maintained in each complementation group.

The mutated gene may then be mapped, using techniques well-known to those of skill in the art. The specifics of mapping, especially the use of linking-markers (whether, for example, morphological or DNA polymorphisms), are unique to the organism being studied. In one approach, mutant individuals are crossed to “mapping populations”—which have genetic markers that are well defined, either genetically or cloned—and mutant individuals are examined for the linkage of the mutant phenotype to the marker. Another very useful mapping population is a distantly related strain of the organism under study; wherein, for example, 1 in 10 bps, 1 in 100 bps, 1 in 1000, or 1 in 10,000 bps in the coding DNA sequences between the two strains differ. Such populations allow for the easy use of PCR-based markers which are exceptionally easy and quick to score.

When mapping becomes more and more fine, other techniques may be exploited to facilitate cloning the mutated gene. For example, if the region wherein the mutation falls has a known sequence, candidate genes can be identified. Such genes can then be sequenced in the mutant individuals to identify deleterious mutations (including changes in amino acid sequence or premature stop codons). If the region has an unknown sequence, cloning by phenotypic rescue can be exploited. The region in which the mutation falls can be isolated from wt individuals, broken into smaller pieces (enzymatically or by physical force), subcloned into appropriate expression vectors, and then transformed into mutant individuals. If the mutant phenotype is rescued—that is, the transformed individual regenerates a fin the screening assay-then this is proof that the segment of DNA that was transformed carries the gene of interest. The introduced DNA can then be sequenced using well-known methods. In the case of dominant mutations, the mutant individual supplies the DNA, and the DNA pieces introduced into wt individuals and the mutant phenotype scored. Rescue is ideally confirmed in at least 2 different lines from each complementation group. In addition, sequencing all members at the candidate gene position is done to confirm that deleterious mutations occur in each line, indicating various alleles of the mutated gene. Noteworthy, however, are mutations that occur in operably-linked regions, such as promoters and enhancers, and those at splice-site junctions, which may be more difficult to identify by simple sequencing. One of skill in the art will know how to approach these issues.

Once the gene is in hand, the sequence can be used to design probes or primers to identify human (or any other creature) homologues. Human cDNA or genomic libraries may be exceptionally useful. PCR-based approaches may require only a human genome template. Alternatively, in silico experiments can be done to search for human homologues, such as BLAST searching. To confirm that human homologues have similar activities as the gene with which they were probed, the human sequence can be transformed into mutant individuals from the original screen and tested for mutant phenotype rescue. However, if that should fail, the human sequence can be subcloned into an expression vector, transformed into a suitable host (such as E. coli, COS cells, or Drosophila S2 cells), expressed in vitro and harvested, and then applied to, for example, a cell dedifferentiation assay or myotube cleavage/proliferation assays, such as those described below.

4. Differential Gene Expression Approach to Identify hRNLE

In a first part, candidate genes that regulate cellular plasticity can be identified by employing both differential display analysis and by preparing a suppression subtractive cDNA library between early newt limb regenerates and nonregenerating limbs. Differential expression of the cloned cDNA fragments can be confirmed by dot blot hybridization or northern blot analysis. Full-length cDNA clones for selected candidate genes can be generated by screening a newt limb regeneration cDNA library. Such cDNA clones are then sequenced and full-length open reading frames identified.

In a second part, the sequences of candidate cellular plasticity genes are analyzed by computerized BLAST and motif searches to determine whether candidate cDNAs are homologues of known genes or if they possess interesting functional domains. The degree of upregulation following limb amputation can be assessed by Phosphorimage analysis of northern blots. Cellular expression patterns of the candidate genes can be determined by whole mount or tissue section in situ hybridization of the regenerating newt limb. Genes that show marked upregulation and contain domains usually found in growth factors, cytokines, or other ligands are likely candidates. Other genes of interest include metalloproteinases (enzymes that break down the extracellular matrix and could aid in cellular dedifferentiation), receptors (which could bind the ligands that initiate the dedifferentiation process), transcription factors (potential regulators of dedifferentiation genes or downstream response genes), and intracellular signaling molecules (could be involved in dedifferentiation or other regenerative processes).

In a third part, candidate genes are assayed for a role in initiating cellular dedifferentiation. In one approach, candidate genes are cloned into a mammalian expression vector and transfected into COS-7 cells. Conditioned media is collected from the transfected COS-7 cells and used to treat C2C12 myotubes. The myotubes are monitored over several days for signs of cellular dedifferentiation, such as reentry into the cell cycle, reduction in the levels of muscle differentiation proteins, and cell cleavage and proliferation. More than one protein may be required for the initiation of cellular dedifferentiation. Therefore, combinations of candidate genes can be assayed by cotransfecting more than one candidate gene into COS-7 cells, or by combining conditioned medium generated from transfections with different candidate genes. If the sequence and expression patterns of a particular candidate gene suggest that the protein it encodes may function intracellularly downstream of the initiating signals, the gene can be ectopically expressed in C2C12 myotubes to determine its ability to induce cellular dedifferentiation.

(a) Differential Expression Analysis Experimental Details

Total RNA is extracted from 30 regenerating newt limbs at 1, 3, and 5 days postamputation. Nonregenerating limb tissue is then collected from the same newts at the time of the initial amputation. Comparing regenerating and nonregenerating tissues from the same newts should eliminate any false positives in differentially-displayed cDNAs that are due to polymorphisms found in the wild newt population. The total volume of tissue is estimated and total RNA is isolated. Residual contaminating DNA is destroyed by treating the RNA with RNase-free DNaseI, extracting the samples with phenol:chloroform:isoamyl alcohol and then precipitating with ethanol. RNA concentration and purity is determined by absorbance spectrophotometry at 260 nm and 280 nm. RNA integrity is assessed by running the samples on a 1% agarose gel in the presence of 0.5 M formaldehyde. Only nondegraded RNA is used for differential display analysis.

Differential display analysis is based on the differential reverse transcribed polymerase chain reaction (RT-PCR) amplification of RNA transcripts originating from genes that are expressed at different levels in the two tissues being compared. In one approach, reverse transcription is performed with anchor primers that bind to the poly(A) tract and are anchored by a single nucleotide (A, C, or G) on the 3′-end. Subsequent PCR amplifications are performed using the 3′-anchor primer and 1 of 80 different random primers designed to anneal to different sequences. Therefore, 240 different sets of primers are used to amplify the first-strand cDNA products. This approach provides nearly complete coverage of all transcripts expressed in the regenerating and nonregenerating newt limb. Differential display analysis is performed using regenerating and nonregenerating tissues collected at days 1, 3, and 5 postamputation. The amplified products are heat-denatured and separated on 0.4 mm 5% polyacrylamide/8M urea gels at 70 W for approximately 3 hours. The gels are dried, and Kodak X-ray BMR film is exposed for 12-16 hours. Reactions that produce differentially-displayed cDNA fragments is repeated using total RNA extracted from an independent set of tissues to confirm the differential display pattern.

The differentially-displayed cDNA fragments are excised from the dried gel and eluted by placing the gel in TE (10 mM Tris-HCl, pH 7.5, 0.1 mM EDTA) and heating to 37° C. with constant shaking overnight. The Whatmann paper and gel debris are removed by centrifugation, and the cDNA-containing supernatant is saved for PCR amplification. Two amplification reactions are then performed. In the first reaction, 4 μl of undiluted cDNA eluate is used as template, and in the second reaction, the eluted cDNA is diluted 1/10 in TE and then used as template. The excised cDNAs are amplified by PCR, and the amplification products are separated on 1.8% low melting point agarose gels. The appropriate fragments are excised and gel purified. Purified fragments are ligated into a T/A cloning vector (such as pBluescript II SK), and transformed bacterial colonies are grown to isolate the plasmid DNAs. Recombinant plasmids are then used for making probes for northern blots and for sequence analysis.

Northern blot analyses are performed to confirm that differentially-displayed cDNA fragments represent genes that are truly differentially expressed between regenerating and nonregenerating tissue. Some differentially-expressed genes may be expressed at low levels and are not detected using northerns prepared from total RNA. Therefore, differentially-displayed cDNAs using northerns prepared from single-selected poly(A) RNA from newt limbs are used. Northern blots are prepared by running 2 μg of nonregenerating limb and early limb regenerate poly(A) RNA (1, 3, and 5 days postamputation) in adjacent lanes. Ten sets of early limb regenerate/nonregenerating limb lanes are run. RNA is separated by electrophoresis at 80 V through 1% agarose gels containing 0.5 M formaldehyde, 20 nM MOPS, pH 7.0, 5 mM sodium acetate, and 1 mM EDTA. The RNA is blotted onto nylon membranes, UV-crosslinked to the membrane, and stained with 0.04% methylene blue in 0.5 M sodium acetate. The RNA is hybridized with cDNA probes prepared by random hexamer priming and ³²P-dCMP incorporation using inserts purified from recombinant plasmids. Differential expression is determined by comparing the intensity of the autoradiographic signals between lanes. Phosphorimage analysis is performed to quantitate the level of up- or down-regulation. Those exhibiting a 3-fold or greater transcriptional induction encode candidate active RNLE components.

(b) Suppression Subtractive cDNA Library Experimental Details

Candidate regeneration and dedifferentiation genes can also be identified by generating a suppression subtractive hybridization cDNA library using RNA isolated from early newt limb regenerates to prepare tester cDNA and RNA isolated from nonregenerating newt limbs to prepare the driver cDNA. Suppression subtractive hybridization is based on two important phenomena: (1) the ability of excess driver cDNA to effectively hybridize nearly all complementary cDNAs found in the tester cDNA population, leaving the unique tester transcripts as unhybridized single strands and (2) the ability of long inverted repeats located at opposite ends of the same cDNA molecule to anneal to each other and prevent primers from binding to the annealed ends.

Single-selected poly(A) RNA is isolated from total RNA that has been extracted from 200 regenerating newt limbs at 1, 3, and 5 days postamputation, and from 600 nonregenerating limbs as described above. A second round of poly(A) selection by binding the once-selected poly(A) RNA to the oligo(dT) cellulose matrix a second time, washing the cellulose, and eluting and concentrating the RNA as described above is performed.

First-strand cDNAs are prepared from both the experimental tester (early limb regenerates) and driver (non-regenerating limb) poly(A) RNAs. Two micrograms of poly(A) RNA are reverse transcribed at 42° C. for 1.5 hours using AMV reverse transcriptase. Second-strand cDNA synthesis is performed for 2 hours at 16° C. in the presence of DNA polymerase I, RNaseH, and E. coli DNA ligase. T4 DNA polymerase is added, and the samples incubated an additional 30 minutes at 16° C. Second-strand cDNA synthesis is terminated by adding an EDTA/glycogen mix, and the samples are extracted with phenol:chloroform:isoamyl alcohol and chloroform and precipitated with ethanol. The cDNAs are resuspended in ddH₂O, digested with RsaI, and purified by phenol:chloroform extraction and ethanol precipitation.

The purified RsaI-digested cDNAs from the regenerating limb are divided into two aliquots. Adaptor 1 is ligated to the cDNA ends of one of these aliquots and Adaptor 2R is ligated to the cDNA ends of the second aliquot. Adaptor-ligated cDNAs from the regenerating limb (adaptor 1-ligated and adaptor 2R-ligated) are mixed separately in two different vials with a 30-fold excess of cDNA (lacking adaptors) from the nonregenerating limb. These samples are denatured at 98° C. for 1.5 minutes and then allowed to anneal at 68° C. for 6-12 hours. The two cDNA samples from the regenerating limb that contain different adaptors are then mixed-together with freshly denatured cDNA from the nonregenerating limb (no adaptors) and annealed overnight at 68° C. Following this second round of hybridization, the single-stranded 5′-ends are filled-in using a thermostable DNA polymerase and dNTPs, and then the hybridized products are subjected to 27 cycles of suppression PCR using a primer specific for both adaptors. The PCR products are then diluted and subjected to nested PCR using a primer that is specific for adaptor 1 and a second primer specific for adaptor 2R. During these steps, templates that have the same adaptor on both ends are not efficiently amplified, because the two ends of each template contain long stretches of complementary base pairs that anneal to each other and form hairpin loops that prevent primers from reaching their target sequences. The amplified cDNA products are then ligated into T/A cloning vectors (such as pBluescript II SK) to construct a library consisting primarily of cDNAs that are preferentially expressed in the early regenerating limb. The same procedure can be followed to produce a library of cDNAs that are preferentially expressed in the nonregenerating limb.

Although this procedure enriches for differentially expressed genes, it can produce false positives. To confirm differential expression, dot blot analysis by probing filters containing subtracted cDNA clones from the regenerating limb with either labeled cDNAs from the subtracted regenerating limb or from the subtracted nonregenerating limb are performed. Clones that show differential hybridization patterns when probed with these two cDNA populations are selected for confirmation of differential expression by northern blot and Phosphorimage analysis. The inserts of confirmed clones are then sequenced using established protocols well known in the art.

(c) Generation and Sequencing of Full-Length Differentially Expressed cDNAs-Experimental Details

The following protocol can be used to identify full-length human cDNAs, using human cDNA libraries. Stringency conditions may need to be adjusted (Ausubel et al., 1987).

Full-length cDNA clones are generated for selected cDNAs by screening the newt early limb regenerate cDNA library using a probe made from either the original differentially-displayed cDNA fragment or the subtracted cDNA. Probes are labeled by random hexamer priming and incorporation of ³²P-CMP. One million cDNAs cloned into a phage vector are plated at high density, and duplicate lifts onto nylon membranes prepared. The membranes are hybridized with the ³²P-labeled cDNA probes. Secondary screens are performed by selecting the positive plaques and then replating them at a density of 300-500 plaques per 150 mm plate. Plaques are lifted onto nylon membranes and hybridized with the specific cDNA probes. Isolated positive plaques from the secondary screen are selected and grown. The cDNA inserts are excised in vivo as pBK-CMV plasmid constructs with RE704 helper phage, and the clones selected on agar with 50 μg/ml kanamycin. Colonies are selected, grown in LB-kanamycin culture, and plasmids isolated. The clones are then digested with EcoRI and XhoI to excise the cDNA inserts, and the digests separated on 1% agarose gels to determine insert sizes. The insert size for each clone is compared to its corresponding transcript size as determined by northern blot analysis to assess whether the clone might contain full-length cDNA. The ends of the clones are sequenced. If a cDNA clone is not full-length, probes are designed from either the 5′- or 3′-end or both (depending on which end of the cDNA is missing) and the library screened again. This process is reiterated until the full-length open reading frame is obtained. In cases where screening the library fails to identify a full-length open reading frame, 5′ or 3′ RACE (Rapid Amplification of cDNA Ends) can be used to clone the missing portion of the cDNA.

(d) Selection of Candidate Cellular Plasticity Genes Based Upon Sequence Analysis, Level of Upregulation, and Cellular Expression Patterns.

Sequence Analysis of Differentially Expressed cDNAs cDNA sequences of differentially expressed genes are analyzed by nucleotide and protein. BLAST searches (Altschul and Gish, 1996; Altschul et al., 1997). Not every candidate cellular plasticity gene will be recognized as belonging to a particular gene family. These novel genes could play important roles in cellular plasticity, and those that exhibit a significant transcriptional induction following amputation are tested for function (see below).

Riboprobe Synthesis Riboprobes are used in whole-mount and tissue section in situ hybridization procedures. These probes are labeled with digoxigenin (DIG), which can later be detected with an anti-DIG antibody conjugated to alkaline phosphatase. Vector constructs containing the cDNA inserts are linearized by digestion with either BamHI for use as templates for T7 RNA polymerase or XhoI for use as templates for T3 RNA polymerase. Riboprobe synthesis is carried out as follows: Briefly, 1 μg of linearized cDNA-containing vector is used as template in a reaction containing DIG labeling mix, T3/T7 RNA polymerase transcription buffer, RNase inhibitor, and T3 or T7 RNA. Transcription is carried out at 37° C. for 2 hours. DNA is destroyed by the addition of DnaseI, and the riboprobes are purified by two successive ethanol precipitation steps. Following the final precipitation, the riboprobes are resuspended in ddH₂O treated with diethyl pyrocarbonate (DEPC) and the concentration and purity is determined by spectrophotometry at 260 and 280 nm. A 1% agarose gel is run in 1×TAE to confirm the presence and concentration of the riboprobes.

Preparation of Newt Limb Powder Newt Limb Powder is. Required to Block alkaline phosphatase-conjugated anti-DIG antibody during the whole-mount in situ hybridization procedure. Use of newt powder to block the antibody reduces background staining due to nonspecific binding of the antibody to newt tissues. Amputated newt limbs are flash frozen in liquid nitrogen and stored at −80° C. until used to prepare newt limb powder. The frozen limbs are crushed into powder over liquid nitrogen using a mortar and pestle. The limb powder is treated with 4 volumes of ice cold acetone, mixed, and placed on ice for 30 minutes. Following centrifugation, the acetone is removed, the sample rinsed with acetone, and transferred to a piece of Whatmann paper, where it is ground into a fine powder. After complete air drying, the limb powder is stored in an airtight container at 4° C.

Whole-Mount in situ Hybridization Whole-mount in situ hybridization on early limb regenerates (days 1-5) is performed to determine the expression patterns of the candidate cellular plasticity genes. Photographs of the stained whole-mount regenerates are taken and the tissues can then be sectioned. Analysis of the whole-mounts before sectioning allows for the assessment of the overall expression patterns of the genes, while analysis of the tissue sections reveals specific cellular expression patterns.

Newt limb amputations are performed as described above. The limbs are reamputated within 5 days of the initial amputation, and the tissue is fixed immediately in 3.7% buffered paraformaldehyde. The tissues are thoroughly washed with phosphate buffered saline containing 0.1% Tween 20 (PBST), dehydrated in a series of methanol/PBST and solutions, and then stored −20° C. in 100% methanol. Tissues are rehydrated in methanol/PBST solutions and then washed three times in PBST. The samples are treated with 20 μg/ml proteinase K at 37° C. for 10, 20, or 30 minutes. The tissues is then washed thoroughly with PBST at 4° C. to eliminate proteinase K activity and will be acetylated with 0.5% acetic anhydride in 0.1 M triethanolamine (pH 7.9) for 10 minutes. The tissues are washed with PBST and refixed for 20 minutes with 4% paraformaldehyde. The samples are washed thoroughly with PBST, washed in hybridization solution (50% formamide, 5×SSC, 1 mg/ml yeast tRNA, 100 μg/ml sodium heparin, 1×Denhardt's solution, 0.1% Tween20, 0.1% CHAPS, and 5 mM EDTA) and then prehybridized in a rotating hybridization oven overnight at 60-65° C. in hybridization solution. The riboprobes prepared above are heated to 95° C. for 30 minutes and added to the limb tissues at a concentration of 1 μg/ml. Hybridization is carried out for 48-72 hours at 60-65° C. To remove unbound riboprobe, the tissues are washed in hybridization solution for 20 minutes at 65° C., followed by three washes in 2×SSC at 65° C. for 20 minutes each and two washes in 0.2×SSC at 65° C. for 30 minutes each.

Hybridized probes are detected by washing the samples in MAB (100 mM maleic acid, 150 mM NaCl, pH 7.5) and then in MAB-B (MAB containing 2 mg/ml BSA). The tissues are treated with antibody blocking solution (20% heat-inactivated sheep serum in MAB-B) overnight at 4° C. At the same time, the alkaline phosphatase conjugated anti-digoxigenin antibody (Roche, Boehringer-Mannheim) is diluted 1:400 in blocking solution and preabsorbed overnight at 4° C. with 10 mg/ml newt limb powder. After preabsorption, the newt powder is removed by centrifugation, and the antibody is diluted to 1:1000 (an additional 2.5-fold dilution) in blocking solution and added to the tissue samples. Antibody incubation proceeds overnight at 4° C. Tissues are washed 10 times with MAB at room temperature (30 minutes each wash) and then washed twice in AP buffer (100 mM Tris-HCl, pH 9.5, 100 mM NaCl, 50 mM MgCl₂). The tissues are incubated in the alkaline phosphatase substrate NBT/BCIP in AP buffer containing 1 mM levamisole) for 1-6 hours in the dark. The tissues are washed several times in PBST and then postfixed overnight in buffered 4% paraformaldehyde. Samples are washed once in 70% ethanol and then stored in methanol at −20° C. Tissues are cleared in a 1:2 benzyl alcohol:benzyl benzoate solution (BABB). The whole-mount tissues are photographed to determine overall expression of the gene.

Following whole-mount in situ hybridization and photography, the cellular expression patterns are assessed by embedding the tissues in paraffin and sectioning the blocks. Tissue sections are examined and photographed.

In situ Hybridization of Tissue Sections If the whole-mount procedure produces a chromogenic signal that is too weak to decipher, in situ hybridization on tissue sections can be performed. Following amputation, tissues are frozen directly in OCT. The tissues are sectioned with a cryostat at 10 μm and fixed for 1 hour in 4% paraformaldehyde DEPC-PBS. The slides are washed in 2×SSC (DEPC-treated) and then treated with 0.2 M HCl for 8 minutes. The tissues are rinsed with 0.1 M triethanolamine (pH 7.9) and acetylated with 0.25% acetic anhydride in 0.1 M triethanolamine for 15 minutes. The slides are washed with 2×SSC and heat-denature riboprobe (80° C., 3 minutes) in hybridization solution (50% formamide, 4×SSC, 1×Denhardt's solution, 500 μg/ml heat denatured herring sperm DNA, 250 μg/ml yeast tRNA, and 10% dextran sulfate) are added to the tissue sections. Cover slips are sealed over the tissues and hybridizations are carried out overnight at 55° C. in a humidified chamber. The tissues are washed in 2×SSC, then in STE (500 mM NaCl, 20 mM Tris-HCl, pH. 7.5, and 1 mM EDTA), and treated with RNase A (40 μg/ml in STE) for 30 minutes at 37° C. Sections are washed with 2×SSC, 50% formamide at 55° C., then with 1×SSC at room temperature, and finally with 0.5×SSC at room temperature.

Bound riboprobes are detected by washing the slides for 1 minute in Buffer 1 (100 mM Tris-HCl, pH 7.5, 150 mM NaCl), then blocking the tissues with 2% sheep serum in Buffer 1. Sheep anti-digoxigenin antibody conjugated to alkaline phosphatase (Roche) is diluted 1:500 in Buffer 1 containing 1% sheep serum, added to the tissues, and incubated in a humidified chamber at room temperature for 1 hour. Slides are then washed in Buffer 1, followed by a wash in Buffer 2 (100 mM Tris-HCl, pH 9.5, 100 mM NaCl, 50 mM MgCl₂). Substrate solution (NBT/BCIP in Buffer 2 with 1 mM levamisole) is added to the sections and the slides incubated in the dark at 4° C. overnight. The reaction is terminated by placing the slides in Buffer 3 (10 mM Tris-HCl. pH 8.0, 1 mM EDTA). The tissues are mounted and observed for chromogenic staining by light microscopy.

Prioritizing Candidate Cellular Plasticity Genes Candidate cellular plasticity genes can be prioritized according to their gene families, degree of transcriptional induction, and cellular expression patterns. Genes that are significantly upregulated and encode potential extracellular signaling molecules, such as growth factors, cytokines, or other ligands, are immediate candidates. Such genes may encode factors that initiate cellular dedifferentiation. Other genes of primary interest include receptors, which could bind the initiating ligands, kinases, which could play a role in the intracellular transduction of the dedifferentiating signals, and transcription factors, which could be response genes that either induce or repress downstream genes involved in dedifferentiation or maintenance of the differentiated state. Metalloproteinases could be involved in cellular dedifferentiation by interrupting the extracellular matrix. Finally, novel genes that are markedly upregulated following amputation but do not belong to any known gene family are of interest, because they could function in regulating cellular plasticity.

Between 30-100 differentially-expressed genes can be expected from this approach, of which up to 50% of the genes are likely to be mitochondrial genes, or other housekeeping genes and therefore unlikely RNLE components. The remaining candidate genes are then tested for function in initiating or inducing cellular dedifferentiation as described below.

(e) Assay to Determine if Candidate Genes Play Roles in Cellular Plasticity

The differentially expressed genes that are candidates for regulating cellular plasticity are then tested to determine whether they function to induce cellular dedifferentiation in cultured mouse C2C12 myotubes, or in another embodiment, dedifferentiation of in vitro cultured human cells. Mouse myotubes can be induced to dedifferentiate either when treated with protein extracts from early limb regenerates (days 1-5 postamputation) or when induced to ectopically express msxl in the presence of growth factors. Using a similar approach, one can determine whether a candidate gene induces cellular dedifferentiation. If the candidate gene appears to encode a secreted protein (possibly a growth factor, cytokine, or other ligand), it is cloned into an expression vector and determined whether treating mouse myotubes with the expressed protein can induce cellular dedifferentiation. If the gene appears to encode a cellular factor and is expressed in the underlying stump tissue, it is cloned into a mammalian expression vector and its expression induced in mouse myotubes and then determined whether the ectopic expression of the gene can induce mouse myotubes to dedifferentiate. If a single gene is unable to induce dedifferentiation, combinations of the various candidate genes are tested for their ability to induce cellular plasticity. If combinations of genes are unable to induce cellular plasticity, nonregenerating limb extracts are prepared, and then one can determine whether these extracts (which do not induce dedifferentiation on their own), in combination with the candidate genes, can induce dedifferentiation.

Testing Candidate Newt Genes for Their Ability to Initiate Dedifferentiation of Mouse Myotubes Genes whose sequences suggest they may be secreted soluble factors will be tested for their ability to initiate cellular dedifferentiation of mouse myotubes. A relatively easy approach to determine whether a secreted gene can initiate cellular dedifferentiation is to transfect cultured COS-7 cells with a plasmid construct containing the candidate gene driven by a mammalian promoter, such as a CMV promoter. A few days following transfection, the cell culture medium is collected. Secreted soluble proteins expressed in the COS-7 cells are present in this conditioned medium. The conditioned medium can then be used to treat terminally-differentiated mouse myotubes or cultured human cells to determine whether the expressed protein can initiate the dedifferentiation process. Controls consist of conditioned medium from mock-transfected COS-7 cells.

A single candidate gene may not be able to initiate cellular dedifferentiation, while combinations of candidate genes may induce such a response. Therefore, if no single gene can initiate dedifferentiation on its own, cotransfection of combinations of candidate dedifferentiation genes into COS-7 cells are performed and then determine whether the resulting conditioned medium can induce cellular dedifferentiation. Alternatively, conditioned medium from singly-transfected COS-7 cells can be combined and the dedifferentiation assays performed using the combined medium.

Transfection of COS-7 cells and Confirmation of the Presence of Candidate Proteins in Conditioned Medium COS-7 cells are grown and passaged in DMEM containing 0.1 mM nonessential amino acids (NEAA) and 10% FBS at 37° C. in 5% CO₂. The day before transfection, 2×10⁶ cells are plated in 12 ml of growth medium on 100 mm poly-D-lysine-coated tissue culture plates. A hemagglutinin tag is added to the 3′-end of the full-length cDNAs so that the presence of protein in the conditioned medium can be ascertained. The entire construct is cloned into the pBK-CMV expression vector and transfected into cultured COS-7 cells using liposome-mediated transfection. Conditioned medium is collected to use in dedifferentiation assays 48 hours after the initiation of transfection.

The conditioned medium is tested for the presence of the candidate dedifferentiation protein using Western blot analysis. Proteins are separated on 4-20% linear gradient gels and then transferred to nylon membranes by electrophoresis. The membranes are air dried, blocked with 5% nonfat dry milk, and then incubated overnight at 4° C. in a solution containing anti-hemagglutinin antibody (mono HA.11, BabCo) diluted 1:1000 in blocking solution. The blots are thoroughly washed and incubated for 1 hour with a peroxidase-conjugated anti-mouse IgG secondary antibody diluted 1:1000 with blocking solution. The blots are thoroughly washed and enhanced chemiluminescence is performed to determine whether the candidate dedifferentiation protein is present in the conditioned medium.

Testing Candidate Proteins for their Ability to Induce Cell Cycle Reentry

To determine whether a candidate protein can induce mouse myotubes to reenter the cell cycle, BrdU-incorporation experiments are performed. Briefly, C2C12 myoblasts (or cultured human cells) are grown to confluency in 24-well plates in growth medium (GM-20% FBS and 4 in M glutamine in DMEM) and then induced to differentiate by replacing GM with differentiation medium (DM-2% horse serum and 4 mM glutamine in DMEM). The myocytes are allowed to differentiate for 4 days. C2C12 myotubes in different wells are then treated with different dilutions of the conditioned medium (undiluted, ½, ¼, ⅛, 1/16, and a control well with no conditioned medium) for up to 4 days. BrdU is added to the cultures at a concentration of 10 nmol/ml 12 hours before testing for cell cycle reentry. BrdU incorporation is assayed using the 5-bromo-2′-deoxy-uridine labeling. Briefly, the cells are thoroughly washed with PBS, fixed for 20 minutes at −20° C. with 70% ethanol/15 mM glycine buffer (pH 2.0), and washed again. Cells are then incubated in a 1:10 dilution of anti-BrdU antibody for 30 minutes at 37° C. The cells are washed and then incubated in fluorescein-conjugated anti-mouse IgG for 30 minutes at 37° C. After washing, the cells are observed microscopically and photographed using a FITC filter. Cells containing nuclei that fluoresce green have incorporated BrdU during DNA synthesis and are regarded as having reentered the cell cycle. Given that cell cycle reentry plays an important role in cellular dedifferentiation, any candidate newt gene that induce reentry into the cell cycle are considered to be potentially important for the initiation of cellular dedifferentiation and plasticity.

Testing Candidate Proteins for Their Ability to Reduce Levels of Muscle Differentiation Proteins To determine whether a candidate gene can reduce the levels of muscle differentiation proteins, mouse myotubes (or cultured human muscle cells) as described above are treated with the conditioned medium from COS-7 cells expressing the candidate gene. After 3 days of treatment, immunofluorescent assays are performed to determine whether there has been a reduction in the levels of MyoD, myogenin, MRF4, troponin T, and p21. MyoD, myogenin, and MRF4 are important regulators of myogenesis, while p21 signals the onset of the postmitotic state and troponin T is a component of the contractile apparatus. All of these factors are normally expressed in C2C12 myotubes, and a reduction in their levels signify a reversal in cell differentiation. The cells are washed with PBS, fixed in Zamboni's fixative for minutes, washed again with PBS, and permeabilized with 0.2% TritonX-100 in DPBS for 20 minutes. The cells are blocked with 5% slim milk in DPBS for 1 hour at room temperature and then exposed to the primary antibodies overnight at 4° C., using primary antibodies that recognize MyoD, myogenin, MRF4, troponin T, and p21. The cells are washed and then treated for 45 minutes at 37° C. with either goat anti-rabbit IgG conjugated to Alexa 488, goat anti-mouse IgG conjugated to biotin, or both secondary antibodies, depending upon the primary antibody(ies) used. The cells are washed and then either observed fluorescently or treated with streptavidin-Alexa 594 for 45 minutes at 37° C. The latter cells are washed and then observed with fluorescent microscopy using FITC and Texas Red filters. Cell nuclei are visually observed to determine whether the levels of the myogenic regulatory factors MyoD, myogenin, MRF4, and p21 have been reduced. Cytoplasm is observed to determine whether troponin T levels are reduced. Reduced levels of these muscle differentiation proteins are another indicator of myotube dedifferentiation. For controls, cells not treated with conditioned media are used. Therefore, any candidate gene that can induce these cellular changes are considered important for the initiation of cellular dedifferentiation and plasticity.

Testing Candidate Proteins for Their Ability to Induce Myotube Cleavage and Cell Proliferation Any candidate gene that initiates reentry into the cell cycle and/or reduction in muscle differentiation protein levels is tested for its ability to induce cell cleavage and proliferation. Myotubes (or human muscle cells) are generated as described above, except large numbers are plated on 100 mm tissue culture plates. These cells are purified and replated at low density. Residual mononucleated cells are eliminated by needle ablation and lethal water injections. The cells are photographed, conditioned medium is added, and the cells monitored by visual inspection and photography for up to 7 days. Cell culture medium containing conditioned medium is changed daily. Cleavage of myotubes to form smaller myotubes or proliferating, mononucleated cells are considered an indication of cellular dedifferentiation. Any candidate gene that can initiate myotube cleavage is considered an important gene for cellular dedifferentiation and plasticity.

(f) Testing Candidate Genes that Encode Cellular Proteins for a Possible Role in Dedifferentiation

Candidate genes that are expressed in the underlying stump and appear to encode cellular proteins, e.g., receptors, transcription factors, or signal transduction proteins are tested for a possible role in cellular dedifferentiation by ectopically expressing them in mouse (or human) myotubes. A retroviral construct (LNX) containing a doxycycline-suppressible candidate gene is transfected into PhoenixAmphotropic cells using the CaPO₄ method, and the resulting recombinant retroviruses are harvested by saving the conditioned medium. Myoblasts are infected with the recombinant retrovirus by adding the conditioned medium to the myoblasts in the presence of 4 mg/ml Polybrene and allowing the infection to occur for 12-18 hours. The infection medium is replaced with myoblast growth medium containing doxycycline to prevent the expression of the candidate gene. The cells are allowed to grow for 48 hours, sub-cultured, and grown in the presence of doxycycline and G418 to select for transduced myoblasts. Selection continues for 14 days, and clonal populations are derived. Candidate genes are induced following myotube formation in the expanded clones by replacing DM-dox with medium lacking dox. The cells are then tested for reentry into the cell cycle, reduction in muscle differentiation proteins, and cell cleavage and proliferation as described above. A candidate gene that induces any of these indicators of cellular dedifferentiation is considered an important response gene in the cellular dedifferentiation pathway.

Alternatively, another approach may include the purification of candidate proteins expressed in either bacterial or eukaryotic cells. These purified proteins could then be used at specified concentrations in the cellular dedifferentiation assays described in this proposal. Additionally, any of the above cited approaches similarly applies to the testing of non-nucleic acid or polypeptide agents that can promote dedifferentiation. Such agents include small organic molecules.

5. Making and Using Antibodies to Identify Active RNLE Components

Because RNLE active components are likely proteins, polypeptides or peptides (see Examples), an antibody approach can be taken, especially if genetic or differential display approaches become difficult or nonproductive.

In this approach, antibodies are raised against antigens in whole RNLE, or in fractions of RNLE, in a host of choice. Preferably, the host is one from which monoclonal antibodies mAbs can be eventually derived. Once antibodies are produced, they are tested, first in vitro, then in vivo, for their ability to block a RNLE dependent process, such as myotube dedifferentiation or newt limb regemation. Such antibodies can then be used to isolate human (or any other organism) homologues using a variety of approaches, such as screening human expression libraries, isolating the antigen-containing polypeptides by antibody affinity chromatography and performing terminal peptide sequencing and using such a sequence to perform in silico experiments or to design nucleic acid probes and primers to isolate nucleic acids encoding the corresponding polypeptides.

“Antibody” (Ab) comprises single Abs directed against an RNLE (anti-RNLE Ab; including agonist, antagonist, and neutralizing Abs), anti-RNLE Ab compositions with poly-epitope specificity, single chain anti-RNLE Abs, and fragments of anti-RNLE Abs. A “monoclonal antibody” is obtained from a population of substantially homogeneous Abs, i.e., the individual Abs comprising the population are identical except for possible naturally-occurring mutations that may be present in minor amounts. Abs include polyclonal (pAb), monoclonal (mAb), humanized, bi-specific (bsAb), and heteroconjugate Abs.

The following outlines one variation of this approach. One of skill in the art may choose other variations, or deviate from the following but will still achieve the same endpoint.

Newt limb extract is prepared as above, in large quantity. Preferably, the extract is concentrated to minimize the aqueous component, such as by dialysis. Alternatively, the proteins may be isolated by any method known in the art, such as, for example, ammonium sulfate or trichloroacetic acid precipitation. This preparation is used as the antigen.

(a) Polyclonal Abs (PAbs)

Polyclonal Abs can be raised in a mammalian host, for example, by one or more injections of immunogens (RNLE) and, if desired, an adjuvant. Typically, the immunogen and/or adjuvant are injected in the mammal by multiple subcutaneous or intraperitoneal injections. Examples of adjuvants include Freund's complete and monophosphoryl Lipid A synthetic-trehalose dicorynomycolate (MPL-TDM). To improve the immune response, an immunogen may be conjugated to a protein that is immunogenic in the host, such as keyhole limpet hemocyanin (KLH), serum albumin, bovine thyroglobulin, and soybean trypsin inhibitor. Protocols for antibody production are well-described (Ausubel et al., 1987; Harlow and Lane, 1988). Alternatively, pAbs may be made in chickens, producing IgY molecules (Schade et al., 1996).

(b). Monoclonal Abs (mAbs)

Anti-RNLE mAbs may be prepared using hybridoma methods (Milstein and Cuello, 1983). Hybridoma methods comprise at least four steps: (1) immunizing a host, or lymphocytes from a host; (2) harvesting the mAb secreting (or potentially, secreting) lymphocytes, (3) fusing the lymphocytes to immortalized cells, and (4) selecting those cells that secrete the desired (anti-RNLE) mAb.

A mouse, rat, guinea pig, hamster, or other appropriate host is immunized to elicit lymphocytes that produce or are capable of producing Abs that will specifically bind to the immunogen. Alternatively, the lymphocytes may be immunized in vitro. If human cells are desired, peripheral blood lymphocytes (PBLs) are generally used; however, spleen cells or lymphocytes from other mammalian sources are preferred. The immunogen typically includes an RNLE or a fusion protein.

The lymphocytes are then fused with an immortalized cell line to form hybridoma cells, facilitated by a fusing agent such as polyethylene glycol (Goding, 1996). Rodent, bovine, or human myeloma cells immortalized by transformation may be used, or rat or mouse myeloma cell lines. Because pure populations of hybridoma cells and not unfused immortalized cells are preferred, the cells after fusion are grown in a suitable medium that contains one or more substances that inhibit the growth or survival of unfused, immortalized cells. A common technique uses parental cells that lack the enzyme hypoxanthine guanine phosphoribosyl transferase (HGPRT or HPRT). In this case, hypoxanthine, aminopterin and thymidine are added to the medium (HAT medium) to prevent the growth of HGPRT-deficient cells while permitting hybridomas to grow. Preferred immortalized cells fuse efficiently, can be isolated from mixed populations by selecting in a medium such as HAT, and support stable and high-level expression of antibody after fission. Preferred immortalized cell lines are murine myeloma lines, available from the American Type Culture Collection (Manassas, Va.). Human myeloma and mouse-human heteromyeloma cell lines also have been described for the production of human mAbs (Kozbor et al., 1984; Schook, 1987). Because hybridoma cells secrete antibody extracellularly, the culture media can be assayed for the presence of mAbs directed against an RNLE (anti-RNLE mAbs). Immunoprecipitation or in vitro binding assays, such as radio immunoassay (RIA) or enzyme-linked immunoabsorbent assay (ELISA), measure the binding specificity of mAbs (Harlow and Lane, 1988; Harlow and Lane, 1999), including Scatchard analysis (Munson and Rodbard, 1980).

Anti-RNLE mAb secreting hybridoma cells may be isolated as single clones by limiting dilution procedures and sub-cultured (Goding, 1996). Suitable culture media include Dulbecco's Modified Eagle's Medium, RPMI-1640, or if desired, a protein-free or -reduced or serum-free medium (e.g., Ultra DOMA PF or HL-1; Biowhittaker; Walkersville, Md.). The hybridoma cells may also be grown in vivo as ascites.

The mAbs may be isolated or purified from the culture medium or ascites fluid by conventional Ig purification procedures such as protein A-Sepharose, hydroxylapatite chromatography, gel electrophoresis, dialysis, ammonium sulfate precipitation or affinity chromatography (Harlow and Lane, 1988; Harlow and Lane, 1999).

The mAbs may also be made by recombinant methods (U.S. Pat. No. 4,166,452). DNA encoding anti-RNLE mAbs can be readily isolated and sequenced using conventional procedures, e.g., using oligonucleotide probes that specifically bind to murine heavy and light antibody chain genes, to probe preferably DNA isolated from anti-RNLE-secreting mAb hybridoma cell lines. Once isolated, the isolated DNA fragments are sub-cloned into expression vectors that are then transfected into host cells such as simian COS-7 cells, Chinese hamster ovary (CHO) cells, or myeloma cells that do not otherwise produce Ig protein, to express mAbs. The isolated DNA fragments can be modified, for example, by substituting the coding sequence for human heavy and light chain constant domains in place of the homologous murine sequences (U.S. Pat. No. 4,816,567; Morrison et al., 1987), or by fusing the Ig coding sequence to all or part of the coding sequence for a non-Ig polypeptide. Such a non-Ig polypeptide can be substituted for the constant domains of an antibody, or can be substituted for the variable domains of one antigencombining site to create a chimeric bivalent antibody.

i. Screening for Function-Blocking Antibodies

If function-blocking antibodies are desired, screening hybridoma supernatants in pools represents an attractive option. Before limiting dilution to single cells, hybridomas after fusion are instead split into pools contains 2 to thousands of cells, representing 2 or more different antibodies. These supernatants, or preparations thereof, can be used to screen for their ability to inhibit RNLE-like activity in any of the assays outlined above, such as myotube dedifferentiation; or preferably, inhibit the ability of newt limbs to regenerate. Those pools that exhibit function blocking activity are then subcloned by dilution into smaller pools, the screen repeated, and dilution of active pools repeated. This process is reiterated until clonal hybridoma cell lines are achieved. Function-blocking, in this case, does not necessarily indicated total inhibition of function; any antibody that shows an effect that is contrary to the activity of RNLE is a candidate.

Once such clonal lines are achieved, the antibodies can be used to isolate the polypeptides they bind, and identification of human or other animals homologues can proceed.

Furthermore, as outlined in detail throughout the application, the invention contemplates the isolation, identification and use of blocking antibodies which inhibit the activity of an agent that prevent dedifferentiation. In this context, a blocking antibody can be a dedifferentiation agent.

ii. Identification of Human Components of RNLE

The antibodies identified above can be used to affinity-purify the antigen containing polypeptide. Once the polypeptides are isolated, they can be analyzed in a number of ways, known to those of skill in the art, to determine their sequence, for example N-terminal sequencing. Once a peptide fragment sequence is known, that sequence can be used to identify identical or similar proteins using protein-protein BLAST searches, or in the design of nucleic acid primers and probes. Such probes, which are degenerate due to the degeneracy of the genetic code, can be used to identify candidate nucleic acid molecules encoding homologues of the antibody antigen. Any appropriate library, or genome, may be screened. Preferably, a cDNA library is screened; most preferably, a cDNA library from human is screened.

Alternatively, the antibodies themselves may be used to directly identify similar or identical proteins from other species. For example, an expression library, preferably from human, may be screened with the antibodies. When binding is observed, that signal indicates a candidate human homologous protein. Alternatively, panning approaches or affinity chromatography may be exploited if protein misconformations prevent antibody binding of proteins produced in a bacterial mediated expression library.

6. Candidate Approach

The inventors believe that the polypeptides, or their homologues, listed in Table C1 are likely dedifferentiation agents.

TABLE C1
Candidate Dedifferentiation Agents

| Extracellular | Intracellular |
|---|---|
| Family members of Fibroblast Growth | msx1 |
| Factors (Fgfs) | |
| Family of Bone Morphophenetic Proteins | msx2 |
| (BMPs) | |
| Wnt proteins | E2F |
| Metalloproteinases | Fgf receptors |
| | Frizzled (wnt receptors) |
| | SMADs (mothers against |
| | decapentaplegic) |
| | fatty acid binding proteins |

Various approaches can be used to identify if the candidate components are active in RE. A skilled artisan will choose the approach. For example, anti-sense or aptamers approaches can be used to inhibit expression of the intracellular candidate components in regenerating newt limb, using technology well-known in the art, and then testing the ability for the limb to regenerate. Alternatively, function-blocking antibodies that are available in the art against the various components can be used to inhibit newt limb regeneration. If the limb fails to fully differentiate, then the component is likely to be contained in RE. Additionally, RNAi constructs, antisense oligonucleotides, ribozymes, and other function blocking reagents can be used to decrease or inhibit the expression and/or activity of an agent, and thereby demonstrate that the agent is required for dedifferentiation.

C. Msx1

The invention provides methods for cellular dedifferentiation and regeneration that use msx1. Because msx1 is an intracellular factor, it must be introduced into cells. Three methods are contemplated: (1) nucleic acid and gene therapy approaches, wherein msx1 is subcloned into a nucleic acid vector and then derived by another vector (such as adenovirus) or directly to the cells of interest; (2) a fusion msx1 polypeptide, wherein msx1 is fused to a polypeptide that usually gains entry to cells, such as HIV tat protein (see Table C); delivery can be affected by incorporation into a suitable pharmaceutical composition; and (3) incorporation of msx1 into a composition that is taken up by cells, such as in liposomes. Details of pharmaceutical compositions and their use can be found herein.

While the following section pertains to msx1 gene therapy and molecular manipulation, the methods are applicable to other parts of the invention that also use nucleic acids, such as in the production of hRNLE by differential expression, etc.

1. Gene Therapy Compositions

The msx1 nucleic acid molecule (or a nucleic acid molecule encoding any active RDF component) can be inserted into vectors and used as gene therapy vectors. Gene therapy vectors can be delivered to a subject by, for example, intravenous injection, local administration (Nabel and Nabel, U.S. Pat. No. 5,328,470), or by stereotactic injection (Chen et al., 1994). The pharmaceutical preparation of a gene therapy vector can include an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

2. vectors

Vectors are tools used to shuttle DNA between host cells or as a means to express a nucleotide sequence. Some vectors function only in prokaryotes, while others function in both prokaryotes and eukaryotes, enabling large-scale DNA preparation from prokaryotes for expression in eukaryotes. Inserting the DNA of interest, such as a msx1 nucleotide sequence or a fragment, is accomplished by ligation techniques and/or mating protocols well known to the skilled artisan. Such DNA is inserted such that its integration does not disrupt any functional components of the vector. Introduced DNA is operably-linked to the vector elements that govern transcription and translation in vectors that express the introduced DNA.

Vectors can be divided into two general classes: Cloning vectors are replicating plasmids or phage with regions that are non-essential for propagation in an appropriate host cell and into which foreign DNA can be inserted; the foreign DNA is replicated and propagated as if it were a component of the vector. An expression vector (such as a plasmid, yeast, or animal virus genome) is used to introduce foreign genetic material into a host cell or tissue in order to transcribe and translate the foreign DNA. In expression vectors, the introduced DNA is operably-linked to elements such as promoters that signal to the host cell to transcribe the inserted DNA. Some promoters are exceptionally useful, such as inducible promoters that control gene transcription in response to specific factors. Operably-linking msxl or anti-sense constructs to an inducible promoter can control the expression of fragments or anti-sense constructs. Examples of classic inducible promoters include those that are responsive to α-interferon, heat-shock, heavy metal ions, and steroids such as glucocorticoids (Kaufman, 1990) and tetracycline. Other desirable inducible promoters include those that are not endogenous to the cells in which the construct is being introduced, but, however, are responsive in those cells when the induction agent is exogenously supplied.

Vectors have many different manifestations. A “plasmid” is a circular double stranded DNA molecule into which additional DNA segments can be introduced. Viral vectors can accept additional DNA segments into the viral genome. Certain vectors are capable of autonomous replication in a host cell (e.g., bacterial vectors having a bacterial origin of replication and episomal mammalian vectors). Other vectors (e.g., non-episomal mammalian vectors) are integrated into the genome of a host cell upon introduction into the host cell, and are replicated along with the host genome. In general, useful expression vectors are often plasmids. However, other forms of expression vectors, such as viral vectors (e.g., replication defective retroviruses, adenoviruses and adeno-associated viruses) are contemplated. Such vectors can be extremely useful in gene therapy applications.

Recombinant expression vectors that comprise msxl (or fragments) regulate msxl transcription by exploiting one or more host cell-responsive (or that can be manipulated in vitro) regulatory sequences that is operably-linked to msxl. “Operably-linked” indicates that a nucleotide sequence of interest is linked to regulatory sequences such that expression of the nucleotide sequence is achieved.

Vectors can be introduced in a variety of organisms and/or cells (Table D). Alternatively, the vectors can be transcribed and translated in vitro, for example using T7 promoter regulatory sequences and T7 polymerase.

TABLE D
Examples of hosts for cloning or expression

| Organisms | Examples | Sources and References* |
|---|---|---|
| Prokaryotes | E. coli | |
| | K 12 strain MM294 | ATCC 31,446 |
| | X1776 | ATCC 31,537 |
| | W3110 | ATCC 27,325 |
| | K5772 | ATCC 53,635 |
| Enterobacteriaceae | Enterobacter | |
| | Erwinia | |
| | Klebsiella | |
| | Proteus | |
| | Salmonella (S. tyhpimurium) | |
| | Serratia (S. marcescans) | |
| | Shigella | |
| | Bacilli (B. subtilis and B. licheniformis) | |
| | Pseudomonas (P. aeruginosa) | |
| | Streptomyces | |
| Eukaryotes | Saccharomyces cerevisiae | |
| Yeasts | Schizosaccharomyces pombe | |
| | Kluyveromyces | (Fleer et al., 1991) |
| | K. lactis MW98-8C, | (de Louvencourt et al., |
| | CBS683, CBS4574 | 1983 |
| | K. fragilis | ATCC 12,424 |
| | K. bulgaricus | ATCC 16,045 |
| | K. wickeramii | ATCC 24,178 |
| | K. waltii | ATCC 56,500 |
| | K. drosophilarum | ATCC 36,906 |
| | K. thermotolerans | |
| | K. marxianus; yarrowia | (EPO 402226, 1990) |
| | Pichia pastoris | (Sreekrishna et al., 1988) |
| | Candida | |
| | Trichoderma reesia | |
| | Neurospora crassa | (Case et al., 1979) |
| | Torulopsis | |
| | Rhodotorula | |
| | Schwanniomyces (S. occidentalis) | |
| Filamentous Fungi | Neurospora | |
| | Penicillium | |
| | Tolypocladium | (WO 91/00357, 1991) |
| | Aspergillus (A. nidulans and | (Kelly and Hynes, 1985; |
| | A niger) | Tilburn et al., 1983; |
| | | Yelton et al., 1984) |
| Invertebrate cells | Drosophila S2 | |
| | Spodoptera Sf9 | |
| Vertebrate cells | Chinese Hamster Ovary | |
| | (CHO) | |
| | simian COS | ATCC CRL 1651 |
| | COS-7 | |
| | HEK 293 | |

*Unreferenced cells are generally available from American Type Culture Collection (Manassas, VA).

Vector choice is dictated by the organism or cells being used and the desired fate of the vector. Vectors may replicate once in the target cells, or may be “suicide” vectors. In general, vectors comprise signal sequences, origins of replication, marker genes, enhancer elements, promoters, and transcription termination sequences. The choice of these elements depends on the organisms in which the vector will be used and are easily determined. Some of these elements may be conditional, such as an inducible or conditional promoter that is turned “on” when conditions are appropriate. Examples of inducible promoters include those that are tissue-specific, which relegate expression to certain cell types, steroid-responsive, or heat-shock reactive. Some bacterial repression systems, such as the lac operon, have been exploited in mammalian cells and transgeruc animals (Fieck et al., 1992; Wyborski et al., 1996; Wyborski and Short, 1991). Vectors often use a selectable marker to facilitate identifying those cells that have incorporated the vector. Many selectable markers are well known in the art for the use with prokaryotes, usually antibiotic-resistance genes or the use of autotrophy and auxotrophy mutants.

If msx1 expression is not desired, using antisense and sense msx1 oligonucleotides can prevent msx1 polypeptide expression. These oligonucleotides bind to target nucleic acid sequences, forming duplexes that block transcription or translation of the target sequence by enhancing degradation of the duplexes, terminating prematurely transcription or translation, or by other means.

Antisense or sense oligonucleotides are singe-stranded nucleic acids, either RNA or DNA, which can bind target msxl mRNA (sense) or msxl DNA (antisense) sequences. According to the present invention, antisense or sense oligonucleotides comprise a fragment of the msxl DNA coding region of at least about 14 nucleotides, preferably from about 14 to 30 nucleotides. In general, antisense RNA or DNA molecules can comprise at least 5, 10, 15, 20, 25, 30, 35, 40, 45, 50, 55, 60, 65, 70, 75, 80, 85, 90, 95, 100 bases in length or more. Among others, (Stein and Cohen, 1988; van der Krol et al., 1988) describe methods to derive antisense or a sense oligonucleotides from a given cDNA sequence.

Modifications of antisense and sense oligonucleotides can augment their effectiveness. Modified sugar-phosphodiester bonds or other sugar linkages (WO 91/06629, 1991), increase in vivo stability by conferring resistance to endogenous nucleases without disrupting binding specificity to target sequences. Other modifications can increase the affinities of the oligonucleotides for their targets, such as covalently linked organic moieties (WO 90/10448) or poly-(L)-lysine. Other attachments modify binding specificities of the oligonucleotides for their targets, including metal complexes or intercalating (e.g. ellipticine) and alkylating agents.

To introduce antisense or sense oligonucleotides into target cells (cells containing the target nucleic acid sequence), any gene transfer method may be used and these methods are well known to those of skill in the art. Examples of gene transfer methods include (1) biological, such as gene transfer vectors like Epstein Barr virus or conjugating the exogenous DNA to a ligand-binding molecule (WO 91/04753), (2) physical, such as electroporation, and (3) chemical, such as CaPQ₄ precipitation and oligonucleotide-lipid complexes (WO 90/10448).

The terms “host cell” and “recombinant host cell” are used interchangeably. Such terms refer not only to a particular subject cell but also to the progeny or potential progeny of such a cell. Because certain modifications may occur in succeeding generations due to either mutation or environmental influences, such progeny may not, in fact, be identical to the parent cell, but are still included within the scope of the term.

Methods of eukaryotic cell transfection and prokaryotic cell transformation are well known in the art. The choice of host cell will dictate the preferred technique for introducing the nucleic acid of interest. Table E, which is not meant to be limiting, summarizes many of the known techniques in the art. Introduction of nucleic acids into an organism may also be done with ex vivo techniques that use an in vitro method of transfection, as well as established genetic techniques, if any, for that particular organism.

TABLE E
Methods to introduce nucleic acid into cells

| Cells | Methods | References | Notes |
|---|---|---|---|
| Prokaryotes | Calcium chloride | (Cohen et al., 1972; | |
| (bacteria) | | Hanahan, 1983; Mandel and | |
| | | Higa, 1970) | |
| | Electroporation | (Shigekawa and Dower, | |
| | | 1988) | |
| Eukaryotes | Calcium phosphate | N-(2- | Cells may be |
| Mammalian | transfection | Hydroxyethyl)piperazine-N′- | “shocked” with |
| cells | | (2-ethanesulfonic acid | glycerol or |
| | | (HEPES) buffered saline | dimethylsulfoxide |
| | | solution (Chen and | (DMSO) to increase |
| | | Okayama, 1988; Graham | transfection |
| | | and van der Eb, 1973; | efficiency (Ausubel |
| | | Wigler et al., 1978) | et al., 1987). |
| | | BES (N,N-bis(2- | |
| | | hydroxyethyl)-2- | |
| | | aminoethanesulfonic acid) | |
| | | buffered solution (Ishiura et | |
| | | al., 1982) | |
| | Diethylaminoethyl | (Fujita et al., 1986; Lopata et | Most useful for |
| | (DEAE)-Dextran | al., 1984; Selden et al., 1986) | transient, but not |
| | transfection | | stable, transfections. |
| | | | Chloroquine can be |
| | | | used to increase |
| | | | efficiency. |
| | Electroporation | (Neumann et al., 1982; | Especially useful for |
| | | Potter, 1988; Potter et al., | hard-to-transfect |
| | | 1984; Wong and Neumann, | lymphocytes. |
| | | 1982) | |
| | Cationic lipid | (Elroy-Stein and Moss, | Applicable to both |
| | reagent | 1990; Felgner et al., 1987; | in vivo and in vitro |
| | transfection | Rose et al., 1991; Whitt et | transfection. |
| | | al., 1990) | |
| | Retroviral | Production exemplified by | Lengthy process, |
| | | (Cepko et al., 1984; Miller | many packaging |
| | | and Buttimore, 1986; Pear et | lines available at |
| | | al., 1993) | ATCC. Applicable |
| | | Infection in vitro and in vivo: | to both in vivo and in |
| | | (Austin and Cepko, 1990; | vitro transfection. |
| | | Bodine et al., 1991; Fekete | |
| | | and Cepko, 1993; Lemischka | |
| | | et al., 1986; Turner et al., | |
| | | 1990; Williams et al., 1984) | |
| | Polybrene | (Chaney et al., 1986; Kawai | |
| | | and Nishizawa, 1984) | |
| | Microinjection | (Capecchi, 1980) | Can be used to |
| | | | establish cell lines |
| | | | carrying integrated |
| | | | copies of msx1 |
| | | | DNA sequences. |
| | | | Applicable to both in |
| | | | vitro and in vivo. |
| | Protoplast fusion | (Rassoulzadegan et al., 1982; | |
| | | Sandri-Goldin et al., 1981; | |
| | | Schaffner, 1980) | |
| Insect cells | Baculovirus | (Luckow, 1991; Miller, | Useful for in vitro |
| (in vitro) | systems | 1988; O'Reilly et al., 1992) | production of |
| | | | proteins with |
| | | | eukaryotic |
| | | | modifications. |
| Yeast | Electroporation | (Becker and Guarente, 1991) | |
| | Lithium acetate | (Gietz et al., 1998; Ito et al., | |
| | | 1983) | |
| | Spheroplast fusion | (Beggs, 1978; Hinnen et al., | Laborious, can |
| | | 1978) | produce aneuploids. |
| Plant cells | Agrobacterium | (Bechtold and Pelletier, | |
| (general | transformation | 1998; Escudero and Hohn, | |
| reference: | | 1997; Hansen and Chilton, | |
| (Hansen and | | 1999; Touraev and al., 1997) | |
| Wright, | Biolistics | (Finer et al., 1999; Hansen | |
| 1999)) | (microprojectiles) | and Chilton, 1999; Shillito, | |
| | | 1999) | |
| | Electroporation | (Fromm et al., 1985; Ou-Lee | |
| | (protoplasts) | et al., 1986; Rhodes et al., | |
| | | 1988; Saunders et al., 1989) | |
| | | May be combined with | |
| | | liposomes (Trick and al., | |
| | | 1997) | |
| | Polyethylene | (Shillito, 1999) | |
| | glycol (PEG) | | |
| | treatment | | |
| | Liposomes | May be combined with | |
| | | electroporation (Trick and | |
| | | al., 1997) | |
| | in planta | (Leduc and al., 1996; Zhou | |
| | microinjection | and al., 1983) | |
| | Seed imbibition | (Trick and al., 1997) | |
| | Laser beam | (Hoffinan, 1996) | |
| | Silicon carbide | (Thompson and al., 1995) | |
| | whiskers | | |

Vectors often use a selectable marker to facilitate identifying those cells that have incorporated the vector, especially in vitro. Many selectable markers are well known in the art for selection, usually antibiotic-resistance genes or the use of autotrophy and auxotrophy mutants. Table F lists common selectable markers for mammalian cell transfection.

TABLE F
Useful selectable markers for eukaryote cell transfection

| Selectable Marker | Selection | Action | Reference |
|---|---|---|---|
| Adenosine deaminase | Media includes 9-(β-D- | Conversion of Xyl-A | (Kaufman |
| (ADA) | xylofuranosyl adenine | to Xyl-ATP, which | et al., 1986) |
| | (Xyl-A) | incorporates into | |
| | | nucleic acids, killing | |
| | | cells. ADA detoxifies | |
| Dihydrofolate | Methotrexate (MTX) | MTX competitive | (Simonsen |
| reductase (DHFR) | and dialyzed serum | inhibitor of DHFR. In | and |
| | (purine-free media) | absence of exogenous | Levinson, |
| | | purines, cells require | 1983) |
| | | DHFR, a necessary | |
| | | enzyme in purine | |
| | | biosynthesis. | |
| Aminoglycoside | G418 | G418, an | (Southern |
| phosphotransferase | | aminoglycoside | and Berg, |
| (“APH”, “neo”, | | detoxified by APH, | 1982) |
| “G418”) | | interferes with | |
| | | ribosomal function | |
| | | and consequently, | |
| | | translation. | |
| Hygromycin-B- | hygromycin-B | Hygromycin-B, an | (Palmer et |
| phosphotransferase | | aminocyclitol | al., 1987) |
| (HPH) | | detoxified by HPH, | |
| | | disrupts protein | |
| | | translocation and | |
| | | promotes | |
| | | mistranslation. | |
| Thymidine kinase | Forward selection | Forward: Aminopterin | (Littlefield, |
| (TK) | (TK+): Media (HAT) | forces cells to | 1964) |
| | incorporates | synthesize dTTP from | |
| | aminopterin. Reverse | thymidine, a pathway | |
| | selection (TK−): Media | requiring TK. | |
| | incorporates 5- | Reverse: TK | |
| | bromodeoxyuridine | phosphorylates BrdU, | |
| | (BrdU). | which incorporates | |
| | | into nucleic acids, | |
| | | killing cells. | |

3. Production of Msx1 In Vitro

A host cell, such as a prokaryotic or eukaryotic host cell, can be used to produce msx1. Host cells that are useful for in vitro production of msx1 or msx1 fusion polypeptides, into which a recombinant expression vector encoding msx1 has been introduced, include as nonlimiting examples, E. coli, COS7, and Drosophila S2. In one embodiment, such cells do not modify the produced polypeptide in such as way that when introduced into a subject, such as a human, an immune response is evoked. For example, certain sugar post-translational modifications may provoke such a response. Preferably, such cells produce active polypeptides. In another embodiment, the cells modify the polypeptide so that it has the same or similar posttranslational modifications as the native polypeptide. The cells are cultured in a suitable medium, such that insxl or the desired polypeptide is produced. If necessary msx1 is isolated from the medium or the host cell. Likewise, Fgfs may be similarly produced, using the appropriate corresponding polynucleotides.

D. Cell Culture

Suitable medium and conditions for generating primary cultures are well known in the art and vary depending on cell type, can be empirically determined. For example, skeletal muscle, bone, neurons, skin, liver, and embryonic stem cells are all grown in media differing in their specific contents. Furthermore, media for one cell type may differ significantly from lab to lab and institution to institution. To keep cells dividing, serum, such as fetal calf serum, is added to the medium in relatively large quantities, 5%-30% by volume, again depending on cell or tissue type. Specific purified growth factors or cocktails of multiple growth factors can also be added or are sometimes substituted for serum. When differentiation is desired and not proliferation, serum with its mitogens is generally limited to about 0-2% by volume. Specific factors or hormones that promote differentiation and/or promote cell cycle arrest can also be used.

Physiologic oxygen and subatmospheric oxygen conditions can be used at any time during the growth and differentiation of cells in culture, as a critical adjunct to selection of specific cell phenotypes, growth and proliferation of specific cell types, or differentiation of specific cell types. In general, physiologic or low oxygen-level culturing is accompanied by methods that limit acidosis of the cultures, such as addition of strong buffer to medium (such as HEPES), and frequent medium changes and changes in CO₂ concentration.

In addition to oxygen, the other gases for culture typically are about 5% carbon dioxide and the remainder is nitrogen, but optionally may contain varying amounts of nitric oxide (starting as low as 3 ppm), carbon monoxide and other gases, both inert and biologically active. Carbon dioxide concentrations typically range around 5%, but may vary between 2-10%. Both nitric oxide and carbon monoxide, when necessary, are typically administered in very small amounts (i.e. in the ppm range), determined empirically or from the literature.

The medium can be supplemented with a variety of growth factors, cytokines, serum, etc. Examples of suitable growth factors are basic fibroblast growth factor (bFGF), vascular endothelial growth factor (VEGF), epidermal growth factor (EGF), transforming growth factors (TGFa and TGF(3), platelet derived growth factors (PDGFs), hepatocyte growth factor (HGF), insulin-like growth factor (IGF-1), insulin-like growth factor (IGF-2), insulin, erythropoietin (EPO), and colony stimulating factor (CSF). Examples of suitable hormone medium additives are estrogen, progesterone, testosterone or glucocorticoids such as dexamethasone. Examples of cytokine medium additives are interferons, interleukins, or tumor necrosis factor-x (TNFα). One skilled in the art will test additives and culture components in different culture conditions, as these may alter cell response, active lifetime of additives or other features affecting their bioactivity. In addition, the surface on which the cells are grown can be plated with a variety of substrates that contribute to survival, growth and/or differentiation of the cells. These substrates include but are not limited to laminin, EHS-matrix, collagen, poly-L-lysine, poly-D-lysine, polyomithine and fibronectin. In some instances, when 3-dimensional cultures are desired, extracellular matrix gels may be used, such as collagen, EHSmatrix, or gelatin. Cells may be grown on top of such matrices, or may be cast within the gels themselves.

E. Dedifferentiating Cells

1. Myotubes In Vitro

Myotubes, isolated from a subject, preferably a human, or generated from murine myoblast cell lines (see examples) are cultured in vitro in suitable media. A skilled artisan will know how to vary the conditions set forth to achieve dedifferentiation. A skilled artisan will know how to vary the conditions set forth to achieve dedifferentiation. The following description is set forth as an illustrative example.

To induce dedifferentiation of myotubes in culture, RE is added to differentiation medium (see Examples) at a suitable time after plating the cells at low density onto an appropriate substrate (e.g. tissue culture plastic, gelatin, fibronectin, laminin, collagen, EHS-matrix, etc.—coated surfaces). Medium and extract are preferably changed daily. To identify morphologic dedifferentiation, individual cells are photographed on day 0, before the addition of extract, and every 24 hrs after the addition of extract for up to 10 days or longer.

2. Differentiated Cells In Vitro

Cells isolated from a subject, preferably a human, or generated from cell lines are cultured in vitro in suitable media.

A skilled artisan will know how to vary the conditions set forth to achieve dedifferentiation. The following description is set forth as an illustrative example. To induce dedifferentiation of cells in culture, RE is added to differentiation medium (see Examples) at a suitable time after plating the cells at low density onto an appropriate substrate (e.g. tissue culture plastic, gelatin, fibronectin, laminin, collagen, EHS-matrix, etc.—coated surfaces or in suspension). Medium and extract are preferably changed daily. To identify morphologic dedifferentiation, individual cells are photographed on day 0, before the addition of extract, and every 24 hrs after the addition of extract for up to 10 days or longer.

3. Cells In Vivo

Cells are contacted with RE or with a dedifferentiation agent. RE or one or more dedifferentiation agent may be formulated within a pharmaceutical composition to ensure delivery. In one embodiment, the cells are contacted at a site of injury.

### V. Methods of Identifying and/or Characterizing Dedifferentiation Agents

This application describes methods and compositions for promoting dedifferentiation of cells in vitro and/or in vivo. The application further describes methods and compositions for promoting regeneration using cells dedifferentiated either in vivo or in vitro. Without being bound by theory, the present application has described many exemplary agents including nucleic acids, peptides, polypeptides, small organic molecules, antibodies, antisense oligonucleotides, RNAi constructs, and ribozymes, which promote dedifferentiation. These agents may promote dedifferentiation via any one (or more than one) of the following mechanisms including: (i) promoting FGF signaling, (ii) promoting BMP signaling, (iii) promoting Wnt signaling, (iv) promoting the expression and/or activity of msx1, (v) promoting the expression and/or activity of msx2, (vi) inhibiting the expression and/or activity of msx3, (vii) promoting the expression and/or activity of cyclinD1, (viii) promoting the expression and/or activity of Cdk4, (ix) inhibiting the expression and/or activity of p16, (x) inhibiting the expression and/or activity of p21, (xi) inhibiting the expression and/or activity of p27, (xii) inhibiting the expression and/or activity of Rb, (xiii) inhibiting the expression and/or activity of Wee1, or (xiv) promoting the expression and/or activity of a G1 Cdk complex. Furthermore, the application contemplates that other mechanisms may exist to promote dedifferentiation, and thus suitable agents may promote dedifferentiation via a mechanism distinct from the above cited mechanisms. An agent which promotes dedifferentiation, regardless of the mechanism, is useful in the methods of the present invention. Accordingly, the invention contemplates the identification and/or characterization of agents which promote dedifferentiation.

Agents screened (e.g., a single agent, a combination of two or more agents, a library of agents) include nucleic acids, peptides, proteins, antibodies, antisense oligonucleotides, RNAi constructs (including siRNAs), DNA enzymes, ribozymes, chemical compounds, and small organic molecules. Agents may be screened individually, in combination, or as a library of agents.

In many drug screening programs that test libraries of nucleic acids, polypeptides, chemical compounds and natural extracts, high throughput assays are desirable to increase the number of agents surveyed in a given period of time. Assays that are performed in cell-free systems, such as may be derived with purified or semi-purified proteins, are often preferred as “primary” screens in that they can be generated to permit rapid development and relatively easy detection of an alteration in a molecular target which is mediated by a test agent. Cell free systems include in vitro systems (preparations of proteins and agents combined in a test tube, Petri dish, etc.), as well as cell free systems such as those prepared from extracts or reticulocyte lysates. Moreover, the effects of cellular toxicity and/or bioavailability of the test agents can be generally ignored in such a system, the assay instead being focused primarily on the effect of the agent.

A primary screen can be used to narrow down agents that are more likely to have an effect on dedifferentiation, in vitro and/or in vivo. Such a cell free system for use in the present invention may include a biochemical assay measuring, for example, BMP signaling, Wnt signaling, or FGF signaling. Although an assay constructed in this way is biased in terms of the mechanism by which the agent is exerting its effect, such an approach does allow rapid screening of libraries of agents.

The efficacy of the agent can be assessed by generating dose response curves from data obtained using various concentrations of the test agent. Moreover, a control assay can also be performed to provide a baseline for comparison. Such candidates can be further tested for efficacy in promoting Wnt, BMP or FGF signaling in a cell-based system, for the ability to promote dedifferentiation of one or more cell types in vitro, and/or for the ability to promote dedifferentiation of one or more cell types in vivo.

In addition to cell-free assays, such as described above, the invention further contemplates the generation of cell-based assays for identifying agents having one or more of the desired activities. Cell-based assays may be performed as either a primary screen, or as a secondary screen to confirm the activity of agents identified in a cell free screen, as outlined in detail above. Such cell based assays can employ any cell-type. Exemplary cell types include neuronal cell lines, primary neural cultures, fibroblasts, lymphocytes, mesenchymal cells, etc. Cells in culture are contacted with one or more agents, and the ability of the one or more agents to promote dedifferentiation is measured. Agents which promote dedifferentiation are candidate agents for use in the subject methods.

In addition to the cell free and cell based assays described above. Agents may be screened in vitro or in vivo using animal models of injury and/or degeneration. Exemplary animal models further include wildtype and mutant zebrafish and zebrafish embryos, newts, mice, and rats, as described throughout the application. The invention further contemplates the use of cells, tissues, and whole animals, and such material can be derived from animals and tissues in which dedifferentiation and/or redifferentiation typically occurs (e.g., newt limbs, zebrafish tail), as well as from animals and tissues in which dedifferentiation and/or redifferentiation does not typically occur (e.g., terminally differentiated mammalian skeletal muscle).

### VI. Pharmaceutical Compositions and Methods of Delivery

The compositions of the invention and derivatives, fragments, analogs and homologues thereof, can be incorporated into pharmaceutical compositions. Such compositions typically comprise the nucleic acid molecule, protein, peptide, antibody, small organic molecule, antisense oligonucleotide, or ribozyme, and a pharmaceutically acceptable carrier. A “pharmaceutically acceptable carrier” includes any and all solvents, dispersion media, coatings, antibacterial and antifungal agents, isotonic and absorption delaying agents, and the like, compatible with pharmaceutical administration (Gennaro, 2000). Preferred examples of such carriers or diluents include, but are not limited to, water, saline, ringer's solutions, dextrose solution, and 5% human serum albumin. Liposomes and non-aqueous vehicles such as fixed oils may also be used. Except when a conventional media or agent is incompatible with an active compound, use of these compositions is contemplated. Supplementary active compounds can also be incorporated into the compositions.

The pharmaceutical compositions for the administration of the active agents may conveniently be presented in dosage unit form and may be prepared by any of the methods well known in the art of pharmacy. All methods include the step of bringing the active agent into association with the carrier that constitutes one or more accessory ingredients. In general, the pharmaceutical compositions are prepared by uniformly and intimately bringing the active compound into association with a liquid carrier or a finely divided solid carrier or both, and then, if necessary, shaping the product into the desired formulation. In the pharmaceutical composition the active agent is included in an amount sufficient to produce the desired effect upon the process or condition of diseases.

1. General Considerations

A pharmaceutical composition of the invention is formulated to be compatible with its intended route of administration, including intravenous, intradermal, subcutaneous, oral (e.g., inhalation), transdermal (i.e., topical), transmucosal, and rectal administration. Solutions or suspensions used for parenteral, intradermal, or subcutaneous application can include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of toxicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

2. Injectable Formulations

Pharmaceutical compositions suitable for injection include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EC (BASF, Parsippany, N.J.) or phosphate buffered saline (PBS). In all cases, the composition must be sterile and should be fluid so as to be administered using a syringe. Such compositions should be stable during manufacture and storage and must be preserved against contamination from microorganisms such as bacteria and fungi. The carrier can be a solvent or dispersion medium containing, for example, water, ethanol, polyol (such as glycerol, propylene glycol, and liquid polyethylene glycol), and suitable mixtures. Proper fluidity can be maintained, for example, by using a coating such as lecithin, by maintaining the required particle size in the case of dispersion and by using surfactants. Various antibacterial and antifungal agents, for example, parabens, chlorobutanol, phenol, ascorbic acid, and thimerosal, can contain microorganism contamination. Isotonic agents, for example, sugars, polyalcohols such as manitol, sorbitol, and sodium chloride can be included in the composition. Compositions that can delay absorption include agents such as aluminum monostearate and gelatin.

Sterile injectable solutions can be prepared by incorporating the active compound or composition in the required amount in an appropriate solvent with one or a combination of ingredients as required, followed by sterilization. Generally, dispersions are prepared by incorporating the active compound into a sterile vehicle that contains a basic dispersion medium, and the other required ingredients as discussed.

3. Oral Compositions

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally. Pharmaceutically compatible binding agents, and/or adjuvant materials can be included. Tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL, or corn starch; a lubricant such as magnesium stearate or STEROTES; a glidant such as colloidal silicon dioxide; a sweetening agent such as sucrose or saccharin; or a flavoring agent such as peppermint, methyl salicylate, or orange flavoring.

4. Compositions for Inhalation

For administration by inhalation, the compounds are delivered as an aerosol spray from a nebulizer or a pressurized container that contains a suitable propellant, e.g., a gas such as carbon dioxide.

5. Systemic Administration, Including Patches

Systemic administration can also be transmucosal or transdermal. For transmucosal or transdermal administration, penetrants that can permeate the target barrier(s) are selected. Transmucosal penetrants include, detergents, bile salts, and fasidic acid derivatives. Nasal sprays or suppositories can be used for transmucosal administration. For transdermal administration, the active compounds are formulated into ointments, salves, gels, or creams.

The compounds can also be prepared in the form of suppositories (e.g., with bases such as cocoa butter and other glycerides) or retention enemas for rectal delivery.

6. Carriers

In one embodiment, the active compounds are prepared with carriers that protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such materials can be obtained commercially from ALZA Corporation (Mountain View, Calif.) and NOVA Pharmaceuticals, Inc; (Lake Elsinore, Calif.), or prepared by one of skill in the art Liposomal suspensions can also be used as pharmaceutically acceptable carriers. These can be prepared according to methods known to those skilled in the art, such as in (U.S. Pat. No. 4,522,811).

7. Unit Dosage

Oral formulations or parenteral compositions in unit dosage form can be created to facilitate administration and dosage uniformity. Unit dosage form refers to physically discrete units suited as single dosages for the subject to be treated, containing a therapeutically effective quantity of active compound in association with the required pharmaceutical carrier. The specification for the unit dosage forms of the invention are dictated by, and directly dependent on, the unique characteristics of the active compound and the particular desired therapeutic effect, and the inherent limitations of compounding the active compound.

8. Dosage

The pharmaceutical composition and method of the present invention may further comprise other therapeutically active compounds as noted herein that are usually applied in the treatment of wounds or other associated pathological conditions.

In the treatment of conditions which require tissue regeneration or cellular dedifferention, an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy. In addition, the site of delivery will also impact dosage and frequency.

Combined therapy to engender tissue regeneration is illustrated by the combination of the compositions of this invention and other compounds that are known for such utilities.

Exemplary Conditions which May be Treated by the Methods of the Present Invention.

a. Injury

A physical injury to cells may result in scar formation, and this' scar formation interferes with the normal function of the cell and/or the normal function of the tissue which comprises the injured cells. Such injuries include physical injuries. Physical injuries include, but are not limited to, crushing, or severing of tissue, such as may occur following a fall, car accident, gun shot, stabbing wound, etc. Further examples of physical injuries include those caused by extremes in temperature such as burning, freezing, or exposure to rapid and large temperature shifts. Still further examples of physical injuries include those that result from deprivation of oxygen such as during a heart attack; strangulation, drowning, or stricture. Additional examples of an injury include those caused by infection such as by a bacterial or viral infection. Examples of bacterial or viral infections include meningitis, staph, HIV, influenza, hepatitis, endocardioitis, herpes simplex I, herpes simplex II, Lyme's disease, and the like. In addition to these non-limiting examples, one of skill in the art will recognize that many different types of bacteria or viruses may infect cells and cause tissue injury.

Additionally, injury may occur as a consequence or side effect of other treatments such as surgery, angioplasty, or insertion of a device such as a stent, catheter, wire, pace maker, implant, or intraluminal device. Further treatment regimens which may cause injury to cells include cancer therapies such as chemotherapeutic agents, radiation therapy, and the like which may cause injury to both cancerous and healthy cells. We additionally note that by treatments is meant to include both necessary and elective surgical and non-surgical interventions. By way of example, elective intervention includes procedures such as tubal ligation, vasectomy, cosmetic surgery, circumcision, and gastric reduction. All of these procedures, although generally considered elective, can result in significant complications due to scarring and other tissue injury.

The foregoing examples of cell and tissue injury may occur in any cell type. Exemplary cells and tissues which may be damaged due to injury, and treated with the methods of the present invention, include skeletal muscle, cardiac muscle, cartilage, bone, connective tissue, neuronal tissue (e.g., brain, spinal cord, retina—including both neurons and glia), skin, lymphatic tissue, kidney, liver, gall bladder, pancreas (e.g., including β-cells), esophagus, stomach, rectum, bladder, urethra, small intestine, and large intestine, tissues of the male and female reproductive tract (e.g., ovary, uterus, Fallopian tube, vagina, penis, vas deferens, seminal vesicle, testicle, etc).

b. Degenerative Diseases

A wide range of diseases cause extensive cell damage (i.e., injury) to cells. These include neurodegenerative diseases such as Parkinson's disease, Huntington' disease, ALS, peripheral neuropathy, Alzheimer's disease, stroke, macular degeneration, and the like. Further degenerative conditions include degenerative heart and vascular diseases such as atherosclerosis and occlusive vascular disease, degenerative conditions of cartilage and connective tissue such as osteoarthritis and rheumatoid arthritis, degenerative conditions of the liver such as cirrohis, degenerative conditions of the kidney such as polycystic kidney disease, degenerative conditions of the pancrease such as diabetes, and degenerative conditions of the digestive system including Inflammatory Bowel disease. Additionally, cancer, of any tissue, can be thought of as both a degenerative disease and as an injury. Tissue is often damaged by a combination of the effects of: progression of the disease; treat regimens including medication, radiation therapy, and chemotherapy; and scarring and other damage caused by surgical intervention.

Agents for use in the methods of the present invention, as well as agents identified by the subject methods may be conveniently formulated for administration with a biologically acceptable medium, such as water, buffered saline, polyol (for example, glycerol, propylene glycol, liquid polyethylene glycol and the like) or suitable mixtures thereof. Optimal concentrations of the active ingredient(s) in the chosen medium can be determined empirically, according to procedures well known to medicinal chemists. As used herein, “biologically acceptable medium” includes solvents, dispersion media, and the like which may be appropriate for the desired route of administration of the one or more agents. The use of media for pharmaceutically active substances is known in the art. Except insofar as a conventional media or agent is incompatible with the activity of a particular agent or combination of agents, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations”.

Methods of introduction may also be provided by rechargeable or biodegradable devices. Various slow release polymeric devices have been developed and tested in vivo in recent years for the controlled delivery of agents, including proteinaceous biopharmaceuticals. A variety of biocompatible polymers (including hydrogels), including both biodegradable and non-degradable polymers, can be used to form an implant for the sustained release of an agent at a particular target site. Delivery of agents to injury site can be attained by vascular administration via liposomal or polymeric nano- or micro-particles; slow-release vehicles implanted at the site of injury or damage; osmotic pumps implanted to deliver at the site of injury or damage; injection of agents at the site of injury or damage directly or via catheters or controlled release devices; injection into the cerebro-spinal fluid; injection intrapericardially.

The agents identified using the methods of the present invention may be given orally, parenterally, or topically. They are of course given by forms suitable for each administration route. For example, they are administered in tablets or capsule form, by injection, inhalation, ointment, controlled release device or patch, or infusion.

One or more agents may be administered to humans and other animals by any suitable route of administration. With regard to administration of agents to the brain, it is known in the art that the delivery of agents to the brain may be complicated due to the blood brain barrier (BBB). Accordingly, the application contemplates that agents may be administered directly to the brain cavity. For example, agents can be administered intrathecally or intraventricularly. Administration may be, for example, by direct injection, by delivery via a catheter or osmotic pump, or by injection into the cerebrospinal fluid.

However, although the BBB may present an impediment to the delivery of agents to the brain, it is also recognized that many agents, including nucleic acids, polypeptides and small organic molecules, are able to cross the BBB following systemic delivery. Therefore, the current application contemplates that agents may be delivered either directly to the sight of injury in the CNS or PNS, or may be delivered systemically.

With regard to administration of agents to myocardial tissue, it is known that agents can be administered in a variety of ways including systemically; via catheter, stent, intraluminal device, or wire; and via direct injection to the pericardium.

Actual dosage levels of the one or more agents may be varied so as to obtain an amount of the active ingredient which is effective to achieve a response in an animal. The actual effective amount can be determined by one of skill in the art using routine experimentation and may vary by mode of administration. Further, the effective amount may vary according to a variety of factors include the size, age and gender of the individual being treated. Additionally the severity of the condition being treated, as well as the presence or absence of other components to the individuals treatment regimen will influence the actual dosage. The effective amount or dosage level will depend upon a variety of factors including the activity of the particular one or more agents employed, the route of administration, the time of administration, the rate of excretion of the particular agents being employed, the duration of the treatment, other drugs, compounds and/or materials used in combination with the particular agents employed, the age, sex, weight, condition, general health and prior medical history of the animal, and like factors well known in the medical arts.

The one or more agents can be administered as such or in admixtures with pharmaceutically acceptable and/or sterile carriers and can also be administered in conjunction with other compounds. Such additional compounds may include factors known to influence the proliferation, differentiation or migration of the particular cell type being manipulated. These additional compounds may be administered sequentially to or simultaneously with the agents for use in the methods of the present invention.

Agents can be administered alone, or can be administered as a pharmaceutical formulation (composition). Said agents may be formulated for administration in any convenient way for use in human or veterinary medicine. In certain embodiments, the agents included in the pharmaceutical preparation may be active themselves, or may be a prodrug, e.g., capable of being converted to an active compound in a physiological setting.

Thus, another aspect of the present invention provides pharmaceutically acceptable compositions comprising an effective amount of one or more agents, formulated together with one or more pharmaceutically acceptable carriers (additives) and/or diluents. As described below, the pharmaceutical compositions of the present invention may be specially formulated for administration in solid or liquid form, including those adapted for the following: (1) local administration to the central nervous system, for example, intrathecal, intraventricular, intraspinal, or intracerobrospinal administration; (2) local administration to the myocardium, for example, via a stent, wire, intraluminal device, catheter, or via intrapericardial administration; (3) oral administration, for example, drenches (aqueous or non-aqueous solutions or suspensions), tablets, boluses, powders, granules, pastes for application to the tongue; (4) parenteral administration, for example, by subcutaneous, intramuscular or intravenous injection as, for example, a sterile solution or suspension; (5) topical application, for example, as a cream, ointment or spray applied to the skin; or (6) opthalamic administration, for example, for administration following injury or damage to the retina. However, in certain embodiments the subject agents may be simply dissolved or suspended in sterile water. In certain embodiments, the pharmaceutical preparation is non-pyrogenic, i.e., does not elevate the body temperature of a patient.

Some examples of the pharmaceutically acceptable carrier materials that may be used include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower-oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, one or more agents may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of agent of the present invention. These salts can be prepared in situ during the final isolation and purification of the agents of the invention, or by separately reacting a purified agent of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the agents include the conventional nontoxic salts or quaternary ammonium salts of the agents, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the one or more agents may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of agents of the present invention. These salts can likewise be prepared in situ during the final isolation and purification of the agents, or by separately reacting the purified agent in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated, hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the agent which produces a therapeutic effect. Generally, out of one hundred percent, this amount will range from about 1 percent to about ninety-nine percent of active ingredient, preferably from about 5 percent to about 70 percent, most preferably from about 10 percent to about 30 per cent.

Methods of preparing these formulations or compositions include the step of bringing into association an agent with the carrier and, optionally, one or more accessory ingredients. In general, the formulations are prepared by uniformly and intimately bringing into association an agent of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a agent of the present invention as an active ingredient. An agent of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration of the agents of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agents, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Transdermal patches have the added advantage of providing controlled delivery of an agent of the present invention to the body. Such dosage forms can be made by dissolving or dispersing the agents in the proper medium. Absorption enhancers can also be used to increase the flux of the agents across the skin. The rate of such flux can be controlled by either providing a rate controlling membrane or dispersing the agent in a polymer matrix or gel.

Ophthalmic formulations, eye ointments, powders, solutions and the like, are also contemplated as being within the scope of this invention. These are particularly useful for injury and degenerative disorders of the eye including retinal detachment and macular degeneration.

Pharmaceutical compositions of this invention suitable for parenteral administration comprise one or more agents of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of an agent, it is desirable to slow the absorption of the agent from subcutaneous or intramuscular injection. This may be accomplished by the use of a liquid suspension of crystalline or amorphous material having poor water solubility. The rate of absorption of the agent then depends upon its rate of dissolution which, in turn, may depend upon crystal size and crystalline form. Alternatively, delayed absorption of a parenterally administered agent form is accomplished by dissolving or suspending the agent in an oil vehicle.

### VII. Kits for Pharmaceutical Compositions

The pharmaceutical compositions can be included in a kit, container, pack, or dispenser together with instructions for administration. When the invention is supplied as a kit, the different components of the composition may be packaged in separate containers and admixed immediately before use. Such packaging of the components separately may permit long-term storage without reduction or lose of activity.

(a) Containers or Vessels

The reagents included in the kits can be supplied in containers of any sort such that the life of the different components are preserved, and are not adsorbed of altered by the materials of the container. For example, sealed glass ampoules may contain lyophilized RE, RDF or buffer that have been packaged under a neutral, non-reacting gas, such as nitrogen. Ampoules may consist of any suitable material, such as glass, organic polymers, such as polycarbonate, polystyrene, etc., ceramic, metal or any other material typically employed to hold reagents. Other examples of suitable containers include simple bottles that may be fabricated from similar substances as ampules, and envelopes, that may consist of foil-lined interiors, such as aluminum or an alloy. Other containers include test tubes, vials, flasks, bottles, syringes, or the like. Containers may have a sterile access port, such as a bottle-having a stopper that can be pierced by a hypodermic injection needle. Other containers may have two compartments that are separated by a readily removable membrane that upon removal permits the components to mix. Removable membranes may be glass, plastic, rubber, etc.

(b) Instructional Materials

Kits may also be supplied with instructional materials. Instructions may be printed on paper or other substrate, and/or may be supplied as an electronic-readable medium, such as a floppy disc, CD-ROM, DVD-ROM, Zip disc, videotape, audiotape, etc. Detailed instructions may not be physically associated with the kit; instead, a user may be directed to an internet web site specified by the manufacturer or distributor of the kit, or supplied as electronic mail, or which is located on a server which can be accessed by the user. Access to a server containing instructions may either be freely available, or may be protected (e.g., by password) such that only specific individuals may have access to said instructional materials.

### VIII. Delivery Methods

1. Interstitial Delivery

The compositions of the invention may be delivered to the interstitial space of tissues of the animal body, including those of skeletal muscle, cardiac muscle, skin, brain, lung, liver, spleen, bone marrow, thymus, heart, lymph, blood, bone, cartilage, pancreas, kidney, gallbladder, stomach, intestine, testis, ovary, uterus, rectum, nervous system, eye, gland, and connective tissue. Interstitial space of the tissues comprises the intercellular, fluid, mucopolysaccharide matrix among the reticular fibers of organs and tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. They may be conveniently delivered by injection into the tissues comprising these cells. They are preferably delivered to sites of injury, preferably to live cells and extracellular matrices directly adjacent to dead and dying tissue. Any apparatus known to the skilled artisan in the medical arts may be used to deliver the compositions of the invention to the site of injury interstitially. These include, but are not limited to, syringes, stents, wires, intraluminal devices, and catheters.

2. Systemic Delivery

In the case of damaged tissue throughout a subject, or in the blood vessels (or lymph system) themselves, then delivery into the circulation system may be desired. Any apparatus known to the skilled artisan in the medical arts may be used to deliver the compositions of the invention to the circulation system. These include, but are not limited to, syringes, stents, wires, intraluminal devices, and catheters. One convenient method is delivery via intravenous drip. Another approach would comprise implants, such as transdermal patches, stents, wires, intraluminal devices, and catheters, that deliver the compositions of the invention over prolonged periods of time. Such implants may or may not be absorbed by the subject overtime.

3. Surgical Delivery

During surgical procedures, the methods and compositions of the invention can be advantageously used to simplify the surgery of interest, such as reducing the amount of intervention, as well as to repair the damage wrought by the surgical procedure. The compositions of the invention may be delivered in a way that is appropriate for the surgery, including by bathing the area under surgery, implantable drug delivery systems, and matrices (absorbed by the body over time) impregnated with the compositions of the invention.

4. Superficial Delivery

In the case of injuries to, or damaged tissues on, the exterior surfaces of a subject, direct application of the compositions of the invention is preferred. For example, a gauze impregnated with a compositions, may be directly applied to the site of damage, and may be held in place, such as by a bandage or other wrapping. Alternatively, the compositions of the invention may be applied in salves, creams, or other pharmaceutical compositions known in the art and meant for topical application.

### IX. Business Methods

The present application further contemplates methods of conducting businesses based on the compositions and methods of the invention. The discovery that terminally differentiated cells can be dedifferentiated, and that this can be used to stimulate regeneration of mammalian tissues once thought to be intractable to regeneration, provides for the first time increased capability to treat a large number of injuries and diseases that damage differentiated cell types.

In another aspect, the invention provides a method of conducting a regenerative medicine business comprising: examining patients with an injury or disease that results in cell, tissue or organ damage; collecting a tissue sample from said patient, or from a genetically related family member; dedifferentiating cells from said tissue sample ex vivo; and transplanting said dedifferentiated cells back to said patient to treat the injury or disease. The method of conducting a regenerative medicine business may optionally include preserving the harvested cells, either prior to or following dedifferentiation, for later use. Similarly the method may optionally comprise a system for logging the harvested tissue samples, a method of expanding the dedifferentiated cells prior to transplantation, and/or a method of billing a patient or the patient's insurance carrier for either collection, storage, dedifferentiation, or transplantation of the cells.

In addition to a regenerative medicine business based on the reimplantation of dedifferentiated cells, the invention contemplates additional methods of conducting a regenerative medicine business. The methods comprise: examining patients with an injury or disease that results in cell, tissue or organ damage; collecting a tissue sample from said patient, or from a genetically related family member; dedifferentiating cells from said tissue sample ex vivo; redifferentiating the cells; and transplanting said redifferentiated cells back to said patient to treat the injury or disease. The method of conducting a regenerative medicine business may optionally include preserving the harvested cells, either prior to or following dedifferentiation or following redifferentiation, for later use. Similarly the method may optionally comprise a system for logging the harvested tissue samples, a method of expanding the dedifferentiated cells or redifferentiated cells prior to transplantation, and/or a method of billing a patient or the patient's insurance carrier for either collection, storage, dedifferentiation, or transplantation of the cells.

In another aspect, the invention provides a method of conducting a gene therapy business comprising: examining patients with an injury or disease that results in cell, tissue or organ damage; administering to said patient an amount of an agent effective to treat the said injury or disease; and monitoring said patient during and after treatment to assess efficacy of the treatment. The method of conducting a gene therapy business may optionally include a method of billing a patient or the patient's insurance carrier. Furthermore, the method includes the use of agents comprising nucleic acids, for example, nucleic acids comprising expression vectors.

In another aspect, the present invention provides a method of conducting a drug discovery business comprising: identifying, by the subject assays, one or more agents which promote dedifferentiation; determining if an agent identified in such an assay, or an analog of such an agent, promotes dedifferentiation in vivo and/or invitro; conducting therapeutic profiling of an agent so identified for efficacy and toxicity in one or more animal models; and formulating a pharmaceutical preparation including one or more agents identified as having an acceptable therapeutic profile and which promote dedifferentiation.

In one embodiment, the drug discovery business further includes the step of establishing a system for distributing the pharmaceutical preparation for sale, and may optionally include establishing a sales group for marketing the pharmaceutical preparation.

In certain embodiments, the initially identified dedifferentiation agents can be subjected to further lead optimization, e.g., to further refine the structure of a lead compound so that potency and activity are maintained but balanced with important pharmacological characteristics including:
Solubility
Permeability
Bioavailability
Toxicity
Mutagenicity
Pharmacokinetics—absorption, distribution, metabolism, elimination of the drug
   Structural modifications are made to a lead compound to address issues with the parameters listed above. These modifications however, must take into account possible effects on the molecule's potency and activity. For example, if the solubility of a lead compound is poor, changes can be made to the molecule in an effort to improve solubility; these modifications, however, may negatively affect the molecule's potency and activity. SAR data are then used to determine the effect of the change upon potency and activity. Using an iterative process of structural modifications and SAR data, a balance is created between these pharmacological parameters and the potency and activity of the compound.

Candidate agents or combinations thereof, must them be tested for efficacy and toxicity in animal models. Such therapeutic profiling is commonly employed in the pharmaceutical arts. Before testing an experimental drug in humans, extensive therapeutic profiling (preclinical testing) must be completed to establish initial parameters for safety and efficacy. Preclinical testing establishes a mechanism of action for the drug, its bioavailability, absorption, distribution, metabolism, and elimination through studies performed in vitro (that is, in test tubes, beakers, petri dishes, etc.) and in animals. Animal studies are used to assess whether the drug will provide the desired results. Varying doses of the experimental drug are administered to test the drug's efficacy, identify harmful side-effects that may occur, and evaluate toxicity.

Briefly, one of skill in the art will recognize that the identification of a candidate agent which promotes dedifferentiation in a drug based screen is a first step in developing a pharmaceutical preparation useful for dedifferentiating cells either in vitro or in vivo. Administration of an amount of said pharmaceutical preparation effective to dedifferentiate cells must be both safe and effective. Early stage drug trials, routinely used in the art, help to address concerns of the safety and efficacy of a potential pharmaceutical. In the specific case of a dedifferentiation agent, efficacy of the pharmaceutical preparation could be readily evaluated in normal or transformed cell lines, or in vivo or in vitro in a mouse or rat model. Briefly, mice or rats could be administered varying doses of said pharmaceutical preparations over various time schedules. The route of administration would be appropriately selected based on the particular characteristics of the agent and on the cell type in which dedifferentiation is desired. Control mice can be administered a placebo (e.g., carrier or excipient alone).

In one embodiment, the step of therapeutic profiling includes toxicity testing of compounds in cell cultures and in animals; analysis of pharmacokinetics and metabolism of the candidate drug; and determination of efficacy in animal models of diseases. In certain instances, the method can include analyzing structure-activity relationship and optimizing lead structures based on efficacy, safety and pharmacokinetic profiles. The goal of such steps is the selection of drug candidates for pre-clinical studies to lead to filing of Investigational New Drug applications (“ND”) with the FDA prior to human clinical trials.

Between lead optimization and therapeutic profiling, one goal of the subject method is to develop a dedifferentiation agent which has minimal side-effects. In the case of agents for in vitro use, the lead compounds should not be exceptionally toxic to cells in culture, should not be mutagenic to cells in culture, and should not be carcinogenic to cells in culture. In the case of agents for in vivo use, lead compounds should not be exceptionally toxic (e.g., should have only tolerable side-effects when administered to patients), should not be mutagenic, and should not be carcinogenic.

By toxicity profiling is meant the evaluation of potentially harmful side-effects which may occur when an effective amount of a pharmaceutical preparation is administered. A side-effect may or may not be harmful, and the determination of whether a side effect associated with a pharmaceutical preparation is an acceptable side effect is made by the Food and Drug Administration during the regulatory approval process. This determination does not follow hard and fast rules, and that which is considered an acceptable side effect varies due to factors including: (a) the severity of the condition being treated, and (b) the availability of other treatments and the side-effects currently associated with these available treatments. For example, the term cancer encompasses a complex family of disease states related to mis-regulated cell growth, proliferation, and differentiation. Many forms of cancer are particularly devastating diseases which cause severe pain, loss of function of the effected tissue, and death. Chemotheraputic drugs are an important part of the standard therapy for many forms of cancer. Although chemotherapeutics themselves can have serious side-effects including hair-loss, severe nausea, weight-loss, and sterility, such side-effects are considered acceptable given the severity of the disease they aim to treat.

In the context of the present invention, whether a side-effect is considered significant will depend on the condition to be treated and the availability of other methods to treat that condition. For example, the dedifferentiation agent may be used to promote regeneration of severely damaged cardiac muscle. However, the level of impairment in the health of individuals with myocardial damage varies greatly depending on the overall health of the individual and the extent of damage. These factors must be considered in assessing whether a side-effect is reasonable. By way of another example, the dedifferentiation agent may be used to promote regeneration of cartilage following an injury, such as a sports injury. In this case, the extent to which a side-effect is considered acceptable may be weighed differently given that this condition, though painful, is not likely life-threatening.

Toxicity tests can be conducted in tandem with efficacy tests, and mice administered effective doses of the pharmaceutical preparation can be monitored for adverse reactions to the preparation.

An agent or agents which promote dedifferentiation, and which are proven safe and effective in animal studies, can be formulated into a pharmaceutical preparation. Such pharmaceutical preparation can then be marketed, distributed, and sold. Sale of these agents may either be alone, or as part of a therapeutic regimen including evaluation by a physician, appropriate treatment, and appropriate after-care in coordination with the treating physician or with another licensed physician or health care provider.

### EXAMPLES

The following examples are included to demonstrate preferred embodiments of the present invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing form the spirit and scope of the invention.

1.1 Animals/Tissue Collection

Adult newts, Notophthalmus viridescens, from Charles Sullivan & Co. (Tennessee), were maintained in a humidified room at 24° C. and fed Tubifex worms 2-3×/wk. Operations were performed on animals anesthetized with 0.1% tricaine for approximately 2-3 minutes. Regenerating limb tissue was collected as follows. Forelimbs were amputated by cutting just proximal to the elbow and soft tissue was pushed up the humorus to expose the bone. The bone and soft tissue were trimmed to produce a flat amputation surface. The newts were placed in 0.5% sulfamerazine solution overnight and then back into a normal water environment. Early regenerating tissue (days 1, 3, and 5 postamputation) was collected by reamputating the limb 0.5-1.0 mm proximal to the wound epithelium and removing any residual bone. Nonregenerating limb tissue was collected from limbs that had not been previously amputated. Tissue was extracted 2-3 mm proximal to the forelimb elbow and all bones were removed. Immediately after collection, all tissues were flash frozen in liquid nitrogen and stored at −80° C.

1.2 Preparation of Protein Extracts

Tissues were thawed and all subsequent manipulations were performed at 4° C. or on ice. Six grams of early regenerating tissue from days 1, 3, and 5 (2 g each) or 6 g of nonregenerating tissue were placed separately into 10 ml of Dulbecco's Modified Eagle's Medium (DMEM; GIBCO-BRL No. 11995-065; Carlsbad, Calif.) containing protease inhibitors (2 μg/ml leupeptin, 2 μg/ml A-protinin, and 1 mM phenylmethylsulfonyl fluoride (PMSF)). The tissues were ground with an electronic tissue homogenizer for 1-2 minutes, hand homogenized for 10-15 minutes, and sonicated for 30 seconds. Cell debris was removed in two centrifugation steps. The homogenate was first spun at 2000 g for 25 minutes and then the supernatant was spun again at 100,000 g for 60 minutes. The nonsoluble lipid layer was aspirated and the remaining supernatant filter sterilized through a 0.45 μm filter. The protein content was assayed with a BCA protein assay kit (Pierce; Rockford, Ill.) and stored in 0.5 ml aliquots at −80° C.

1.3 Cell Culture

Newt A1 limb cells were obtained as a gift from Jeremy Brockes (Department of Biochemistry and Molecular Biology, University College London, London, United Kingdom). Mouse C2C12 myoblast cell line was purchased from ATCC. Newt A1 cells were passaged, myogenesis induced, and myotubes isolated and plated at low density (Ferretti and Brockes, 1988; Lo et al., 1993). Newt A1 cells were grown at 24° C. in 2% CO₂. The culture medium was adjusted to the axolotl plasma osmolality of 225 Osm (Ferretti and Brockes, 1988) using an Osmette A Automated Osmometer (Precision Scientific, Inc.; Winchester, Va.). Culture medium contained Minimal Essential Medium (MEM) with Eagle's salt, 10% fetal bovine serum (FBS, Clontech No. 8630-1), 100 U/ml penicillin, 100 μg/ml, streptomycin, 0.28 IU/ml bovine pancreas insulin, 2 mM glutamine, and distilled water.

To induce myotube formation in newt A1 cells, mononucleated cells were grown to confluency and the above medium was replaced with medium containing 0.5% FBS (Differentiation Medium; DM) for 4-6 days. These myotubes were isolated from remaining mononucleated cells by gentle trypsinization (0.05% trypsin) and sequentially sieved through 100 μm and 35 μm nylon meshes. Larger debris and clumped cells were retained on the first sieve, most myotubes were retained on the second sieve, and most mononucleated cells passed through both sieves. Myotubes were gently washed off the 35 μm sieve and plated at either 1-2 myotubes/hpf or <0.25 myotube/hpf onto 35 mm plates precoated with 0.75% gelatin.

C2C12 cells were passaged and myogenesis induced as previously described (Guo et al., 1995). C2C12 myotubes were isolated and plated at low density after gentle trypsinization and sieving through 100 μm mesh. Myotubes were retained on this sieve while mononucleated cells passed through. Myotubes were washed off the sieve and plated at either 1-2 myotubes/hpf or <0.25 myotubes/hpf onto 35 mm plates precoated with 0.75% gelatin.

To induce dedifferentiation of myotubes, 0.1-0.3 mg/ml of RNLE was added to DM 24 hrs after plating at low density (<0.25 myotubes/hpf) in 35 mm gelatin coated plates. Medium and extract were changed daily. To identify morphologic dedifferentiation, individual myotubes were photographed on day 0, before the addition of extract, and every 24 hrs after the addition of extract for up to 10 days. To test for myotube downregulation of muscle specific markers as well as reentry into the S phase of the cell cycle, the cells were plated at slightly higher density (1-2 cells/hpf) with medium and extract changed daily. The cells were stained as described below on day four. Cells cultured in DM alone or in DM with nonRNLE were used as negative controls.

1.4 Immunofluorescence Microscopy

Cells plated at low density in 35 mm plates were washed three times with phosphate buffered saline (PBS) before fixation and immunostaining. Unless otherwise specified, all manipulations were at room temperature, all dilutions of antibodies were prepared in 2% normal goat serum (NGS)/0.1% nonylphenoxy polyethoxy ethanol (NP-40) in PBS, and incubations were followed by washes with 0.1% NP-40 in PBS. Cells were fixed in cold methanol at −20° C. for 10 minutes, rehydrated with PBS, and blocked with 10% NGS for 15 minutes.

TABLE I
Primary antibodies

| Antigen | Antibody type | Dilution | Source |
|---|---|---|---|
| Troponin T | mAb | 1:50 | Sigma #T6277 |
| Myogenin | mAb (F5D clone) | 1:50 | Pharmingen |
| | | | #65121A |
| myoD | NCL-myoD1 | 1:10 | Vector |
| | mouse mAb | | Laboratories, Inc. |
| p21 | WAF1 rabbit | 1:100 | Oncogene Research |
| | Polyclonal antibody | | Products |

Primary antibodies were incubated for 1 hour at 37° C. After three washes, cells were incubated 45 minutes at 37° C. with secondary antibody. For troponin T, a goat anti-mouse IgG conjugated to Alexa 594 (1:100 dilution, Molecular Probes; Eugene, Oreg.) was used, while myogenin and myoD required biotin-xx goat antimouse IgG (1:200 dilution, Molecular Probes), followed by 45 minute incubation with streptavidin Alexa 594 (1:100 dilution, Molecular Probes). No cross-reactivity of the secondary antibodies was observed in control experiments in which primary antibodies were omitted.

In some experiments, cells were counterstained with bromodeoxyuridine (BrdU) for 12 hours, using a 5-bromo-2-deoxy-uridine labeling and detection kit according to manufacturer's instructions (Boehringer Mannheim (Roche); Indianapolis, Ind.). Cells were examined microscopically and photographed using a Zeiss Axiovert 100 equipped with a mounted camera and fluorescent source.

For cells transformed with msx1 (see below), inducing C2C12 cells, Fwd clones, and the Rev clone to differentiate in the presence of DM-doxycycline (DMdox) produced myotubes. Myotubes were then gently trypsinized and replated at low density in DM-dox. The following day, the medium was replaced with growth medium (GM) to induce msx1 expression in the presence of growth factors. Cells were analyzed for myoD, myogenin and p21 expression by immunofluorescence on day 0 (before induction) through day 3 (postinduction). Secondary antibodies were used at 1:200 dilution and included a biotinylated goat anti-mouse IgG antibody (B2763, Molecular Probes) and an Alexa 488-conjugated goat anti-rabbit IgG antibody (A-11034, Molecular Probes). Myotubes were rinsed three times with Dulbecco's phosphate buffered saline (DPBS), treated with Zamboni's fixative for 10 minutes, washed once with DPBS, and permeabilized with 0.2% Triton-X-100 in DPBS for minutes. The myotubes were blocked with 5% skim milk in DPBS for 1 hour and then exposed to two primary antibodies (one was a mouse monoclonal, the other a rabbit polyclonal overnight at 4° C.). The cells were washed three times with DPBS and then treated with two secondary antibodies (a goat anti-rabbit IgG conjugated to Alexa 488 (Molecular Probes) and a goat anti-mouse IgG conjugated to biotin) for 45 minutes at 37° C. Myotubes were washed three times with DPBS and then exposed to 1 μg/ml streptavidin-Alexa 594 (S-11227, Molecular Probes) for 45 minutes at 37° C. The cells were washed three times with DPBS and observed with a Zeiss Axiovert 100 inverted microscope using FITC and Texas Red filters.

1.5 Characterization of the Newt Regeneration Lysate Activity

C2C12 myotubes were plated at low density in DM as described above. Regeneration extract was treated in one of three ways: (1) boiled for 5 minutes; (2) digested with 1% trypsin for 30 minutes at 37° C.; or (3) taken through several freeze/thaw cycles. In three separate experiments, the treated extracts were applied to cultured myotubes at a concentration of 0.3 mg/ml with media and extract changed daily. Immediately after the extract was digested with 1% trypsin, the trypsin was inactivated by dilution in DM in which the cells were cultured. In the freeze/thaw experiments, extract activity was tested after both 2 and 3 freeze/thaw cycles. The effect of the pretreated extracts on myotube S phase reentry was assessed after 4 days of treatment by performing BrdU incorporation assays. The results were compared to BrdU incorporation in myotubes cultured in DM containing RNLE (positive control) and myotubes cultured in DM alone or DM containing nonRNLE (negative controls).

1.6 Construction of msx1 in a Retroviral Vector

A 1.2 kb DNA fragment containing the entire coding region of the mouse msx1 gene was excised from the plasmid phox7XS using SacI and XbaI, blunt-ended with dNTPs and Klenow fragment, and ligated into the LINX retroviral vector at the blunted ClaI site. Clones containing the msx1 gene in both the forward (LINX-msx1-fwd) and reverse (LINX-msx1-rev) orientations were identified and used for the transduction studies.

1.7 Transduction of C2C12 Cells and Selection of Clones Harboring Inducible Msxl

Phoenix-Ampho cells (ATCC No. SD3443) were grown to 70-80% confluency in growth medium (GM) containing 10% tetracycline-tested FBS, 2 mM glutamine, 100 μg/ml penicillin, 100 units/ml streptomycin, and DMEM. Cells were transfected for 10 hours. Medium was replaced and cells were grown an additional 48 hours. The retroviral-containing conditioned medium was then harvested and live cells were removed by centrifugation at 500 g.

C2C12 cells were grown to 20% confluency in GM containing 20% tetracycline-tested FBS, 4 mM glutamine, 2 μg/ml doxycycline, and DMEM. C2C12 cells were infected with the LINX-msx1-fwd or LINX-msx1-rev recombinant retroviruses in T25 tissue culture flasks by replacing GM with retroviral-containing medium comprised of 1 ml retroviral conditioned medium, 2 ml GM, and 4 μg/ml Polybrene. Cells were incubated at 37° C./5% CO₂ for 12-18 hours, and the medium was replaced with fresh GM. The cells were incubated an additional 48 hours and then switched to a 37° C./10% CO₂ incubator. Cells were split just before they reached confluency and selection in G418 (750 μg/ml) was initiated. Selection continued for 6 days and then the cells were split into 100 mm tissue culture plates at a density of 50 cells/plate. Selection was continued for an additional 8 days. Individual cell colonies were isolated using cloning cylinders, and these clones were expanded in GM-G418. Clones were tested for inducible msx1 expression by Northern analysis of total RNA and inhibition of myocyte differentiation in reduced growth factor medium.

1.8 Morphological Dedifferentiation Assays

Myotubes were prepared as described above, gently trypsinized with 0.25% trypsin/1 mM EDTA and replated in DM-dox at a density of 2-4 myotubes/mm² on gridded 35 mm gelatinized plates. The following day residual mononucleated cells were destroyed by lethal injection of water and/or needle ablation using an Eppendorf microinjection system (Westbury, N.Y.). The myotubes were then induced to express msx1 in the presence of growth factors by replacing the culture medium with GM (minus doxycycline). The cells were observed and photographed every 12-24 hours for up to seven days.

1.9 Transdetermination and Pluripotency Assays for Dedifferentiated Cells

Msx1 expression was induced in Fwd clones for five days in the absence of doxycycline (dox) and then suppressed an additional five days in the presence of 2 μg/ml doxycycline. Control msx1-rev and C2C12 cells were similarly treated. In addition, two clonal populations of cells derived from a dedifferentiated Fwd-2 myotube were obtained by plating at limiting dilution in 96-well plates. The above cells were used in the following assays for transdetermination and pluripotency.

Chondrogenic Potential

Chondrogenic potential was assessed in the presence of 2 pg/ml doxycycline according to published protocols (Dennis et al., 1999; Mackay et al., 1998). The cell pellets were treated with O.C.T. compound (Tissue-Tek), frozen in a dry ice/ethanol bath, and then stored at −80° C. wrapped in plastic wrap. A cryostat was used to prepare 6 μm sections. Alternatively, the cell pellets were fixed overnight at 4° C. in freshly prepared 4% paraformaldehyde, processed through a series of ethanol/Hemo DE washes, and embedded in paraffin. A microtome was used to prepare 5 μm sections. Sections prepared from paraffin embedded pellets were stained with alcian blue using the following procedure. Samples were cleared and hydrated, stained with 1% alcian blue (either in 3% acetic acid, pH 2.5 or in 10% sulfuric acid, pH 0.2) for 30 minutes, washed three times with ddH₂O, dehydrated with alcohols, and cleared in HemoDE. Frozen sections were stained for collagen type II using the Vectastain Elite ABC kit according to the manufacturer's instructions (Vector Laboratories), except that samples were treated with 3% H₂O₂ in methanol for 30 minutes following hydration and then with 50 μU/ml chondroitinase ABC for 30 minutes. Anti-collagen type II antibody (NeoMarkers, Lab Vision Corp.; Fremont, Calif.) was used at a 1:50 dilution and the secondary biotinylated antibody was used at 1:200. Samples were counterstained with hematoxylin. Hypertrophic chondrocytes were induced as described (Mackay et al., 1998) and the pellets were stained with alcian blue and for collagen type X (1:50; NeoMarkers, Lab Vision Corp.).

Adipogenic Potential

To assess adipogenic potential, cells were cultured for up to 20 days in GM containing 2 μg/ml doxycycline, 50 pg/ml ascorbic acid 2-phosphate, 10 mM β-glycerophosphate, and 10⁻⁶ or 10⁻⁷M dexamethasone. Medium was changed every two days and cultures were monitored for morphological signs of adipogenic differentiation. At 14-19 days following induction of differentiation, the cells were fixed with 10% neutral buffered formalin for 5 minutes, rinsed three times with ddH₂O, stained with either 0.3% w/v Oil Red 0 for 7 minutes or 100 ng/ml Nile Red for 5 minutes, and rinsed three times with ddH₂O. Cells stained with Oil Red 0 were counterstained with hematoxylin for 2 minutes, rinsed three times in tap water, and once in ddH₂O. Cells stained with Nile Red were observed with fluorescent microscopy using a rhodamine or FITC filter.

Osteogenic Potential

Osteogenic potential was assessed in the presence of 2 μg/ml doxycycline (Jaiswal et al., 1997). Cells were stained for alkaline phosphatase according to manufacturer's instructions using Sigma Kit 85.

Myogenic Potential

Myogenic potential was assessed by morphological observation and immunofluorescence using an antibody that recognizes myogenin (see section entitled Immunofluorescent Studies). Myotubes were observed in cultures treated to assess adipogenic or osteogenic potential.

1.10 Zebrafish Animals and Fin Amputations

Zebrafish 3-6 months of age were obtained from EKKWill Waterlife Resources (Gibsonton, Fla.) and used for caudal fin amputations. Fish were anaesthetized in tricaine and amputations were made using a razor blade, removing one-half of the fin. Animals were allowed to regenerate for various times in water kept at 31-33° C.; these temperatures facilitate more rapid regeneration than more commonly used temperatures of 25-28° C. (Johnson and Weston, 1995). Fish were then anaesthetized and the fin regenerate was removed for analyses.

1.11 Whole Mount In Situ Hybridization of Zebrafish

Probes

To generate antisense RNA probes with a dioxigenin labeling kit (Boehringer Mannheim), a 2.8 kb fgfr1 cDNA fragment, a 1.7 kb fgfr2 cDNA fragment, a 0.6 kb fgfr3 cDNA fragment, a 1.5 kb fgfr4 cDNA fragment (Thisse et al., 1995), a 1.2 kb msxb cDNA fragment, a 2.0 kb msxc cDNA (Akimenko et al., 1995), a 0.6 kb fgf8(ace) cDNA fragment (Reifers et al., 1998), a 2.2 kb fgf4.1 cDNA (Draper et al., 1999), a 2.4 kb wfgf cDNA (Draper et al., 1999), a 3.8 kb β-catenin cDNA (Kelly et al., 1995), a 2.6 kb flkl cDNA fragment (Liao et al., 1997), and a 1.8 kb shh cDNA (Krauss et al., 1993) were used. Fragments containing zebrafish fgfr cDNA sequences were isolated by degenerate PCR using known fgfr tyrosine kinase domain sequences of other species. The assignment of fgfr genes was based on homology comparisons; these sequences have been deposited in Genbank.

In Situ Hybridization

Fin regenerates were fixed overnight at 4° C. in 4% paraformaldehyde in phosphate-buffered saline (PBS), washed briefly in 2 changes of PBS, and transferred to methanol for storage at −20° C. Fins were rehydrated stepwise through ethanol in PBS and then washed in 4 changes of PBS-0.1% polyoxyethylenesorbitan monolaurate (Tween-20; PBT). Then, fins were incubated with 10 μg/ml proteinase K in PBT for 30 minutes and rinsed twice in PBT before 20 minutes refixation. After five washes with PBT, fins were prehybridized at 65° C. for one hour in buffer consisting of 50% formamide, 5×SSC (750 mM NaCl, 75 mM sodium citrate, pH 7.0), 0.1% Tween-20, 50 μg/ml heparin, and 500 μg/ml yeast RNA (pH to 6.0 with citric acid), and then hybridized overnight in hybridization buffer including 0.5 μg/ml dioxigenin-labeled RNA probe. Fins were washed at 65° C. for 10 minutes each in 75% hybridization buffer/25% 2×SSC, 50% hybridization buffer/50% 2×SSC, and 25% hybridization buffer/75% 2×SSC, followed by 2 washes for 30 minutes each in 0.2×SSC at 65° C. Further washes for 5 minutes each were done at room temperature in 75% 0.2×SSC/25% PBT, 50% 0.2×SSC/50% PBT, and 25% 0.2×SSC/75% PBT. After a one hour incubation period in PBT with 2 mg/ml bovine serum albumin, fins were incubated for 2 hours in the same solution with a 1:2000 dilution of fin-preabsorbed, anti-dioxigenin antibody coupled to alkaline phosphatase (Boehringer Mannheim). For the alkaline phosphatase reaction, fins were first washed 3 times in reaction buffer (100 mM Tris-HCl pH 9.5, 50 mM MgCl₂, 100 mM NaCl, 0.1% Tween-20, 1 mM levamisol) and then incubated in reaction buffer with 1× nitro blue terazolium/5-bromo-4-chloro-3-indolyl-phosphate (NBT/BCIP) substrate. In general, positive signals were obtained in 0.5-3 hours. Following the staining reaction, fins were washed in several changes of PBT and fixed in 4% paraformaldehyde in PBS. To obtain sections of fin regenerates, fins were first mounted in 1.5% agarose/5% sucrose and then incubated in 30% sucrose overnight. Frozen blocks were sectioned at 14 μm and observed using Nomarski optics.

For each probe, at least 7 fins were examined for expression at 0, 6, 12, 18, 24, 48, and 96 hours post-amputation. For 18, 24, and 48 hour timepoints with fgfr1, msxb, msxc, and wfgf probes, 25-100 fins were examined in several different experiments. Experiments with sense strand RNA probes were performed with initial antisense experiments to estimate the specificity of signals. To assess gene expression in pharmacologically treated fins, an equal number of untreated fins were also examined. Then, all staining reactions were stopped after strong signals were seen in untreated fins under low magnification.

1.12 Fgfrl Inhibitor Treatments in Zebrafish

SU5402 (Ri; SUGEN, South San Francisco, Calif.) was dissolved in dimethylsulfoxide (DMSO) and added to fish water at a final concentration of 1.7 μM or 17 μM (0.01% DMSO). Up to 10 fish were treated in one liter of water, and tanks were maintained in the dark at 31-33° C. with SU5402 solutions replaced every 24 hours. Zebrafish survived normally and demonstrated no unusual behavior while in the inhibitor solution.

1.13 BrdU Incorporation in Zebrafish

BrdU was dissolved in PBS and fish were treated at a final concentration of 100 μg/ml. For one experiment, fins were amputated and allowed to regenerate for 18 or 24 hours in the absence or presence of 17 μM Ri, with BrdU present during the final 6 hours of regeneration. To test the effects of Ri on proliferation in the established blastema, fins were first allowed to regenerate for 40 hours. Then, untreated fish regenerated an additional 2 hours before a 6 hour incubation with BrdU, while Ri-treated fish underwent a 2 hour R1 preincubation period before a 6 hour period with both Ri and BrdU.

Fins were collected and fixed in 70% ethanol/2 mM glycine overnight, and 10 μm sections were made from frozen blocks. These sections were stained for BrdU incorporation using a detection kit (Roche; Basel, Switzerland), and counterstained with hematoxylin. Sections from untreated and Ri-treated fins were simultaneously processed and developed. Approximately 100 sections from 8 fins were examined from 18 and 24 hour timepoint experiments, while approximately 50 sections from 6 fins were examined from the 48 hour timepoint experiment.

2.1 Regeneration Extract Induces Newt Myotubes to Dedifferentiate

To determine if factors contained in regenerating newt tissue can induce cellular morphologic changes indicative of dedifferentiation, a regenerating newt limb extract (RNLE) was prepared, applied to cultured newt myotubes, and the myotubes followed with light microscopy.

Wound epithelium and proximally-adjacent tissues from day 1-5 newt limb regenerates were used to prepare RNLE as described above. A1 myotubes were cultured at very low density (<0.25 cell/hpf) in DM with 0.3 mg/ml RNLE, and each individual myotube was followed closely for 10 days and photographed every 12-24 hours. The first signs of morphologic dedifferentiation were evident on day 3 when myotubes altered their shape and cleaved into smaller myotubes. By day 10, 16% of the myotubes cleaved to form smaller myotubes or mononucleated cells (Table II). No morphological changes or cellular cleavage was seen in myotubes cultured in DM alone or in DM plus non-regeneration limb extract (negative controls). These findings indicate that RNLE can induce morphologic dedifferentiation in cultured newt myotubes.

To determine the effect of RNLE on normally quiescent multinucleated newt myotubes, RNLE was applied to the cells and tested for BrdU incorporation to assay DNA synthesis. Newt A1 myotubes were plated at low density (1-2 cells/hpf in DM and cultured with 0.3 mg/ml RNLE on day 0. Medium and extract were changed daily and myotubes were assayed for BrdU incorporation on day 4. When quiescent newt A1 myotubes were cultured in DM with RNLE, 25% of the cells were stimulated to enter the S phase of the cell cycle (Table II). By contrast, only 2% of myotubes cultured in DM alone and 3% in DM with 0.3 mg/ml non-regenerating extract incorporated BrdU. These data indicate that regenerating newt tissue contains factors that can induce newt myotubes to reenter the cell cycle.

TABLE II
Newt myotube dedifferentiation induced by RNLE

| Media | MD¹ | BrdU² |
|---|---|---|
| Lysate | 9/56 (16%) | 25/102 (25%) |
| DM w/non-RNLE | 0/50 (0%) | 2/59 (3%) |
| DM alone | 0/43 (0%) | 2/96 (2%) |

¹Morphological dedifferentiation, indicated by cleavage of multinucleated myotubes into smaller myotubes and/or in proliferating mononucleated cells.
²BrdU incorporation to determine entry into S phase.

2.2 RNLE Induces Molecular and Cellular Dedifferentiation of Mammalian Myotubes

To determine if RNLE contains factors that can induce morphologic dedifferentiation of mammalian myotubes, RNLE was applied to C2C12 myotubes and the cells followed by light microscopy.

The myotubes were plated at very low density (<0.25 cell/hpf), cultured in DM with 0.3 mg/ml. RNLE on day 0, and individually photographed every 12-24 hours to document cellular morphologic changes that occurred over the next 10 days. The medium and extract were changed daily. Cellular cleavage was noted by day 2-3 in 11% of the myotubes plated, and cleavage was followed by cellular proliferation in half of these myotubes (Table III). These cellular phenomena were not seen in any C2C12 myotubes cultured with DM alone or DM with 0.3 mg/ml non-RNLE. Thus, murine myotubes cultured with RNLE undergo cytokinetic cleavage to smaller myotubes at nearly the same frequency as newt myotubes (11% vs. 16%). In addition, cleavage was often followed by cellular proliferation in the C2C12 myotubes, an unexpected finding since RNLE-treated newt myotubes did not proliferate. These data indicate that RNLE induces dedifferentiation and proliferation of cultured mammalian myotubes.

To determine if RNLE affects expression of muscle determination and differentiation proteins, RNLE was applied to C2C12 myotubes and indirect immunofluorescence assays were performed to determine altered expression of the muscle differentiation proteins myogenin and myoD and of the muscle contractile protein, troponin-T. Each of these myogenic markers was downregulated in C2C12 myotubes when cultured with the RNLE for four days. Nuclear downregulation of myogenin and MyoD was seen respectively in 15% and 19% of the myotubes. Troponin-T was downregulated in the cytoplasm of 30% of the myotubes. By contrast, myoD and myogenin were consistently present in the controls, and troponin T was identified in approximately 94-97% of the controls (Table III). Downregulation of all markers in RNLE-treated myotubes was greatest by day 4. These data indicate that newt RNLE downregulates skeletal muscle differentiation factors in cultured mammalian myotubes.

To determine if regenerating newt tissue could induce S phase reentry in terminally differentiated mammalian myotubes, BrdU incorporation was assayed in RNLE treated. C2C12 myotubes. C2C12 myotubes were plated at low density (1-2 cells/hpf) and cultured in DM with 0.3 mg/ml of the RNLE. The extract was added on day 0, medium and extract were changed daily, and cells were assayed for BrdU incorporation on the fourth day. Eighteen percent of RNLE-treated C2C12 myotubes showed S phase reentry (FIG. 3, Table 1B). By contrast, no BrdU incorporation was seen in cells cultured in DM alone or in DM with non-RNLE (Table II). RNLE can therefore induce cell cycle reentry in cultured mammalian myotubes.

TABLE III
Mammalian myotube dedifferentiation induced by RNLE

| Media | MD¹ | BrdU² | MyoD³ | Myogenin³ | Troponin-T³ |
|---|---|---|---|---|---|
| Lysate | 10/92 | 14/76 | 18/93 | 12/82 | 20/66 |
| | (11%) | (18%) | (19%) | (15%) | (30%) |
| DM w/non- | 0/63 | 0/30 | 0/46 | 0/54 | 1/32 |
| RNLE | (0%) | (0%) | (0%) | (0%) | (3%) |
| DM alone | 0/61 | 0/32 | 0/40 | 0/48 | 3/47 |
| | (0%) | (0%) | (0%) | (0%) | (6%) |

¹Morphological dedifferentiation, indicated by cleavage of multinucleated myotubes into smaller myotubes and/or in proliferating monucleated cells.
²BrdU incorporation to determine entry into S phase.
³Downregulation of muscle cell specific markers compared to untreated myotubes. Cells were stained on the fourth day of the experiment.

2.3 Dedifferentiation Signal is Likely Comprised of Proteins

The dedifferentiation signal(s) found in the RNLE could belong to a number of different types of biomolecules, including proteins, lipids, nucleic acids, and polysaccharides. To characterize the nature of one or more of the active components of the RNLE, the inventors subjected the extract to a number of different conditions. The results are summarized in Table IV.

The preparation of RNLE reduced the likelihood that the dedifferentiation factor(s) were lipids, since nonsoluble lipids were removed following a high-speed centrifugation step. Repeated freezing and thawing of RNLE reduced the dedifferentiation activity, while boiling for 5 minutes eradicated all activity. When the RNLE was treated with the protease, trypsin, the dedifferentiation signal was abolished, indicating that proteins were a primary component of the factor. The dedifferentiation signal may comprise a single protein or a group of proteins; such proteins may contain certain post-translational modifications, e.g. glycosylation.

TABLE IV
RNLE active component characterization by measuring effect
on S phase reentry

| Treatment | BrdU |
|---|---|
| Heat inactivation¹ | Inhibition |
| Freeze/thaw | Inhibition |
| Protease² | Inhibition |
| SU5402 (Ri)³ | No effect |

¹100° C. for 5 minutes,
²10% trypsin,
³Inhibits Fgfr.

2.4 Generation of C2C12 Clones Containing an Inducible msxl Gene

The mouse msxl gene (SEQ ID NO: 1) (Hill et al., 1989) was cloned into the LINX vector in both the forward (LINX-msx1-fwd) and reverse (LINX-msx1-rev) orientations. LINX is a retroviral vector containing a minimal CMV promoter regulated by the tetracycline-controlled transactivator (tTA) (Gossen and Bujard, 1992; Hoshimaru et al., 1996). Tetracycline or its analog, doxycycline (dox), binds to and inactivates tTA, preventing transcription from the minimal. CMV promoter. In the absence of these antibiotics, tTA binds to the tetracycline response element (TRE) and induces transcription.

LINX-msx1-fwd and LINX-msx1-rev were transduced into C2C12 myoblasts and clones (Fwd-2, Fwd-3, and Rev-2) grown in selective medium were either induced or suppressed for msx1 expression, using dox. Total RNA was extracted and Northern blots were probed with a 40-nucleotide oligomer complimentary to the msxl transcript. Msxl was induced, suppressed, or induced and then suppressed. After five days of induction, a 2.1 kb msxl signal was observed in C2C12-LINX-msx1-fwd(Fwd) clones. Phosphorimage analysis revealed a 25-fold induction in msxl expression. Inducible expression can be reversed when msx1 was again suppressed by growth in medium containing 2 μg/ml doxycycline. C2C12 myoblasts and clones containing the LINX-msxl-rev construct (Rev) did not express msx1.

Ectopic expression of msx1 has been shown to inhibit the differentiation of mouse myoblasts into myotubes (Song et al., 1992). To assess whether induced msx1 protein was functional, the transfected myoblasts were tested for their ability to differentiate. Clones were grown in the presence or absence of dox to either induce or suppress msx1 expression. Once confluency was reached, GM was replaced with DM, and induction or suppression of msx1 was continued. Over ten days, the clones were observed for morphological signs of differentiation by phase contrast microscopy. Fwd clones that were cultured in conditions that suppressed msx1 expression readily produced myotubes, while those expressing msx1 failed to produce myotubes. Control C2C12 myoblasts and Rev clones differentiated normally when cultured under either the induction or suppression conditions. These results indicate that the Fwd clones contained an inducible msx1 gene that produces functional msx1. Two Fwd clones (Fwd-2 and Fwd-3) and one Rev clone (Rev-2) were chosen for further study.

2.5 Msx1 Reverses Expression of Muscle Differentiation Proteins in Mouse Myotubes

One biochemical indicator of myotube dedifferentiation would be the reduction in levels of myogenic differentiation proteins. To determine if the myogenic factors MyoD, myogenin, MRF4, and p21 are reduced as a consequence of msx1 expression, indirect immunofluorescence assays were performed on myotubes that had been induced to express msx1 in the presence of GM. All of these myogenic factors were reduced to varying degrees in murine myotubes. Within 1 day of msx1 induction, MRF4 was reduced to undetectable levels in 34% of induced myotubes. Likewise, myogenin was undetectable in approximately 26% of all induced myotubes. The percentage of myotubes showing undetectable levels of MRF4 and myogenin rose through days 2 and 3 to 50% and 38%, respectively. MyoD expression was not affected until the second day of msx1 induction. On day 2, 10% of all myotubes exhibited a marked reduction of MyoD levels and this percentage rose to 28% by day 3. The percentage of myotubes exhibiting undetectable levels of p21 rose from 10% on day 1 postinduction to 20% by day 3. To ensure that the observed reduction of myogenic protein levels of test myotubes was not the result of myotube aging, control myotubes were matched for age. Normal expression of muscle proteins was observed in 90%-100% of control C2C12 myotubes. These results indicate that ectopic msx1 expression can cause a reduction in the levels of myogenic proteins in terminally differentiated mammalian myotubes.

2.6 Msx1 Induces Mouse Myotube Cleavage and Cellular Proliferation

To test whether ectopic msxl expression and growth factor stimulation could induce cleavage of terminally differentiated mammalian myotubes, isolated myotubes were plated at low density, and the remaining mononucleated cells were eliminated by lethal injection and/or needle ablation (Kumar et al., 2000). Fresh DM was added to the myotubes, and they were incubated overnight. The cultures were again examined for residual mononucleated cells and those present were eliminated before photographing the entire gridded region. No residual mononucleated cells were observed following this procedure in either Fwd or control myotubes. msxl expression was then induced in one set of Fwd myotubes, while a control set of myotubes remained suppressed. Both sets of myotubes were stimulated with GM and followed daily for up to 7 days by microscopic observation and photography. Dedifferentiation was assessed by morphologic examination using the following criteria: (1) cleavage of the myotubes into mononucleated cells or smaller myotubes, and (2) proliferation of the myotube-derived mononucleated cells. FIG. 3A shows an example of a large multinucleated myotube that cleaved to form two smaller multinucleated myotubes. Cleavage of this large myotube was almost complete at day 6 of msx1 induction. Once cleaved, the two myotubes remained separated and viable through the duration of the experiment. Of the 148 test myotubes treated with the induction conditions, 13 (8.8%) underwent cleavage to form either smaller myotubes or mononucleated cells. The first signs of dedifferentiation were evident two days following induction of msx1. At this time, the dedifferentiating myotubes had completely cleaved to form mononucleated cells. Signs of impending cleavage were also observed, such as cell stretching and cleavage initiation. Such cleavages eventually produced proliferating, mononucleated cells by day 4.5. The mononucleated cells arising from these myotubes continued to proliferate and reached cellular confluence by day 7. Proliferation of the resulting mononucleated cells was evident by day 5, and on day 6, numerous myotube-derived mononucleated cells were present. Of 148 test myotubes treated with the induction conditions, 8 (5.4%) dedifferentiated to a pool of proliferating mononucleated cells. Thus, msxl can induce myotubes to stretch and cleave, giving rise to smaller myotubes or mononucleated cells that proliferate.

To ensure that myotube cleavage to mononucleated cells and subsequent proliferation resulted from msx1 expression and was not an artifact of hidden, reserve mononucleated cells, these experiments were repeated, using control cells consisting of uninduced Fwd, Rev, and nontransduced C2C12 myotubes. Of the 151 control myotubes studied, only one atypical myotube cleaved to form a few mononucleated cells. However, these cells did not proliferate even after 7 days in GM. No other control myotubes showed evidence of stretching and cleaving, and no proliferating mononucleated cells were observed. The Fisher-Irwin exact test indicates that the difference in cleavage frequency between msxl-expressing and control myotubes is significant at p=0.0006. Likewise, the difference in cleavage/proliferation frequency between msx1-expressing and control myotubes is significant at p=0.003. Thus the combination of ectopic msx1 expression and stimulation with growth factors can induce a percentage of mouse myotubes to dedifferentiate into smaller myotubes or proliferating, mononucleated cells.

2.7 Msxl Induces Dedifferentiation of Mouse Myotubes to Pluripotent Stem Cells

To determine if the dedifferentiated, proliferating mononucleated cells were pluripotent, two clonal populations of cells derived from a single Fwd-2 myotube were isolated. The clones were cultured under conditions that were favorable for adipogenesis, chondrogenesis, osteogensis, or myogenensis (Dennis et al., 1999; Grigoriadis et al., 1988; Jaiswal et al., 1997; Mackay et al., 1998; Pittenger et. al., 1999). Msx1 expression was suppressed during these redifferentiation assays.

The dedifferentiated clones were tested for chondrogenic potential by pelleting 2.5×10⁵ cells in chondrogenic differentiation medium and feeding the cell pellets every two days with fresh medium. These cells readily differentiated into chondrocytes that produced an extracellular matrix staining faintly with alcian blue and containing collagen type II. Differentiated cells could be further induced to form hypertrophic chondrocytes that stained with alcian blue and reacted with type X collagen. No chondrocytes or hypertrophic chondrocytes were identified in control C2C12 or msx1-rev-2 cells.

When cultured in adipogenic differentiation medium (ADM) for 7-16 days, the dedifferentiated clones produced cells that exhibited adipocyte morphology. These cells were characterized by the presence of numerous vacuoles that stained bright orange upon treatment with the lipophilic dyes, oil red O and Nile red (FIG. 4A). Control C2C12 or Rev-2 cells that had been treated with ADM did not show these characteristic features of adipogenesis (FIG. 4A). The combination of morphologic features and lipid-staining vacuoles suggests that some of the cells had differentiated into adipocytes.

Dedifferentiated clones could also be induced to differentiate into cells expressing an osteogenic marker by treatment with osteogenic-inducing medium (OIM). We observed numerous cell foci per 35 mm plate that stained positive for alkaline phosphatase activity, while very little alkaline phosphatase was identified in control C2C12 or Rev-2 cells (FIG. 4A). Myotubes readily formed in ADM or OIM and were identified by morphology and reactivity to an anti-myogenin antibody (FIG. 4A). As expected, control C2C12 and Rev cells also readily differentiated into myotubes (FIG. 4A; data not shown).

Thus, the combination of ectopic msx1 expression and growth factor treatment can induce terminally-differentiated mouse myotubes to dedifferentiate to a pool of proliferating, pluripotent stem cells that are capable of redifferentiating into several cell lineages.

2.8 Msx1 Induces Transdetermination of Mouse Myoblasts

The inventors contemplated that if msx1 expression caused terminally-differentiated myotubes to completely dedifferentiate, ectopic expression of msx1 might promote transdetermination of C2C12 myoblasts. Msx1 expression was induced in Fwd myoblasts for five days and then suppressed. When treated with the appropriate media, these cells readily differentiated into chondrocytes, adipocytes, myotubes, and cells expressing an osteogenic marker (FIG. 5). No evidence of transdetermination was observed in control cells. These results indicate that transdetermination of myoblasts resulted from ectopic expression of msx1.

2.9 Expression of Fgf Signaling Pathway Members During Zebrafish Fin Blastema Formation and Regenerative Outgrowth

The zebrafish fin is composed of several segmented bony fin rays, or lepidotrichia, each consisting of a pair of concave, facing hemirays that surround connective tissue, including fibroblasts, as well as nerves and blood vessels. Lepidotrichia are connected by vascularized and innervated soft mesenchymal tissue. The early, events that occur during lepidotrichium regeneration can be separated into four stages (A-D) when raised at 33° C. (Goss and Stagg, 1957; Johnson and Weston, 1995; Santamaria and Becerra, 1991). During the first stage (0-12 hours after amputation), a wound epidermis derived from fin epidermal cells forms over the stump. During stage B (approximately 12-24 hours after amputation), wound epidermal cells continue to accumulate. Meanwhile, fibroblasts and scleroblasts (or osteoblasts) located 1-2 segments proximal to the amputation site and between hemirays loosen and disorganize, assume a longitudinal orientation, and appear to migrate toward the wound epidermis. By stage C (24-48 hours), distal migration and proliferation of these cells have resulted in a blastema. During stage D (48 hours and throughout the remainder of regeneration), the blastema is thought to have two prominent functions: (1) the distal portion facilitates outgrowth via cell division; (2) the proximal portion differentiates to form specific structures of the regenerating fin. Following caudal fin amputation, complete regeneration occurs in 1-2 weeks.

To demonstrate that Fgf signaling participates in zebrafish caudal fin regeneration, the expression of four fgfr genes in the early fin regenerate at timepoints ranging from 0 to 96 hours postamputation was assessed using in situ hybridization. The earliest point at which faint but consistent expression of fgfr1 was detected in fin regenerates was 18 hours postamputation, in cells that appeared to be in the process of forming the blastema. Longitudinal fin sections indicated that, at 18-24 hours postamputation, fgfr1 transcripts localize in fibroblast-like cells between hemirays just proximal and distal to the amputation plane. At 48 hours postamputation, during regenerative outgrowth, whole mount analyses consistently revealed expression of fgfr1 in both distal and proximal portions of the regenerate. Sections at this stage indicated transcripts in a small population of cells comprising the distal blastema, as well as in a significant portion of the basal layer of the regeneration epidermis. The epidermal domain appeared to overlap with cells that express sonic hedgehog (shh) at this stage (Laforest et al., 1998). These expression domains were also conspicuous at 96 hours postamputation. In addition, weak but consistent expression of fgfr2 and fgfr3 was observed in the proximal fin regenerate as early as 48 hours after amputation. These receptors were similarly expressed in diffuse domains. fgfr4 expression was not detected in the regenerating fin. These data indicate that cells of the fin regenerate, including blastemal progenitor cells as well as mature blastemal cells, express receptors for Fgfs.

Because msx genes have been implicated as downstream transcriptional targets in Fgf signaling pathways (Kettunen and Thesleff, 1998; Vogel et al., 1995; Wang and Sassoon, 1995), and have been postulated to be important for the undifferentiated state of embryonic mesenchymal tissue (Song et al., 1992), as well as the adult urodele limb blastema (Koshiba et al., 1998), the onset and domain of expression of zebrafish msxb and msxc in the fin regenerate was examined. Detectable msxb expression in fin regenerates was 18 hours postamputation. Sections indicated that during blastema formation, msxb transcripts were distributed in a similar manner as fgfr1 transcripts, in fibroblast-like cells just proximal and distal to the amputation plane. By 48 hours and throughout the remainder of regeneration, all msxb-positive cells were contained within the distal blastemal region, as previously reported (Akimenko et al., 1995). Msxc expression domains were virtually identical to those of msxb at all timepoints. Colocalization of fgfr1 transcripts with msxb and msxc transcripts during blastema formation and regenerative outgrowth supports the hypothesis that Fgf signaling is important for these processes.

To demonstrate that Fgfs are synthesized in the regenerating fin, probes representing characterized zebrafish fgf genes were used for in situ hybridization experiments. No fgf4.1 or fgf8 (ace) transcripts were detected in fin regenerates. However, a member of the Fgf8, Fgf17, and Fgf18 subclass of Fgf ligands, “Wound (W)fgf”, was expressed in the fin regenerate (Draper et al., 1999). wfgf expression was consistently observed at 48 hours postamputation in the distal-most cells of the regeneration epidermis, where it was maintained throughout outgrowth. Experiments examining wfgf expression during blastema formation were equivocal, showing faint expression in approximately 50% of the regenerates. These data indicate that at least one Fgf member is present in the regenerating fin.

2.10 Inhibition of Fgfrl Blocks Blastema Formation

To functionally assess roles of Fgfs in fin regeneration, the lipophilic drug SU5402 (R1), which has been shown to disrupt Fgfrl autophosphorylation and substrate phosphorylation by binding specifically to its tyrosine kinase domain, was used. The IC₅₀ of Ri with respect to Fgfrl activity in mammalian cells was shown previously to be 10-20 μl (Mohammadi et al., 1997). This concentration of Ri causes a dramatic truncation of posterior structures when applied to developing zebrafish embryos. Such embryos appear remarkably similar to those injected with mRNA encoding a dominant-negative Fgfrl (Griffin et al., 1995). Therefore, R1 effectively blocked zebrafish Fgfrl activity.

Previous studies have shown that R1 does not block platelet-derived growth factor, epidermal growth factor, and insulin receptors at concentrations greater than 50 μM in mammalian cells, and has no effects on activities of numerous serine threonine kinases (Mohammadi et al., 1997, Sun et al., 1999). However, R1 does inhibit Flkl, a vascular endothelial growth factor receptor and the earliest known marker for endothelial progenitor cells (Liao et al., 1997), at 10-20 μM. In zebrafish fin regenerates, consistent expression of flkl was not observed until 96 hours postamputation, when it appeared in blastemal cells (n=22). flkl expression was not apparent during blastema formation by in situ hybridization 24 hours postamputation (n=14).

To determine if signaling through Fgfrl is required for regeneration, zebrafish were treated for 96 hours with Ri immediately following amputation. Treatment of zebrafish with 1.7 μM Ri (0.5 mg/liter) inhibited fin regeneration to varying degrees. Of 10 fins examined, 4 regenerated normally, 5 showed slight regenerative defects, and one had a regenerative block. However, all animals exposed to 17 μM Ri (5 mg/liter) demonstrated complete regenerative blocks (n=9). These results indicated that Fgf signaling is required for zebrafish fin regeneration.

To determine if a blastema forms in the absence of Fgf signaling, Ri-treated fin regenerates were examined morphologically. While a wound epidermis consistently formed over the fin stumps of Ri-treated fish, blastemal morphogenesis did not occur. However, mesenchymal cells proximal to the amputation plane showed disorganization, as well as longitudinal orientation suggestive of distal migration.

BrdU incorporation was used to analyze DNA replication and cellular proliferation. Normal proximal mesenchymal cell labeling in Ri-treated fins during 12-18 hours and 18-24 hours postamputation was observed. To determine if blastemal cells underwent DNA replication in the presence of Ri, BrdU incorporation in fins briefly treated with Ri during regenerative outgrowth (40-48 hours postamputation) was analyzed. Blastemal cells of these fins demonstrated greatly reduced incorporation of BrdU. While distal blastemal cells were routinely labeled in sections of untreated fins, labeling of these cells was never observed in sections from Ri treated fns. Furthermore, labeled proximal blastemal cells, which likely had incorporated BrdU through division in the distal blastema, were heavily distributed in sections of untreated fins but sparsely distributed in sections of Ri-treated fins. Nevertheless, proliferation in mesenchymal cells proximal to the amputation plane again was similar in untreated and Ri treated groups. The lack of effect by Ri on proximal mesenchymal tissue was not due to poor tissue penetration, as fins treated for 48 hours with Ri before BrdU treatment also showed normal proximal mesenchymal incorporation. These results indicate that Fgf signaling is essential for blastema formation, likely by facilitating mesenchymal cellular proliferation near the wound epidermis.

To assess molecular effects of the regenerative block in Ri-treated fins, the expression of β-catenin, msxb, and msxc was analyzed. β-catenin was expressed at high levels in the wound epidermis of untreated regenerating fins as early as 3 hours postamputation and throughout the regeneration process. β-catenin expression was normal in Ri-treated fins, suggesting that such fins have no gross defects in wound healing (n=7). However, expression of the blastemal markers msxb and msxc in R1 treated fins was extremely low or undetectable in 24 hour regenerates, and undetectable in 48 hour regenerates (msxb: 21 fins, msxc: 8 fins). These data indicate that Fgf signaling is necessary for msxb/c transcription in the fin regenerate.

2.11 Fgfrl Inhibition Blocks Regenerative Outgrowth

Because wfgf and fgfr1 expression domains were maintained in the fin regenerate during outgrowth, and as blastemal cell BrdU incorporation was blocked by Ri, Fgf signaling likely participates in blastemal maintenance/regenerative outgrowth. To test this hypothesis, the effects of Ri on ongoing regenerates were examined. Ri treatment inhibited further outgrowth of 24-72 hour fin regenerates and often caused the accumulation of an unusually thick regeneration epidermis, as well as dorsoventral migration of melanocytes into adjacent rays. This result may be a consequence of cellular migratory processes by the epidermal and pigment cells that usually pair with new distal growth. In addition, new bone deposition was not interrupted by Ri treatment despite the lack of outgrowth, as lepidotrichial material was observed at unusually distal locations in sections of these fins.

To investigate the molecular effects of this outgrowth inhibition by Ri, marker expression was examined following a 24 hour R1 application period. No significant reduction of 48 or 72 hour epidermal wfgf expression was seen (n=16). However, expression of msxb was diminished in Ri-treated fins that had already regenerated normally for 24 or 48 hours (10 of 18 Ri-treated fins had no detectable msxb expression, while the remaining 8 fins showed low levels). Similar effects on msxc expression were observed (n=8). msxb expression was not detected in 24 or 48 hour fin regenerates exposed to R1 for 48 hours (n=18). Thus, Fgf signaling is required for blastema maintenance and regenerative outgrowth, but is not crucial for other processes including melanocyte migration or bone deposition.

Finally, because fgfr1 also was expressed in epidermal cells during regenerative outgrowth (see FIG. 2C, D), Fgf signaling may be important for patterning the regenerate. To test this hypothesis, the effects of Ri treatment on expression of the patterning gene shh were determined. As previously reported, shh localized to bilateral domains of the basal layer of the fin epidermis as early as 48 hours postamputation (Laforest et al., 1998). Release of Shh from these cells is thought to direct differentiation of blastemal cells into scleroblasts, which deposit bone in forming the new segrnents of the regenerate. Treatment of 48 or 72 hour fin regenerates with Ri for 24 hours dramatically reduced shh expression (0 of 18 fins had detectable shh transcripts; FIG. 6H). These data indicate that intact Fgf signaling is required for normal expression of shh in the fin regenerate.

### REFERENCES

U.S. Pat. No. 4,166,452
U.S. Pat. No. 4,816,567
WO 90/10448
EPO 402226
WO 91/04753
WO 91/00357
WO 91/06629
Akimenko et al. (1995) Development 121: 347-357.
Alam and Cook. (1990) Anal. Biochem. 188: 245-254.
Altschul and Gish. (1996) Methods Enzymol. 266: 460-80.
Altschul et al. (1997) Nucleic Acids Res. 25: 3389-402.
Andres and Walsh. (1996) J Cell Biol. 132: 657-666.
Austin et al. (1990) Development 110: 713-732.
Ausubel et al. (1987) Current protocols in molecular biology. John Wiley & Sons, New York.
Baird and Klagsbrun. (1991) The fibroblast growth factor family. New York Academy of Sciences, New York, N.Y.
Bechtold and Pelletier. (1998) Methods Mol. Biol. 82: 259-66.
Becker and Guarente. (1991) Methods Enzymol. 194: 182-187.
Becker et al. (1974) Nature 248: 145-147.
Beggs. (1978) Nature 275: 104-109.
Bei and Maas. (1998) Development 125: 4325-4333.
Berger et al. (1988) Gene 66:1-10.
Bodine et al. (1991) Exp. Hematol. 19: 206-212.
Brockes. (1997) Science 276: 81-7.
Capecchi. (1980) Cell 22: 479.
Carter. (1986) Biochem J. 237: 1-7.
Case et al. (1979) Proc Natl Acad Sci USA. 76: 5259-63.
Cepko et al. (194) Cell 37: 1053-1062.
Chalfie et al. (1994) Science 263: 802-805.
Chaney et al. (1986) Somatic Cell Mol. Genet. 12: 237.
Chen and Okayama. (1988) BioTechniques. 6: 632-638.
Chen et al. (1994) PNAS 91: 3054-3057.
Cohen et al. (1972) PNAS 69: 2110.
Crossley et al. (1996) Cell 84: 127-136.
Davis et al. (1990) Exp Neurol. 108: 198-213.
de Louvencourt et al. (1983) J Bacteriol 154: 737-742.
de Wet et al. (1987) Mol Cell Biol 7: 725-737.
Dennis et al. (1999) J Bone Miner Res 14: 700-709.
Draper et al. (1999) Developmental Biology 210: 151-180.
Elroy-Stein and Moss. (1990) PNAS 87: 6743-6747.
Endo and Nadal-Ginard. (1989) SV40 large T antigen induces reentry of terminally differentiated myotubes into the cell cycle. In Cellular Molecular Biology of Muscle Development. Alan R. Liss, Inc., NY. 95-104.
Endo and Nadal-Ginard. (1998) J Cell Science 111: 1081-1093.
U.S. Pat. No. 4,522,811
Escudero and Hohn. (1997) Plant Cell. 9: 2135-2142.
Fekete and Cepko. (1993) PNAS. 90: 2350-2354.
Felgner et al. (1987) PNAS. 84: 7413-7417.
Ferretti and Brockes. (1988) The Journal of Experimental Zoology. 247: 77-91.
Fieck et al. (1992) Nucleic Acids Res. 20: 1785-91.
Finer et al. (1999) Current Topics in Microbiology and Immunology. 240: 59-80.
Fleer et al. (1991) Biotechnology (NY). 9: 968-75.
U.S. Pat. No. 5,804,604.
Fromm et al. (1985) PNAS 82: 5824-5828.
Fujita et al. (1986) Cell. 46:401-407.
Gallagher. (1992) GUS protocols: Using the GUS gene as a reporter of gene expression. Academic Press, San Diego, Calif.
Gennaro. (2000) Remington: The science and practice of pharmacy. Lippincott, Williams & Wilkins, Philadelphia, Pa.
Gietz, et al. (1998). Growth and transformation of Saccharomyces cerevisiae. In Cells: A laboratory Manual. Cold Spring Harbor Press, Cold Spring Harbor, N.Y.
Gilbert. (1991) Developmental Biology. Sinauer Associates, Inc, Sunderland, Mass.
Goding. (1996) Monoclonal antibodies: Principles and Practice. Academic Press, San Diego. 492 pp.
Gorman et al. (1982) Mol. Cell. Biol. 2: 1044-1051.
Goss and Stagg. (1957) J. Exp. Zool. 136: 487-508.
Gossen and Bujard. (1992) PNAS. 89: 5547-5551.
Graham and van der Eb. (1973) Virology. 52: 456.
Griffin et al. (1995) Development. 121: 2983-94.
Grigoriadis et al. (1988) J Cell Biol. 106:2139-2151.
Guo et al. (1995) Molecular and Cellular Biology. 15: 3823-3829.
Hanahan. (1983) J. Mol. Biol. 166:557-580.
Hansen and Chilton (1999) Curr. Top. Microbiol. Immunol. 240: 21-57.
Hansen and Wright (1999) Trends Plant Sci. 4: 226-231.
Harlow and Lane. (1988) Antibodies: A laboratory manual. Cold Spring Harbor Laboratory Press, Cold Spring-Harbor. 726 pp.
Harlow and Lane. (1999) Using antibodies: A laboratory manual. Cold Spring Harbor Laboratory Press, Cold Spring Harbor, N.Y.
Hay and Fischman (1961) Developmental Biology. 3: 26-59.
Hill et al. (1989) Genes Dev. 3: 26-37.
Hinnen et al. (1978) PNAS 75: 1929-1933.
Hoffman. (1996) Plant Sci. 113:1-11.
Hoshimaru, et al. (1996) PNAS 93: 1518-1523.
Hu et al. (2001) Development 128: 2373-2384.
Ishiura et al. (1982) Molecular and Cellular Biology. 2: 607-616.
Ito et al. (1983), J. Bacteriol. 153: 163-168.
Iujvidin et al. (1990) Differentiation 43: 192-203.
Jaiswal et al. (1997) J Cell Biochem. 64: 295-312.
Johnson and Weston (1995) Genetics 141: 1583-95.
Kaufman, (1990) Methods Enzymol. 185: 487-511.
Kaufman, et al. (1986) PNAS 83: 3136-3140.
Kawai and Nishizawa. (1984) Mol. Cell. Biol. 4: 1172.
Kelly, et al. (1995) Mech. Dev. 53: 261-273.
Kelly and Hynes (1985) EMBO J. 4: 475-9.
Kettunen and Thesleff. (1998). Dev. Dyn. 211: 256-268.
Koshiba et al. (1998) J Exp Zool. 282: 70314.
Kozbor et al. (1984) J Immunol. 133: 3001-5.
Krauss et al. (1993) Cell. 75: 1431-44.
Kriegler. (1990) Gene transfer and expression: A laboratory manual. Stockton Press, New York. 242 pp.
Kumar et al. (2000) Developmental Biology. 218: 125-136.
Laforest et al. (1998) Development. 125: 4175-84.
Leduc et al. (1996) Developmental Biology. 10: 190-203.
Lemischka, et al. (1986) Cell. 45: 917-927.
Liao et al. (1997) Development. 124: 381-9.
Littlefield. (1964) Science. 145: 709-710.
Lo et al. (1993) PNAS 90: 7230-7234.
Lopata et al. (1984) Nucleic Acids Research. 12: 5707.
Luckow. (1991) Cloning and expression of heterologous genes in insect cells with baculovirus vectors. In Recombinant DNA technology and applications. A. Prokop, R. K. Bajpai, and C. Ho, editors. McGraw-Hill, New York. 97-152.
Mackay et al. (1998) Tissue Eng. 4: 415-28.
Mandel and Higa. (1970) J Mol Biol. 53: 159-162.
Martin. (1998) Genes Dev. 12:1571-86.
McGann et al. (2001) PNAS 98: 13699-13704.
Mehra-Chaudhary et al. (2001) Biochem J. 353: 13-22.
Miller and Buttimore. (1986) Mol Cell Biol. 6: 2895-2902.
Miller. (1988) Annu. Rev. Microbiol. 42:177-199.
Milstein and Cuello (1983) Nature. 305: 537-40.
Mohammadi et al. (1997) Science. 276: 955-60.
Moody. (1993) Growth factors, peptides, and receptors. Plenum Press, New York. 467
Morrison et al. (1987) Ann NY Acad Sci. 507: 187-98.
Munson and Rodbard. (1980) Anal Biochem. 107: 220-39.
U.S. Pat. No. 5,328,470
Neumann et al. (1982) EMBO J. 1: 841-845.
Novitch et al. (1996) J Cell Biol. 135:441-456.
Odelburg et al. (2000) Cell 103: 1099-1109.
Ohuchi et al. (1997) Development. 124: 2235-44.
O'Reilly et al. (1993) Baculovirus expression vectors. W.H. Freeman and Company, New York.
Ortega et al. (1998) PNAS. 95: 5672-7.
Ortega et al. (2002) Biochim Biophys Acta 1602: 73-87.
Ou-Lee, et al. (1986) PNAS 83: 6815-6819.
Palmer et al. (1987) PNAS. 84: 1055-1059.
Pear et al. (1993) PNAS 90: 8392-8396.
Peters and Balling (1999) Trends Genet. 15: 59-65.
Pittenger et al. (1999) Science. 284: 143-147.
Poss et al. (2000) Developmental Biology 222: 347-358.
Poss et al. (2000) Developmental Dynamics 219: 282-286.
Potter. (1988) Analytical Biochemistiy. 174: 361-373.
Potter et al. (1984) PNAS 81: 7161-7165.
Rassoulzadegan et al. (1982) Nature. 295: 257.
Reifers et al. (1998) Development. 125: 2381-95.
Rhodes et al. (1988) Science. 240: 204-207.
Robert et al. (1989) EMBO J. 8: 91100.
Rose et al. (1991) BioTechniques. 10: 520-525.
Sambrook, J. (1989) Molecular cloning: a laboratory manual. Cold Spring Harbor Laboratory, Cold Spring Harbor.
Sandri-Goldin et al. (1981) Mol. Cell. Biol. 1: 7453-752.
Santamaria and Becerra. (1991) J Anat. 176: 9-21.
Scaal et al. (2002) Mechanisms of Development 110: 51-60.
Schaffner. (1980) PNAS 77: 2163.
Schneider et al. (1994) Science. 264: 14671471.
Schook, L. B. 1987. Monoclonal antibody production techniques and applications. Marcel Dekker, Inc., New York. 336 pp.
Selden et al (1986) Molecular and Cellular Biology. 6: 3173-3179.
Shilo and Weinberg. (1981) PNAS 78: 6789-6792.
Simon et al. (1995) Dev Dyn. 202: 1-12.
Simonson et al. (1983)PNAS 80: 2495-2499.
Song et al. (1992) Nature 360: 477-481.
Southern and Berg (1982) J. Mol. Appl. Gen. 1: 327-341.
Sreekrishna et al. (1988) J. Basic Microbiol. 28: 265-78.
Stein and Cohen (1988) Cancer Res. 48: 2659-68.
Sun et al. (1999) J Med Chem. 42: 5120-30.
Tanaka et al. (1997) J Cell Biol. 136: 155-65.
Thisse et al. (1995) Dev Dyn. 203: 377-91.
Thompson-Jaeger (2000) Mol Cell Biochem 208: 63-69.
Thompson et al. (1995) Euphytica. 85: 75-80.
Tiainen et al. (1996) Mol Cell Biol. 16: 5302-5312.
Tilburn et al. (1983) Gene 26: 205-221.
Touraev et al. (1997) Plant J. 12: 949-956.
Trick et al. (1997) Tissue Cult. Biotechnol. 3: 9-26.
Turner et al. (1990) Neuron. 4: 833-845.
van der Krol et al. (1988) Biotechniques. 6: 958-76.
Vogel et al. (1995) Dev Biol. 171: 507-20.
Vogel et al. (1996) Development. 122: 1737-50.
Walsh and Perlnan. (1997) Curr Opin Genet Dev. 7: 597-602.
Wang and Sassoon. (1995) Dev Biol. 168: 374-82.
Wells et al. (1985) Gene. 34: 315-23.
Whitt et al. (1990) Focus. 13: 8-12.
Wigler et al. (1978) Cell. 14: 725.
Williams et al. (1984) Nature. 310: 476-480.
Woloshin et al. (1995) Cell. 82:611-620.
Wong and Neumann. (11982) Biochemical and Biophysical Research Communications. 107: 584-587.
Wyborski et al. (1996) Environ Mol Mutagen. 28: 447-58.
Wyborski and Short (1991) Nucleic Acids Res. 19: 4647-53.
Yelton et al. (1984) PNAS 81: 1470-4.
Zetter. (1998) Annu Rev Med. 49: 407-24.
Zhang et al. (2002) Development 129: 4135-4146.
Zhou et al. (1983) Methods Enzymol. 101: 433-481.
Zhu et al. (1996) Dev Dyn. 207: 42938.
Zoller and Smith. (1987) Methods Enzymol. 154: 329-50.
Zottoli et al. (1994) Prog Brain Res. 103: 219-28.

All publications, patents and patent applications are herein incorporated by reference in their entirety to the same extent as if each individual publication, patent or patent application was specifically and individually indicated to be incorporated by reference in its entirety.

### EQUIVALENTS

Those skilled in the art will recognize, or be able to ascertain using no more than routine experimentation, many equivalents to the specific embodiments of the invention described herein. Such equivalents are intended to be encompassed by the following claims.

SUPPLEMENTAL TABLE

| | nucleic acid sequence | amino acid sequence |
|---|---|---|
| Mouse Msx-1 | SEQ ID NO: 1 | SEQ ID NO: 2 |
| Rat Msx-1 | SEQ ID NO: 3 | SEQ ID NO: 4 |
| Human Msx-1 | SEQ ID NO: 5 | SEQ ID NO: 6 |
| Axolotl Msx-1 | SEQ ID NO: 7 | SEQ ID NO: 8 |
| Mouse Msx-2 | SEQ ID NO: 9 | SEQ ID NO: 10 |
| Rat Msx-2 | SEQ ID NO: 11 | SEQ ID NO: 12 |
| Human Msx-2 | SEQ ID NO: 13 | SEQ ID NO: 14 |
| Mouse Msx-3 | SEQ ID NO: 15 | SEQ ID NO: 16 |
| Mouse BMP-2 | SEQ ID NO: 17 | SEQ ID NO: 18 |
| Human BMP-2 | SEQ ID NO: 19 | SEQ ID NO: 20 |
| Mouse BMP-4 | SEQ ID NO: 21 | SEQ ID NO: 22 |
| Human BMP-4 | SEQ ID NO: 23 | SEQ ID NO: 24 |
| Mouse BMP-7 | SEQ ID NO: 25 | SEQ ID NO: 26 |
| Human BMP-7 | SEQ ID NO: 27 | SEQ ID NO: 28 |
| Human FGF-2 | SEQ ID NO: 29 | SEQ ID NO: 30 |
| Human FGF-4 | SEQ ID NO: 31 | SEQ ID NO: 32 |
| Human FGF-8 | SEQ ID NO: 33 | SEQ ID NO: 34 |
| Human FGF-10 | SEQ ID NO: 35 | SEQ ID NO: 36 |
| Human FGF-17 | SEQ ID NO: 37 | SEQ ID NO: 38 |
| Human FGF-18 | SEQ ID NO: 39 | SEQ ID NO: 40 |
| Human FGFR1 | SEQ ID NO: 41 | SEQ ID NO: 42 |
| Human FGFR2 | SEQ ID NO: 43 | SEQ ID NO: 44 |
| Human FGFR3 | SEQ ID NO: 45 | SEQ ID NO: 46 |
| Human FGFR4 | SEQ ID NO: 47 | SEQ ID NO: 48 |
| Human Wnt1 | SEQ ID NO: 49 | SEQ ID NO: 50 |
| Human Wnt2 | SEQ ID NO: 51 | SEQ ID NO: 52 |
| Human Wnt3 | SEQ ID NO: 53 | SEQ ID NO: 54 |
| Human Wnt5a | SEQ ID NO: 55 | SEQ ID NO: 56 |
| Human Wnt8 | SEQ ID NO: 57 | SEQ ID NO: 58 |
| Human Wnt11 | SEQ ID NO: 59 | SEQ ID NO: 60 |
| Human GSKβ | SEQ ID NO: 61 | SEQ ID NO: 62 |
| Human β-catenin | SEQ ID NO: 63 | SEQ ID NO: 64 |
| Human Lefl/Tcf | SEQ ID NO: 65 | SEQ ID NO: 66 |
| Human cyclinD1 | SEQ ID NO: 67 | SEQ ID NO: 68 |
| Human Cdk4 | SEQ ID NO: 69 | SEQ ID NO: 70 |
| Human p16 variant 2 | SEQ ID NO: 71 | SEQ ID NO: 72 |
| Human p16 variant 3 | SEQ ID NO: 73 | SEQ ID NO: 74 |
| Human p21 | SEQ ID NO: 75 | SEQ ID NO: 76 |
| Human p27 | SEQ ID NO: 77 | SEQ ID NO: 78 |

1. A method of dedifferentiating a differentiated mammalian cell, comprising administering an amount of one or more agents effective to promote dedifferentiation of a differentiated mammalian cell, wherein said agent has a function selected from at least one of:
(a) increases the expression and/or activity of a G₁ Cdk complex,
(b) decreases expression of one or more markers of differentiation,
(c) promotes cell cycle reentry, or
(d) increases the expression of one or more progenitor or stem cell markers.

2. The method of claim 1, wherein said dedifferentiation occurs in vivo.

3. The method of claim 2, wherein said dedifferentiation occurs in vivo at a site of injury or cell damage.

4. The method of claim 3, wherein said injury or cell damage is caused by disease or trauma.

5. The method of claim 1, wherein administration of said one or more agents comprises systemic administration.

6. The method of claim 1, wherein administration of said one or more agents comprises local administration at a site of injury or cell damage.

7. The method of claim 1, wherein administration of said one or more agents comprises implanting a delivery device.

8-9. (canceled)

10. The method of claim 1, wherein said differentiated mammalian cell is a terminally differentiated mammalian cell.

11. The method of claim 1, wherein said differentiated mammalian cell is selected from the group consisting of a skeletal muscle cell, a cardiac muscle cell, a smooth muscle cell, a skin cell, a chondrocyte, an adipocyte, or an osteocyte.

12. The method of claim 1, wherein said differentiated mammalian cell is selected from the group consisting of a cell of connective tissue, a neuronal cell, a lymphatic cell, a cell of vasculature, a cell of kidney, a cell of pancreas, a cell of lung, a cell of urethra, a cell of bladder, a cell of stomach, a cell of liver, a cell of small intestine, a cell of large intestine, or a cell of esophagus.

13-14. (canceled)

15. The method of claim 1, wherein said one or more agents is independently selected from the group consisting of an agent that promotes FGF signaling, an agent that promotes BMP signaling, an agent that promotes Wnt signaling, an agent that promotes expression and/or activity of msx1, an agent that promotes expression and/or activity of msx2, an agent that inhibits expression and/or activity of msx3, an agent that promotes expression and/or activity of cyclinD1, an agent that promotes expression and/or activity of Cdk4, an agent that promotes expression and/or activity of cdc25, an agent that inhibits expression and/or activity of p16, an agent that inhibits expression and/or activity of p21, an agent that inhibits expression and/or activity of p27, an agent that inhibits expression and/or activity of Rb, and an agent that inhibits expression and/or activity of Wee1.

16-18. (canceled)

19. The method of claim 15, wherein said one or more agents promotes the expression and/or activity of msx1, and wherein said one or more agents is selected from the group consisting of a nucleic acid comprising a nucleotide sequence that encodes an msx1 polypeptide, a polypeptide comprising an amino acid sequence of an msx1 polypeptide, a small organic molecule that promotes the expression and/or activity of msx 1.

20-25. (canceled)

26. A method of regenerating mammalian tissues and/or organs, comprising contacting differentiated mammalian cells with an amount of an agent effective to dedifferentiate said differentiated mammalian cells, wherein said agent is capable of inducing dedifferentiation, and wherein following dedifferentiation the mammalian cells are capable of redifferentiating to regenerate said mammalian tissues and/or organs.

27. The method of claim 26, wherein dedifferentiation occurs in vivo.

28. The method of claim 27, wherein dedifferentiation occurs in vivo at a site of injury or cell damage.

29. The method of claim 28, wherein said injury or cell damage is caused by disease or trauma.

30. The method of claim 26, wherein administration of said one or more agents comprises systemic administration.

31. The method of claim 26, wherein administration of said one or more agents comprises local administration at a site of injury or cell damage.

32-36. (canceled)

37. The method of claim 26, wherein said differentiated mammalian cell is a terminally differentiated mammalian cell.

38. The method of claim 26, wherein said differentiated mammalian cell is selected from the group consisting of a skeletal muscle cell, a cardiac muscle cell, a smooth muscle cell, a skin cell, a chondrocyte, an adipocyte, or an osteocyte.

39-41. (canceled)

42. A method of screening to identify and/or characterize a dedifferentiation agent, wherein said dedifferentiation agent promotes dedifferentiation of one or more cell types, comprising
(a) contacting a cell with one or more agents;
(b) comparing dedifferentiation of said cell in the presence of said one or more agents in comparison to the absence of said one or more agents,
wherein an agent that promotes dedifferentiation of a cell is a dedifferentiation agent.

43-62. (canceled)

63. A packaged pharmaceutical comprising: a preparation of expression constructs encoding a protein or transcript which upregulates the activity of a G1 phase cyclin dependent kinase (cdk); a pharmaceutically acceptable carrier; and instructions, written and/or pictorial, describing the use of the preparation for causing dedifferentiation of cells in a patient.

64. A method of promoting regeneration of a mammalian tissue, comprising
dedifferentiating differentiated mammalian cells of said mammalian tissue by contacting said differentiated mammalian cells with an effective amount of a composition capable of inducing dedifferentiation, and
culturing said dedifferentiated mammalian cells for a time sufficient to promote proliferation and redifferentiation of said dedifferentiated mammalian cells, thereby promoting regeneration of said mammalian tissue.
